SEQUENCE LISTING (1 sequence)

SEQ ID NO: 1 - protein, 24 aa, Artificial
  Biotinylated peptide substrate
XKKANQVFLGFTYVAPSVLESVKX

(19) United States
(12) Patent Application Publication
(10) Pub. No.: US 2011/0034454 A1
(43) Pub. Date: Feb. 10, 2011

(54) MORPHOLINO PYRIMIDINE DERIVATIVES AND THEIR USE IN THERAPY

(76) Inventors: Allan Paul Dishington, Macclesfield (GB); Shaun Michael Fillery, Macclesfield (GB); Maurice Raymond Verschoyle Finlay, Macclesfield (GB); Kurt Gordon Pike, Macclesfield (GB)

Correspondence Address: ASTRAZENECA R&D BOSTON, 35 GATEHOUSE DRIVE, WALTHAM, MA, 02451-1215, US

(21) Appl. No.: 12/160,752

(22) PCT Filed: Jan. 8, 2007
(86) PCT No.: PCT/GB07/00037

§ 371 Date: Jul. 11, 2008

(30) Foreign Application Priority Data
Jan. 11, 2006 (GB) 0600483.2
Aug. 24, 2006 (GB) 0616747.2

(51) Int. Cl. A61K 31/5377; C07D 413/14; C07D 413/04; A61P 35/00; A61P 29/00; A61P 37/00; A61P 9/00; A61P 11/00

(52) U.S. Cl. 514/232.5; 544/122; 514/235.8; 544/120; 514/235.2; 514/233.8

(57) ABSTRACT

A compound of formula (I) or a salt, ester or prodrug thereof, processes for their preparation, pharmaceutical compositions containing them and their use in therapy, for example in the treatment of proliferative disease such as cancer and particularly in disease mediated by an mTOR kinase and/or one or more PI3K enzyme.

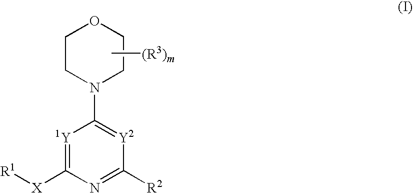

The present invention relates to morpholino pyrimidine derivatives, processes for their preparation, pharmaceutical compositions containing them and their use in therapy, for example in the treatment of proliferative disease such as cancer and particularly in disease mediated by an mTOR kinase and/or one or more PI3K enzyme.

It is now well understood that deregulation of oncogenes and tumour-suppressor genes contributes to the formation of malignant tumours, for example by way of increased cell proliferation or increased cell survival. It is also known that signalling pathways mediated by the PI3K/mTOR families have a central role in a number of cell processes including proliferation and survival, and deregulation of these pathways is a causative factor in a wide spectrum of human cancers and other diseases.

The mammalian target of the macrolide antibiotic Rapamycin (sirolimus) is the enzyme mTOR. This enzymes belongs to the phosphatidylinositol (PI) kinase-related kinase (PIKK) family of protein kinases, which also includes ATM, ATR, DNA-PK and hSMG-1. mTOR, like other PIKK family members, does not possess detectable lipid kinase activity, but instead functions as a serine/threonine kinase. Much of the knowledge of mTOR signalling is based upon the use of Rapamycin. Rapamycin first binds to the 12 kDa immunophilin FK506-binding protein (FKBP12) and this complex inhibits mTOR signalling (Tee and Blenis, Seminars in Cell and Developmental Biology, 2005, 16, 29-37). The mTOR protein consists of a catalytic kinase domain, an FKBP12-Rapamycin binding (FRB) domain, a putative repressor domain near the C-terminus and up to 20 tandemly-repeated HEAT motifs at the N-terminus, as well as FRAP-ATM-TRRAP (FAT) and FAT C-terminus domain (Huang and Houghton, Current Opinion in Pharmacology, 2003, 3, 371-377).

mTOR kinase is a key regulator of cell growth and has been shown to regulate a wide range of cellular functions including translation, transcription, mRNA turnover, protein stability, actin cytoskeleton reorganisation and autophagy (Jacinto and Hall, Nature Reviews Molecular and Cell Biology, 2005, 4, 117-126). mTOR kinase integrates signals from growth factors (such as insulin or insulin-like growth factor) and nutrients (such as amino acids and glucose) to regulate cell growth. mTOR kinase is activated by growth factors through the PI3K-Akt pathway. The most well characterised function of mTOR kinase in mammalian cells is regulation of translation through two pathways, namely activation of ribosomal S6K1 to enhance translation of mRNAs that bear a 5′-terminal oligopyrimidine tract (TOP) and suppression of 4E-BP1 to allow CAP-dependent mRNA translation.

Generally, investigators have explored the physiological and pathological roles of mTOR using inhibition with Rapamycin and related Rapamycin analogues based on their specificity for mTOR as an intracellular target. However, recent data suggests that Rapamycin displays variable inhibitory actions on mTOR signalling functions and suggest that direct inhibition of the mTOR kinase domain may display substantially broader anti-cancer activities than that achieved by Rapamycin (Edinger et al., Cancer Research, 2003, 63, 8451-8460). For this reason, potent and selective inhibitors of mTOR kinase activity would be useful to allow a more complete understanding of mTOR kinase function and to provide useful therapeutic agents.

There is now considerable evidence indicating that the pathways upstream of mTOR, such as the PI3K pathway, are frequently activated in cancer (Vivanco and Sawyers, Nature Reviews Cancer, 2002, 2, 489-501; Bjornsti and Houghton, Nature Reviews Cancer, 2004, 4, 335-348; Inoki et al., Nature Genetics, 2005, 37, 19-24). For example, components of the PI3K pathway that are mutated in different human tumours include activating mutations of growth factor receptors and the amplification and/or overexpression of PI3K and Akt.

In addition there is evidence that endothelial cell proliferation may also be dependent upon mTOR signalling. Endothelial cell proliferation is stimulated by vascular endothelial cell growth factor (VEGF) activation of the PI3K-Akt-mTOR signalling pathway (Dancey, Expert Opinion on Investigational Drugs, 2005, 14, 313-328). Moreover, mTOR kinase signalling is believed to partially control VEGF synthesis through effects on the expression of hypoxia-inducible factor-1α (HIF-1α) (Hudson et al., Molecular and Cellular Biology, 2002, 22, 7004-7014). Therefore, tumour angiogenesis may depend on mTOR kinase signalling in two ways, through hypoxia-induced synthesis of VEGF by tumour and stromal cells, and through VEGF stimulation of endothelial proliferation and survival through PI3K-Akt-mTOR signalling.

These findings suggest that pharmacological inhibitors of mTOR kinase should be of therapeutic value for treatment of the various forms of cancer comprising solid tumours such as carcinomas and sarcomas and the leukaemias and lymphoid malignancies. In particular, inhibitors of mTOR kinase should be of therapeutic value for treatment of, for example, cancer of the breast, colorectum, lung (including small cell lung cancer, non-small cell lung cancer and bronchioalveolar cancer) and prostate, and of cancer of the bile duct, bone, bladder, head and neck, kidney, liver, gastrointestinal tissue, oesophagus, ovary, pancreas, skin, testes, thyroid, uterus, cervix and vulva, and of leukaemias (including ALL and CML), multiple myeloma and lymphomas.

In addition to tumourigenesis, there is evidence that mTOR kinase plays a role in an array of hamartoma syndromes. Recent studies have shown that the tumour suppressor proteins such as TSC1, TSC2, PTEN and LKB1 tightly control mTOR kinase signalling.

Loss of these tumour suppressor proteins leads to a range of hamartoma conditions as a result of elevated mTOR kinase signalling (Tee and Blenis, Seminars in Cell and Developmental Biology, 2005, 16, 29-37). Syndromes with an established molecular link to dysregulation of mTOR kinase include Peutz-Jeghers syndrome (PJS), Cowden disease, Bannayan-Riley-Ruvalcaba syndrome (BRRS), Proteus syndrome, Lhermitte-Duclos disease and Tuberous Sclerosis (TSC) (Inoki et al., Nature Genetics, 2005, 37, 19-24). Patients with these syndromes characteristically develop benign hamartomatous tumours in multiple organs.

Recent studies have revealed a role for mTOR kinase in other diseases (Easton & Houghton, Expert Opinion on Therapeutic Targets, 2004, 8, 551-564). Rapamycin has been demonstrated to be a potent immunosuppressant by inhibiting antigen-induced proliferation of T cells, B cells and antibody production (Sehgal, Transplantation Proceedings, 2003, 35,

7S-14S) and thus mTOR kinase inhibitors may also be useful immunosuppressives. Inhibition of the kinase activity of mTOR may also be useful in the prevention of restenosis, that is the control of undesired proliferation of normal cells in the vasculature in response to the introduction of stents in the treatment of vasculature disease (Morice et al., New England Journal of Medicine, 2002, 346, 1773-1780). Furthermore, the Rapamycin analogue, everolimus, can reduce the severity and incidence of cardiac allograft vasculopathy
(Eisen et al., New England Journal of Medicine, 2003, 349, 847-858). Elevated mTOR kinase activity has been associated with cardiac hypertrophy, which is of clinical importance as a major risk factor for heart failure and is a consequence of increased cellular size of cardiomyocytes (Tee & Blenis, Seminars in Cell and Developmental Biology, 2005, 16,
29-37). Thus mTOR kinase inhibitors are expected to be of value in the prevention and treatment of a wide variety of diseases in addition to cancer.

It is also believed that a number of these morpholino pyrimidine derivatives may have inhibitory activity against the phosphatidylinositol (PI) 3-kinases family of kinases.

Phosphatidylinositol (PI) 3-kinases (PI3Ks) are ubiquitous lipid kinases that function both as signal transducers downstream of cell-surface receptors and in constitutive intracellular membrane and protein trafficking pathways. All PI3Ks are dual-specificity enzymes with a lipid kinase activity that phosphorylates phosphoinositides at the 3-hydroxy position, and a less well characterised protein kinase activity. The lipid products of PI3K-catalysed reactions comprising phosphatidylinositol 3,4,5-trisphosphate [PI(3,4,5)P₃], phosphatidylinositol 3,4-bisphosphate [PI(3,4)P₂] and phosphatidylinositol 3-monophosphate [PI(3)P] constitute second messengers in a variety of signal transduction pathways, including those essential to cell proliferation, adhesion, survival, cytoskeletal rearrangement and vesicle trafficking. PI(3)P is constitutively present in all cells and its levels do not change dramatically following agonist stimulation. Conversely, PI(3,4)P₂ and PI(3,4,5)P₃ are nominally absent in most cells but they rapidly accumulate on agonist stimulation.

The downstream effects of PI3K-produced 3-phosphoinositide second messengers are mediated by target molecules containing 3-phosphoinositide binding domains such as the pleckstrin homology (PH) domain and the recently identified FYVE and phox domains. Well-characterised protein targets for PI3K include PDK1 and protein kinase B (PKB). In addition, tyrosine kinases like Btk and Itk are dependent on PI3K activity.

The PI3K family of lipid kinases can be classified into three groups according to their physiological substrate specificity (Vanhaesebroeck et al., Trends in Biol. Sci., 1997, 22, 267). Class III PI3K enzymes phosphorylate PI alone. In contrast, Class II PI3K enzymes phosphorylate both PI and PI 4-phosphate [PI(4)P]. Class I PI3K enzymes phosphorylate PI, PI(4)P and PI 4,5-bisphosphate [PI(4,5)P₂], although only PI(4,5)P₂ is believed to be the physiological cellular substrate. Phosphorylation of PI(4,5)P₂ produces the lipid second messenger PI(3,4,5)P₃. More distantly related members of the lipid kinase superfamily are Class IV kinases such as mTOR (discussed above) and DNA-dependent kinase that phosphorylate serine/threonine residues within protein substrates. The most studied and understood of the PI3K lipid kinases are the Class I PI3K enzymes.

Class I PI3Ks are heterodimers consisting of a p110 catalytic subunit and a regulatory subunit. The family is further divided into Class Ia and Class Ib enzymes on the basis of regulatory partners and the mechanism of regulation. Class Ia enzymes consist of three distinct catalytic subunits (p110α, p110β and p110δ) that dimerise with five distinct regulatory subunits (p85α, p55α, p50α, p85β and p55γ), with all catalytic subunits being able to interact with all regulatory subunits to form a variety of heterodimers. Class Ia PI3Ks are generally activated in response to growth factor-stimulation of receptor tyrosine kinases via interaction of their regulatory subunit SH2 domains with specific phospho-tyrosine residues of activated receptor or adaptor proteins such as IRS-1. Both p110α and p110β are constitutively expressed in all cell types, whereas p110δ expression is more restricted to leukocyte populations and some epithelial cells. In contrast, the single Class Ib enzyme consists of a p110γ catalytic subunit that interacts with a p101 regulatory subunit. Furthermore, the Class Ib enzyme is activated in response to G-protein coupled receptor systems (GPCRs) and its expression appears to be limited to leukocytes and cardiomyocytes.

There is now considerable evidence indicating that Class Ia PI3K enzymes contribute to tumourigenesis in a wide variety of human cancers, either directly or indirectly (Vivanco and Sawyers, Nature Reviews Cancer, 2002, 2, 489-501). For example, the p110α subunit is amplified in some tumours such as those of the ovary (Shayesteh et al., Nature Genetics, 1999, 21, 99-102) and cervix (Ma et al., Oncogene, 2000, 19, 2739-2744). More recently, activating mutations within the catalytic site of the p110α catalytic subunit have been associated with various other tumours such as those of the colorectal region and of the breast and lung (Samuels et al., Science, 2004, 304, 554). Tumour-related mutations in the p85α regulatory subunit have also been identified in cancers such as those of the ovary and colon (Philp et al., Cancer Research, 2001, 61, 7426-7429). In addition to direct effects, it is believed that activation of Class Ia PI3Ks contributes to tumourigenic events that occur upstream in signalling pathways, for example by way of ligand-dependent or ligand-independent activation of receptor tyrosine kinases, GPCR systems or integrins (Vara et al., Cancer Treatment Reviews, 2004, 30, 193-204). Examples of such upstream signalling pathways include over-expression of the receptor tyrosine kinase erbB2 in a variety of tumours leading to activation of PI3K-mediated pathways (Harari et al., Oncogene, 2000, 19, 6102-6114) and over-expression of the ras oncogene (Kauffmann-Zeh et al., Nature, 1997, 385, 544-548). In addition, Class Ia PI3Ks may contribute indirectly to tumourigenesis caused by various downstream signalling events. For example, loss of the effect of the PTEN tumour-suppressor phosphatase that catalyses conversion of PI(3,4,5)P₃ back to PI(4,5)P₂ is associated with a very broad range of tumours via deregulation of PI3K-mediated production of PI(3,4,5)P₃ (Simpson and Parsons, Exp. Cell Res., 2001, 264, 29-41). Furthermore, augmentation of the effects of other PI3K-mediated signalling events is believed to contribute to a variety of cancers, for example by activation of Akt (Nicholson and Anderson, Cellular Signalling, 2002, 14, 381-395).

In addition to a role in mediating proliferative and survival signalling in tumour is cells, there is evidence that Class Ia PI3K enzymes contribute to tumourigenesis in tumour-associated stromal cells. For example, PI3K signalling is known to play an important role in mediating angiogenic events in endothelial cells in response to pro-angiogenic factors such as VEGF (Abid et al., Arterioscler. Thromb. Vasc. Biol., 2004, 24, 294-300). As Class I PI3K enzymes are also involved in motility and migration (Sawyer, Expert Opinion Investig. Drugs, 2004, 13, 1-19), PI3K enzyme inhibitors should provide therapeutic benefit via inhibition of tumour cell invasion and metastasis. In addition, Class I PI3K enzymes play an important role in the regulation of immune cells contributing to pro-tumourigenic effects of inflammatory cells (Coussens and Werb, Nature, 2002, 420, 860-867).

These findings suggest that pharmacological inhibitors of Class I PI3K enzymes will be of therapeutic value for the treatment of various diseases including different forms of the disease of cancer comprising solid tumours such as carcinomas and sarcomas and the leukaemias and lymphoid malignancies. In particular, inhibitors of Class I PI3K enzymes should be of therapeutic value for treatment of, for example, cancer of the breast, colorectum, lung (including small cell lung cancer, non-small cell lung cancer and bronchioalveolar cancer) and prostate, and of cancer of the bile duct, bone, bladder, head and neck, kidney, liver, gastrointestinal tissue, oesophagus, ovary, pancreas, skin, testes, thyroid, uterus, cervix and vulva, and of leukaemias (including ALL and CML), multiple myeloma and lymphomas.

PI3Kγ, the Class Ib PI3K, is activated by GPCRs, as was finally demonstrated in mice lacking the enzyme. Thus, neutrophils and macrophages derived from PI3Kγ-deficient animals failed to produce PI(3,4,5)P₃ in response to stimulation with various chemotactic substances (such as IL-8, C5a, fMLP and MIP-1a), whereas signalling through protein tyrosine kinase-coupled receptors to Class Ia PI3Ks was intact (Hirsch et al., Science, 2000, 287(5455), 1049-1053; Li et al., Science, 2002, 287(5455), 1046-1049; Sasaki et al., Science 2002, 287(5455), 1040-1046). Furthermore, PI(3,4,5)P₃-mediated phosphorylation of PKB was not initiated by these GPCR ligands in PI3Kγ-null cells. Taken together, the results demonstrated that, at least in resting haematopoietic cells, PI3Kγ is the sole PI3K isoform that is activated by GPCRs in vivo. When murine bone marrow-derived neutrophils and peritoneal macrophages from wild-type and PI3Kγ^{-/-} mice were tested in vitro, a reduced, but not completely abrogated, performance in chemotaxis and adherence assays was observed. However, this translated into a drastic impairment of IL-8 driven neutrophil infiltration into tissues (Hirsch et al., Science, 2000, 287(5455), 1049-1053.). Recent data suggest that PI3Kγ is involved in the path finding process rather than in the generation of mechanical force for motility, as random migration was not impaired in cells that lacked PI3Kγ (Hannigan et al., Proc. Nat. Acad. of Sciences of U.S.A., 2002, 99(6), 3603-8). Data linking PI3Kγ to respiratory disease pathology came with the demonstration that PI3Kγ has a central role in regulating endotoxin-induced lung infiltration and activation of neutrophils leading to acute lung injury (Yum et al., J. Immunology, 2001, 167(11), 6601-8). The fact that although PI3Kγ is highly expressed in leucocytes, its loss seems not to interfere with haematopoiesis, and the fact that PI3Kγ-null mice are viable and fertile further implicates this PI3K isoform as a potential drug target. Work with knockout mice also established that PI3Kγ is an essential amplifier of mast cell activation (Laffargue et al., Immunity, 2002, 16(3), 441-451).

Thus, in addition to tumourigenesis, there is evidence that Class I PI3K enzymes play a role in other diseases (Wymann et al., Trends in Pharmacological Science, 2003, 24, 366-376). Both Class Ia PI3K enzymes and the single Class Ib enzyme have important roles in cells of the immune system (Koyasu, Nature Immunology, 2003, 4, 313-319) and thus they are therapeutic targets for inflammatory and allergic indications. Recent reports demonstrate that mice deficient in PI3Kγ and PI3Kδ are viable, but have attenuated inflammatory and allergic responses (Ali et al., Nature, 2004, 431(7011), 1007-11). Inhibition of PI3K is also useful to treat cardiovascular disease via anti-inflammatory effects or directly by affecting cardiac myocytes (Prasad et al., Trends in Cardiovascular Medicine, 2003, 13, 206-212). Thus, inhibitors of Class I PI3K enzymes are expected to be of value in the prevention and treatment of a wide variety of diseases in addition to cancer.

Several compounds that inhibit PI3Ks and phosphatidylinositol (PI) kinase-related kinase (PI3KKs) have been identified, including wortmannin and the quercetin derivative LY294002. These compounds are reasonably specific inhibitors of PI3Ks and PI3KKs over other kinases but they lack potency and display little selectivity within the PI3K families.

Accordingly, it would be desirable to provide further effective mTOR and/or PI3K inhibitors for use in the treatment of cancer, inflammatory or obstructive airways diseases, immune or cardiovascular diseases.

Morpholino pyrimidine derivatives and PI3K inhibitors are known in the art.

International Patent Application WO 2004/048365 discloses compounds that possess PI3K enzyme inhibitory activity and are useful in the treatment of cancer. These compounds are arylamino- and heteroarylamino-substituted pyrimidines which differ from the compounds of the present invention with respect to their arylamino- and heteroarylamino substituents. These substituents are not equivalent to the —XR¹ substituents of the present invention. Inhibitors of PI3K activity useful in the treatment of cancer are also disclosed in European Patent Application 1 277 738 which mentions 4-morpholino-substituted bicyclic heteroaryl compounds such as quinazoline and pyrido[3,2-d]pyrimidine derivatives and 4-morpholino-substituted tricyclic heteroaryl compounds but not monocyclic pyrimidine derivatives.

A number of compounds such as 4-morpholin-4-yl-6-(phenylsulfonylmethyl)-2-pyridin-4-yl-pyrimidine and 4-{6-[(phenylsulfonyl)methyl]-2-pyridin-2-ylpyrimidin-4-yl}morpholine have been registered in the Chemical Abstracts database but no utility has been indicated and there is no suggestion that these compounds have mTOR and/or PI3K inhibitory activity or useful therapeutic properties.

Surprisingly, we have found that certain morpholino pyrimidine derivatives, including some previously known compounds possess useful therapeutic properties. Without wishing to be bound by theoretical constraints, it is believed that the therapeutic usefulness of the derivatives is derived from their inhibitory activity against mTOR kinase and/or one or more PI3K enzyme (such as the Class Ia enzyme and/or the Class Ib enzyme). Because signalling pathways mediated by the PI3K/mTOR families have a central role in a number of cell processes including proliferation and survival, and because deregulation of these pathways is a causative factor in a wide spectrum of human cancers and other diseases, it is expected that the derivatives will be therapeutically useful. In particular, it is expected that the derivatives will have anti-proliferative and/or apoptotic properties which means that they will be useful in the treatement of proliferative disease such as cancer. The compounds of the present invention may also be useful in inhibiting the uncontrolled cellular proliferation which arises from various non-malignant diseases such as inflammatory diseases, obstructive airways diseases, immune diseases or cardiovascular diseases.

Generally, the compounds of the present invention possess potent inhibitory activity against mTOR kinase but the compound may also possess potent inhibitory activity against one or more PI3K enzyme (such as the Class Ia enzyme and/or the Class Ib enzyme).

In accordance with one aspect of the present invention, there is provided a compound of formula (I)

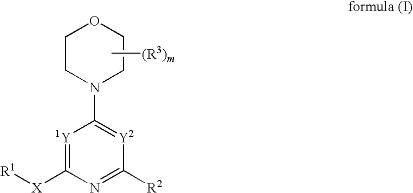

or a salt, ester or prodrug thereof; wherein
m is 0, 1, 2, 3 or 4;
X is a linker group selected from —CR⁴═CR⁵—, —CR⁴═CR⁵CR⁶R⁷—, —CR⁶R⁷CR⁵═CR⁴—, —C≡C—, —C≡CCR⁶R⁷—, —CR⁶R⁷C≡C—, —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷—, —NR⁴S(O)₂CR⁶R⁷—, —S(O)₂NR⁴CR⁶R⁷—, —C(O)NR⁴—, —NR⁴C(O)—, —NR⁴C(O)NR⁵—, —S(O)₂NR⁴— and —NR⁴S(O)₂—;
¹Y and Y² are independently N or CR⁸ provided that one of ¹Y and Y² is N and the other is CR⁸;
R¹ is a group selected from C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —SR⁹, —SOR⁹, —SO₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹⁵, —NR⁹COCONR¹⁰R¹⁵ and —NR⁹SO₂R¹⁰;
R² is a group selected from C₁₋₆alkyl, carbocyclyl and heterocyclyl which group is optionally substituted by one or more substituent group independently selected from halo, cyano, nitro,
—R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², and —NR¹¹COCONR¹²R¹⁶;
each R³, when present, is independently selected from halo, cyano, nitro, —R¹³, —OR¹³, —SR¹³, —SOR¹³, —SO₂R¹³, —COR¹³, —CO₂R¹³, —CONR¹³R¹⁴, —NR¹³COR¹⁴, —NR¹³COR¹⁴, —NR¹³CO₂R¹⁴ and —NR¹³SO₂R¹⁴;
R⁴ and R⁵ are independently hydrogen or C₁₋₆alkyl;
or R¹ and R⁴ together with the atom or atoms to which they are attached form a 5- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ are independently selected from hydrogen, halo, cyano, nitro and C₁₋₆alkyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹ and R¹² are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵ and R¹⁶ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl; provided that when X is —C(O)NH—, R¹ is not the group

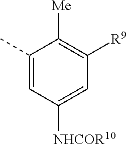

for use as a medicament in the treatment of proliferative disease.

In accordance with one aspect of the present invention, there is provided a compound of formula (I)

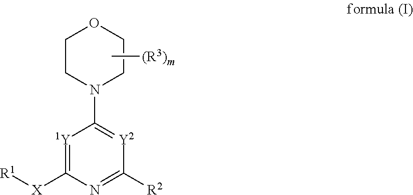

or a salt, ester or prodrug thereof; wherein
m is 0, 1, 2, 3 or 4;
X is a linker group selected from —CR⁴═CR⁵—, —CR⁴═CR⁵CR⁶R⁷—, —CR⁶R⁷CR⁵═CR⁴—, —C≡C—, —C≡CR⁶R⁷—, —CR⁶R⁷C≡C—, —NR⁴CR⁶R⁷—, OCR⁶R⁷—, SCR⁶R⁷—, —S(O)CR⁶R⁷—, —S(O)₂CR⁶R⁷—, —C(O)NR⁴CR⁶R⁷—, —NR⁴C(O)NR⁵CR⁶R⁷—, —S(O)₂NR⁴CR⁶R⁷—, —C(O)NR⁴—, —NR⁴C(O)—, —NR⁴C(O)NR⁵—, —S(O)₂NR⁴— and —NR⁴S(O)₂—;
¹Y and Y² are independently N or CR⁸ provided that one of ¹Y and Y² is N and the other is CR⁸;
R¹ is a group selected from C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —SR⁹, —SOR⁹, —SO₂R⁹, —COR⁹, —CO₂R⁹, —CONR⁹R¹⁰, —NR⁹R¹⁰, —NR⁹COR¹⁰, NR⁹CO₂R¹⁰, —NR⁹CONR¹⁰R¹⁵, —NR⁹COCONR¹⁰R¹⁵ and —NR⁹SO₂R¹⁰;
R² is a group selected from C₁₋₆alkyl, carbocyclyl and heterocyclyl which group is optionally substituted by one or more substituent group independently selected from halo, cyano, nitro,
—R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², and —NR¹¹COCONR¹²R¹⁶;
each R³, when present, is independently selected from halo, cyano, nitro, —R¹³, —OR¹³, —SR¹³, —SOR¹³, —SO₂R¹³, —COR¹³, —CO₂R¹³, —CONR¹³R¹⁴, —NR¹³R¹⁴, —NR¹³COR¹⁴, —NR¹³CO₂R¹⁴ and —NR¹³SO₂R¹⁴;
R⁴ and R⁵ are independently hydrogen or C₁₋₆alkyl;
or R¹ and R⁴ together with the atom or atoms to which they are attached form a 5- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ are independently selected from hydrogen, halo, cyano, nitro and C₁₋₆alkyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹ and R¹² are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵ and R¹⁶ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
provided that when X is —C(O)NH—, R¹ is not the group

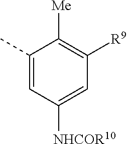

for use as a medicament in the treatment of proliferative disease.

In accordance with one aspect of the present invention, there is provided a compound of formula (I)

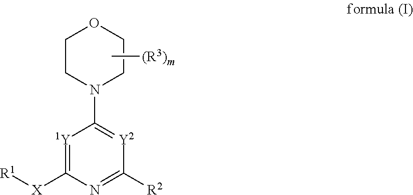

or a salt, ester or prodrug thereof; wherein
m is 0, 1, 2, 3 or 4;
X is a linker group selected from —CR⁴═CR⁵—, —CR⁴═CR⁵CR⁶R⁷—, —CR⁶R⁷CR⁵═CR⁴—, —C≡C—, —C≡CCR⁶R⁷—, —CR⁶R⁷C≡C—, —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)NR⁵CR⁶R⁷—, —S(O)₂NR⁴CR⁶R⁷—, —C(O)NR⁴—, —NR⁴C(O)—, —NR⁴C(O)NR⁵—, —S(O)₂NR⁴— and —NR⁴S(O)₂—;
¹Y and Y² are independently N or CR⁸ provided that one of ¹Y and Y² is N and the other is CR⁸;
R¹ is a group selected from C₁₋₆alkyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —COR⁹, —CONR⁹R¹⁰, —NR⁹R¹⁰ and —NR⁹COR¹⁰;
R² is a group selected from C₁₋₆alkyl, carbocyclyl and heterocyclyl which group is optionally substituted by one or more substituent group independently selected from halo, cyano, nitro,

### —R¹¹, —OR¹¹, —COR¹¹, —CONR¹¹R¹², —NR¹¹R¹² and —NR¹¹COR¹²;

each R³, when present, is independently selected from halo, cyano, nitro, —R¹³, —OR¹³, —COR¹³, —CONR¹³R¹⁴, NR¹³R¹⁴ and —NR¹³COR¹⁴;
R⁴ and R⁵ are independently hydrogen or C₁₋₆alkyl;
R⁶ and R⁷ are independently selected from hydrogen, halo, cyano, nitro and C₁₋₆alkyl;
R⁸ is selected from hydrogen, halo, cyano and C₁₋₆alkyl;
R⁹ and R¹⁰ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹¹ and R¹² are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino,
C₁₋₆alkylamino and bis(C₁₋₆alkyl)amino;
R¹³ and R¹⁴ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino,
C₁₋₆alkylamino and bis(C₁₋₆alkyl)amino;
provided that when X is —C(O)NH—, R¹ is not the group

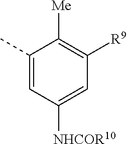

for use as a medicament in the treatment of proliferative disease.

In accordance with another aspect of the present invention, there is provided the use of a compound of formula (I)

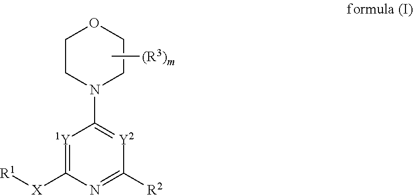

or a salt, ester or prodrug thereof; wherein
m is 0, 1, 2, 3 or 4;
X is a linker group selected from —CR⁴═CR⁵—, —CR⁴═CR⁵CR⁶R⁷—, —CR⁶R⁷CR⁵═CR⁴—, —C≡C—, —C≡CCR⁶R⁷—, —CR⁶R⁷C≡C—, —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷—, —NR⁴S(O)₂CR⁶R⁷—, —S(O)₂NR⁴CR⁶R⁷—, —C(O)NR⁴—, —NR⁴C(O)—, —NR⁴C(O)NR⁵—, —S(O)₂NR⁴— and —NR⁴S(O)₂—;
¹Y and Y² are independently N or CR⁸ provided that one of ¹Y and Y² is N and the other is CR⁸;
R¹ is a group selected from C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, —R⁹, —OR⁹, —SR⁹, —SOR⁹, —SO₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹⁵, —NR⁹COCONR¹⁰R¹⁵ and —NR⁹SO₂R¹⁰;
R² is a group selected from C₁₋₆alkyl, carbocyclyl and heterocyclyl which group is optionally substituted by one or more substituent group independently selected from halo, cyano, nitro,
—R¹¹, —OR¹¹, —SR¹¹, —SOR¹¹, —SO₂R¹¹, —COR¹¹, —CO₂R¹¹, —CONR¹¹R¹², —NR¹¹R¹², NR¹¹COR¹², and —NR¹¹COCONR¹²R¹⁶;
each R³, when present, is independently selected from halo, cyano, nitro, —R¹³, —OR¹³, —SR¹³, —SOR¹³, —SO₂R¹³, —COR¹³, —CO₂R¹³, —CONR¹³R¹⁴, —NR¹³R¹⁴, —NR¹³COR¹⁴, —NR¹³CO₂R¹⁴ and —NR¹³SO₂R¹⁴;
R⁴ and R⁵ are independently hydrogen or C₁₋₆alkyl;
or R¹ and R⁴ together with the atom or atoms to which they are attached form a 5- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ are independently selected from hydrogen, halo, cyano, nitro and C₁₋₆alkyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹ and R¹² are independently hydrogen or a group selected from C₁₋₆alkyl, carbocyclyl, carbocyclylC₁₋₆alkyl, heterocyclyl and heterocyclylC₁₋₆alkyl which group is optionally substituted, by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵ and R¹⁶ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl (C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl; provided that when X is —C(O)NH—, R¹ is not the group

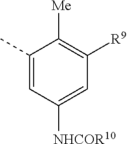

in the manufacture of a medicament for use in the treatment of proliferative disease.

In accordance with another aspect of the present invention, there is provided the use of a compound of formula (I)

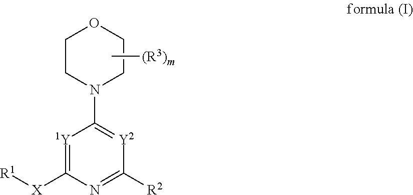

or a salt, ester or prodrug thereof; wherein
m is 0, 1, 2, 3 or 4;
X is a linker group selected from —CR⁴═CR⁵—, —CR⁴═CR⁵CR⁶R⁷—, —CR⁶R⁷CR⁵═CR⁴—, —C≡C—, —C≡CCR⁶R⁷—, —CR⁶R⁷C≡C—, —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)NR⁵CR⁶R⁷—, —S(O)₂NR⁴CR⁶R⁷—, —C(O)NR⁴—, —NR⁴C(O)—, —NR⁴C(O)NR⁵—, —S(O)₂NR⁴— and —NR⁴S(O)₂—;

¹Y and Y² are independently N or CR⁸ provided that one of ¹Y and Y² is N and the other is CR⁸;

R¹ is a group selected from C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, —R⁹, —OR⁹, —SR⁹, —SOR⁹, —SO₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹⁵, —NR⁹COCONR¹⁰R¹⁵ and —NR⁹SO₂R¹⁰;
R² is a group selected from C₁₋₆alkyl, carbocyclyl and heterocyclyl which group is optionally substituted by one or more substituent group independently selected from halo, cyano, nitro,
—R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², and —NR¹¹COCONR¹²R¹⁶;
each R³, when present, is independently selected from halo, cyano, nitro, —R¹³, —OR¹³, —SR¹³, —SOR¹³, —SO₂R¹³, —COR¹³, —CO₂R¹³, —CONR¹³R¹⁴, —NR¹³R¹⁴; NR¹³COR¹⁴, —R¹³CO₂R¹⁴ and —NR¹³SO₂R¹⁴;
R⁴ and R⁵ are independently hydrogen or C₁₋₆alkyl;
or R¹ and R⁴ together with the atom or atoms to which they are attached form a 5- to 10-membered carbocyclic or heterocyclic ring wherein 1, 2 or 3 ring carbon atoms is optionally replaced with N, O or S and which ring is optionally substituted by one or more substituent groups selected from halo, cyano; nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ are independently selected from hydrogen, halo, cyano, nitro and C₁₋₆alkyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹ and R¹² are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₄alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵ and R¹⁶ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl; provided that when X is —C(O)NH—, R¹ is not the group

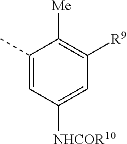

in the manufacture of a medicament for use in the treatment of proliferative disease.

In accordance with another aspect of the present invention, there is provided the use of a compound of formula (I)

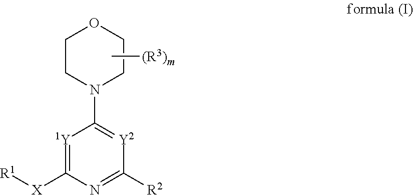

or a salt, ester or prodrug thereof; wherein
m is 0, 1, 2, 3 or 4;
X is a linker group selected from —CR⁴═CR⁵—, —CR⁴═CR⁵CR⁶R⁷—, —CR⁶R⁷CR⁵═CR⁴—, —C≡C—, —C≡CCR⁶R⁷—, —CR⁶R⁷≡C—, —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)NR⁵CR⁶R⁷—, —S(O)₂NR⁴CR⁶R⁷—, —C(O)NR⁴—, —NR⁴C(O)—, —NR⁴C(O)NR⁵—, —S(O)₂NR⁴— and —NR⁴S(O)₂—;
¹Y and Y² are independently N or CR⁸ provided that one of ¹Y and Y² is N and the other is CR⁸;
R¹ is a group selected from C₁₋₆alkyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —COR⁹, —CONR⁹R¹⁰, —NR⁹R¹⁰ and —NR⁹COR¹⁰;
R² is a group selected from C₁₋₆alkyl, carbocyclyl and heterocyclyl which group is optionally substituted by one or more substituent group independently selected from halo, cyano, nitro,
—R¹¹, —OR¹¹, —COR¹¹, —CONR¹¹R¹², —NR¹¹R¹², and —NR¹¹COR¹²;
each R³, when present, is independently selected from halo, cyano, nitro, —R¹³, —OR¹³, —COR¹³, —CONR¹³R¹⁴, —NR¹³R¹⁴ and —NR¹³COR¹⁴;
R⁴ and R⁵ are independently hydrogen or C₁₋₆alkyl;
R⁶ and R⁷ are independently selected from hydrogen, halo, cyano, nitro and C₁₋₆alkyl;
R⁸ is selected from hydrogen, halo, cyano and C₁₋₆alkyl;
R⁹ and R¹⁰ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹¹ and R¹² are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino,
C₁₋₆alkylamino and bis(C₁₋₆alkyl)amino;
R¹³ and R¹⁴ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino,
C₁₋₆alkylamino and bis(C₁₋₆alkyl)amino;
provided that when X is —C(O)NH—, R¹ is not the group

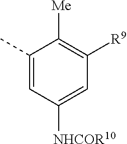

in the manufacture of a medicament for use in the treatment of proliferative disease.

In accordance with a further aspect of the present invention, there is also provided a compound of formula (I)

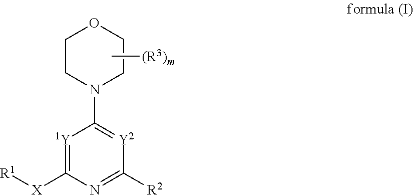

or a salt, ester or prodrug thereof; wherein
m is 0, 1, 2, 3 or 4;
X is a linker group selected from —CR⁴═CR⁵—, —CR⁴═CR⁵CR⁶R⁷—, —CR⁶R⁷CR⁵═CR⁴—, —C≡C—, —C≡CCR⁶R⁷—, —CR⁶R⁷C≡C—, —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷—, —NR⁴S(O)₂CR⁶R⁷—, —S(O)₂NR⁴CR⁶R⁷—, —C(O)NR⁴—, —NR⁴C(O)—, —NR⁴C(O)NR⁵—, —(O)₂NR⁴— and and —NR⁴S(O)₂—;

¹Y and Y² are independently N or CR⁸ provided that one of ¹Y and Y² is N and the other is CR⁸;

R¹ is a group selected from C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, —R⁹, —OR⁹, —SR⁹, —SOR⁹, —O₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹⁵, —NR⁹COCONR¹⁰R¹⁵ and NR⁹SO₂R¹⁰;
R² is a group selected from C₁₋₆alkyl, carbocyclyl and heterocyclyl which group is optionally substituted by one or more substituent group independently selected from halo, cyano, nitro,
—R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², and —NR¹¹COCONR¹²R¹⁶;
each R³, when present, is independently selected from halo, cyano, nitro, —R¹³, —OR¹³, —R¹³, —SOR¹³, —SO₂R¹³, —COR¹³, —CO₂R¹³, —CONR¹³R¹⁴, —NR¹³R¹⁴, —NR¹³COR¹⁴, —NR¹³CO2R¹⁴ and —NR¹³SO₂R¹⁴;
R⁴ and R⁵ are independently hydrogen or C₁₋₆alkyl;
or R¹ and R⁴ together with the atom or atoms to which they are attached form a 5- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ are independently selected from hydrogen, halo, cyano, nitro and C₁₋₆alkyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹ and R¹² are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₄alkoxy, amino, C₁₋₆alkylamino, bis(C₁₋₆alkyl)amino, aminoC₁₋₄alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵, and R¹⁶ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl; provided that the compound of formula (I) is not a compound listed in Excluded Compound List 1
and provided that when X is —C(O)NH—, R¹ is not the group

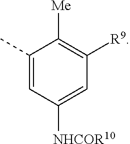

In accordance with a further aspect of the present invention, there is also provided a compound of formula (I)

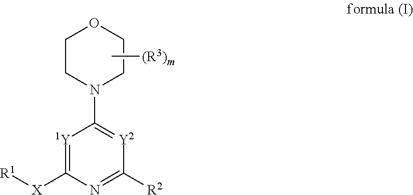

or a salt, ester or prodrug thereof; wherein
m is 0, 1, 2, 3 or 4;
X is a linker group selected from —CR⁴═CR⁵—, —CR⁴═CR⁵CR⁶R⁷—, —CR⁶R⁷CR⁵═CR⁴—, —C≡C—, —C≡CCR⁶R⁷—, —CR⁶R⁷C≡C—, —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)NR⁵CR⁶R⁷—, —S(O)₂NR⁴CR⁶R⁷—, —C(O)NR⁴—, —NR⁴C(O)—, —NR⁴C(O)NR⁵—, —S(O)₂NR⁴— and —NR⁴S(O)₂—;
¹Y and Y² are independently N or CR⁸ provided that one of ¹Y and Y² is N and the other is CR⁸;
R¹ is a group selected from C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, —R⁹, —OR⁹, —SR⁹, —SOR⁹, —O₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹⁵, —NR⁹COCONR¹⁰R¹⁵ and NR⁹SO₂R¹⁰;
R² is a group selected from C₁₋₆alkyl, carbocyclyl and heterocyclyl which group is optionally substituted by one or more substituent group independently selected from halo, cyano, nitro,
—R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², and —NR¹¹COCONR¹²R¹⁶;
each R³, when present, is independently selected from halo, cyano, nitro, —R¹³, —OR¹³, —R¹³, —SOR¹³, —SO₂R¹³, —COR¹³, —CO₂R¹³, —CONR¹³R¹⁴, —NR¹³R¹⁴, —NR¹³COR¹⁴, —NR¹³CO2R¹⁴ and —NR¹³SO₂R¹⁴;
R⁴ and R⁵ are independently hydrogen or C₁₋₆alkyl;
or R¹ and R⁴ together with the atom or atoms to which they are attached form a 5- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, C₁₋₆alkyl, C₁₋₆alkoxy, C₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ are independently selected from hydrogen, halo, cyano, nitro and C₁₋₆alkyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹ and R¹² are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵ and R¹⁶ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
provided that the compound of formula (I) is not a compound listed in Excluded Compound List 1
and provided that when X is —C(O)NH—, R¹ is not the group

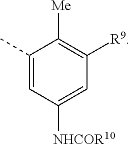

In accordance with a further aspect of the present invention, there is also provided a compound of formula (I)

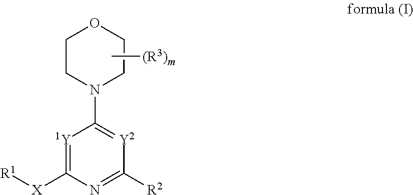

or a salt, ester or prodrug thereof; wherein
m is 0, 1, 2, 3 or 4;
X is a linker group selected from —CR⁴═CR⁵—, —CR⁴═CR⁵CR⁶R⁷—, —CR⁶R⁷CR⁵═CR⁴—, —C≡C—, —C≡CCR⁶R⁷—, —CR⁶R⁷≡C—, —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)NR⁵CR⁶R⁷—, —S(O)₂NR⁴CR⁶R⁷—, —C(O)NR⁴—, —NR⁴C(O)—, —NR⁴C(O)NR⁵—, —S(O)₂NR⁴— and —NR⁴S(O)₂—;
¹Y and Y² are independently N or CR⁸ provided that one of ¹Y and Y² is N and the other is CR⁸;
R¹ is a group selected from C₁₋₆alkyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —COR⁹, —CONR⁹R¹⁰, —NR⁹R¹⁰ and —R⁹COR¹⁰;
R² is a group selected from C₁₋₆alkyl, carbocyclyl and heterocyclyl which group is optionally substituted by one or more substituent group independently selected from halo, cyano, nitro,

### —R¹¹, —OR¹¹, —COR¹¹, —CONR¹¹R¹², —NR¹¹R¹² and —NR¹¹COR¹²;

each R³, when present, is independently selected from halo, cyano, nitro, —R¹³, —OR¹³, —COR¹³, —CONR¹³R¹⁴, —NR¹³R¹⁴ and —NR¹³COR¹⁴;
R⁴ and R⁵ are independently hydrogen or C₁₋₆alkyl;
R⁶ and R⁷ are independently selected from hydrogen, halo, cyano, nitro and C₁₋₆alkyl;
R⁸ is selected from hydrogen, halo, cyano and C₁₋₆alkyl;
R⁹ and R¹⁰ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹¹ and R¹² are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino,
C₁₋₆alkylamino and bis(C₁₋₆alkyl)amino;
R¹³ and R¹⁴ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino,
C₁₋₆alkylamino and bis(C₁₋₆alkyl)amino;
provided that the compound of formula (I) is not a compound listed in Excluded Compound List 1
and provided that when X is —C(O)NH—, R¹ is not the group

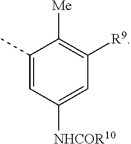

### Excluded Compound List 1:

4-{6-[(methylthio)methyl]-2-methylpyrimidin-4-yl}morpholine;
4-(6-{[(4-chlorophenyl)thio]methyl}-2-methylpyrimidin-4-yl)morpholine;
4-(6-{[(4-chlorophenyl)thio]methyl}-2-methylpyrimidin-4-yl)-2,6-dimethylmorpholine;
4-{6-[(phenylsulfinyl)methyl]-2-methylpyrimidin-4-yl}morpholine;
4-(6-{[(4-chlorophenyl)sulfinyl]methyl}-2-methylpyrimidin-4-yl)morpholine;
4-{6-[(phenylsulfonyl)methyl]-2-methylpyrimidin-4-yl}morpholine;
4-(6-{[(4-chlorophenyl)sulfonyl]methyl}-2-methylpyrimidin-4-yl)morpholine;
4-{6-[(methylthio)methyl]-2-phenylpyrimidin-4-yl}morpholine;
4-{6-[(phenylthio)methyl]-2-phenylpyrimidin-4-yl}morpholine;
4-(6-{[(4-chlorophenyl)thio]methyl}-2-phenylpyrimidin-4-yl)morpholine;
4-(6-{[(4-chlorobenzyl)thio]methyl}-2-phenylpyrimidin-4-yl)morpholine;
4-(6-{[(4-chlorobenzyl)thio]methyl}-2-phenylpyrimidin-4-yl)-2,6-dimethylmorpholine;
4-{6-[(methylsulfinyl)methyl]-2-phenylpyrimidin-4-yl}morpholine;
4-{6-[(phenylsulfinyl)methyl]-2-phenylpyrimidin-4-yl}morpholine;
4-(6-{[(4-chlorophenyl)sulfinyl]methyl}-2-phenylpyrimidin-4-yl)morpholine;
4-{6-[(methylsulfonyl)methyl]-2-phenylpyrimidin-4-yl}morpholine;
4-{6-[(phenylsulfonyl)methyl]-2-phenylpyrimidin-4-yl}morpholine;
4-{6-[(methylthio)methyl]-2-pyridin-2-ylpyrimidin-4-yl}morpholine;
4-{6-[(phenylthio)methyl]-2-pyridin-4-ylpyrimidin-4-yl}morpholine;
4-(6-{[(4-chlorophenyl)thio]methyl}-2-pyridin-2-ylpyrimidin-4-yl)morpholine;
4-{6-[(methylsulfonyl)methyl]-2-pyridin-3-ylpyrimidin-4-yl}morpholine;
4-{6-[(methylsulfonyl)methyl]-2-pyridin-4-ylpyrimidin-4-yl}morpholine;
4-{6-[(phenylsulfonyl)methyl]-2-pyridin-2-ylpyrimidin-4-yl}morpholine;
4-{6-[(phenylsulfonyl)methyl]-2-pyridin-3-ylpyrimidin-4-yl}morpholine;
4-{6-[(phenylsulfonyl)methyl]-2-pyridin-4-ylpyrimidin-4-yl}morpholine;
4-{6-[(methoxy)methyl]-2-methylpyrimidin-4-yl}morpholine;
4-{6-[(methoxy)methyl]-2-phenylpyrimidin-4-yl}morpholine;
4-{6-[(methoxy)methyl]-2-phenylpyrimidin-4-yl}-2,6-dimethylmorpholine;
4-{6-[(phenoxy)methyl]-2-(6-methylpyrid-2-yl)pyrimidin-4-yl}-2,6-dimethylmorpholine;
N-[5-[[3-(1-cyano-1-methylethyl)benzoyl]amino]-2-methylphenyl]-2,6-di-4-morpholinyl-4-pyrimidinecarboxamide;
N-[5-[[3-(1-cyano-1-methylethyl)benzoyl]amino]-2-methylphenyl]-6-(4-morpholinyl)-2-(trifluoromethyl)-4-pyrimidinecarboxamide;
N-[4-fluoro-3-[(pyrazinyloxy)methyl]phenyl]-2,6-di-4-morpholinyl-4-pyrimidinecarboxamide;
4-[2-methyl-6-[(1E)-2-[3-(trifluoromethyl)phenyl]ethenyl]-4-pyrimidinyl]-morpholine;
4-[6-methyl-2-[(1E)-2-[3-(trifluoromethyl)phenyl]ethenyl]-4-pyrimidinyl]-morpholine;
3,4,5-trimethoxy-N-[4-methyl-6-(4-morpholinyl)-2-pyrimidinyl]-benzamide;
N-(2,3-dimethyl-1H-indol-5-yl)-2,6-di-4-morpholinyl-4-pyrimidinecarboxamide;
N-(2,3-dimethyl-1H-indol-5-yl)-4,6-di-4-morpholinyl-2-pyridinecarboxamide;
N-(3,4-dimethylphenyl)-2,6-di-4-morpholinyl-4-pyrimidinecarboxamide;
N-[3-(aminocarbonyl)phenyl]-2,6-di-4-morpholinyl-4-pyrimidinecarboxamide;
N-(4,6-di-4-morpholinyl-2-pyridinyl)-N′-(3-methylphenyl)-urea;
N-(2,3-dimethyl-1H-indol-5-yl)-4,6-di-4-morpholinyl-2-pyridinecarboxamide;
4,6-di-4-morpholinyl-N-(1,2,3-trimethyl-1H-indol-5-yl)-2-pyridinecarboxamide;
N-(2,3-dimethyl-1H-indol-5-yl)-2-[(2R,6S)-2,6-dimethyl-4-morpholinyl]-6-(4-morpholinyl)-4-pyrimidinecarboxamide;
2,6-di-4-morpholinyl-N-(1,2,3-trimethyl-1H-indol-5-yl)-4-pyrimidinecarboxamide;
N-[3-(dimethylamino)phenyl]-2,6-di-4-morpholinyl-4-pyrimidinecarboxamide;
N-[3,4,5-trimethoxyphenyl]-2,6-di-4-morpholinyl-4-pyrimidinecarboxamide;
2,6-di-4-morpholinyl-N-(6,7,8,9-tetrahydro-5H-benzocyclohepten-6-yl)-4-pyrimidinecarboxamide; and
4-[2-methyl-6-[2-(5-nitro-2-furyl)vinyl]-4-pyrimidinyl]-morpholine.

Additionally, the invention provides a compound of formula (I) as defined herein, or a salt, ester or prodrug thereof, provided that

(a) when ¹Y is CH, Y² is N, X is —SCH₂—, —S(O)CH₂— or —S(O)₂CH₂— and R² is methyl, phenyl or pyridyl, then R¹ is not methyl, phenyl, 4-chlorophenyl or 4-chlorobenzyl; and
(b) when ¹Y is CH, Y² is N, X is —OCH₂— and R² is methyl, phenyl or 2-methylpyrid-2yl then R¹ is not methyl or phenyl.

The following compounds from Excluded Compound List 1 may also be identified by their Chemical Abstracts Number N-[5-[[3-(1-cyano-1-methylethyl)benzoyl]amino]-2-methylphenyl]-2,6-di-4-morpholinyl-4-pyrimidinecarboxamide (873449-41-3); N-[5-[[3-(1-cyano-1-methylethyl)benzoyl]amino]-2-methylphenyl]-6-(4-morpholinyl)-2-(trifluoromethyl)-4-pyrimidinecarboxamide (873449-50-4); N-[4-fluoro-3-[(pyrazinyloxy)methyl]phenyl]-2,6-di-4-morpholinyl-4-pyrimidinecarboxamide (642085-32-3); 4-[2-methyl-6-[(1E)-2-[3-(trifluoromethyl)phenyl]ethenyl]-4-pyrimidinyl]-morpholine (425423-56-9); 4-[6-methyl-2-[(1E)-2-[3-(trifluoromethyl)phenyl]ethenyl]-4-pyrimidinyl]-morpholine (425423-57-0); 3,4,5-trimethoxy-N-[4-methyl-6-(4-morpholinyl)-2-pyrimidinyl]-benzamide (168197-68-0); N-(2,3-dimethyl-1H-indol-5-yl)-2,6-di-4-morpholinyl-4-pyrimidinecarboxamide (887133-39-3); N-(2,3-dimethyl-1H-indol-5-yl)-4,6-di-4-morpholinyl-2-pyridinecarboxamide (887133-47-3); N-(3,4-dimethylphenyl)-2,6-di-4-morpholinyl-4-pyrimidinecarboxamide (887133-68-8); N-[3-(aminocarbonyl)phenyl]-2,6-di-4-morpholinyl-4-pyrimidinecarboxamide (87133-69-9); N-(4,6-di-4-morpholinyl-2-pyridinyl)-N′-(3-methylphenyl)-urea (87133-93-9); N-(2,3-dimethyl-1H-indol-5-yl)-4,6-di-4-morpholinyl-2-pyridinecarboxamide (887134-72-7); 4,6-di-4-morpholinyl-N-(1,2,3-trimethyl-1H-indol-5-yl)-2-pyridinecarboxamide (887134-74-9); N-(2,3-dimethyl-1H-indol-5-yl)-2-[(2R,6S)-2,6-dimethyl-4-morpholinyl]-6-(4-morpholinyl)-4-pyrimidinecarboxamide (887136-28-9); 2,6-di-4-morpholinyl-N-(1,2,3-trimethyl-1H-indol-5-yl)-4-pyrimidinecarboxamide (887136-30-3); N-[3-(dimethylamino)phenyl]-2,6-di-4-morpholinyl-4-pyrimidinecarboxamide (887136-53-0); 2,6-di-4-morpholinyl-N-(6,7,8,9-tetrahydro-5H-benzocyclohepten-6-yl)-4-pyrimidinecarboxamide (450367-63-2); and 4-[2-methyl-6-[2-(5-nitro-2-furyl)vinyl]-4-pyrimidinyl]-morpholine (4592-48-7).

The following compound from Excluded Compound List 1 N-[3,4,5-trimethoxyphenyl]-2,6-di-4-morpholinyl-4-pyrimidinecarboxamide may also be referred to as 2,6-dimorpholin-4-yl-N-(3,4,5-trimethoxyphenyl)pyrimidine-4-carboxamide.

Certain compounds of formula (I) are capable of existing in stereoisomeric forms. It will be understood that the invention encompasses all geometric and optical isomers of the compounds of formula (I) and mixtures thereof including racemates. Tautomers and mixtures thereof also form an aspect of the present invention. Solvates and mixtures thereof also form an aspect of the present invention. For example, a suitable solvate of a compound of formula (I) is, for example, a hydrate such as a hemi-hydrate, a mono-hydrate, a di-hydrate or a tri-hydrate or an alternative quantity thereof.

The present invention relates to the compounds of formula (I) as herein defined as well as to salts thereof. Salts for use in pharmaceutical compositions will be pharmaceutically acceptable salts, but other salts may be useful in the production of the compounds of formula (I) and their pharmaceutically acceptable salts. Pharmaceutically acceptable salts of the invention may, for example, include acid addition salts of compounds of formula (I) as herein defined which are sufficiently basic to form such salts. Such acid addition salts include but are not limited to furmarate, methanesulfonate, hydrochloride, hydrobromide, citrate and maleate salts and salts formed with phosphoric and sulfuric acid. In addition where compounds of formula (I) are sufficiently acidic, salts are base salts and examples include but are not limited to, an alkali metal salt for example sodium or potassium, an alkaline earth metal salt for example calcium or magnesium, or organic amine salt for example triethylamine, ethanolamine, diethanolamine, triethanolamine, morpholine, N-methylpiperidine, N-ethylpiperidine, dibenzylamine or amino acids such as lysine.

The compounds of formula (I) may also be provided as in vivo hydrolysable esters. An in vivo hydrolysable ester of a compound of formula (I) containing carboxy or hydroxy group is, for example a pharmaceutically acceptable ester which is cleaved in the human or animal body to produce the parent acid or alcohol. Such esters can be identified by administering, for example, intravenously to a test animal, the compound under test and subsequently examining the test animal's body fluid.

Suitable pharmaceutically acceptable esters for carboxy include C₁₋₆alkoxymethyl esters for example methoxymethyl, C₁₋₆alkanoyloxymethyl esters for example pivaloyloxymethyl, phthalidyl esters, C₃₋₈cycloalkoxycarbonyloxyC₁₋₆alkyl esters for example 1-cyclohexylcarbonyloxyethyl, 1,3-dioxolen-2-onylmethyl esters for example 5-methyl-1,3-dioxolen-2-onylmethyl, and C₁₋₆alkoxycarbonyloxyethyl esters for example 1-methoxycarbonyloxyethyl; and may be formed at any carboxy group in the compounds of this invention.

Suitable pharmaceutically acceptable esters for hydroxy include inorganic esters such as phosphate esters (including phosphoramidic cyclic esters) and α-acyloxyalkyl ethers and related compounds which as a result of the in vivo hydrolysis of the ester breakdown to give the parent hydroxy group/s. Examples of α-acyloxyalkyl ethers include acetoxymethoxy and 2,2-dimethylpropionyloxymethoxy. A selection of in vivo hydrolysable ester forming groups for hydroxy include C₁₋₁₀alkanoyl, for example formyl, acetyl, benzoyl, phenylacetyl, substituted benzoyl and phenylacetyl; C₁₋₁₀alkoxycarbonyl (to give alkyl carbonate esters), for example ethoxycarbonyl; di-C₁₋₄alkylcarbamoyl and N-(di-C₁₋₄alkylaminoethyl)-N—C₁₋₄alkylcarbamoyl (to give carbamates); di-C₁₋₄alkylaminoacetyl and carboxyacetyl. Examples of ring substituents on phenylacetyl and benzoyl include aminomethyl, C₁₋₄alkylaminomethyl and di-(C₁₋₄alkyl)aminomethyl, and morpholino or piperazino linked from a ring nitrogen atom via a methylene linking group to the 3- or 4-position of the benzoyl ring. Other interesting in vivo hydrolysable esters include, for example, R^{A}C(O)OC₁₋₆alkyl-CO—, wherein R^{A} is for example, benzyloxy-C₁-₄-alkyl, or phenyl. Suitable substituents on a phenyl group in such esters include, for example, 4-C₁₋₄piperazino-C₁₋₄alkyl, piperazino-C₁₋₄alkyl and morpholino-C₁₋₄alkyl.

The compounds of the formula (I) may be also be administered in the form of a prodrug which is broken down in the human or animal body to give a compound of the formula (I). Various forms of prodrugs are known in the art. For examples of such prodrug derivatives, see:
a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen and H. Bundgaard, Chapter 5 “Design and Application of Prodrugs”, by H. Bundgaard p. 113-191 (1991);
c) H. Bundgaard, Advanced Drug Delivery Reviews, 8, 1-38 (1992);
d) H. Bundgaard, et al., Journal of Pharmaceutical Sciences, 77, 285 (1988); and
e) N. Kakeya, et al., Chem Pharm Bull, 32, 692 (1984).

In this specification the generic term “C_{p-q}alkyl” includes both straight-chain and branched-chain alkyl groups. However references to individual alkyl groups such as “propyl” are specific for the straight chain version only (i.e. n-propyl and isopropyl) and references to individual branched-chain alkyl groups such as “tert-butyl” are specific for the branched chain version only.

The prefix C_{p-q} in C_{p-q}alkyl and other terms (where p and q are integers) indicates the range of carbon atoms that are present in the group, for example C₁₋₄alkyl includes C₁alkyl (methyl), C₂alkyl (ethyl), C₃alkyl (propyl as n-propyl and isopropyl) and C₄alkyl (n-butyl, sec-butyl, isobutyl and tert-butyl).

The term C_{p-q}alkoxy comprises —O—C_{p-q}alkyl groups.

The term C_{p-q}alkanoyl comprises —C(O)alkyl groups.

The term halo includes fluoro, chloro, bromo and iodo.

“Carbocyclyl” is a saturated, unsaturated or partially saturated monocyclic, bicyclic or tricyclic ring system containing from 3 to 14 ring atoms, wherein a ring CH₂ group may be replaced with a C═O group. “Carbocyclyl” includes “aryl”, “C_{p-q}cycloalkyl” and “C_{p-q}cycloalkenyl”.

“aryl” is an aromatic monocyclic, bicyclic or tricyclic carbcyclyl ring system.

“C_{p-q}cycloalkenyl” is an unsaturated or partially saturated monocyclic, bicyclic or tricyclic carbocyclyl ring system containing at least 1 C≡C bond and wherein a ring CH₂ group may be replaced with a C═O group.

“C_{p-q}cycloalkyl” is a saturated monocyclic, bicyclic or tricyclic carbocyclyl ring system and wherein a ring CH₂ group may be replaced with a C═O group.

“Heterocyclyl” is a saturated, unsaturated or partially saturated monocyclic, bicyclic or tricyclic ring system containing from 3 to 14 ring atoms of which 1, 2, 3 or 4 ring atoms are chosen from nitrogen, sulfur or oxygen, which ring may be carbon or nitrogen linked and wherein a ring nitrogen or sulfur atom may be oxidised and wherein a ring CH₂ group may be replaced with a C═O group. “Heterocyclyl” includes “heteroaryl”, “cycloheteroalkyl” and “cycloheteroalkenyl”.

“Heteroaryl” is an aromatic monocyclic, bicyclic or tricyclic heterocyclyl, particularly having 5 to 10 ring atoms, of which 1, 2, 3 or 4 ring atoms are chosen from nitrogen, sulfur or oxygen where a ring nitrogen or sulfur may be oxidised.

“Cycloheteroalkenyl” is an unsaturated or partially saturated monocyclic, bicyclic or tricyclic heterocyclyl ring system, particularly having 5 to 10 ring atoms, of which 1, 2, 3 or 4 ring atoms are chosen from nitrogen, sulfur or oxygen, which ring may be carbon or nitrogen linked and wherein a ring nitrogen or sulfur atom may be oxidised and wherein a ring CH₂ group may be replaced with a C═O group.

“Cycloheteroalkyl” is a saturated monocyclic, bicyclic or tricyclic heterocyclic ring system, particularly having 5 to 10 ring atoms, of which 1, 2, 3 or 4 ring atoms are chosen from nitrogen, sulfur or oxygen, which ring may be carbon or nitrogen linked and wherein a ring nitrogen or sulfur atom may be oxidised and wherein a ring CH₂ group may be replaced with a C═O group.

This specification may make use of composite terms to describe groups comprising more than one functionality. Unless otherwise described herein, such terms are to be interpreted as is understood in the art. For example carbocyclylC_{p-q}alkyl comprises C_{p-q}alkyl substituted by carbocyclyl, heterocyclylC_{p-q}alkyl comprises C_{p-q}alkyl substituted by heterocyclyl, and bis(C_{p-q}alkyl)amino comprises amino substituted by 2 C_{p-q}alkyl groups which may be the same or different.

HaloC_{p-q}alkyl is a C_{p-q}alkyl group that is substituted by 1 or more halo substituents and particularly 1, 2 or 3 halo substituents. Similarly, other generic terms containing halo such as haloC_{p-q}alkoxy may contain 1 or more halo substituents and particularly 1, 2 or 3 halo substituents.

HydroxyC_{p-q}alkyl is a C_{p-q}alkyl group that is substituted by 1 or more hydroxyl substituents and particularly by 1, 2 or 3 hydroxy substituents. Similarly other generic terms containing hydroxy such as hydroxyC_{p-q}alkoxy may contain 1 or more and particularly 1, 2 or 3 hydroxy substituents.

C_{p-q}alkoxyC_{p-q}alkyl is a C_{p-q}alkyl group that is substituted by 1 or more C_{p-q}alkoxy substituents and particularly 1, 2 or 3 C_{p-q}alkoxy substituents. Similarly other generic terms containing C_{p-q}alkoxy such as C_{p-q}alkoxyC_{p-q}alkoxy may contain 1 or more C_{p-q}alkoxy substituents and particularly 1, 2 or 3 C_{p-q}alkoxy substituents.

Where optional substituents are chosen from “1 or 2”, from “1, 2, or 3” or from “1, 2, 3 or 4” groups or substituents it is to be understood that this definition includes all substituents being chosen from one of the specified groups i.e. all substitutents being the same or the substituents being chosen from two or more of the specified groups i.e. the substitutents not being the same.

Compounds of the present invention have been named with the aid of computer software (ACD/Name version 8.0).

“Proliferative disease(s)” includes malignant disease(s) such as cancer as well as non-malignant disease(s) such as inflammatory diseases, obstracutive airways diseases, immune diseases or cardiovascular diseases.

Suitable values for any R group or any part or substitutent for such groups include:
for C₁₋₄alkyl: methyl, ethyl, propyl, butyl, 2-methylpropyl and tert-butyl;
for C₁₋₆alkyl: C₁₋₄alkyl, pentyl, 2,2-dimethylpropyl, 3-methylbutyl and hexyl;
for C₃₋₆cycloalkyl: cyclopropyl, cyclobutyl, cyclopentyl and cyclohexyl;
for C₃₋₆cycloalkylC₁₋₄alkyl: cyclopropylmethyl, cyclopropylethyl, cyclobutylmethyl, cyclopentylmethyl and cyclohexylmethyl;
for aryl: phenyl and naphthyl;
for arylC₁₋₄alkyl: benzyl, phenethyl, naphthylmethyl and naphthylethyl;
for carbocylyl: aryl, cyclohexenyl and C₃₋₆cycloalkyl;
for halo: fluoro, chloro, bromo and iodo;
for C₁₋₄alkoxy: methoxy, ethoxy, propoxy and isopropoxy;
for C₁₋₆alkoxy: C₁₋₄alkoxy, pentyloxy, 1-ethylpropoxy and hexyloxy;
for C₁₋₆alkanoyl: acetyl, propanoyl and 2-methylpropanoyl;
for heteroaryl: pyridyl, imidazolyl, quinolinyl, cinnolyl, pyrimidinyl, thienyl, pyrrolyl, pyrazolyl, thiazolyl, thiazolyl, triazolyl, oxazolyl, isoxazolyl, furanyl, pyridazinyl, pyrazinyl, indolyl, benzofuranyl, dibenzofuranyl and benzothienyl;
for heteroarylC₁₋₄alkyl: pyrrolylmethyl, pyrrolylethyl, imidazolylmethyl, imidazolylethyl, pyrazolylmethyl, pyrazolylethyl, furanylmethyl, furanylethyl, thienylmethyl, theinylethyl, pyridylmethyl, pyridylethyl, pyrazinylmethyl, pyrazinylethyl, pyrimidinylmethyl, pyrimidinylethyl, pyrimidinylpropyl, pyrimidinylbutyl, imidazolylpropyl, imidazolylbutyl, quinolinylpropyl, 1,3,4-triazolylpropyl and oxazolylmethyl;
for heterocyclyl: heteroaryl, pyrrolidinyl, isoquinolinyl, quinoxalinyl, benzothiazolyl, benzoxazolyl, piperidinyl, piperazinyl, azetidinyl, morpholinyl, tetrahydroisoquinolinyl, tetrahydroquinolinyl, indolinyl, dihydro-2H-pyranyl and tetrahydrofuranyl.

It should be noted that examples given for terms used in the description are not limiting.

Particular values of m, X, ¹Y and Y², R¹, R² and R³ are as follows. Such values may be used where appropriate, in connect with any aspect of the invention, or part thereof, and with any of the definitions, claims or embodiments defined herein.

m

In one aspect of the invention m is 0, 1, 2 or 3.

In another aspect m is 0, 1 or 2.

In a further aspect m is 0 or 1.

In yet another aspect m is 0 so that R³ is absent.

X

In one aspect of the invention X is a linker group selected from —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)NR⁵CR⁶R⁷—, —S(O)₂NR⁴CR⁶R⁷—, —NR⁴C(O)—, —C(O)NR⁴—, —S(O)₂NR⁴— and —NR⁴S(O)₂—.

In another aspect X is a linker group selected from —NR⁴CR⁶R⁷—, —OCR⁶R⁷—, —CR⁶R⁷—, —S(O)CR⁶R⁷—, —S(O)₂CR⁶R⁷—, —C(O)NR⁴CR⁶R⁷—, —NR⁴C(O)NR⁵CR⁶R⁷—, —(O)₂NR⁴CR⁶R⁷, —C(O)NR⁴— and —NR⁴C(O)—.

In a further aspect X is a linker group selected from —NR⁴CR⁶R⁷—, —OCR⁶R⁷—, —SCR⁶R⁷—, —S(O)CR⁶R⁷—, —S(O)₂CR⁶R⁷—, —C(O)NR⁴—, and —NR⁴C(O)—.

In a further aspect X is a linker group selected from —NR⁴CR⁶R⁷—, —OCR⁶R⁷—, —SCR⁶R⁷—, —S(O)CR⁶R⁷— and —S(O)₂CR⁶R⁷—.

In yet another aspect X is a linker group selected from —SCR⁶R⁷—, —S(O)CR⁶R⁷— and —S(O)₂CR⁶R⁷—.

In another aspect X is a linker group selected from —NR⁴CH₂—, —OCH₂—, —SCH₂—, —S(O)CH₂—, —S(O)₂CH₂—, —C(O)NR⁴—, and —NR⁴C(O)—.

In another aspect X is a linker group selected from —NR⁴CH₂—, —OCH₂—, —SCH₂—, —S(O)CH₂— and —S(O)₂CH₂—.

In a further aspect X is a linker group selected from —NHCH₂—, —N(CH₃)CH₂—, —OCH₂—, —SCH₂—, —S(O)CH₂—, —S(O)₂CH₂—, —C(O)NH—, —C(O)N(CH₃)—, —NHC(O)— and —N(CH₃)C(O)—.

In yet a further aspect X is a linker group selected from —NHCH₂—, —N(CH₃)CH₂—, —OCH₂—, —SCH₂— and —S(O)₂CH₂—.

In another aspect X is —SCH₂— or —S(O)₂CH₂—.

In another aspect X is —S(O)₂CH₂—.

¹Y and Y²

In one aspect of the invention ¹Y is N and Y² is CR⁸.

In another aspect ¹Y is N and Y² is CH.

In yet another aspect ¹Y is CR⁸ and Y² is N.

In a further aspect ¹Y is CH or CF and Y² is N.

In yet a further aspect ¹Y is CH and Y² is N.

R¹

In one aspect of the invention R¹ is a group selected from C₁₋₄alkyl, C₃₋₆cycloalkyl, aryl, C₃₋₆cycloalkylC₁₋₄alkyl, arylC₁₋₄alkyl, cycloheteroalkyl, heteroaryl, cycloheteroalkylC₁₋₄alkyl, heteroarylC₁₋₄alkyl, which group is optionally substituted by one or more substituent group selected from halo, cyano, nitro, R⁹, —OR⁹, —COR^{S}, —CONR⁹R¹⁰, —NR⁹R¹⁰ and —NR⁹COR¹⁰.

In another aspect, R¹ is a group selected from methyl, ethyl, propyl, butyl, isobutyl, tert-butyl, cyclopentyl, cyclohexyl, phenyl, benzyl, phenethyl, pyrrolidinyl, pyrrolyl, imidazolyl, pyrazolyl, furanyl, thienyl, pyridinyl, pyrimidinyl, pyrazinyl, pyrrolidinylmethyl, pyrrolidinylethyl, pyrrolylmethyl, pyrrolylethyl, imidazolylmethyl, imidazolylethyl, pyrazolylmethyl, pyrazolylethyl, furanylmethyl, furanylethyl, thienylmethyl, thienylethyl, pyridinylmethyl, pyridinylethyl, pyrimidinylmethyl, pyrimidinylethyl, pyrazinylmethyl and pyrazinylethyl, which group is optionally substituted by 1, 2 or 3 substituent group selected from halo, cyano, nitro, R⁹, —OR⁹, —COR⁹, —CONR⁹R¹⁰, —NR⁹R¹⁰ and —NR⁹COR¹⁰.

In a further aspect, R¹ is a group selected from methyl, ethyl, propyl, butyl, isobutyl, tert-butyl, cyclohexyl, phenyl, benzyl, phenethyl, pyridinyl, pyrazolylethyl, furanylmethyl, thienylmethyl, and pyrazinylethyl, which group is optionally substituted by 1 or 2 substituent group selected from halo, cyano, methyl, methoxy, trifluoromethyl, trifluoromethoxy, —CONH₂ and —CONHCH₃.

In yet another aspect R¹ is a group selected from methyl, ethyl, n-propyl, isopropyl, n-butyl, sec-butyl, isobutyl, tert-butyl, cyclohexyl, —CH₂CN, —CH₂C(O)NH₂, —CH₂CH₂NC(O)CH₃, phenyl; 4-fluorophenyl, 2-chlorophenyl, 3-chlorophenyl, 2-chloro-6-fluorophenyl, 3-chloro-4-fluorophenyl, 4-bromo-2-fluorophenyl, 4-trifluoromtheylphenyl, 4-trifluoromethoxyphenyl, 4-cycanophenyl, 3-methoxyphenyl, 4-methoxyphenyl, 3,4-dimethoxyphenyl, 4-(N-methylaminocarbonyl)phenyl, benzyl, 4-fluorobezyl, 2-chlorobenzyl, 2-chloro-6-fluorobenzyl, 4-methoxybenzyl, phenethyl, 3-trifluorophenethyl, furan-2-ylmethyl, thien-2-ylmethyl, 2-pyrazin-2-ylethyl, pyidin-3-yl, 2-methylpyridin-3-yl and 2-aminocarbonylpyridin-3-yl.

R²

In one aspect of the invention R² is selected from aryl and heteroaryl which group is optionally substituted by one or more substituent group independently selected from halo, cyano, nitro, —R¹¹, —OR¹¹, —COR¹¹, —CONR¹¹R¹², —NR¹¹R¹² and —NR¹¹COR¹².

In another aspect R² is selected from phenyl, naphthyl, pyrrolyl, imidazolyl, pyrazolyl, furanyl, thienyl, pyridinyl, pyrimidinyl, pyridazinyl, azaindolyl, indolyl, quinolinyl, benzimidazolyl, benzofuranyl, dibenzofuranyl, benzothienyl which group is optionally substituted by one or more substituent group independently selected from halo, cyano, nitro, —R¹¹, —OR¹¹, —COR¹¹, —CONR¹¹R¹², —NR¹¹R¹² and —NR¹¹COR¹².

In another aspect R² is selected from phenyl, naphthyl, pyrrolyl, imidazolyl, pyrazolyl, furanyl, thienyl, pyridinyl, pyrimidinyl, pyridazinyl, azaindolyl, indolyl, quinolinyl, benzimidazolyl, benzofuranyl, dibenzofuranyl, benzothienyl which group is optionally substituted by one or more substituent group independently selected from halo, methyl, methoxy, hydroxymethyl, cyanomethyl, phenoxy, pyrrolidinyl, —CONH₂, —CONHCH₃ and —CON(CH₃)₂.

In yet another aspect R² is 3-(hydroxymethyl)phenyl, 4-(hydroxymethyl)phenyl, 4-(cyanomethyl)phenyl, 3,4-dimethoxyphenyl, 3-fluoro-4-methoxyphenyl, 4-phenoxyphenyl, 3-pyrrolidin-1ylphenyl, 3-(aminocarbonyl)phenyl, 4-(dimethylaminocarbonyl)phenyl, furan-3-yl, thien-3-yl, 5-(hydroxymethyl)thien-2-yl, pyridin-2-yl, pyridin-4-yl, 2-methoxypyridin-5-yl, 2-methoxypyrimidin-5-yl, 2-methoxynaphth-6-yl, 5,7-diazabicyclo[4.3.0]nona-2,4,8,10-tetraenyl, azaindolyl, indol-5-yl, 1-methylindol-5-yl, quinolin-6-yl, benzimidazolyl, benzofuran-2-yl, dibenzofuran-1-yl and benzothien-3-yl.

In another aspect R² is phenyl optionally substituted by —NR¹¹COR¹².

In yet a further aspect R² is pyridin-2-yl, 3-hydroxyphenyl, 4-hydroxyphenyl, 3-hydroxymethylphenyl, 4-hydroxymethylphenyl or indol-5-yl.

In yet a further aspect R² is azaindolyl, indol-5-yl, benzimidazolyl, 3-hydroxyphenyl, 4-hydroxyphenyl, 3-hydroxymethylphenyl or 4-hydroxymethylphenyl

In another aspect R² is pyridin-2-yl.

In a further aspect R² is 3-hydroxyphenyl or 4-hydroxyphenyl.

In yet another aspect R² is 3-hydroxymethylphenyl or 4-hydroxymethylphenyl.

In yet a further aspect R² is indol-5-yl.

In one aspect R² is morpholinyl.

In another aspect R² is morpholino.

R⁴

In one aspect of the invention R⁴ is hydrogen or methyl.

In another aspect R⁴ is hydrogen.

R⁵

In one aspect of the invention R⁵ is hydrogen or methyl.

In another aspect R⁵ is hydrogen.

R⁶

In one aspect of the invention R⁶ is hydrogen or methyl.

In another aspect R⁶ is hydrogen.

R⁷

In one aspect of the invention R⁷ is hydrogen or methyl.

In another aspect R⁷ is hydrogen.

R⁸

In one aspect of the invention R⁸ is hydrogen or halo.

In another aspect R⁸ is hydrogen or fluoro.

In a further aspect R⁸ is hydrogen.

R⁹

In one aspect of the invention R⁹ is hydrogen or C₁₋₄alkyl optionally substituted by 1, 2 or 3 substituent groups selected from halo, cyano, nitro, hydroxy, C₁₋₄alkoxy, amino, C₁₋₄alkylamino and bis(C₁₋₄alkyl)amino.

In another aspect R⁹ is hydrogen or C₁₋₄alkyl optionally substituted by 1, 2 or 3 halo substituents.

In a further aspect R⁹ is hydrogen, methyl or trifluoromethyl.

R¹⁰

In one aspect of the invention R¹⁰ is hydrogen.

R¹¹

In one aspect of the invention R¹¹ is hydrogen or a group selected from C₁₋₄alkyl, aryl and cycloheteroalkyl which group is optionally substituted by 1, 2 or 3 groups selected from halo, hydroxy and cyano.

In another aspect R¹¹ is hydrogen, methyl optionally substituted with hydroxy or cyano, phenyl or pyrrolidinyl.

In another aspect R¹¹ is hydrogen or methyl.

R¹²

In one aspect of the invention R¹² is hydrogen or methyl.

In a particular class of compound of formula (I), or a salt, ester or prodrug thereof;

m is 0, 1, 2, 3 or 4;
X is a linker group selected from —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)NR⁵CR⁶R⁷—, —S(O)₂NR⁴CR⁶R⁷—, —NR⁴C(O)—, —S(O)₂NR⁴— and —NR⁴S(O)₂—;
¹Y and Y² are independently N or CR⁸ provided that one of ¹Y and Y² is N and the other is CR⁸;
R¹ is a group selected from C₁₋₆alkyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —COR⁹, —CONR⁹R¹⁰, —NR⁹R¹⁰ and —NR⁹COR¹⁰;
R² is selected from aryl and heteroaryl which group is optionally substituted by one or more substituent group independently selected from halo, cyano, nitro, —R¹¹, —OR¹¹, —COR¹¹,

### —CONR¹¹R¹², —NR¹¹R¹² and —NR¹¹COR¹²;

each R³, when present, is independently selected from halo, cyano, nitro, —R¹³, —OR¹³, —COR¹³, —CONR¹³R¹⁴, —NR¹³R¹⁴ and —NR¹³COR¹⁴;
R⁴ and R⁵ are independently hydrogen or C₁₋₆alkyl;
R⁶ and R⁷ are independently selected from hydrogen, halo, cyano, nitro and C₁₋₆alkyl;
R⁸ is selected from hydrogen, halo, cyano and C₁₋₆alkyl;
R⁹ and R¹⁰ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹¹ and R¹² are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino,
C₁₋₆alkylamino and bis(C₁₋₆alkyl)amino;
R¹³ and R¹⁴ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino,
C₁₋₆alkylamino and bis(C₁₋₆alkyl)amino;
provided that
(a) when ¹Y is CH, Y² is N, X is —SCH₂—, —S(O)CH₂— or —S(O)₂CH₂— and R² is methyl, phenyl or pyridyl, then R¹ is not methyl, phenyl, 4-chlorophenyl or 4-chlorobenzyl; and
(b) when ¹Y is CH, Y² is N, X is —OCH₂— and R² is methyl, phenyl or 2-methylpyrid-2yl then R¹ is not methyl or phenyl.

In another particular class of compound of formula (I), or a salt, ester or prodrug thereof;

m is 0, 1, 2, 3 or 4;
X is a linker group selected from —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)NR⁵CR⁶R⁷—, —S(O)₂NR⁴CR⁶R⁷ and —NR⁴C(O)—;

### ¹Y is CR⁸ and Y² is N;

R¹ is a group selected from C₁₋₄alkyl, C₃₋₆cycloalkyl, aryl, C₃₋₆cycloalkylC₁₋₄alkyl, arylC₁₋₄alkyl, cycloheteroalkyl, heteroaryl, cycloheteroalkylC₁₋₄alkyl, heteroarylC₁₋₄alkyl, which group is optionally substituted by one or more substituent group selected from halo, cyano, nitro, R⁹, —OR⁹, —COR⁹, —CONR⁹R¹⁰, —NR⁹R¹⁰ and —NR⁹COR¹⁰.
R² is selected from aryl and heteroaryl which group is optionally substituted by one or more substituent group independently selected from halo, cyano, nitro, —R¹¹, —OR¹¹, —COR¹¹, —CONR¹¹R¹², —NR¹¹R¹² and —NR¹¹COR¹².
each R³, when present, is independently selected from halo, cyano, nitro, —R¹³, —OR¹³, —COR¹³, —CONR¹³R¹⁴, —NR¹³R¹⁴ and —NR¹³COR¹⁴;
R⁴ and R⁵ are independently hydrogen or C₁₋₆alkyl;
R⁶ and R⁷ are independently selected from hydrogen, halo, cyano, nitro and C₁₋₆alkyl;
R⁸ is selected from hydrogen, halo, cyano and C₁₋₆alkyl;
R⁹ and R¹⁰ are independently hydrogen or a group selected from C₁₋₆alkyl, carbocyclyl and heterocyclyl which group is optionally substituted by one or more substituent groups selected from halo, cyano, nitro, hydroxy, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino and bis(C₁₋₆alkyl)amino;
R¹¹ and R¹² are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino,
C₁₋₆alkylamino and bis(C₁₋₆alkyl)amino;
R¹³ and R¹⁴ are independently hydrogen or a group selected from C₁₋₆alkyl, carbocyclyl and heterocyclyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino,
C₁₋₆alkylamino and bis(C₁₋₆alkyl)amino;
provided that
(a) when ¹Y is CH, Y² is N, X is —SCH₂—, —S(O)CH₂— or —S(O)₂CH₂— and R² is methyl, phenyl or pyridyl, then R¹ is not methyl, phenyl, 4-chlorophenyl or 4-chlorobenzyl; and
(b) when ¹Y is CH, Y² is N, X is —OCH₂— and R² is methyl, phenyl or 2-methylpyrid-2yl then R¹ is not methyl or phenyl.

In a further particular class of compound of formula (I), or a salt, ester or prodrug thereof;

m is 0 so that R³ is absent
X is a linker group selected from —NR⁴CR⁶R⁷—, —OCR⁶R⁷—, —SCR⁶R⁷—, —S(O)CR⁶R⁷— and —S(O)₂CR⁶R⁷—.

### ¹Y is CH or CF and Y² is N.

R¹ is a group selected from methyl, ethyl, propyl, butyl, isobutyl, tert-butyl, cyclohexyl, phenyl, benzyl, phenethyl, pyridinyl, pyrazolylethyl, furanylmethyl, thienylmethyl, and pyrazinylethyl, which group is optionally substituted by 1 or 2 substituent group selected from halo, cyano, methyl, methoxy, trifluoromethyl, trifluoromethoxy, —CONH₂ and —CONHCH₃.
R² is selected from phenyl, naphthyl, pyrrolyl, imidazolyl, pyrazolyl, furanyl, thienyl, pyridinyl, pyrimidinyl, pyridazinyl, idolyl, quinolinyl, benzofuranyl, dibenzofuranyl, benzothienyl which group is optionally substituted by one or more substituent group independently selected from halo, methyl, methoxy, hydroxymethyl, cyanomethyl, phenoxy, pyrrolidinyl, —CONH₂, —CONHCH₃ and —CON(CH₃)₂.
R⁴ is hydrogen or methyl;
R⁶ is hydrogen or methyl;
R⁷ is hydrogen or methyl;
provided that
(a) when ¹Y is CH, Y² is N, X is —SCH₂—, —S(O)CH₂— or —S(O)₂CH₂— and R² is methyl, phenyl or pyridyl, then R¹ is not methyl, phenyl, 4-chlorophenyl or 4-chlorobenzyl; and
(b) when ¹Y is CH, Y² is N, X is —OCH₂— and R² is methyl, phenyl or 2-methylpyrid-2yl then R¹ is not methyl or phenyl.

Another aspect of the invention provides a compound, or a combination of compounds, selected from:
4-(methylsulfonylmethyl)-6-morpholin-4-yl-2-thiophen-3-yl-pyrimidine;
2-benzofuran-2-yl-4-(methylsulfonylmethyl)-6-morpholin-4-yl-pyrimidine;
2-dibenzofuran-1-yl-4-(methylsulfonylmethyl)-6-morpholin-4-yl-pyrimidine;
5-[4-(methylsulfonylmethyl)-6-morpholin-4-yl-pyrimidin-2-yl]-1H-indole;
2-(6-methoxypyridin-3-yl)-4-(methylsulfonylmethyl)-6-morpholin-4-yl-pyrimidine;
2-(6-methoxynaphthalen-2-yl)-4-(methylsulfonylmethyl)-6-morpholin-4-yl-pyrimidine;
[3-[4-(methylsulfonylmethyl)-6-morpholin-4-yl-pyrimidin-2-yl]phenyl]methanol;
[4-[4-(methylsulfonylmethyl)-6-morpholin-4-yl-pyrimidin-2-yl]phenyl]methanol;
N,N-dimethyl-4-[4-(methylsulfonylmethyl)-6-morpholin-4-yl-pyrimidin-2-yl]-benzamide;
2-(2-methoxypyrimidin-5-yl)-4-(methyl sulfonylmethyl)-6-morpholin-4-yl-pyrimidine;
6-[4-(methylsulfonylmethyl)-6-morpholin-4-yl-pyrimidin-2-yl]quinoline;
3-[4-(methylsulfonylmethyl)-6-morpholin-4-yl-pyrimidin-2-yl]benzamide;
4-(benzenesulfonylmethyl)-6-morpholin-4-yl-2-thiophen-3-yl-pyrimidine;
4-(benzenesulfonylmethyl)-2-(3,4-dimethoxyphenyl)-6-morpholin-4-yl-pyrimidine;
4-(benzenesulfonylmethyl)-2-(3-furyl)-6-morpholin-4-yl-pyrimidine;
4-(benzenesulfonylmethyl)-2-benzothiophen-3-yl-6-morpholin-4-yl-pyrimidine;
4-(benzenesulfonylmethyl)-6-morpholin-4-yl-2-(4-phenoxyphenyl)pyrimidine;
2-[4-[4-(benzenesulfonylmethyl)-6-morpholin-4-yl-pyrimidin-2-yl]phenyl]acetonitrile;
4-(benzenesulfonylmethyl)-2-(3-fluoro-4-methoxy-phenyl)-6-morpholin-4-yl-pyrimidine;
[5-[4-(benzenesulfonylmethyl)-6-morpholin-4-yl-pyrimidin-2-yl]thiophen-2-yl]methanol;
4-(benzenesulfonylmethyl)-6-morpholin-4-yl-2-(3-pyrrolidin-1-ylphenyl)pyrimidine;
5-[4-(benzenesulfonylmethyl)-6-morpholin-4-yl-pyrimidin-2-yl]-1-methyl-indole;
5-[4-(benzenesulfonylmethyl)-6-morpholin-4-yl-pyrimidin-2-yl]-1H-indole;
4-(benzenesulfonylmethyl)-2-(6-methoxypyridin-3-yl)-6-morpholin-4-yl-pyrimidine;
4-morpholin-4-yl-6-(phenylsulfanylmethyl)-2-pyridin-2-yl-pyrimidine;
4-(2-furylmethylsulfanylmethyl)-6-morpholin-4-yl-2-pyridin-2-yl-pyrimidine;
4-[(4-methoxyphenyl)sulfanylmethyl]-6-morpholin-4-yl-2-pyridin-2-yl-pyrimidine;
4-(butan-2-ylsulfanylmethyl)-6-morpholin-4-yl-2-pyridin-2-yl-pyrimidine;
4-(butylsulfanylmethyl)-6-morpholin-4-yl-2-pyridin-2-yl-pyrimidine;
4-morpholin-4-yl-2-pyridin-2-yl-6-(tert-butylsulfanylmethyl)pyrimidine;
4-morpholin-4-yl-6-(propan-2-ylsulfanylmethyl)-2-pyridin-2-yl-pyrimidine;
4-[(2-chloro-6-fluoro-phenyl)methylsulfanylmethyl]-6-morpholin-4-yl-2-pyridin-2-yl-pyrimidine;
4-(cyclohexylsulfanylmethyl)-6-morpholin-4-yl-2-pyridin-2-yl-pyrimidine;
4-[(4-fluorophenyl)sulfanylmethyl]-6-morpholin-4-yl-2-pyridin-2-yl-pyrimidine;
4-(ethylsulfanylmethyl)-6-morpholin-4-yl-2-pyridin-2-yl-pyrimidine;
4-[(4-fluorophenyl)methylsulfanylmethyl]-6-morpholin-4-yl-2-pyridin-2-yl-pyrimidine;
4-[(4-methoxyphenyl)methylsulfanylmethyl]-6-morpholin-4-yl-2-pyridin-2-yl-pyrimidine;
4-morpholin-4-yl-6-(phenethylsulfanylmethyl)-2-pyridin-2-yl-pyrimidine;
4-[(6-morpholin-4-yl-2-pyridin-2-yl-pyrimidin-4-yl)methylsulfanyl]benzonitrile;
4-(2-methylpropylsulfanylmethyl)-6-morpholin-4-yl-2-pyridin-2-yl-pyrimidine;
4-morpholin-4-yl-6-(2-pyrazin-2-ylethylsulfanylmethyl)-2-pyridin-2-yl-pyrimidine;
4-morpholin-4-yl-2-pyridin-2-yl-6-(thiophen-2-ylmethylsulfanylmethyl)pyrimidine;
4-(2-furylmethylsulfonylmethyl)-6-morpholin-4-yl-2-pyridin-2-yl-pyrimidine;
4-[(4-methoxyphenyl)sulfonylmethyl]-6-morpholin-4-yl-2-pyridin-2-yl-pyrimidine;
4-(butan-2-ylsulfonylmethyl)-6-morpholin-4-yl-2-pyridin-2-yl-pyrimidine;
4-(2-methylpropylsulfonylmethyl)-6-morpholin-4-yl-2-pyridin-2-yl-pyrimidine;
4-morpholin-4-yl-6-(propylsulfonylmethyl)-2-pyridin-2-yl-pyrimidine;
4-(butylsulfonylmethyl)-6-morpholin-4-yl-2-pyridin-2-yl-pyrimidine;
4-morpholin-4-yl-6-(propan-2-ylsulfonylmethyl)-2-pyridin-2-yl-pyrimidine;
4-morpholin-4-yl-2-pyridin-2-yl-6-[[3-(trifluoromethyl)phenyl]sulfonylmethyl]pyrimidine;
4-morpholin-4-yl-6-(2-pyrazin-2-ylethylsulfonylmethyl)-2-pyridin-2-yl-pyrimidine;
4-morpholin-4-yl-2-pyridin-2-yl-6-(thiophen-2-ylmethylsulfonylmethyl)pyrimidine;
4-(cyclohexylsulfonylmethyl)-6-morpholin-4-yl-2-pyridin-2-yl-pyrimidine;
4-[(4-fluorophenyl)sulfonylmethyl]-6-morpholin-4-yl-2-pyridin-2-yl-pyrimidine;
4-(ethylsulfonylmethyl)-6-morpholin-4-yl-2-pyridin-2-yl-pyrimidine;
4-[(4-fluorophenyl)methylsulfonylmethyl]-6-morpholin-4-yl-2-pyridin-2-yl-pyrimidine;
4-morpholin-4-yl-2-pyridin-2-yl-6-[[4-(trifluoromethoxy)phenyl]sulfonylmethyl]pyrimidine;
4-[(4-methoxyphenyl)methylsulfonylmethyl]-6-morpholin-4-yl-2-pyridin-2-yl-pyrimidine;
4-[(3,4-dimethoxyphenyl)sulfonylmethyl]-6-morpholin-4-yl-2-pyridin-2-yl-pyrimidine;
4-[(4-bromo-2-fluoro-phenyl)sulfonylmethyl]-6-morpholin-4-yl-2-pyridin-2-yl-pyrimidine;
N-methyl-2-[(6-morpholin-4-yl-2-pyridin-2-yl-pyrimidin-4-yl)methylsulfonyl]benzamide;
4-morpholin-4-yl-6-(phenethylsulfonylmethyl)-2-pyridin-2-yl-pyrimidine;
4-morpholin-4-yl-2-pyridin-2-yl-6-[2-[3-(trifluoromethyl)phenyl]ethylsulfonylmethyl]pyrimidine;
4-[(6-morpholin-4-yl-2-pyridin-2-yl-pyrimidin-4-yl)methylsulfonyl]benzonitrile;
4-[(2-chloro-4-fluoro-phenyl)sulfonylmethyl]-6-morpholin-4-yl-2-pyridin-2-yl-pyrimidine;
4-[(3-methoxyphenoxy)methyl]-6-morpholin-4-yl-2-pyridin-2-yl-pyrimidin;
4-morpholin-4-yl-6-(phenoxymethyl)-2-pyridin-2-yl-pyrimidine;
4-morpholin-4-yl-6-(phenylmethoxymethyl)-2-pyridin-2-yl-pyrimidine;
4-(ethoxymethyl)-6-morpholin-4-yl-2-pyridin-2-yl-pyrimidine;
4-[(2-chlorophenoxy)methyl]-6-morpholin-4-yl-2-pyridin-2-yl-pyrimidine;
4-[(3-chlorophenoxy)methyl]-6-morpholin-4-yl-2-pyridin-2-yl-pyrimidine;
4-[(3-methoxyphenoxy)methyl]-6-morpholin-4-yl-2-pyridin-2-yl-pyrimidine;
4-[(4-methoxyphenoxy)methyl]-6-morpholin-4-yl-2-pyridin-2-yl-pyrimidine;
4-[(2-chlorophenyl)methoxymethyl]-6-morpholin-4-yl-2-pyridin-2-yl-pyrimidine;
3-[(6-morpholin-4-yl-2-pyridin-2-yl-pyrimidin-4-yl)methoxy]pyridine-2-carboxamide;
4-[(2-methylpyridin-3-yl)oxymethyl]-6-morpholin-4-yl-2-pyridin-2-yl-pyrimidine;
4-morpholin-4-yl-2-pyridin-2-yl-6-(pyridin-3-yloxymethyl)pyrimidine;
N-benzyl-N-methyl-1-(6-morpholin-4-yl-2-pyridin-2-yl-pyrimidin-4-yl)methanamine;
N-[(6-morpholin-4-yl-2-pyridin-2-yl-pyrimidin-4-yl)methyl]propan-2-amine;
1-(2-chlorophenyl)-N-[(6-morpholin-4-yl-2-pyridin-2-yl-pyrimidin-4-yl)methyl]methanamine;
4-(benzenesulfonylmethyl)-5-fluoro-6-morpholin-4-yl-2-pyridin-2-yl-pyrimidine;
5-fluoro-4-(methylsulfonylmethyl)-6-morpholin-4-yl-2-pyridin-2-yl-pyrimidine;
6-morpholin-4-yl-N-phenyl-2-pyridin-2-yl-pyrimidine-4-carboxamide;
N,N-dimethyl-6-morpholin-4-yl-2-pyridin-2-yl-pyrimidine-4-carboxamide;
5-[4-(methylsulfonylmethyl)-6-morpholin-4-yl-pyrimidin-2-yl]-1,3-dihydroindol-2-one;
methyl 2-amino-5-[4-(methylsulfonylmethyl)-6-morpholin-4-yl-pyrimidin-2-yl]benzoate;
[2-methoxy-5-[4-(methylsulfonylmethyl)-6-morpholin-4-yl-pyrimidin-2-yl]phenyl]methanol;
2-methyl-5-[4-(methylsulfonylmethyl)-6-morpholin-4-yl-pyrimidin-2-yl]-1H-benzoimidazole;
5-[4-(methylsulfonylmethyl)-6-morpholin-4-yl-pyrimidin-2-yl]-1,3-dihydrobenzoimidazol-2-one;
[5-[4-(methylsulfonylmethyl)-6-morpholin-4-yl-pyrimidin-2-yl]-1H-indazol-3-yl]methanol;
6-[4-(methylsulfonylmethyl)-6-morpholin-4-yl-pyrimidin-2-yl]chroman-4-ol;
1-acetyl-5-[4-(methylsulfonylmethyl)-6-morpholin-4-yl-pyrimidin-2-yl]-2H-indol-3-one;
1-methyl-4-[4-(methylsulfonylmethyl)-6-morpholin-4-yl-pyrimidin-2-yl]piperazin-2-one;
1-(4-chlorophenyl)-4-[4-(methylsulfonylmethyl)-6-morpholin-4-yl-pyrimidin-2-yl]piperazin-2-one;
2-[3-(4,4-dimethyl-5H-1,3-oxazol-2-yl)-4-methoxy-phenyl]-4-(methylsulfonylmethyl)-6-morpholin-4-yl-pyrimidine;
N-(1H-benzoimidazol-5-yl)-2,6-dimorpholin-4-yl-pyrimidine-4-carboxamide;
N-(5-methyl-2H-pyrazol-3-yl)-2,6-dimorpholin-4-yl-pyrimidine-4-carboxamide;
N-(1H-indol-5-yl)-2,6-dimorpholin-4-yl-pyrimidine-4-carboxamide;
N-[5-(methoxymethyl)-1,3,4-thiadiazol-2-yl]-2,6-dimorpholin-4-yl-pyrimidine-4-carboxamide;
5-[4-(methylsulfonylmethyl)-6-morpholin-4-yl-pyrimidin-2-yl]-1H-indazole;
3-methyl-5-[4-(methylsulfonylmethyl)-6-morpholin-4-yl-pyrimidin-2-yl]-1H-indazole;
5-[2-(methylsulfonylmethyl)-6-morpholin-4-yl-pyrimidin-4-yl]-1H-indole;
5-[4-(methylsulfonylmethyl)-6-morpholin-4-yl-pyrimidin-2-yl]-1H-benzoimidazole;
4-[4-(methylsulfonylmethyl)-6-morpholin-4-yl-pyrimidin-2-yl]-1H-indole;
3-[4-(methylsulfonylmethyl)-6-morpholin-4-yl-pyrimidin-2-yl]-5,7-diazabicyclo[4.3.0]nona-1,3,5,8-tetraene;
4-[4-(methylsulfonylmethyl)-6-morpholin-4-yl-pyrimidin-2-yl]aniline;
2-(1H-indol-5-yl)-6-morpholin-4-yl-pyrimidine-4-carboxylic acid;
[2-(1H-indol-5-yl)-6-morpholin-4-yl-pyrimidin-4-yl]methanol;
5-[4-morpholin-4-yl-6-(morpholin-4-ylmethyl)pyrimidin-2-yl]-1H-indole;
N-[[2-(1H-indol-5-yl)-6-morpholin-4-yl-pyrimidin-4-yl]methyl]-1-(4-methoxyphenyl)methanamine;
1-(4-chlorophenyl)-N-[[2-(1H-indol-5-yl)-6-morpholin-4-yl-pyrimidin-4-yl]methyl]methanamine;
5-[4-[(2-methylpyridin-3-yl)oxymethyl]-6-morpholin-4-yl-pyrimidin-2-yl]-1H-1-indole;
5-[4-(methoxymethyl)-6-morpholin-4-yl-pyrimidin-2-yl]-1H-indole;
5-[4-(2-furylmethylsulfonylmethyl)-6-morpholin-4-yl-pyrimidin-2-yl]-1H-indole;
5-[4-(ethylsulfonylmethyl)-6-morpholin-4-yl-pyrimidin-2-yl]-1H-indole;
5-[4-[(4-methoxyphenyl)sulfonylmethyl]-6-morpholin-4-yl-pyrimidin-2-yl]-1H-1-indole;
5-[4-morpholin-4-yl-6-(propan-2-ylsulfonylmethyl)pyrimidin-2-yl]-1H-indole;
5-[4-(butan-2-ylsulfonylmethyl)-6-morpholin-4-yl-pyrimidin-2-yl]-1H-indole;
5-[4-[(2-chloro-4-fluoro-phenyl)sulfonylmethyl]-6-morpholin-4-yl-pyrimidin-2-yl]-1H-indole;
2-[[2-(1H-indol-5-yl)-6-morpholin-4-yl-pyrimidin-4-yl]methyl sulfonyl]-N,N-dimethyl-acetamide;
5-[4-[(5-chloro-1,2,4-thiadiazol-3-yl)methyl sulfonylmethyl]-6-morpholin-4-yl-pyrimidin-2-yl]-1H-indole;
5-[4-morpholin-4-yl-6-(1,3-thiazol-4-ylmethylsulfonylmethyl)pyrimidin-2-yl]-1H-indole;
3-[[2-(1H-indol-5-yl)-6-morpholin-4-yl-pyrimidin-4-yl]methylsulfonyl]propanenitrile;
2-[[2-(1H-indol-5-yl)-6-morpholin-4-yl-pyrimidin-4-yl]methylsulfonyl]-1-morpholin-4-yl-ethanone;
5-[4-[(3,5-dimethyl-1,2-oxazol-4-yl)methylsulfonylmethyl]-6-morpholin-4-yl-pyrimidin-2-yl]-1H-indole;
(2S)-1-[2-[[2-(1H-indol-5-yl)-6-morpholin-4-yl-pyrimidin-4-yl]methylsulfonyl]acetyl]pyrrolidine-2-carbonitrile;
5-[4-morpholin-4-yl-6-(pyridin-3-ylmethylsulfonylmethyl)pyrimidin-2-yl]-1H-indole;
5-[4-(2-imidazol-1-ylethylsulfonylmethyl)-6-morpholin-4-yl-pyrimidin-2-yl]-1H-indole;
5-[4-[(5-ethyl-1H-imidazol-4-yl)methylsulfonylmethyl]-6-morpholin-4-yl-pyrimidin-2-yl]-1H-indole;
5-[4-(2-fluoro ethyl sulfonylmethyl)-6-morpholin-4-yl-pyrimidin-2-yl]-1H-indole;
4-[[2-(1H-indol-5-yl)-6-morpholin-4-yl-pyrimidin-4-yl]methylsulfonylmethyl]-2H-phthalazin-1-one;
4-[[2-(1H-indol-5-yl)-6-morpholin-4-yl-pyrimidin-4-yl]methylsulfonyl]butanenitrile;
2-[[2-(1H-indol-5-yl)-6-morpholin-4-yl-pyrimidin-4-yl]methylsulfonyl]-1-pyrrolidin-1-yl-ethanone;
2-[[2-(1H-indol-5-yl)-6-morpholin-4-yl-pyrimidin-4-yl]methylsulfonyl]-N-propan-2-yl-acetamide;
5-[4-[2-(2-methoxyethoxy)ethylsulfonylmethyl]-6-morpholin-4-yl-pyrimidin-2-yl]-1H-indole;
5-[4-[(2-methyl-1,3-thiazol-4-yl)methylsulfonylmethyl]-6-morpholin-4-yl-pyrimidin-2-yl]-1H-indole;
2-[[2-(1H-indol-5-yl)-6-morpholin-4-yl-pyrimidin-4-yl]methylsulfonyl]-N-propyl-acetamide;
5-[4-(2,2-difluoroethylsulfonylmethyl)-6-morpholin-4-yl-pyrimidin-2-yl]-1H-indole;
5-[4-morpholin-4-yl-6-[(5-tert-butyl-1,3,4-thiadiazol-2-yl)methylsulfonylmethyl]pyrimidin-2-yl]-1H-indole;
5-[4-(3-methoxypropylsulfonylmethyl)-6-morpholin-4-yl-pyrimidin-2-yl]-1H-indole;
5-[4-morpholin-4-yl-6-(prop-2-ynylsulfonylmethyl)pyrimidin-2-yl]-1H-indole;
5-[4-morpholin-4-yl-6-(2-morpholin-4-ylethylsulfonylmethyl)pyrimidin-2-yl]-1H-indole;
N-[4-[[2-(1H-indol-5-yl)-6-morpholin-4-yl-pyrimidin-4-yl]methylsulfonylmethyl]phenyl]acetamide;
2-[[2-(1H-indol-5-yl)-6-morpholin-4-yl-pyrimidin-4-yl]methylsulfonyl]-N-tert-butyl-acetamide;
5-[4-morpholin-4-yl-6-(3-morpholin-4-ylpropylsulfonylmethyl)pyrimidin-2-yl]-1H-indole;
2-[[2-(1H-indol-5-yl)-6-morpholin-4-yl-pyrimidin-4-yl]methylsulfonyl]-1-(1-piperidyl)ethanone;
5-[4-(2-ethoxyethylsulfonylmethyl)-6-morpholin-4-yl-pyrimidin-2-yl]-1H-indole;
5-[4-morpholin-4-yl-6-(oxolan-2-ylmethylsulfonylmethyl)pyrimidin-2-yl]-1H-indole;
3-[[2-(1H-indol-5-yl)-6-morpholin-4-yl-pyrimidin-4-yl]methylsulfonyl]-N,N-dimethyl-propan-1-amine;
N,N-diethyl-2-[[2-(1H-indol-5-yl)-6-morpholin-4-yl-pyrimidin-4-yl]methylsulfonyl]acetamide;
5-[4-morpholin-4-yl-6-(propylsulfonylmethyl)pyrimidin-2-yl]-1H-indole;
2-[[2-(1H-indol-5-yl)-6-morpholin-4-yl-pyrimidin-4-yl]methylsulfonylmethyl]-1H-benzoimidazole;
3-[[2-(1H-indol-5-yl)-6-morpholin-4-yl-pyrimidin-4-yl]methylsulfonylmethyl]benzonitrile;
8-[[2-(1H-indol-5-yl)-6-morpholin-4-yl-pyrimidin-4-yl]methylsulfonylmethyl]-5-methyl-1,7-diazabicyclo[4.3.0]nona-2,4,6,8-tetraene;
N-benzyl-2-[[2-(1H-indol-5-yl)-6-morpholin-4-yl-pyrimidin-4-yl]methylsulfonyl]acetamide;
2-[[2-(1H-indol-5-yl)-6-morpholin-4-yl-pyrimidin-4-yl]methylsulfonyl]-N-methyl-N-phenyl-acetamide;
5-[4-(butylsulfonylmethyl)-6-morpholin-4-yl-pyrimidin-2-yl]-1H-indole;
5-[4-[(5-methyl-1,3,4-oxadiazol-2-yl)methylsulfonylmethyl]-6-morpholin-4-yl-pyrimidin-2-yl]-1H-indole;
2-[[2-(1H-indol-5-yl)-6-morpholin-4-yl-pyrimidin-4-yl]methylsulfonyl]acetamide;
3-[[2-(1H-indol-5-yl)-6-morpholin-4-yl-pyrimidin-4-yl]methylsulfonyl]propanamide;
2-[[2-(1H-indol-5-yl)-6-morpholin-4-yl-pyrimidin-4-yl]methylsulfonyl]acetonitrile;
5-amino-1-[2-[[2-(1H-indol-5-yl)-6-morpholin-4-yl-pyrimidin-4-yl]methylsulfonyl]ethyl]pyrazole-4-carbonitrile;
2-[[2-(1H-indol-5-yl)-6-morpholin-4-yl-pyrimidin-4-yl]methylsulfonyl]-N-(2-methoxyethyl)acetamide;
5-[4-(2-cyclohexylethylsulfonylmethyl)-6-morpholin-4-yl-pyrimidin-2-yl]-1H-indole;
5-[4-[3-(4-chlorophenyl)propylsulfonylmethyl]-6-morpholin-4-yl-pyrimidin-2-yl]-1H-indole;
N-[2-[[2-(1H-indol-5-yl)-6-morpholin-4-yl-pyrimidin-4-yl]methylsulfonyl]ethyl]acetamide;
2-[[2-(1H-indol-5-yl)-6-morpholin-4-yl-pyrimidin-4-yl]methylsulfonylmethyl]-3H-quinazolin-4-one;
5-[4-(cyclohexylmethylsulfonylmethyl)-6-morpholin-4-yl-pyrimidin-2-yl]-1H-indole;
5-[4-[3-(4-fluorophenoxy)propylsulfonylmethyl]-6-morpholin-4-yl-pyrimidin-2-yl]-1H-indole;
5-[4-(5-methylhexylsulfonylmethyl)-6-morpholin-4-yl-pyrimidin-2-yl]-1H-indole;
4-morpholin-4-yl-2-pyridin-2-yl-6-(tert-butylsulfonylmethyl)pyrimidine;
2-methyl-5-[4-(methylsulfonylmethyl)-6-morpholin-4-yl-pyrimidin-2-yl]-1H-indole;
4-[(5-methyl-2H-pyrazol-3-yl)oxymethyl]-6-morpholin-4-yl-2-pyridin-2-yl-pyrimidine;
2-(3-furyl)-4-(methylsulfonylmethyl)-6-morpholin-4-yl-pyrimidine;
4-(methylsulfonylmethyl)-6-morpholin-4-yl-2-naphthalen-1-yl-pyrimidine;
   or a salt, ester or prodrugs thereof and particularly a pharmaceutically salt thereof.

Further compounds of the invention include:
N-(1H-benzoimidazol-5-yl)-2,6-dimorpholin-4-yl-pyrimidine-4-carboxamide;
N-(5-methyl-2H-pyrazol-3-yl)-2,6-dimorpholin-4-yl-pyrimidine-4-carboxamide;
   or a salt, ester or prodrugs thereof and particularly a pharmaceutically salt thereof.

In certain aspects of the invention such as a compound of formula (I) for use as a medicament for the treatment of proliferative disease; or the use of a compound of formula (I) in the manufacture of a medicament for use in the treatment of proliferative disease; a compound of formula (I) may be 4-morpholin-4-yl-6-(phenylsulfonylmethyl)-2-pyridin-4-yl-pyrimidine or 4-{6-[(phenylsulfonyl)methyl]-2-pyridin-2-ylpyrimidin-4-yl}morpholine.

The invention also provides processes for the preparation of a compound of formula (I) or a salt, ester or prodrug thereof.

A compound of formula (I), wherein X is —S(O)₂CR⁶R⁷—, may be prepared by oxidising a compound of formula (I), wherein X is —SCR⁶R⁷—, for example by using Oxone® at room temperature in a mixed solvent system of water and ethanol.

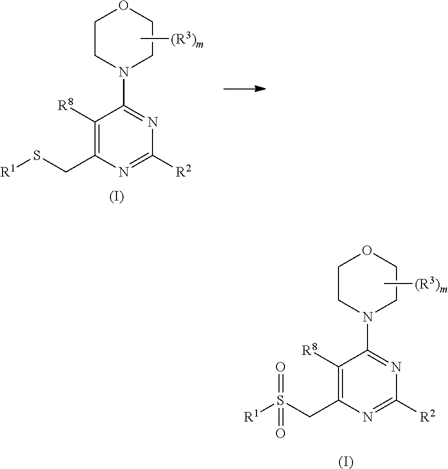

According to a further aspect of the present invention there is provided a process for preparing a compound of formula (I) as defined in Claim 1, wherein X is —S(O)₂CR⁶R⁷—, by reacting a compound of formula (I), wherein X is —SCR⁶R⁷—, with an oxidising agent (for example by using Oxone® at room temperature in a mixed solvent system of water and ethanol).

A compound of formula (I), wherein X is —X¹CR⁶R⁷— and X¹ is —NR⁴—, —O—, —S—, —S(O)—, or —S(O)₂— may be prepared from a compound of formula (II), wherein L¹ is a leaving group such as halo (for example chloro), tosyl, mesyl etc., by reaction with a compound of formula (III) in the presence of a suitable base such as triethylamine and a solvent such as tetrahydrofuran or N,N-dimethylformamide:

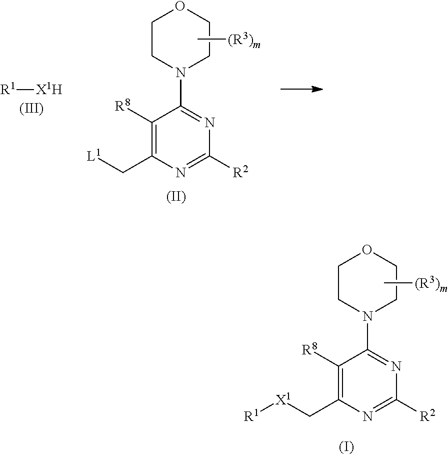

According to a further aspect of the present invention there is provided a process for preparing a compound of formula (I) according to Claim 1, wherein X is —X¹CR⁶R⁷— and and X¹ is —NR⁴—, —O—, —S—, —S(O)—, or —S(O)₂—,

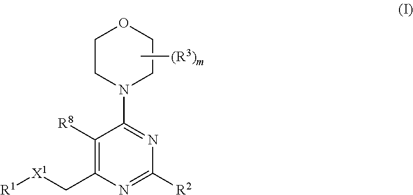

comprising reaction a compound of formula (II), wherein L¹ is a leaving group (such as halo (for example chloro), tosyl, mesyl etc.,)

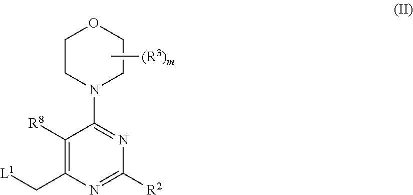

with a compound of formula (III)

R¹—X¹H (III)

(optionally in the presence of a suitable base such as triethylamine and a solvent such as tetrahydrofuran or N,N-dimethylformamide).

A compound of formula (II) may be prepared from a compound of formula (IV), wherein L² is a leaving group such as halo (for example chloro), tosyl, mesyl etc.:

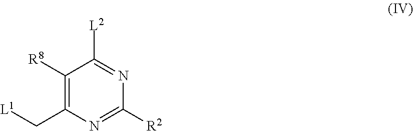

by reaction with a compound of formula (V)

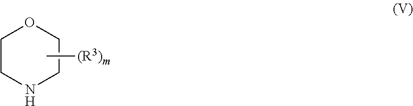

This reaction may be performed in solvent such as tetrahydrofuran in the presence of a suitable base such as triethylamine.

Compounds of formula (V) are commercially available or may be prepared using convenient methods described in the literature, known to the skilled person or described in the Examples herein.

A compound of formula (IV) may be prepared from a compound of formula (VI):

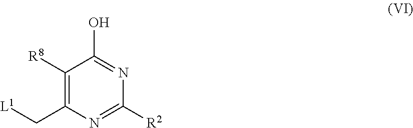

When L² is halo such as chloro, a compound of formula (IV) may be prepared using a chlorinating agent such as phosphorous oxychloride at a high temperature such as from 50° C. to 150° C., particularly from 75° C. to 125° C. and more particularly at approximately 100° C.

A compound of formula (VI) may be prepared by reacting a compound of formula (VII):

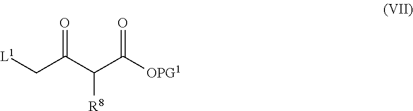

with a compound of formula (VIII)

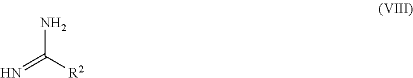

Compounds of formula (VII) and compounds of formula (VIII) are commercially available or may be prepared using convenient methods described in the literature, known to the skilled person or described in the Examples herein.

A compound of formula (I), wherein X is —S(O)₂CR⁶R⁷—, may also be prepared by reacting a compound of formula (IX) with a suitable organo-metallic reagent (such as the activated ester of boronic acid R²B(OR)₃ wherein R is C₁₋₄alkyl such as methyl), in the presence of a suitable metal catalyst (such as palladium or copper) using a solvent (such as an organic solvent eg 1,4-dioxane).

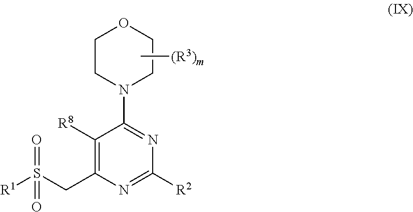

A compound of formula (IX) may be prepared by reacting a compound of formula (X)

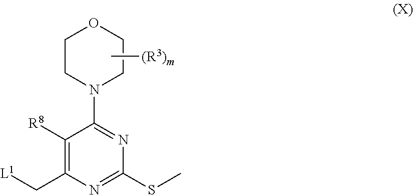

with a compound of formula (XI) in solvent such as tetrahydrofuran or N,N-dimethylformamide.

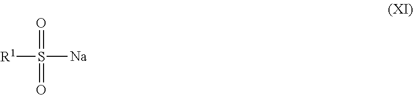

A compound of formula (X) may be prepared by reacting a compound of formula (XII)

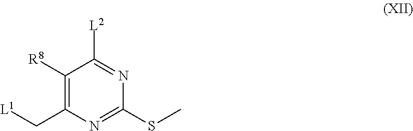

with a compound of formula (V)

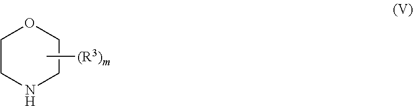

This reaction may be performed in solvent such as tetrahydrofuran in the presence of a suitable base such as triethylamine.

A compound of formula (XII) may be prepared from a compound of formula (XIII):

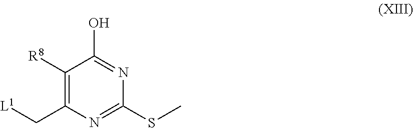

When L² is halo such as chloro, a compound of formula (XII) may be prepared using a chlorinating agent such as phosphorous oxychloride at a high temperature such as from 50° C. to 150° C., particularly from 75° C. to 125° C. and more particularly at approximately 100° C.

A compound of formula (XII) may be prepared by reacting a compound of formula (VII)

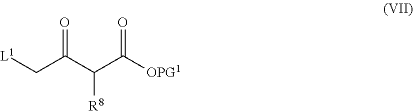

with a compound of formula (XIV)

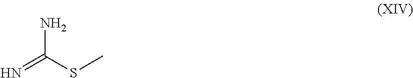

Compounds of formula (VII) and compounds of formula (XIV) are commercially available or may be prepared using convenient methods described in the literature, known to the skilled person or described in the Examples herein.

A compound of formula (I) wherein X is —C(O)NR⁴CR⁶R⁷—, —NR⁴C(O)NR⁵CR⁶R⁷—or

—S(O)₂NR⁴CR⁶R⁷— may be prepared by reacting a compound of formula (I) wherein X is —NH₂CR⁶R⁷— with the appropriate compound of formula (XVI) in the presence of a suitable base such as triethylamine.

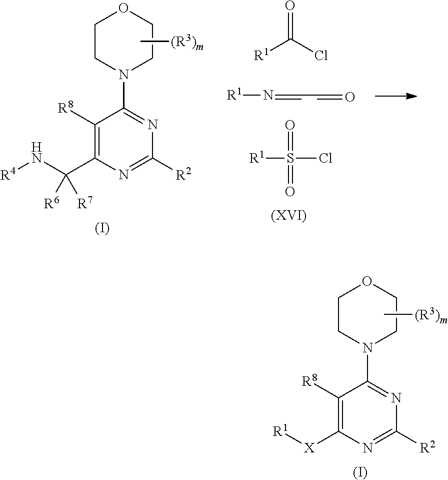

Similarly, a compound of formula (I), wherein X is —C(O)NR⁴—, —NR⁴C(O)NR⁵— or —S(O)₂NR⁴—, may be prepared by reacting a compound of formula (XV) with the appropriate compound of formula (XVI) in the presence of a suitable base such as triethylamine.

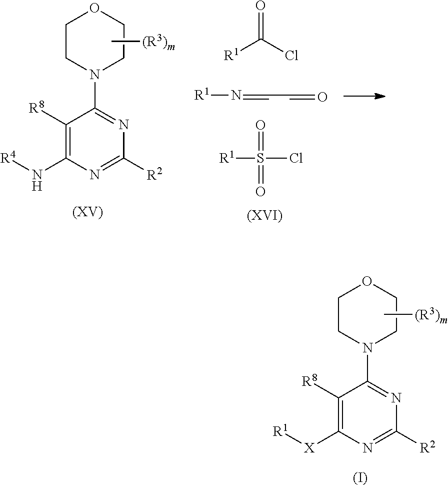

A compound of formula (XV) may be prepared by reacting a compound of formula (XVII) with diphenylphosphoryl azide and triethylamine in a solvent such as N,N-dimethylacetamide.

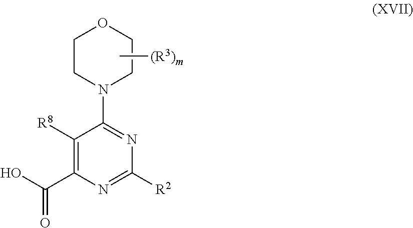

Where R⁴ is C₁₋₆alkyl, this step may be followed by alkylation of the resulting amine using reductive amination conditions, such as an aldehyde in the presence of sodium cyanoborohydride in a solvent such as dichloromethane.

A compound of formula (XVII) may be prepared by reacting a compound of formula (XVIII) with a base such as sodium hydroxide

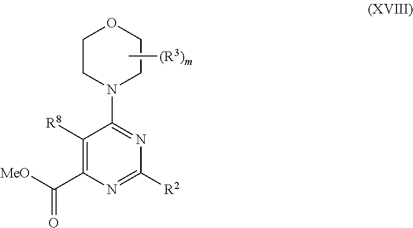

A compound of formula (XVIII) may be prepared by reacting a compound of formula (XIX) wherein L³ is a leaving group such as halo (for example chloro) or trifluoromethane sulfonate.

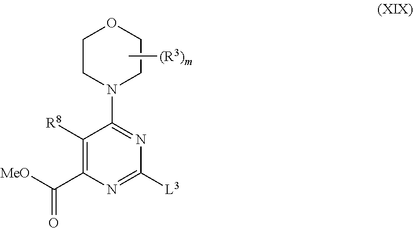

with a suitable organo-metallic reagent such as the tributyltin derivative or the zincate of formula (XX) wherein Y can be halo such as chloro. Where R² is unsaturated such as optionally substituted aryl or heteroaryl, the tributyltin derivative should be used whilst the zincate should be used for cases when R² is saturated.

R²—SnR₃ or R²—Zn—Y (XX)

This reaction is performed in the presence of a suitable metal catalyst such as palladium or copper in a solvent such as tetrahydrofuran, at a high temperature such as 100° C.

A compound of formula (XIX) may be prepared by reacting a compound of formula (XXI) wherein L² is a leaving group such as halo (for example chloro), tosyl, mesyl etc.

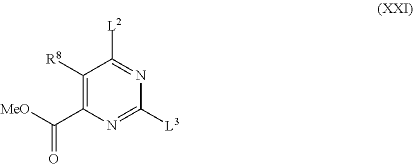

with a compound of formula (V)

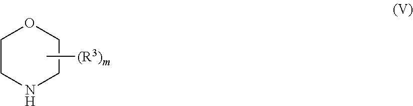

This reaction may be performed in solvent such as tetrahydrofuran in the presence of a suitable base such as triethylamine.

A compound of formula (XXI) may be prepared from a compound of formula (XXII)

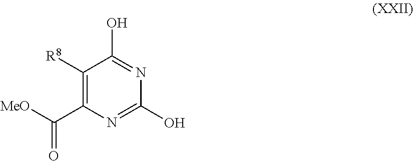

When L² and L³ are chloro, chlorination may be performed using phosphorous oxychloride at a high temperature such as 100° C.

Compounds of formula (VII) and compounds of formula (VIII) are commercially available or may be prepared using convenient methods described in the literature, known to the skilled person or described in the Examples herein.

A compound of formula (I) may also be prepared by reacting a compound of formula (XXIII)

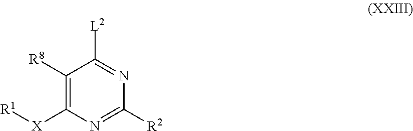

with a compound of formula (V)

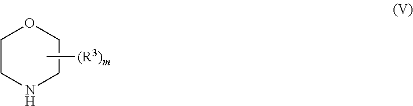

This reaction may be performed in solvent such as tetrahydrofuran in the presence of a suitable base such as triethylamine.

A compound of formula (XXIII), wherein X is —CR⁴═CR⁵—, —CR⁴═CR⁵CR⁶R⁷—, —CR⁶R⁷CR⁵═CR⁴—, —C≡C—, —C≡CCR⁶R⁷— or —CR⁶R⁷C≡C—, may be prepared by reacting a compound of formula (XXIV)

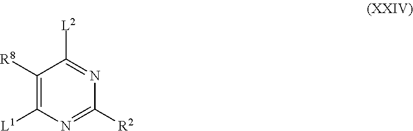

with the appropriate compound of formula (XXV) where M is a metal. For alkynyl compounds M may be hydrogen as well as a metal.

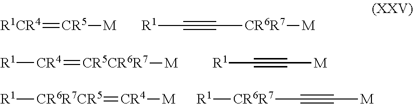

Typically a tributyltin derivative is used in the presence of a suitable metal catalyst such as palladium or copper in a organic solvent such as tetrahydrofuran at a high temperature such as 100° C.

A compound of formula (XXIV) may be prepared from a compound of formula (XXVI)

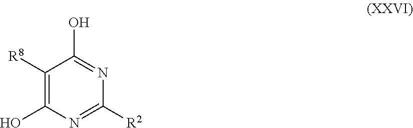

Where L¹ and L² are chloro, a chlorinating agent such as phosphorous oxychloride may be used.

A compound of formula (XXVI) may be prepared by reacting a compound of formula (XXVII) wherein PG¹ and PG² are C₁₋₆alkyl groups such as methyl or ethyl:

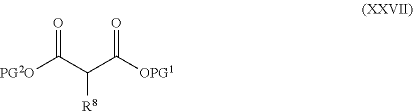

with a compound of formula (VIII)

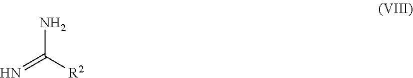

Compounds of formula (XXVII) and compounds of formula (VIII) are commercially available or may be prepared using convenient methods described in the literature, known to the skilled person or described in the Examples herein.

A compound of formula (I) wherein X is —NR⁴C(O)— may be prepared by reacting a compound of formula (XVII)

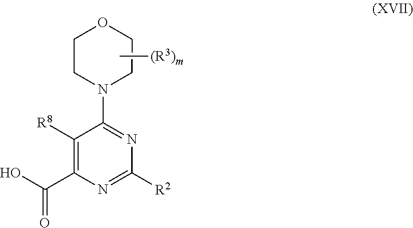

with an amine R⁴NH₂ and a suitable activating reagent such as O-(7-azabenzotriazol-1-yl)-N,N,N′,N′-tetramethyluronium hexafluorophosphate using a base such as diisopropylethyl amine and a solvent such as tetrahydrofuran.

A compound of formula (XVII) may be prepared as described herein.

A compound of formula (I), wherein X is —S(O)₂CR⁶R⁷—, may be prepared by oxidising a compound of formula (I), wherein X is —SCR⁶R⁷—, for example by using Oxone® at room temperature in a mixed solvent system of water and ethanol.

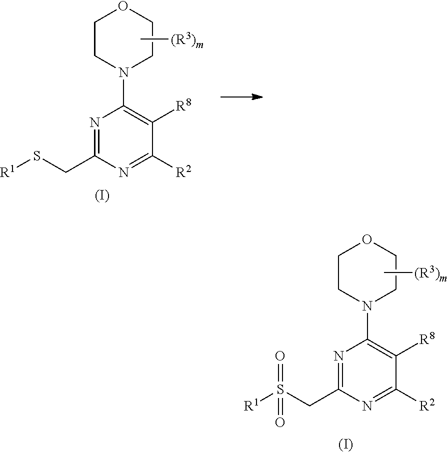

A compound of formula (I), wherein X is —X¹CR⁶R⁷ and X¹ is —NR⁴—, —O—, —S—, —S(O)—, may be prepared by reacting a compound of formula (XXVIII)

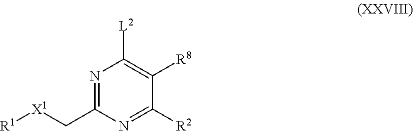

with a compound of formula (V)

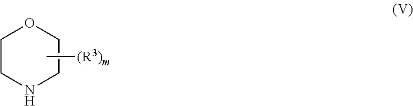

This reaction may be performed in solvent such as tetrahydrofuran in the presence of a suitable base such as triethylamine.

A compound of formula (XXVIII) may be prepared by reacting a compound of formula (XXIX) wherein L³ is a leaving group such as halo (for example chloro),

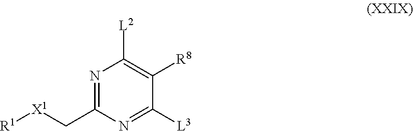

with a suitable organo-metallic reagent such as the tributyltin derivative or the zincate of formula (XX) wherein Y can be halo such as chloro. Where R² is unsaturated such as optionally substituted aryl or heteroaryl, the tributyltin derivative should be used whilst the zincate should be used for cases when R² is saturated.

R²—SnR₃ or R²—Zn—Y (XX)

A compound of formula (XXIX) may be prepared from a compound of formula (XXX)

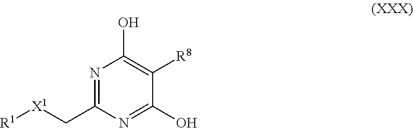

When L² and L³ are chloro, a chlorinating agent such as phosphorous oxychloride may be used.

A compound of formula (XXX) may be prepared by reacting a compound of formula (XXVII) wherein PG¹ and PG² are C₁₋₆alkyl such as methyl or ethyl:

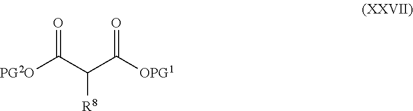

with a compound of formula (XXXI)

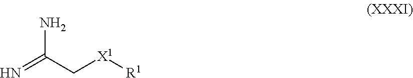

Compounds of formula (XXVII) and compounds of formula (XXXI) are commercially available or may be prepared using convenient methods described in the literature, known to the skilled person or described in the Examples herein.

A compound of formula (I) wherein X is —C(O)NR⁴CR⁶R⁷—, —NR⁴C(O)NR⁵CR⁶R⁷— or

—S(O)₂NR⁴CR⁶R⁷— may be prepared by reacting a compound of formula (I) wherein X is —NH₂CR⁶R⁷— with the appropriate compound of formula (XVI) in the presence of a suitable base such as triethylamine.

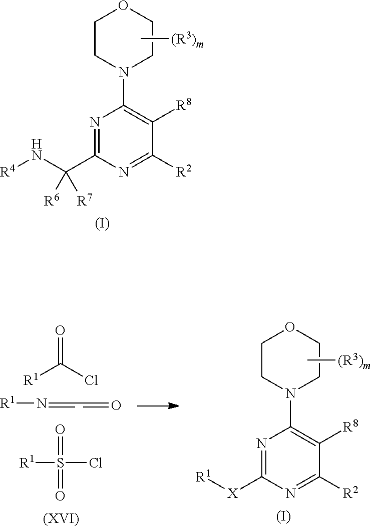

Similarly, a compound of formula (I) wherein X is —C(O)NR⁴—, —NR⁴C(O)NR⁵— or —S(O)₂NR⁴— may be prepared by reacting a compound of formula (XXXII) with an appropriate compound of formula (XVI):

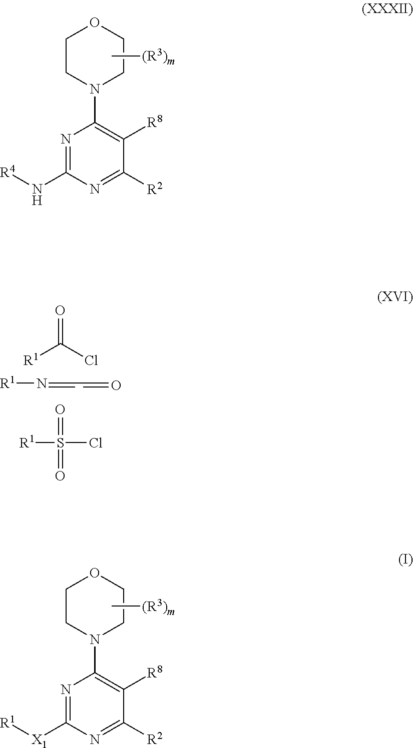

A compound of formula (XXXII) may be prepared by reacting a compound of formula (XXXIII)

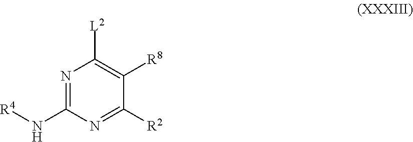

with a compound of formula (V)

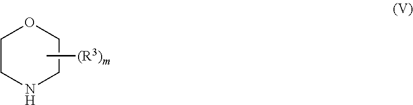

This reaction may be performed in solvent such as tetrahydrofuran in the presence of a suitable base such as triethylamine.

A compound of formula (XXXIII) may be prepared by reacting a compound of formula (XXXVI) wherein L³ is a leaving group such as halo (for example chloro),

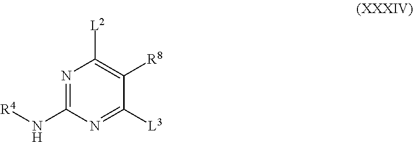

with a suitable organo-metallic reagent such as the tributyltin derivative or the zincate of formula (XX) wherein Y can be halo such as chloro. Where R² is unsaturated such as optionally substituted aryl or heteroaryl, the tributyltin derivative should be used whilst the zincate should be used for cases when R² is saturated.

R²—SnR₃ or R²—Zn—Y (XX)

A compound of formula (XXXIV) may be prepared from a compound of formula (XXXV)

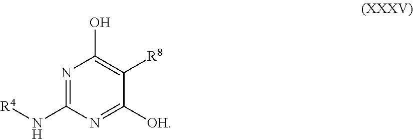

When L² and L³ are chloro, a chlorinating agent such as phosphorous oxychloride may be used.

A compound of formula (XXXV) may be prepared by reacting a compound of formula (XXVII) wherein PG¹ and PG² are C₁₋₄alkyl such as methyl or ethyl.

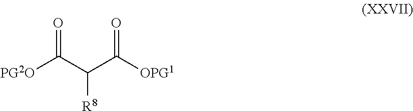

with a compound of formula (XXXVI)

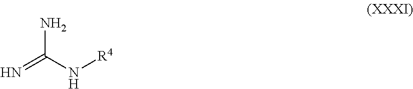

In an analogous manner, compounds wherein X is —NR⁴S(O)₂— may be prepared starting from a compound of formula (XXVII) and a compound of formula (XXXVI) wherein PG3 is a thiol protecting group.

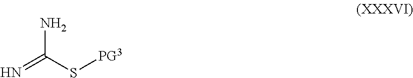

A compound of formula (I), wherein X is —X¹CR⁶R⁷— and X¹ is —NR⁴—, —O—, —S—, —S(O)—, or —S(O)₂— may be prepared from a compound of formula (XXXVII), wherein L¹ is a leaving group such as halo (for example chloro), tosyl, mesyl etc., by reaction with a compound of formula (XXXVIII) in the presence of a suitable base such as triethylamine or sodium hydride and a solvent such as tetrahydrofuran or N,N-dimethylformamide:

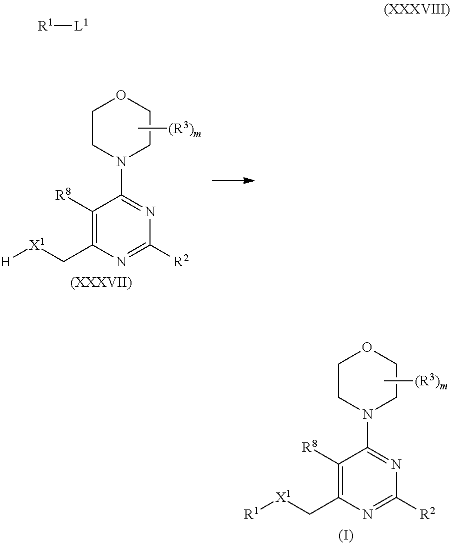

A compound of formula (I), wherein X is —X¹CR⁶R⁷— and X¹ is —S— may be prepared from a compound of formula (XXXIX), by reaction with a compound of formula (XXXVIII) in the presence of a suitable base such as sodium hydroxide and a solvent such as N,N-dimethylformamide:

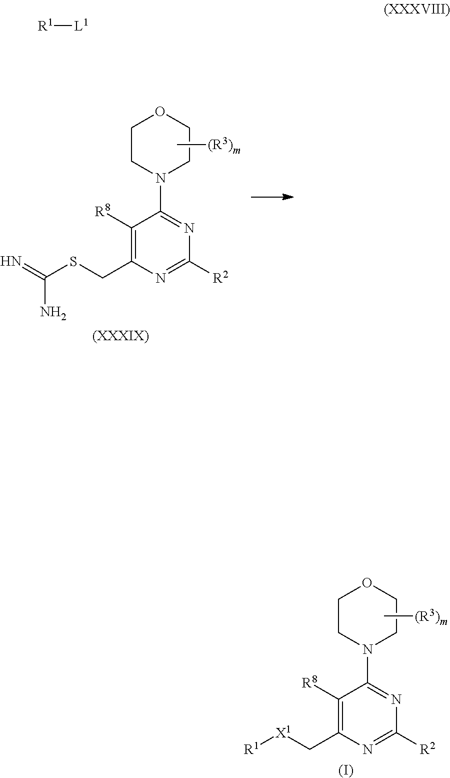

A compound of formula (XXXIX), may be prepared from a compound of formula (II), by reaction with thiourea in a suitable solvent such as ethanol.

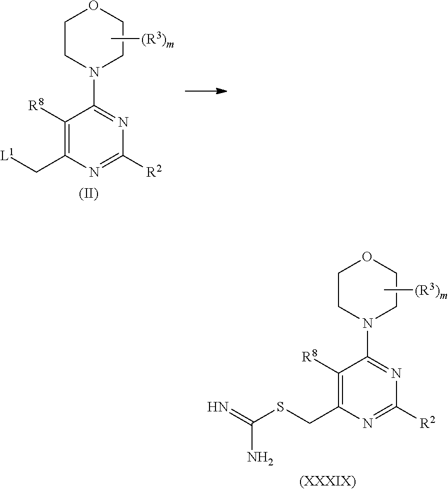

A compound of formula (I), wherein X is —X¹CR⁶R⁷— and X¹ is —NR⁴—, —O—, —S—, —S(O)—, or —S(O)₂— may be prepared by the reaction of a compound of formula (XXXX), with a suitable organo-metallic reagent (such as a the activated ester of boronic acid R²B(OR)₃ wherein R is C₁₋₄alkyl such as methyl), in the presence of a suitable metal catalyst (such as palladium or copper) using a solvent such as 1,4-dioxane.

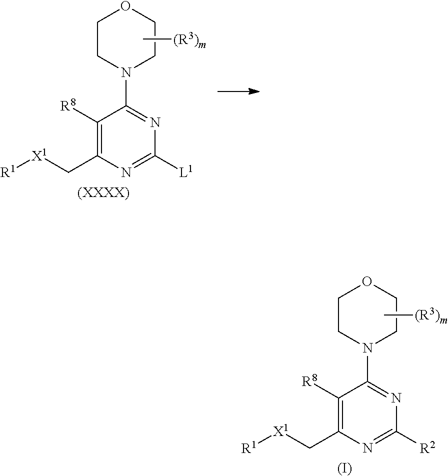

A compound of formula (XXXX) may be prepared by reacting a compound of formula (XXXXI) with a compound of formula (V).

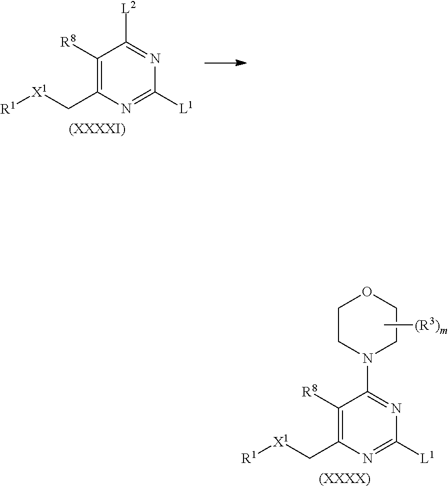

A compound of formula (XXXXII), wherein X¹ is —S—, —S(O)—, —S(O)₂—, —NR⁴SO₂— or or —NR⁴C(O)— may be prepared from a compound of formula (I) by reaction with compounds of formula (XXXXIII) and formula (XXXXIV), wherein L¹ and L² are leaving groups such as halo (for example chloro), tosyl, mesyl etc., in the presence of a suitable base such as sodium hydride and a solvent such as tetrahydrofuran.

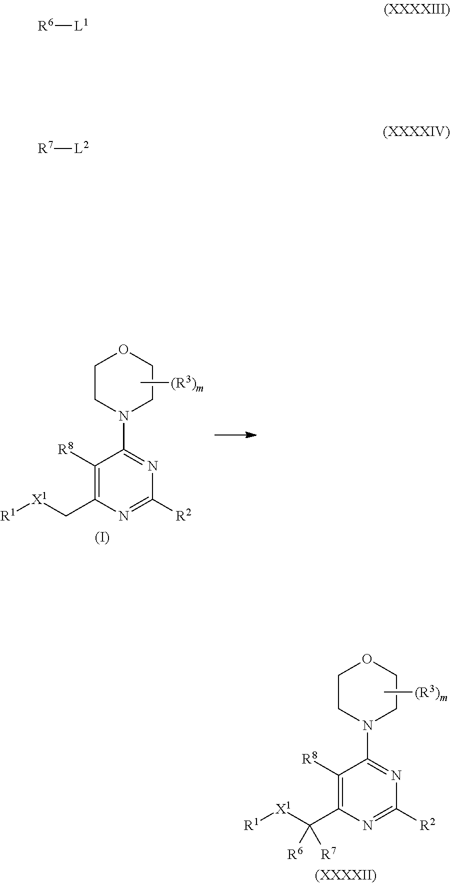

A compound of formula (XXXXII) may be prepared from a compound of formula (XXXXV) by the reaction with a compound of formula (III)

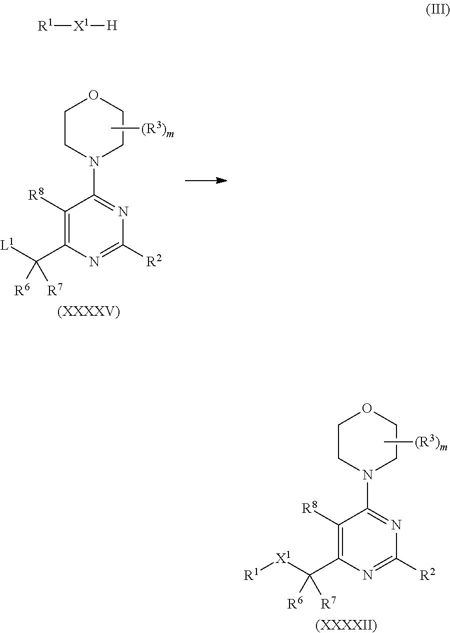

or by the reaction of a compound of formula (XXXXVI) with a compound of formula (XXXVIII).

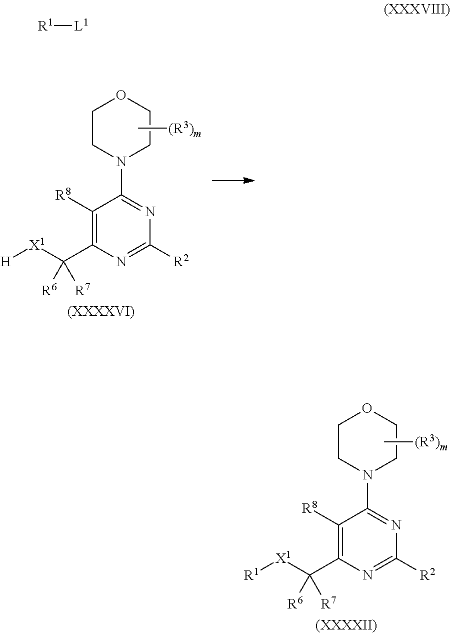

A compound of formula (XXXXV) may be prepared by standard functional group interconversions well known in the literature, from a compound of formula (XXXXVII).

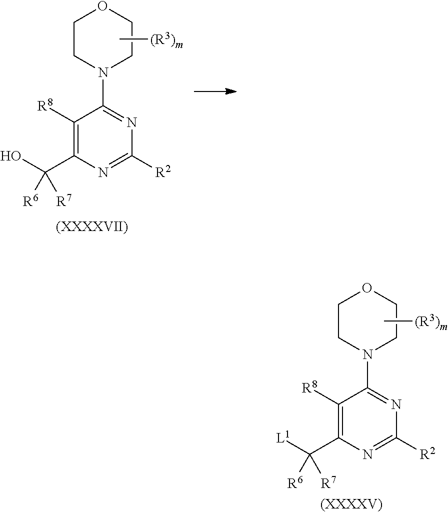

A compound of formula (XXXXVII) may be prepared from a compound of formula (XVIII), or suitable derivative thereof, such as an N-methoxy-N-methyl amide, with suitable organometallic reagents, such as R⁶MgBr and R⁷MgBr, either in a single or a two stage process.

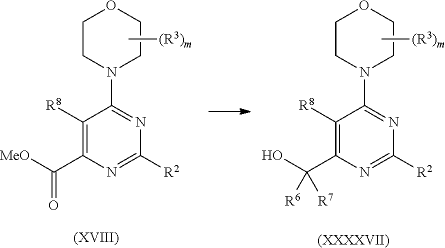

A compound of formula (I), wherein X is —NR⁴C(O)—, —NR⁴C(O)CR⁶R⁷—, —NR⁴S(O)₂—, or —NR⁴S(O)₂CR⁶R⁷—, may be prepared from a compound of formula (XXXXVIII), wherein X¹ is —C(O)—, —C(O)CR⁶R⁷—, —S(O)₂—, or —S(O)₂CR⁶R⁷— and L¹ is a suitable leaving groups such as chloro or an activated ester, with an amine of formula (XXXXIX), in the presence of a suitable base such as triethylamine.

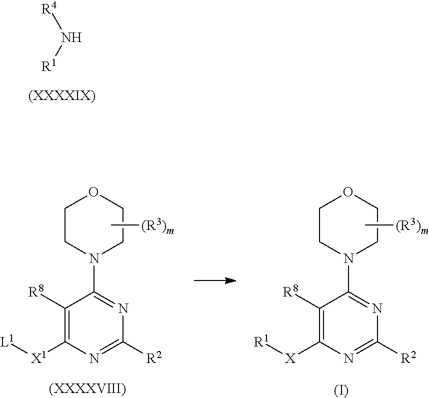

A compound of formula (I), wherein X is —NR⁴CHR₆— may be prepared by the reaction of a compound of formula (XXXXX) with an amine of formula (XXXXIX) in the presence of a suitable reducing agent such as NaCNBH₃.

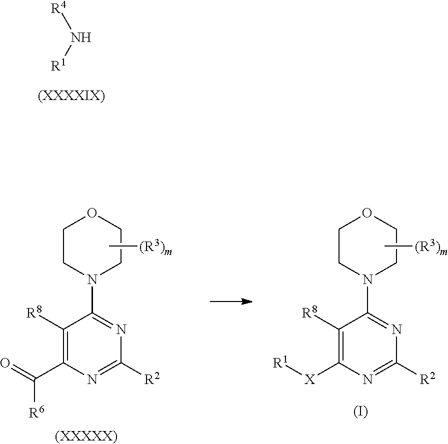

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. For example compounds of formula (I) my be converted into further compounds of formula (I) by standard aromatic substitution reactions or by conventional functional group modifications. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogen group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulfinyl or alkylsulfonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or tert-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a tert-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

Many of the intermediates defined herein are novel and these are provided as a further feature of the invention.

### Biological Assays

The following assays can be used to measure the effects of the compounds of the present invention as mTOR kinase inhibitors, as P13 kinase inhibitors, as inhibitors in vitro of the activation of PI3 kinase signalling pathways and as inhibitors in vitro of the proliferation of MDA-MB-468 human breast adenocarcinoma cells.

(a) In Vitro mTOR Kinase Assay

The assay used AlphaScreen technology (Gray et al., Analytical Biochemistry, 2003, 313: 234-245) to determine the ability of test compounds to inhibit phosphorylation by recombinant mTOR.

A C-terminal truncation of mTOR encompassing amino acid residues 1362 to 2549 of mTOR (EMBL Accession No. L34075) was stably expressed as a FLAG-tagged fusion in HEK293 cells as described by Vilella-Bach et al., Journal of Biochemistry, 1999, 274, 4266-4272. The HEK293 FLAG-tagged mTOR (1362-2549) stable cell line was routinely maintained at 37° C. with 5% CO₂ up to a confluency of 70-90% in Dulbecco's modified Eagle's growth medium (DMEM; Invitrogen Limited, Paisley, UK Catalogue No. 41966-029) containing 10% heat-inactivated foetal calf serum (FCS; Sigma, Poole, Dorset, UK, Catalogue No. F0392), 1% L-glutamine (Gibco, Catalogue No. 25030-024) and 2 mg/ml Geneticin

(G418 sulfate; Invitrogen Limited, UK Catalogue No. 10131-027). Following expression in the mammalian HEK293 cell line, expressed protein was purified using the FLAG epitope tag using standard purification techniques.

Test compounds were prepared as 10 mM stock solutions in DMSO and diluted into water as required to give a range of final assay concentrations. Aliquots (2 μl) of each compound dilution were placed into a well of a Greiner 384-well low volume (LV) white polystyrene plate (Greiner Bio-one). A 30 μl mixture of recombinant purified mTOR enzyme, 1 μM biotinylated peptide substrate (Biotin-Ahx-Lys-Lys-Ala-Asn-Gln-Val-Phe-Leu-Gly-Phe-Thr-Tyr-Val-Ala-Pro-Ser-Val-Leu-Glu-Ser-Val-Lys-Glu-NH₂; Bachem UK Ltd), ATP (20 μM) and a buffer solution [comprising Tris-HCl pH7.4 buffer (50 mM), EGTA (0.1 mM), bovine serum albumin (0.5 mg/mL), DTT (1.25 mM) and manganese chloride (10 mM)] was agitated at room temperature for 90 minutes.

Control wells that produced a maximum signal corresponding to maximum enzyme activity were created by using 5% DMSO instead of test compound. Control wells that produced a minimum signal corresponding to fully inhibited enzyme were created by adding EDTA (83 mM) instead of test compound. These assay solutions were incubated for 2 hours at room temperature.

Each reaction was stopped by the addition of 10 μl of a mixture of EDTA (50 mM), bovine serum albumin (BSA; 0.5 mg/mL) and Tris-HCl pH7.4 buffer (50 mM) containing p70 S6 Kinase (T389) 1A5 Monoclonal Antibody (Cell Signalling Technology, Catalogue No. 9206B) and AlphaScreen Streptavidin donor and Protein A acceptor beads (200 ng; Perkin Elmer, Catalogue No. 6760002B and 6760137R respectively) were added and the assay plates were left for about 20 hours at room temperature in the dark. The resultant signals arising from laser light excitation at 680 nm were read using a Packard Envision instrument.

Phosphorylated biotinylated peptide is formed in situ as a result of mTOR mediated phosphorylation. The phosphorylated biotinylated peptide that is associated with AlphaScreen Streptavidin donor beads forms a complex with the p70 S6 Kinase (T389) 1A5 Monoclonal Antibody that is associated with Alphascreen Protein A acceptor beads. Upon laser light excitation at 680 nm, the donor bead: acceptor bead complex produces a signal that can be measured. Accordingly, the presence of mTOR kinase activity results in an assay signal. In the presence of an mTOR kinase inhibitor, signal strength is reduced.

mTOR enzyme inhibition for a given test compound was expressed as an IC₅₀ value.

### (b) In Vitro PI3K Enzyme Assay

The assay used AlphaScreen technology (Gray et al., Analytical Biochemistry, 2003, 313: 234-245) to determine the ability of test compounds to inhibit phosphorylation by recombinant Type I PI3K enzymes of the lipid PI(4,5)P2.

DNA fragments encoding human PI3K catalytic and regulatory subunits were isolated from cDNA libraries using standard molecular biology and PCR cloning techniques. The selected DNA fragments were used to generate baculovirus expression vectors. In particular, full length DNA of each of the p110α, p110β and p110δ Type Ia human PI3K p110 isoforms (EMBL Accession Nos. HSU79143, S67334, Y10055 for p110 α, p110β and p110δ respectively) were sub-cloned into a pDEST10 vector (Invitrogen Limited, Fountain Drive, Paisley, UK). The vector is a Gateway-adapted version of Fastbac1 containing a 6-His epitope tag. A truncated form of Type Ib human PI3K p110γ isoform corresponding to amino acid residues 144-1102 (EMBL Accession No. X8336A) and the full length human p85α regulatory subunit (EMBL Accession No. HSP13KIN) were also sub-cloned into pFastBac1 vector containing a 6-His epitope tag. The Type Ia p110 constructs were co-expressed with the p85α regulatory subunit. Following expression in the baculovirus system using standard baculovirus expression techniques, expressed proteins were purified using the His epitope tag using standard purification techniques.

DNA corresponding to amino acids 263 to 380 of human general receptor for phosphoinositides (Grp1) PH domain was isolated from a cDNA library using standard molecular biology and PCR cloning techniques. The resultant DNA fragment was sub-cloned into a pGEX 4T1 E. coli expression vector containing a GST epitope tag (Amersham Pharmacia Biotech, Rainham, Essex, UK) as described by Gray et al., Analytical Biochemistry, 2003, 313: 234-245). The GST-tagged Grp1 PH domain was expressed and purified using standard techniques.

Test compounds were prepared as 10 mM stock solutions in DMSO and diluted into water as required to give a range of final assay concentrations. Aliquots (2 μl) of each compound dilution were placed into a well of a Greiner 384-well low volume (LV) white polystyrene plate (Greiner Bio-one, Brunel Way, Stonehouse, Gloucestershire, UK Catalogue No. 784075). A mixture of each selected recombinant purified PI3K enzyme (15 ng),

DiC8-PI(4,5)P2 substrate (40 μM; Cell Signals Inc., Kinnear Road, Columbus, USA, Catalogue No. 901), adenosine triphosphate (ATP; 4 μM) and a buffer solution [comprising Tris-HCl pH7.6 buffer (40 mM, 10 μl), 3-[(3-cholamidopropyl)dimethylammonio]-1-propanesulfonate (CHAPS; 0.04%), dithiothreitol (DTT; 2 mM) and magnesium chloride (10 mM)] was agitated at room temperature for 20 minutes.

Control wells that produced a minimum signal corresponding to maximum enzyme activity were created by using 5% DMSO instead of test compound. Control wells that produced a maximum signal corresponding to fully inhibited enzyme were created by adding wortmannin (6 μM; Calbiochem/Merck Bioscience, Padge Road, Beeston, Nottingham, UK, Catalogue No. 681675) instead of test compound. These assay solutions were also agitated for 20 minutes at room temperature.

Each reaction was stopped by the addition of 10 μl of a mixture of EDTA (100 mM), bovine serum albumin (BSA, 0.045%) and Tris-HCl pH7.6 buffer (40 mM).

Biotinylated-DiC8-PI(3,4,5)P3 (50 nM; Cell Signals Inc., Catalogue No. 107), recombinant purified GST-Grp1 PH protein (2.5 nM) and AlphaScreen Anti-GST donor and acceptor beads (100 ng; Packard Bioscience Limited, Station Road, Pangbourne, Berkshire, UK, Catalogue No. 6760603M) were added and the assay plates were left for about 5 to

20 hours at room temperature in the dark. The resultant signals arising from laser light excitation at 680 nm were read using a Packard AlphaQuest instrument.

PI(3,4,5)P3 is formed in situ as a result of PI3K mediated phosphorylation of PI(4,5)P2. The GST-Grp1 PH domain protein that is associated with AlphaScreen Anti-GST donor beads forms a complex with the biotinylated PI(3,4,5)P3 that is associated with Alphascreen Streptavidn acceptor beads. The enymatically-produced PI(3,4,5)P3 competes with biotinylated PI(3,4,5)P3 for binding to the PH domain protein. Upon laser light excitation at 680 nm, the donor bead: acceptor bead complex produces a signal that can be measured. Accordingly, PI3K enzyme activity to form PI(3,4,5)P3 and subsequent competition with biotinylated PI(3,4,5)P3 results in a reduced signal. In the presence of a PI3K enzyme inhibitor, signal strength is recovered.

PI3K enzyme inhibition for a given test compound was expressed as an IC₅₀ value.

### (c) In Vitro Phospho-Ser473 Akt Assay

This assay determines the ability of test compounds to inhibit phosphorylation of Serine 473 in Akt as assessed using Acumen Explorer technology (Acumen Bioscience Limited), a plate reader that can be used to rapidly quantitate features of images generated by laser-scanning.

A MDA-MB-468 human breast adenocarcinoma cell line (LGC Promochem, Teddington, Middlesex, UK, Catalogue No. HTB-132) was routinely maintained at 37° C. with 5% CO₂ up to a confluency of 70-90% in DMEM containing 10% heat-inactivated FCS and 1% L-glutamine.

For the assay, the cells were detached from the culture flask using ‘Accutase’ (Innovative Cell Technologies Inc., San Diego, Calif., USA; Catalogue No. AT104) using standard tissue culture methods and resuspended in media to give 1.7×10⁵ cells per mL. Aliquots (90 μl) were seeded into each of the inner 60 wells of a black Packard 96 well plate (PerkinElmer, Boston, Mass., USA; Catalogue No. 6005182) to give a density of ˜15000 cells per well. Aliquots (90 μl) of culture media were placed in the outer wells to prevent edge effects. The cells were incubated overnight at 37° C. with 5% CO₂ to allow them to adhere.

On day 2, the cells were treated with test compounds and incubated for 2 hours at 37° C. with 5% CO₂. Test compounds were prepared as 10 mM stock solutions in DMSO and serially diluted as required with growth media to give a range of concentrations that were 10-fold the required final test concentrations. Aliquots (10 μl) of each compound dilution were placed in a well (in triplicate) to give the final required concentrations. As a minimum reponse control, each plate contained wells having a final concentration of 100 μM LY294002 (Calbiochem, Beeston, UK, Catalogue No. 440202). As a maximum response control, wells contained

1% DMSO instead of test compound. Following incubation, the contents of the plates were fixed by treatment with a 1.6% aqueous formaldehyde solution (Sigma, Poole, Dorset, UK, Catalogue No. F1635) at room temperature for 1 hour.

All subsequent aspiration and wash steps were carried out using a Tecan 96 well plate washer (aspiration speed 10 mm/sec). The fixing solution was removed and the contents of the plates were washed with phosphate-buffered saline (PBS; 50 μl; Gibco, Catalogue

No. 10010015). The contents of the plates were treated for 10 minutes at room temperature with an aliquot (50 μl) of a cell permeabilisation buffer consisting of a mixture of PBS and
0.5% Tween-20. The ‘permeabilisation’ buffer was removed and non-specific binding sites were blocked by treatment for 1 hour at room temperature of an aliquot (50 μl) of a blocking buffer consisting of 5% dried skimmed milk [‘Marvel’ (registered trade mark); Premier Beverages, Stafford, GB] in a mixture of PBS and 0.05% Tween-20. The ‘blocking’ buffer was removed and the cells were incubated for 1 hour at room temperature with rabbit anti phospho-Akt (Ser473) antibody solution (50 μl per well; Cell Signalling, Hitchin, Herts, U.K., Catalogue No 9277) that had been diluted 1:500 in ‘blocking’ buffer. Cells were washed
three times in a mixture of PBS and 0.05% Tween-20. Subsequently, cells were incubated for 1 hour at room temperature with Alexafluor488 labelled goat anti-rabbit IgG (50 μl per well; Molecular Probes, Invitrogen Limited, Paisley, UK, Catalogue No. A11008) that had been diluted 1:500 in ‘blocking’ buffer. Cells were washed 3 times with a mixture of PBS and 0.05% Tween-20. An aliquot of PBS (50 μl) was added to each well and the plates were sealed with black plate sealers and the fluorescence signal was detected and analysed.

Fluorescence dose response data obtained with each compound were analysed and the degree of inhibition of Serine 473 in Akt was expressed as an IC₅₀ value.

### (d) In Vitro MDA-MB-468 Human Breast Adenocarcinoma Proliferation Assay

This assay determines the ability of test compounds to inhibit cell proliferation as assessed using Cellomics Arrayscan technology. A MDA-MB-468 human breast adenocarcinoma cell line (LGC Promochem, Catalogue No. HTB-132) was routinely maintained as described in Biological Assay (b) herein.

For the proliferation assay, the cells were detached from the culture flask using Accutase and seeded into the inner 60 wells of a black Packard 96 well plate at a density of 8000 cells per well in 100 μl of complete growth media. The outer wells contained 100 μl of sterile PBS. The cells were incubated overnight at 37° C. with 5% CO₂ to allow them to adhere.

On day 2, the cells were treated with test compounds and incubated for 48 hours at 37° C. with 5% CO₂. Test compounds were prepared as 10 mM stock solutions in DMSO and serially diluted as required with growth media to give a range of test concentrations. Aliquots (50 μl) of each compound dilution were placed in a well and the cells were incubated for

2 days at 37° C. with 5% CO₂. Each plate contained control wells without test compound.

On day 4, BrdU labelling reagent (Sigma, Catalogue No. B9285) at a final dilution of 1:1000 was added and the cells were incubated for 2 hours at 37° C. The medium was removed and the cells in each well were fixed by treatment with 100 μl of a mixture of ethanol and glacial acetic acid (90% ethanol, 5% glacial acetic acid and 5% water) for 30 minutes at room temperature. The cells in each well were washed twice with PBS (100 μl). Aqueous hydrochloric acid (2M, 100 μl) was added to each well. After 20 minutes at room temperature, the cells were washed twice with PBS. Hydrogen peroxide (3%, 50 μl; Sigma, Catalogue No. H1009) was added to each well. After 10 minutes at room temperature, the wells were washed again with PBS.

BrdU incorporation was detected by incubation for 1 hour at room temperature with mouse anti-BrdU antibody (50 μl; Caltag, Burlingame, Calif., US; Catalogue No. MD5200) that was diluted 1:40 in PBS containing 1% BSA and 0.05% Tween-20. Unbound antibody was removed with two washes of PBS. For visualisation of incorporated BrdU, the cells were treated for 1 hour at room temperature with PBS (50 μl) and 0.05% Tween-20 buffer containing a 1:1000 dilution of Alexa fluor 488-labelled goat anti-mouse IgG. For visualisation of the cell nucleus, a 1:1000 dilution of Hoechst stain (Molecular Probes, Catalogue No. H3570) was added. Each plate was washed in turn with PBS. Subsequently, PBS (100 μl) was added to each well and the plates were analysed using a Cellomics array scan to assess total cell number and number of BrdU positive cells.

Fluorescence dose response data obtained with each compound were analysed and the degree of inhibition of MDA-MB-468 cell growth was expressed as an IC₅₀ value.

Although the pharmacological properties of the compounds of formula (I) vary with structural change as expected, in general, it is believed that activity possessed by compounds of formula (I) may be demonstrated at the following concentrations or doses in one or more of the above tests (a) to (d):—
Test (a):—IC₅₀ versus mTOR kinase at less than 10 μM, in particular 0.001-0.5 μM for many compounds; for example 65 the IC50 was measured on three occasions, the values were 3.9, 4.1 and 8.2 μM, resulting in a mean value of 5.4 μM.
Test (b):—IC₅₀ versus p110γ Type Ib human PI3K at less than 10 μM, in particular 0.001-0.5 μM for many compounds; and IC₅₀ versus p110α Type Ia human PI3K at less than 10 μM, in particular 0.001-0.5 μM for many compounds;
   for example 65 the IC50 was measured on three occasions, the values were 1.9, 13.0 and 5.7 resulting in a mean value of 6.8 μM.
Test (c):—IC₅₀ versus Serine 473 in Akt at less than 10 μM, in particular 0.1-20 μM for many compounds); for example 44 the IC50 was measured on five occasions, the values were 12.5, 5.6, 9.7, 10.3 and 6.1 μM, resulting in a mean value of 8.84 μM
Test (d):—IC₅₀ at less than 20 μM;

The compounds of the present invention are advantageous in that they possess pharmacological activity. In particular, the compounds of the present invention modulate (in particular, inhibit) mTOR kinase and/or phosphatidylinositol-3-kinase (PI3K) enzymes, such as the Class Ia PI3K enzymes (e.g. PI3Kalpha, PI3Kbeta and PI3Kdelta) and the Class Ib PI3K enzyme (PI3Kgamma). More particularly compounds of the present invention modulate (in particular, inhibit) mTOR kinase. More particularly compounds of the present invention modulate (in particular, inhibit) one or more PI3K enzyme. The inhibitory properties of compounds of formula (I) may be demonstrated using the test procedures set out herein and in the experimental section. Accordingly, the compounds of formula (I) may be used in the treatment (therapeutic or prophylactic) of conditions/diseases in human and non-human animals which are mediated by mTOR kinase and/or one or more PI3K enzyme(s), and in particular by mTOR kinase.

The invention also provides a pharmaceutical composition comprising a compound of formula (I), or a pharmaceutically acceptable salt thereof, as defined herein in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

The amount of active ingredient that is combined with one or more excipients to produce a single dosage form will necessarily vary depending upon the host treated and the particular route of administration. For example, a formulation intended for oral administration to humans will generally contain, for example, from 1 mg to 1 g of active agent (more suitably from 1 to 250 mg, for example from 1 to 100 mg) compounded with an appropriate and convenient amount of excipients which may vary from about 5 to about 98 percent by weight of the total composition.

The size of the dose for therapeutic or prophylactic purposes of a compound of formula I will naturally vary according to the nature and severity of the disease state, the age and sex of the animal or patient and the route of administration, according to well known principles of medicine.

In using a compound of formula (I) for therapeutic or prophylactic purposes it will generally be administered so that a daily dose in the range, for example, 1 mg/kg to 100 mg/kg body weight is received, given if required in divided doses. In general, lower doses will be administered when a parenteral route is employed. Thus, for example, for intravenous administration, a dose in the range, for example, 1 mg/kg to 25 mg/kg body weight will generally be used. Similarly, for administration by inhalation, a dose in the range, for example, 1 mg/kg to 25 mg/kg body weight will be used. Typically, unit dosage forms will contain about 10 mg to 0.5 g of a compound of this invention.

As stated herein, it is known that mTOR kinase and the PI3K enzymes have roles in tumourigenesis as well as numerous other diseases. We have found that the compounds of formula (I) possess potent anti-tumour activity which it is believed is obtained by way of inhibition of mTOR kinase and/or one or more of the PI3K enzymes.

Accordingly, the compounds of the present invention are of value as anti-tumour agents. Particularly, the compounds of the present invention are of value as anti-proliferative, apoptotic and/or anti-invasive agents in the containment and/or treatment of solid and/or liquid tumour disease. Particularly, the compounds of the present invention are expected to be useful in the prevention or treatment of those tumours which are sensitive to inhibition of mTOR and/or one or more of the PI3K enzymes such as the Class Ia PI3K enzymes and the Class Ib PI3K enzyme. Further, the compounds of the present invention are expected to be useful in the prevention or treatment of those tumours which are mediated alone or in part by mTOR and/or one or more of the PI3K enzymes such as the Class Ia PI3K enzymes and the Class Ib PI3K enzyme. The compounds may thus be used to produce an mTOR enzyme inhibitory effect in a warm-blooded animal in need of such treatment. Certain compounds may be used to produce an PI3K enzyme inhibitory effect in a warm-blooded animal in need of such treatment.

As stated herein, inhibitors of mTOR kinase and/or one or more PI3K enzymes should be of therapeutic value for the treatment of proliferative disease such as cancer and in particular solid tumours such as carcinoma and sarcomas and the leukaemias and lymphoid malignancies and in particular for treatment of, for example, cancer of the breast, colorectum, lung (including small cell lung cancer, non-small cell lung cancer and bronchioalveolar cancer) and prostate, and of cancer of the bile duct, bone, bladder, head and neck, kidney, liver, gastrointestinal tissue, oesophagus, ovary, pancreas, skin, testes, thyroid, uterus, cervix and vulva, and of leukaemias [including acute lymphoctic leukaemia (ALL) and chronic myelogenous leukaemia (CML)], multiple myeloma and lymphomas.

According to a further aspect of the invention there is provided a compound of formula (I), or a pharmaceutically acceptable salt thereof, as defined herein for use as a medicament in a warm-blooded animal such as man.

According to a further aspect of the invention, there is provided a compound of formula (I), or a pharmaceutically acceptable salt thereof, as defined herein for use in the production of an anti-proliferative effect in a warm-blooded animal such as man.

According to a further aspect of the invention, there is provided a compound of formula (I), or a pharmaceutically acceptable salt thereof, as defined herein for use in the production of an apoptotic effect in a warm-blooded animal such as man.

According to a further feature of the invention there is provided a compound of formula (I), or a pharmaceutically acceptable salt thereof, as defined herein for use in a warm-blooded animal such as man as an anti-invasive agent in the containment and/or treatment of proliferative disease such as cancer.

According to a further aspect of the invention, there is provided the use of a compound of formula (I), or a pharmaceutically acceptable salt thereof, as defined herein for the production of an anti-proliferative effect in a warm-blooded animal such as man.

According to a further feature of this aspect of the invention there is provided the use of a compound of formula (I), or a pharmaceutically acceptable salt thereof, as defined herein in the manufacture of a medicament for use in the production of an anti-proliferative effect in a warm-blooded animal such as man.

According to a further aspect of the invention, there is provided the use of a compound of formula (I), or a pharmaceutically acceptable salt thereof, as defined herein for the production of an apoptotic effect in a warm-blooded animal such as man.

According to a further feature of this aspect of the invention there is provided the use of a compound of formula (I), or a pharmaceutically acceptable salt thereof, as defined herein in the manufacture of a medicament for use in the production of an apoptotic effect in a warm-blooded animal such as man.

According to a further feature of the invention there is provided the use of a compound of formula (I), or a pharmaceutically acceptable salt thereof, as defined herein in the manufacture of a medicament for use in a warm-blooded animal such as man as an anti-invasive agent in the containment and/or treatment of proliferative disease such as cancer.

According to a further feature of this aspect of the invention there is provided a method for producing an anti-proliferative effect in a warm-blooded animal, such as man, in need of such treatment which comprises administering to said animal an effective amount of a compound of formula (I), or a pharmaceutically acceptable salt thereof, as defined herein.

According to a further feature of this aspect of the invention there is provided a method for producing an anti-invasive effect by the containment and/or treatment of solid tumour disease in a warm-blooded animal, such as man, in need of such treatment which comprises administering to said animal an effective amount of a compound of formula (I), or a pharmaceutically acceptable salt thereof, as defined herein.

According to a further aspect of the invention there is provided the use of a compound of formula (I), or a pharmaceutically acceptable salt thereof, as defined herein in the manufacture of a medicament for use in the prevention or treatment of proliferative disease such as cancer in a warm-blooded animal such as man.

According to a further feature of this aspect of the invention there is provided a method for the prevention or treatment of proliferative disease such as cancer in a warm-blooded animal, such as man, in need of such treatment which comprises administering to said animal an effective amount of a compound of formula (I), or a pharmaceutically acceptable salt thereof, as defined herein.

According to a further aspect of the invention there is provided a compound of formula (I), or a pharmaceutically acceptable salt thereof, as defined herein for use in the prevention or treatment of those tumours which are sensitive to inhibition of mTOR kinase and/or one or more PI3K enzymes (such as the Class Ia enzymes and/or the Class Ib PI3K enzyme) that are involved in the signal transduction steps which lead to the proliferation, survival, invasiveness and migratory ability of tumour cells.

According to a further feature of this aspect of the invention there is provided the use of a compound of formula (I), or a pharmaceutically acceptable salt thereof, as defined herein in the manufacture of a medicament for use in the prevention or treatment of those tumours which are sensitive to inhibition of mTOR kinase and/or one or more PI3K enzymes (such as the Class Ia enzymes and/or the Class Ib PI3K enzyme) that are involved in the signal transduction steps which lead to the proliferation, survival, invasiveness and migratory ability of tumour cells.

According to a further feature of this aspect of the invention there is provided a method for the prevention or treatment of those tumours which are sensitive to inhibition of mTOR kinase and/or one or more PI3K enzymes (such as the Class Ia enzymes and/or the Class Ib PI3K enzyme) that are involved in the signal transduction steps which lead to the proliferation, survival, invasiveness and migratory ability of tumour cells which comprises administering to said animal an effective amount of a compound of formula (I), or a pharmaceutically acceptable salt thereof, as defined herein.

According to a further aspect of the invention there is provided a compound of formula (I), or a pharmaceutically acceptable salt thereof, as defined herein for use in providing a mTOR kinase inhibitory effect and/or a PI3K enzyme inhibitory effect (such as a Class Ia PI3K enzyme or Class Ib PI3K enzyme inhibitory effect).

According to a further feature of this aspect of the invention there is provided the use of a compound of formula (I), or a pharmaceutically acceptable salt thereof, as defined herein in the manufacture of a medicament for use in providing a mTOR kinase inhibitory effect and/or a PI3K enzyme inhibitory effect (such as a Class Ia PI3K enzyme or Class Ib PI3K enzyme inhibitory effect).

According to a further aspect of the invention there is also provided a method for providing a mTOR kinase inhibitory effect and/or a PI3K enzyme inhibitory effect (such as a Class Ia PI3K enzyme or Class Ib PI3K enzyme inhibitory effect) which comprises administering an effective amount of a compound of formula I, or a pharmaceutically acceptable salt thereof, as defined herein.

According to a further feature of the invention there is provided a compound of formula I, or a pharmaceutically acceptable salt thereof, as defined herein for use in the treatment of cancer, inflammatory diseases, obstructive airways diseases, immune diseases or cardiovascular diseases.

According to a further feature of the invention there is provided a compound of formula I, or a pharmaceutically acceptable salt thereof, as defined herein for use in the treatment of solid tumours such as carcinoma and sarcomas and the leukaemias and lymphoid malignancies.

According to a further feature of the invention there is provided a compound of formula I, or a pharmaceutically acceptable salt thereof, as defined herein for use in the treatment of cancer of the breast, colorectum, lung (including small cell lung cancer, non-small cell lung cancer and bronchioalveolar cancer) and prostate.

According to a further feature of the invention there is provided a compound of formula (I), or a pharmaceutically acceptable salt thereof, as defined herein for use in the treatment of cancer of the bile duct, bone, bladder, head and neck, kidney, liver, gastrointestinal tissue, oesophagus, ovary, pancreas, skin, testes, thyroid, uterus, cervix and vulva, and of leukaemias (including ALL and CML), multiple myeloma and lymphomas.

According to a further feature of the invention there is provided the use of a compound of formula (I), or a pharmaceutically acceptable salt thereof, as defined herein in the manufacture of a medicament for use in the treatment of cancer, inflammatory diseases, obstructive airways diseases, immune diseases or cardiovascular diseases.

According to a further feature of the invention there is provided the use of a compound of formula (I), or a pharmaceutically acceptable salt thereof, as defined herein in the manufacture of a medicament for use in the treatment of solid tumours such as carcinoma and sarcomas and the leukaemias and lymphoid malignancies.

According to a further feature of the invention there is provided the use of a compound of formula (I), or a pharmaceutically acceptable salt thereof, as defined herein in the manufacture of a medicament for use in the treatment of cancer of the breast, colorectum, lung (including small cell lung cancer, non-small cell lung cancer and bronchioalveolar cancer) and prostate.

According to a further feature of the invention there is provided the use of a compound of formula (I), or a pharmaceutically acceptable salt thereof, as defined herein in the manufacture of a medicament for use in the treatment of cancer of the bile duct, bone, bladder, head and neck, kidney, liver, gastrointestinal tissue, oesophagus, ovary, pancreas, skin, testes, thyroid, uterus, cervix and vulva, and of leukaemias (including ALL and CML), multiple myeloma and lymphomas.

According to a further feature of the invention there is provided a method for treating cancer, inflammatory diseases, obstructive airways diseases, immune diseases or cardiovascular diseases in a warm blooded animal such as man that is in need of such treatment which comprises administering an effective amount of a compound of formula (I), or a pharmaceutically acceptable salt thereof, as defined herein.

According to a further feature of the invention there is provided a method for treating solid tumours such as carcinoma and sarcomas and the leukaemias and lymphoid malignancies in a warm blooded animal such as man that is in need of such treatment which comprises administering an effective amount of a compound of formula (I), or a pharmaceutically acceptable salt thereof, as defined herein.

According to a further feature of the invention there is provided a method for treating cancer of the breast, colorectum, lung (including small cell lung cancer, non-small cell lung cancer and bronchioalveolar cancer) and prostate in a warm blooded animal such as man that is in need of such treatment which comprises administering an effective amount of a compound of formula (I), or a pharmaceutically acceptable salt thereof, as defined herein.

According to a further feature of the invention there is provided a method for treating cancer of the bile duct, bone, bladder, head and neck, kidney, liver, gastrointestinal tissue, oesophagus, ovary, pancreas, skin, testes, thyroid, uterus, cervix and vulva, and of leukaemias (including ALL and CML), multiple myeloma and lymphomas in a warm blooded animal such as man that is in need of such treatment which comprises administering an effective amount of a compound of formula (I), or a pharmaceutically acceptable salt thereof, as defined herein.

As stated herein, the in vivo effects of a compound of formula (I) may be exerted in part by one or more metabolites that are formed within the human or animal body after administration of a compound of formula (I).

The invention further relates to combination therapies wherein a compound of formula (I), or a pharmaceutically acceptable salt thereof, or a pharmaceutical composition or formulation comprising a compound of formula (I) is administered concurrently or sequentially or as a combined preparation with another treatment of use in the control of oncology disease.

In particular, the treatment defined herein may be applied as a sole therapy or may involve, in addition to the compounds of the invention, conventional surgery or radiotherapy or chemotherapy. Accordingly, the compounds of the invention can also be used in combination with existing therapeutic agents for the treatment of cancer.

Suitable agents to be used in combination include:—

(i) antiproliferative/antineoplastic drugs and combinations thereof, as used in medical oncology such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside, hydroxyurea and gemcitabine); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like paclitaxel and taxotere); and topoisomerase inhibitors (for example epipodophyllotoxins like etoposide and teniposide, amsacrine, topotecan and camptothecins);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat and inhibitors of urokinase plasminogen activator receptor function);
(iv) inhibitors of growth factor function: for example such inhibitors include growth factor antibodies and growth factor receptor antibodies (for example the anti-erbB2 antibody trastuzumab [Herceptin™] and the anti-erbB1 antibody cetuximab [C225]); such inhibitors also include, for example, tyrosine kinase inhibitors, for example inhibitors of the epidermal growth factor family (for example EGFR family tyrosine kinase inhibitors such as
N-(3-chloro-4-fluorophenyl)-7-methoxy-6-(3-morpholinopropoxy)quinazolin-4-amine (gefitinib, ZD 1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033) and erbB2 tyrosine kinase inhibitors such as lapatinib), inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)) and inhibitors of cell signalling through MEK and/or Akt kinases;
(v) antiangiogenic agents such as those which inhibit the effects of vascular endothelial growth factor, [for example the anti-vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), and compounds that work by other mechanisms (for example linomide, inhibitors of integrin αvβ3 function and angiostatin)];
(vi) vascular damaging agents such as combretastatin A4 and compounds disclosed in International Patent Applications WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense agent;
(viii) gene therapy approaches, including approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapeutic approaches, including ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The invention will now be further explained by reference to the following illustrative examples.

Unless stated otherwise, starting materials were commercially available. All solvents and commercial reagents were of laboratory grade and were used as received.

In the examples ¹H NMR spectra were recorded on a Bruker DPX 300 (300 MHz), Bruker DRX 400 (400 MHz) instrument or a Bruker DRX 500 (500 MHz) instrument. The central peaks of chloroform-d (δ_{H} 7.27 ppm), dimethylsulfoxide-d₆ (δ_{H} 2.50 ppm) or acetone-d₆ (δ_{H} 2.05 ppm) were used as internal references. The following abbreviations have been used: s, singlet; d, doublet; t, triplet; q, quartet; m, multiplet; br, broad.

Column chromatography was carried out using silica gel (0.04-0.063 mm, Merck). In general, a Kromasil KR-100-5-C18 reversed-phase column (250×20 mm, Akzo Nobel) was used for preparative HPLC with mixtures of acetonitrile and water [containing 0.1% trifluoroacetic acid (TFA)] used as the eluent at a flow rate of 10 mL/min.

The following methods were used for liquid chromatography (LC)/mass spectral (MS) analysis:—

### HPLC: Agilent 1100 or Waters Alliance HT (2790 & 2795)

### Mass Spectrometer Waters ZQ ESCi

### HPLC Column

The standard HPLC column used is the Phemonenex Gemini C18 5 μm, 50×2 mm.

### Acidic HPLC Methods

The mobile phases used are:
Mobile phase A: Water
Mobile Phase B: Acetonitrile
Mobile Phase C: 1% Formic Acid in 50:50 Water:MeCN (v/v)

Each method is followed by a rapid equilibration using a 5 mL flow rate for 0.45 min.

Four generic HPLC methods are available:

### 5 Minute Monitor Acidic Method

| | | | | | Flow |
|---|---|---|---|---|---|
| Time/ | Mobile | Mobile Phase | Mobile Phase | | Rate/ |
| min | Phase A: | B: | C: | Curve | mL/min |
| 0.00 | 95 | 0 | 5 | 1 | 1.1 |
| 4 | 0 | 95 | 5 | 6 | 1.1 |
| 4.5 | 0 | 95 | 5 | 6 | 1.1 |

### Early Acidic Method for Early Eluting Compounds

| | | | | | Flow |
|---|---|---|---|---|---|
| Time/ | Mobile | Mobile Phase | Mobile Phase | | Rate/ |
| min | Phase A: | B: | C: | Curve | mL/min |
| 0.00 | 95 | 0 | 5 | 1 | 1.1 |
| 4 | 57.5 | 37.5 | 5 | 6 | 1.1 |
| 4.5 | 57.5 | 37.5 | 5 | 6 | 1.1 |

### Mid Acidic Method for Middle Eluting Compounds

| | | | | | Flow |
|---|---|---|---|---|---|
| Time/ | Mobile | Mobile Phase | Mobile Phase | | Rate/ |
| min | Phase A: | B: | C: | Curve | mL/min |
| 0.00 | 95 | 0 | 5 | 1 | 1.1 |
| 0.01 | 67.5 | 27.5 | 5 | 6 | 1.1 |
| 4.5 | 27.5 | 67.5 | 5 | 6 | 1.1 |

### Late Acidic Method for Late Eluting Compounds

| | | | | | Flow |
|---|---|---|---|---|---|
| Time/ | Mobile | Mobile Phase | Mobile Phase | | Rate/ |
| min | Phase A: | B: | C: | Curve | mL/min |
| 0.00 | 95 | 0 | 5 | 1 | 1.1 |
| 0.01 | 27.5 | 67.5 | 5 | 6 | 1.1 |
| 4.5 | 5 | 95 | 5 | 6 | 1.1 |

### Basic HPLC Methods

In some instances the standard acidic methods may be unsuitable for either the compound ionisation or the chromatography separation required. In such cases four comparable Basic HPLC methods are available.

The mobile phases used are:
Mobile phase A: Water
Mobile Phase B: Acetonitrile
Mobile Phase D: 0.1% 880 Ammonia in acetonitrile

Each method is followed by a rapid equilibration using a 5 mL flow rate for 0.45 min.

### Minute Monitor Basic Method

| | | | | | Flow |
|---|---|---|---|---|---|
| Time/ | Mobile | Mobile Phase | Mobile Phase | | Rate/ |
| min | Phase A: | B: | D: | Curve | mL/min |
| 0.00 | 95 | 0 | 5 | 1 | 1.1 |
| 4 | 0 | 95 | 5 | 6 | 1.1 |
| 4.5 | 0 | 95 | 5 | 6 | 1.1 |

### Early Basic Method for Early Eluting Compounds

| | | | | | Flow |
|---|---|---|---|---|---|
| Time/ | Mobile | Mobile Phase | Mobile Phase | | Rate/ |
| min | Phase A: | B: | D: | Curve | mL/min |
| 0.00 | 95 | 0 | 5 | 1 | 1.1 |
| 4 | 57.5 | 37.5 | 5 | 6 | 1.1 |
| 4.5 | 57.5 | 37.5 | 5 | 6 | 1.1 |

### Mid Basic Method for Middle Eluting Compounds

| | | | | | Flow |
|---|---|---|---|---|---|
| Time/ | Mobile | Mobile Phase | Mobile Phase | | Rate/ |
| min | Phase A: | B: | D: | Curve | mL/min |
| 0.00 | 95 | 0 | 5 | 1 | 1.1 |
| 0.01 | 67.5 | 27.5 | 5 | 6 | 1.1 |
| 4.5 | 27.5 | 67.5 | 5 | 6 | 1.1 |

### Late Basic Method for Late Eluting Compounds

| | | | | | Flow |
|---|---|---|---|---|---|
| Time/ | Mobile | Mobile Phase | Mobile Phase | | Rate/ |
| min | Phase A: | B: | C: | Curve | mL/min |
| 0.00 | 95 | 0 | 5 | 1 | 1.1 |
| 0.01 | 27.5 | 67.5 | 5 | 6 | 1.1 |
| 4.5 | 5 | 95 | 5 | 6 | 1.1 |

The following method was used for liquid chromatography (LC)/mass spectral (MS) analysis:—Instrument: Agilent 1100; Column: Waters ‘Symmetry’ 2.1×30 mm;

Mass Spectral analysis using chemical ionisation (APCI); Flow rate: 0.7 mL/min; Absorption Wavelength: 254 nm; Solvent A: water+0.1% TFA; Solvent B: acetonitrile+0.1% TFA; Solvent Gradient: 15-95% Solvent B for 2.7 minutes followed by 95% Solvent B for
0.3 minutes.

The following methods were used for LC analysis:—

Method A:—Instrument: Agilent 1100; Column: Kromasil C18 reversed-phase silica,

100×3 mm, 5 μm particle size; Solvent A: 0.1% TFA/water, Solvent B: 0.08% TFA/acetonitrile; Flow Rate: 1 mL/min; Solvent Gradient: 10-100% Solvent B for 20 minutes followed by 100% Solvent B for 1 minute; Absorption Wavelengths: 220, 254 and 280 nm. In general, the retention time of the product was noted.

Method B:—Instrument: Agilent 1100; Column: Waters ‘Xterra’ C8 reversed-phase silica, 100×3 mm, 5 μm particle size; Solvent A: 0.015M ammonia in water, Solvent B: acetonitrile; Flow Rate: 1 ml/min, Solvent Gradient: 10-100% Solvent B for 20 minutes followed by 100% Solvent B for 1 minute; Absorption Wavelength: 220, 254 and 280 nm. In general, the retention time of the product was noted.

The following abbreviations are used herein or within the following illustrative examples:—

### HPLC High Performance Liquid Chromatography

HBTU O-(benzotriazol-1-yl)-N,N,N′,N′-tetramethyluronium hexafluorophosphate;
HATU O-(7-azabenzotriazol-1-yl)-N,N,N′,N′-tetramethyluronium hexafluorophosphate;
HOBT 1-hydroxybenzotriazole;
HOAT 1-hydroxy-7-azabenzotriazole;

### DIEA N,N-diisopropylethylamine;

NMP N-methylpyrrolidin-2-one;
DMSO dimethylsulfoxide;

### DMF N,N-dimethylformamide;

### DMA N,N-dimethylacetamide;

THF tetrahydrofuran;
DME 1,2-dimethoxyethane;
DCCI dicyclohexylcarbodiimide;
MeOH methanol;
MeCN acetonitrile;
DCM dichloromethane;

### DIPEA N,N-diisopropylethylamine.

The chemical names were generated by software which used the Lexichem Toolkit (v. 1.40) from OpenEye Scientific Software (www.eyesopen.com) to generate IUPAC conforming names.

### EXAMPLE 1

### 4-(methylsulfonylmethyl)-6-morpholin-4-yl-2-thiophen-3-yl-pyrimidine

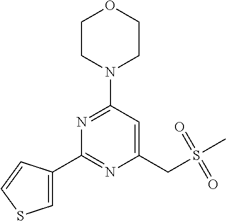

2-methylsulfanyl-4-(methylsulfonylmethyl)-6-morpholin-4-yl-pyrimidine (151 mg, 0.5 mmol), thiophene-3-boronic acid (141 mg, 1.1 mmol), copper(I)thiophene-2-carboxylate (248 mg, 1.3 mmol), palladium tetrakis triphenylphosphine (47 mg, 0.04 mmol) and 1,4-dioxane added (5 ml) were added to a microwave vessel. The system was degassed with nitrogen, sealed and heated in a microwave reactor at 130° C. for 45 minutes. The resulting products were solubilised with NMP and purified by SCX chromatography, eluting the desired compounds with 7N methanol ammonia. The product was further purified using reverse phase preparative HPLC (see purification details after table) to afford the title compound, (4.3 mg).

LCMS Spectrum: MH+ 340.5, Retention Time 1.86, Method: See details after table below.

NMR Spectrum: ¹H NMR (300.132 MHz, DMSO) δ 3.20 (s, 3H), 3.71 (s, 8H), 4.47 (s, 2H), 6.83 (s, 1H), 7.60 (dd, 1H), 7.76 (dd, 1H), 8.29 (dd, 1H)

The starting material 2-methylsulfanyl-4-(methylsulfonylmethyl)-6-morpholin-4-yl-pyrimidine was prepared as follows:

### 2-methylsulfanyl-4-(methylsulfonylmethyl)-6-morpholin-4-yl-pyrimidine

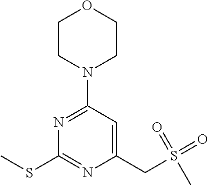

2-methylsulfanyl-6-(methylsulfonylmethyl)pyrimidin-4-ol (15 g, 63.97 mmol) was heated at reflux in phosphorous oxychloride (100 ml) for approximately 1 hour. Phosphorous oxychloride was evaporated and the residue was neutralised with sodium hydroxide solution and extracted into ethyl acetate. The resultant mixture was then dried over magnesium sulfate, filtered and evaporated to dryness to afford the crude 4-chloro-2-methylsulfanyl-6-(methylsulfonylmethyl)pyrimidine. This was then dissolved in DCM, morpholine (319 mmol, 28 ml) was added and the reaction stirred at room temperature. Upon completion the resulting precipitate was collected as a white solid. Concentration of the filtrate afforded more solid 2-methylsulfanyl-4-(methylsulfonylmethyl)-6-morpholin-4-yl-pyrimidine (total 13.7 g).

LCMS Spectrum: MH+ 304.50, Retention Time 1.49, Method: Monitor Base

NMR Spectrum: ¹H NMR (300.132 MHz, DMSO) 62.45 (s, 3H), 3.49-3.74 (m, 8H), 4.37 (s, 2H), 6.66 (s, 1H) ppm.

### 2-methylsulfanyl-6-(methylsulfonylmethyl)pyrimidin-4-ol

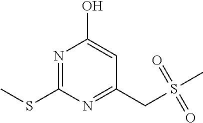

6-(chloromethyl)-2-methylsulfanyl-pyrimidin-4-ol (19.07 g, 100 mmol) was suspended in acetonitrile (400 ml). To this stirring suspension was added methanesulfinic acid sodium salt (12.255 g, 120 mmol) and DMF (100 ml). The reaction was then heated to 100° C. to give a dark suspension and monitored by LCMS. Once complete, the solvents were removed and the resultant product added to 1:1 MeOH:DCM (200 ml) and acidified with acetic acid (10 ml). The resultant precipitate was collected, washed with water (200 ml) and MeOH (100 ml) and dried overnight in vacuo to afford the title compound as a white solid, (16.45 g).

LCMS Spectrum: MH+ 235.2, Retention Time 0.5, Method: Early Base

NMR Spectrum: ¹H NMR (300.132 MHz, DMSO) δ 2.50 (s, 3H), 3.12 (s, 3H), 4.39 (s, 2H), 6.25 (s, 1H), 13.09 (s, 1H) ppm.

### 6-(chloromethyl)-2-methylsulfanyl-pyrimidin-4-ol

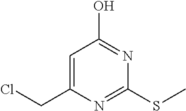

S-Methyl-2-thiopseudourea sulfate (20 g, 71.85 mmol), ethyl 4-chloroacetoacetate (10.755 ml, 79.04 mmol) and sodium carbonate (13.925 g, 107.78 mmol) were dissolved in water (100 ml) and stirred at room temperature overnight. The reaction was monitored by TLC, and once complete, the reaction precipitate was collected and the supernatant was neutralised with 6N hydrochloric acid to yield more reaction precipitate which was also collected. The accumulated precipitate was then washed with water (×3) and an off-white solid was obtained. This was dried in vacuo at 60° C. for 48 hours to yield the desired compound as a pale yellow/white solid, (43.2 g).

Mass Spectrum: M⁺ 190

NMR Spectrum: ¹H NMR (300.132 MHz, CDCl₃) δ 2.59 (s, 3H), 4.35 (s, 2H), 6.41 (s, 1H), 12.70 (s, 1H) ppm

The compounds shown in table 1 were prepared in an analogous manner to 4-(methylsulfonylmethyl)-6-morpholin-4-yl-2-thiophen-3-yl-pyrimidine (example 1), except where noted.

TABLE 1

| | | | | Retention | |
|---|---|---|---|---|---|
| | | | LCMS | Time | |
| Ex. | Structure | NAME | MH+ | (min) | Notes |
| 2 | 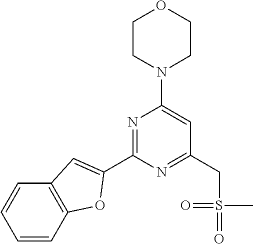 | 2-benzofuran-2-yl-4- (methylsulfonylmethyl)-6- morpholin-4-yl-pyrimidine | 374.57 | 2.05 | |
| 3 | 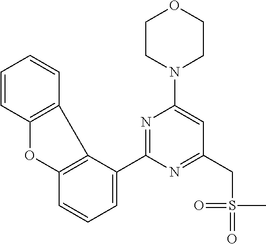 | 2-dibenzofuran-l-yl-4- (methylsulfonylmethyl)-6- morpholin-4-yl-pyrimidine | 424.61 | 2.51 | |
| 4 | 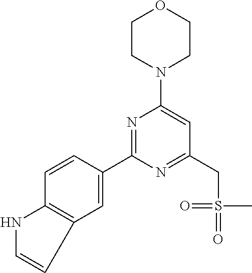 | 5-[4-(methylsulfonylmethyl)- 6-morpholin-4-yl-pyrimidin- 2-yl]-1H-indole | 373.61 | 1.89 | Zinc acetate (1.1 mmol, 175 mg) added to this reaction |
| 5 | 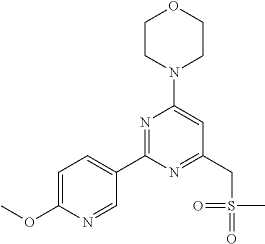 | 2-(6-methoxypyridin-3-yl)-4- (methylsulfonylmethyl)-6- morpholin-4-yl-pyrimidine | 365.61 | 1.78 | Zinc acetate (1.1 mmol, 175 mg) added to this reaction |
| 6 | 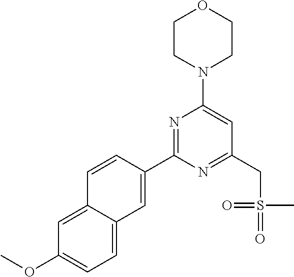 | 2-(6-methoxynaphthalen-2- yl)-4-(methylsulfonylmethyl)- 6-morpholin-4-yl-pyrimidine | 414.63 | 2.39 | |
| 7 | 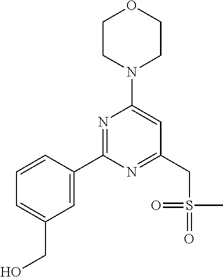 | [3-[4-(methylsulfonylmethyl)- 6-morpholin-4-yl-pyrimidin- 2-yl]phenyl]methanol | 364.6 | 1.51 | Zinc acetate (1.1 mmol, 175 mg) added to this reaction |
| 8 | 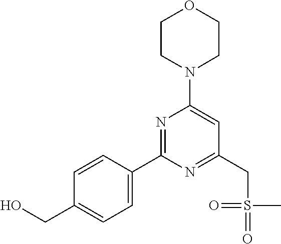 | [4-[4-(methylsulfonylmethyl)- 6-morpholin-4-yl-pyrimidin- 2-yl]phenyl]methanol | 364.61 | 1.48 | Zinc acetate (1.1 mmol, 175 mg) added to this reaction |
| 9 | 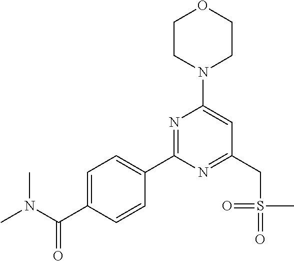 | N,N-dimethyl-4-[4- (methylsulfonylmethyl)-6- morpholin-4-yl-pyrimidin-2- yl]benzamide | 405.64 | 1.55 | |
| 10 | 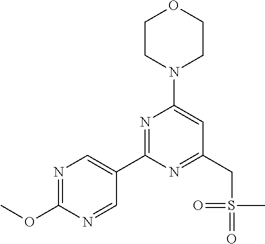 | 2-(2-methoxypyrimidin-5-yl)- 4-(methylsulfonylmethyl)-6- morpholin-4-yl-pyrimidine | 366.59 | 1.50 | Zinc acetate (1.1 mmol, 175 mg) added to this reaction |
| 11 | 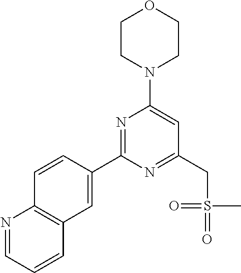 | 6-[4-(methylsulfonylmethyl)- 6-morpholin-4-yl-pyrimidin- 2-yl]quinoline | 385.62 | 1.79 | Zinc acetate (1.1 mmol, 175 mg) added to this reaction |
| 12 | 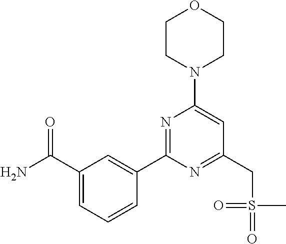 | 3-[4-(methylsulfonylmethyl)- 6-morpholin-4-yl-pyrimidin- 2-yl]benzamide | 377.61 | 1.38 | |

### EXAMPLE 2

¹H NMR (300.132 MHz, DMSO) 53.24 (s, 3H), 3.74 (s, 8H), 4.54 (s, 2H), 6.93 (s, 1H), 7.32 (t, 1H), 7.42 (t, 1H), 7.49-7.82 (m, 3H)

### EXAMPLE 4

¹H NMR (300.132 MHz, DMSO) δ 3.25 (s, 3H), 3.74 (s, 8H), 4.50 (s, 2H), 6.55 (d, 1H), 6.81 (s, 1H), 7.39 (dd, 1H), 7.45 (d, 1H), 7.96 (s, 1H), 8.17 (dd, 1H), 8.61 (s, 1H), 11.24 (s, 1H)

### EXAMPLE 5

¹H NMR (300.132 MHz, DMSO) δ 3.20 (s, 3H), 3.72 (s, 8H), 3.93 (s, 3H), 4.50 (s, 2H), 6.88 (s, 1H), 6.92 (d, 1H), 8.53 (dd, 1H), 9.11 (d, 1H)

### EXAMPLE 6

¹H NMR (300.132 MHz, DMSO) δ 3.25 (s, 3H), 3.76 (s, 8H), 3.91 (s, 3H), at 4.54 (s, 2H), 6.90 (s, 1H), 7.21 (dd, 1H), 7.38 (d, 1H), 7.90 (d, 1H), 7.99 (d, 1H), 8.42 (dd, 1H), 8.83 (s, 1H)

### EXAMPLE 7

¹H NMR (300.132 MHz, DMSO) δ 3.22 (s, 3H), 3.73 (s, 8H), 4.52 (s, 2H), 4.58 (d, 2H), 5.25 (t, 1H), 6.90 (s, 1H), 7.43 (s, 1H), 7.45 (s, 1H), 8.22 (td, 1H), 8.31 (s, 1H)

### EXAMPLE 9

¹H NMR (300.132 MHz, DMSO) δ 2.93 (s, 3H), 2.99 (s, 3H), 3.21 (s, 3H), 3.74 (s, 8H), 4.53 (s, 2H), 6.93 (s, 1H), 7.51 (d, 2H), 8.38 (d, 2H)

### EXAMPLE 10

¹H NMR (300.132 MHz, DMSO) δ 3.19 (s, 3H), 3.72 (s, 8H), 4.01 (s, 3H), 4.50 (s, 2H), 6.94 (s, 1H), 9.38 (s, 2H)

### EXAMPLE 11

¹H NMR (300.132 MHz, DMSO) δ 3.26 (s, 3H), 3.78 (s, 8H), 4.57 (s, 2H), 6.97 (s, 1H), 7.59 (dd, 1H), 8.12 (d, 1H), 8.55 (d, 1H), 8.71 (dd, 1H), 8.96 (m, 2H)

### Purification/Analysis Details for Examples 1 to 12:

| | |
|---|---|
| Dissolution | 4 ml DMF |
| Solvent | |
| Instrument | Waters XBridge Prep, C18 5 μm 100 × 19 mm |
| Column | Phenomenex Gemini 5μ, C18 100 × 21.2 mm |
| Fraction Trigger | uv @ 254 nm |
| Gradient | 0-1 min 30% MeCN, 9.5 min 60% MeCN |
| Solvent A | Water |
| Solvent B | Acetonitrile |
| Solvent C - | 4:3:3 880 Ammonia:Acetonitrile:Water |
| Modifier 5% | |
| Flow Rate | 20 ml/min |
| At Column Dilution | Acetonitrile |
| Solvent | |
| At Column Dilution | 1.0 ml/min |
| Flow Rate | |
| Transfer solvent | 1 ml DMF per tube + MeOH wash |
| LCMS | 50 μl made upto 1 ml with MeCN |
| Analytical | Phenomenex Gemini 5μ, C18 50 × 2 mm, |
| LCMS Method | 1.2 ml/min |
| | 0 min 95:0:5 A:B:C, 4 min 0:95:5 A:B:C |
| | A MeCN, B H₂O, C 1:1 MeCN:H₂O 1% |
| | Ammonia acid |

### EXAMPLE 13

### 4-(benzenesulfonylmethyl)-6-morpholin-4-yl-2-thiophen-3-yl-pyrimidine

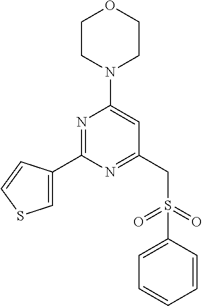

A suspension of 4-(benzenesulfonylmethyl)-2-methylsulfanyl-6-morpholin-4-yl-pyrimidine (183 mg), 3-thiopheneboronic acid (129.5 mg), copper(I)thiophene-2-carboxylate (248 mg) and tetrakis(triphenylphosphine)palladium (0) (47 mg) in 1,4-dioxane (5 ml) was degassed with a stream of dry nitrogen. This suspension was heated in a microwave reactor (Emrys Optimizer, Personal Chemistry, Sweden) at 130° C. for 45 minutes. The reaction mixture was then diluted with methanol:DCM 1:9 and this mixture was purified by chromatography on an ‘Isolute SCX-2’ column (10 g; International Sorbent Technology Limited, Mid Glamorgan, UK) by initially washing the column with a gradient of 10 to 100% methanol in DCM, followed by elution of crude product with a mixture of methanolic ammonia (7M):DCM, 1:3. The methanolic ammonia solution was evaporated and the residues further purified by HPLC using a Phenomenex ‘Gemini’ preparative reversed-phase column (5 microns silica, 21.2 mm diameter, 100 mm length) using decreasingly polar mixtures of water and acetonitrile (containing 2% formic acid) as eluent, to yield the title compound. (87.3 mg).

LCMS Spectrum: MH+ 402.73, Retention Time 1.96, Method: Monitor Acid

NMR Spectrum: ¹H NMR (300.132 MHz, DMSO) δ 3.56-3.74 (m, 8H), 4.68 (s, 2H), 6.66 (s, 1H), 7.37 (dd, 1H), 7.50 (dd, 1H), 7.54-7.69 (m, 2H), 7.75 (tt, 1H), 7.78-7.84 (m, 2H), 7.90 (dd, 1H)

The starting material 4-(benzenesulfonylmethyl)-2-methylsulfanyl-6-morpholin-4-yl-pyrimidine was prepared as follows.

### 4-(benzenesulfonylmethyl)-2-methylsulfanyl-6-morpholin-4-yl-pyrimidine

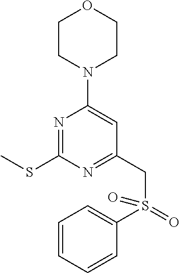

6-(benzenesulfonylmethyl)-2-methylsulfanyl-pyrimidin-4-ol (15.99 g,) and phosphorous oxychloride (87.4 ml) were heated at reflux for 4 hours. Phosphorous oxychloride was removed by evaporation and the residue adjusted to pH 7 with aqueous sodium hydroxide solution. The crude product was extracted into ethyl acetate, the ethyl acetate layer separated and dried over magnesium sulfate. The solvent was removed by evaporation to afford the crude 4-(benzenesulfonylmethyl)-6-chloro-2-methylsulfanyl-pyrimidine. This was dissolved in DCM (100 ml) and morpholine (23.6 ml) was added. The reaction mixture was stirred at ambient temperature for 1 hour. The solvent was removed by evaporation, the residue dissolved in DCM and purified on silica eluting with a gradient of 0% to 20% methanol in DCM to yield the title compound as a white solid (11.26 g).

LCMS Spectrum: MH+ 366, Retention Time 1.97, Method: Monitor Base

NMR Spectrum: (DMSOd₆ 2.14 (3H, s), 3.51-3.53 (4H, m), 3.64-3.66 (4H, m), 3.67 (1H, s), 4.57 (2H, s), 6.47 (1H, s), 7.61-7.65 (2H, m), 7.72-7.76 (1H, m), 7.77-7.80 (2H, m);

### 6-(benzenesulfonylmethyl)-2-methylsulfanyl-pyrimidin-4-ol

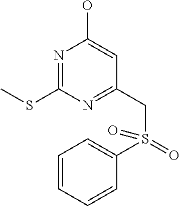

6-(chloromethyl)-2-methylsulfanyl-pyrimidin-4-ol (19.07 g, from example 1) was suspended in acetonitrile (400 ml). To this suspension was added benzenesulfinic acid sodium salt (19.7 g) and DMF (100 ml). The mixture was heated to 100° C. to give a dark suspension. The solvent was removed in vacuo until nearly dry and a 1:1 mixture of methanol:DCM (200 ml) was added. Acetic acid (10 ml) was then added and the resulting precipitate collected and washed with water (200 ml) and methanol (100 ml). This material was dried overnight in vacuo to afford the title compound as a white solid. (19.55 g).

LCMS Spectrum: MH+ 297, Retention Time 0.72, Method: Monitor Base

NMR Spectrum: (DMSOd₆) 2.01 (s, 3H), 4.59 (s, 2H), 6.15 (s, 1H), 7.62 (t, 2H), 7.74 (tt, 1H), 7.81 (dd, 2H), 12.31-13.08 (m, 1H);

The compounds in table 2 were prepared in an analogous manner to 4-(benzenesulfonylmethyl)-6-morpholin-4-yl-2-thiophen-3-yl-pyrimidine (example 13) using the appropriate boronic acid.

TABLE 2

| | | | LCMS | Retention |
|---|---|---|---|---|
| Example | Structure | NAME | MH+ | time (min) |
| 14 | 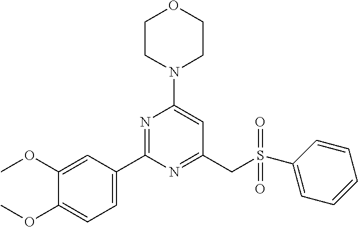 | 4-(benzenesulfonylmethyl)-2- (3,4-dimethoxyphenyl)-6- morpholin-4-yl-pyrimidine | 456.84 | 1.84 |
| 15 | 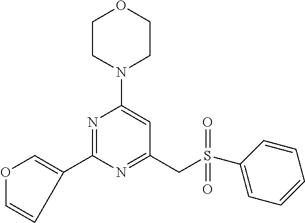 | 4-(benzenesulfonylmethyl)-2-(3- furyl)-6-morpholin-4-yl- pyrimidine | 386.79 | 1.77 |
| 16 | 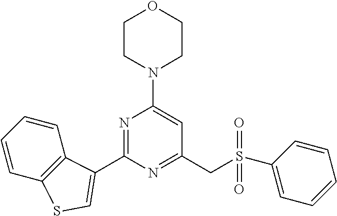 | 4-(benzenesulfonylmethyl)-2- benzothiophen-3-yl-6-morpholin- 4-yl-pyrimidine | 452.83 | 2.59 |
| 17 | 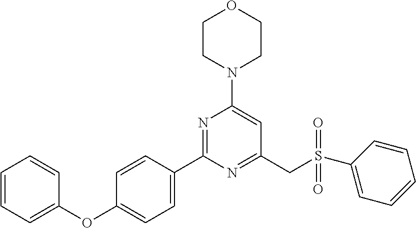 | 4-(benzenesulfonylmethyl)-6- morpholin-4-yl-2-(4- phenoxyphenyl)pyrimidine | 488.91 | 2.78 |
| 18 | 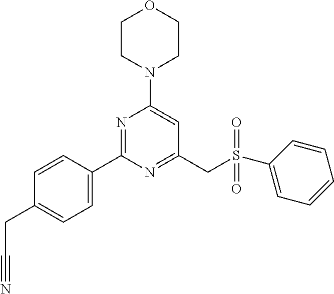 | 4-[4-(benzenesulfonylmethyl)- 6-morpholin-4-yl-pyrimidin-2- yl]phenyl]acetonitile | 435.85 | 2.08 |
| 19 | 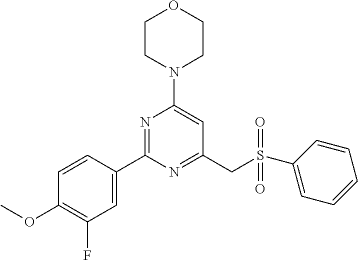 | 4-(benzenesulfonylmethyl)-2-(3- fluoro-4-methoxy-phenyl)-6- morpholin-4-yl-pyrimidine | 444.86 | 2.32 |
| 20 | 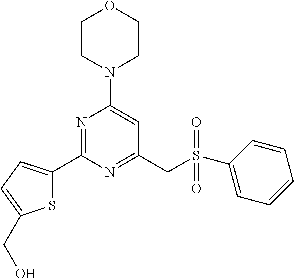 | [5-[4-(benzenesulfonylmethyl)-6- morpholin-4-yl-pyrimidin-2- yl]thiophen-2-yl]methanol | 432.82 | 1.82 |
| 21 | 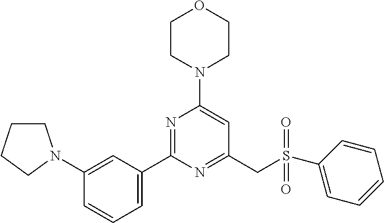 | 4-(benzenesulfonylmethyl)-6- morpholin-4-yl-2-(3-pyrrolidin-l- ylphenyl)pyrimidine | 465.94 | 2.38 |
| 22 | 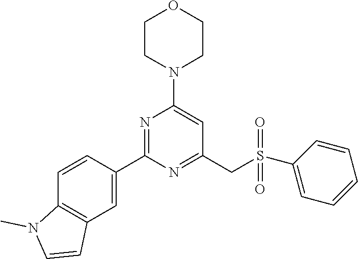 | 5-[4-(benzenesulfonylmethyl)-6- morpholin-4-yl-pyrimidin-2-yl]- 1-methyl-indole | 449.91 | 1.87 |
| 23 | 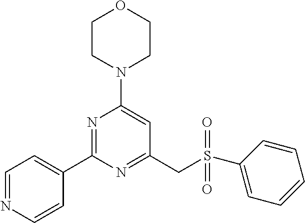 | 4-(benzenesulfonylmethyl)-6- morpholin-4-yl-2-pyridin-4-yl- pyrimidine | 397.51 | 1.19 |
| 24 | 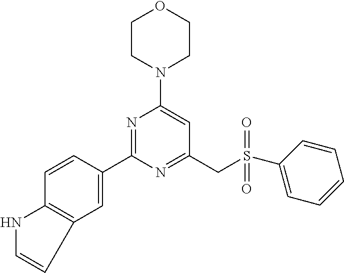 | 5-[4-(benzenesulfonylmethyl)-6- morpholin-4-yl-pyrimidin-2-yl]- 1H-indole | 435.61 | 1.58 |
| 25 | 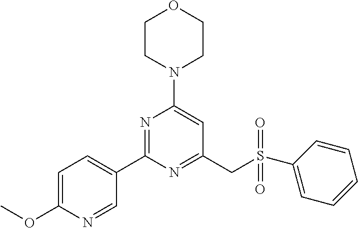 | 4-(benzenesulfonylmethyl)-2-(6- methoxypyridin-3-yl)-6- morpholin-4-yl-pyrimidine | 427.58 | 2.09 |

### EXAMPLE 15

¹H NMR (300.132 MHz, DMSO) δ 3.43-3.74 (m, 8H), 4.65 (s, 2H), 6.60 (d, 1H), 6.63 (s, 1H), 7.50-7.87 (m, 6H), 7.93 (s, 1H)

### EXAMPLE 16

¹H NMR (300.132 MHz, DMSO) δ 3.61-3.78 (m, 8H), 4.77 (s, 2H), 6.75 (s, 1H), 7.30-7.43 (m, 2H), 7.56-7.75 (m, 3H), 7.81-7.88 (m, 2H), 8.00 (d, 1H), 8.33 (s, 1H), 8.56 (dd, 1H)

### EXAMPLE 20

¹H NMR (300.132 MHz, DMSO) δ 2.13 (s, 2H), 3.45-3.71 (m, 8H), 4.57 (s, 2H), 6.47 (s, 1H), 7.62 (t, 2H), 7.70-7.83 (m, 5H). 1×OH not observed

### EXAMPLE 21

¹H NMR (300.132 MHz, DMSO) δ 3.12-3.43 (m, 8H), 3.56-3.78 (m, 8H), 4.71 (s, 2H), 6.59-6.61 (m, 1H), 6.72 (s, 1H), 7.14 (t, 1H), 7.24 (d, 2H), 7.46-7.89 (m, 5H)

### EXAMPLE 22

¹H NMR (300.132 MHz, DMSO) δ 3.58-3.77 (m, 8H), 3.80 (s, 3H), 4.72 (s, 2H), 6.48 (t, 1H), 6.67 (s, 1H), 7.34 (s, 1H), 7.36 (d, 1H), 7.57-7.69 (m, 3H), 7.69-7.89 (m, 4H)

### EXAMPLE 26

### 4-morpholin-4-yl-6-(phenylsulfanylmethyl)-2-pyridin-2-yl-pyrimidine

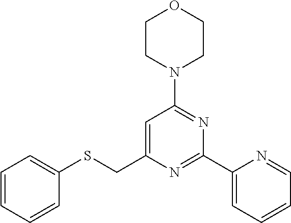

Sodium ethoxide (49 mg, 0.72 mmol) was added potion wise to a stirred solution of thiophenol (79.4 mg, 0.72 mmole) in acetonitrile (2.5 ml) at room temperature under an inert atmosphere portionwise. This mixture was stirred for 30 minutes before 4-(chloromethyl)-6-morpholin-4-yl-2-pyridin-2-yl-pyrimidine (174 mg, 0.60 mmole) as a solution in acetonitrile (2.5 ml) was added dropwise. Stirring was continued overnight at room temperature and under a nitrogen atmosphere after which the reaction mixture was evaporated to dryness and the residue partitioned between ethyl acetate and water. The combined organics were then dried over magnesium sulfate, filtered and evaporated to dryness to afford crude product. The product was purified by basic preparative HPLC chromatography (gradient elution 35-55% MeCN in water) and the desired product obtained as a clear gum (94 mg, 43%).

LCMS Spectrum: MH+ 365.5 Retention time 2.15, Method: Monitor Base

NMR Spectrum: ¹H NMR (300.132 MHz, DMSO) 3.67 (d, 8H), 4.22 (s, 2H), 6.83 (s, 1H), 7.20 (t, 1H), 7.32 (t, 2H), 7.42-7.50 (m, 3H), 7.91 (td, 1H), 8.25 (d, 1H), 8.70 (d, 1H)

The starting material 4-(chloromethyl)-6-morpholin-4-yl-2-pyridin-2-yl-pyrimidine was prepared as follows:

### 4-(chloromethyl)-6-morpholin-4-yl-2-pyridin-2-yl-pyrimidine

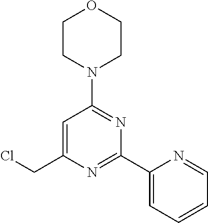

6-(chloromethyl)-2-pyridin-2-yl-pyrimidin-4-ol (14.07 g, 63.46 mmol) was dissolved in phosphorus oxychloride (50 mL) and heated to reflux for one hour. Phosphorous oxychloride was then evaporated, and azeotroped with toluene (100 mL). Water (100 mL) was added and the mixture was adjusted to pH 10 with sodium hydroxide. The reaction mixture was then extracted with ethyl acetate (2×200 mL), washed with brine (100 mL) and dried over magnesium sulfate. Evaporation afforded a beige solid, 4-chloro-6-(chloromethyl)-2-pyridin-2-yl-pyrimidine (3.563 g,). 4-chloro-6-(chloromethyl)-2-pyridin-2-yl-pyrimidine (3.563 g, 14.84 mmol), morpholine (1.295 g, 14.84 mmol) and DIPEA (5.745 g, 44.52 mmol) were dissolved in THF (20 mL) and the reaction stirred at room temperature for 2 hours. PS-Isocyanate resin (5 g) was then added and stirring continued for 3 hours, after which the reaction mixture was filtered and washed with THF followed by methanol. The combined organics were evaporated onto silica and the product was purified by flash chromatography. The clean fractions were evaporated to afford the desired product as a crystalline solid, (2.7 g).

LCMS Spectrum: MH+ 291.51, Retention time 1.69, Method: Monitor Acid.

NMR Spectrum: ¹HNMR (300.132 MHz, DMSO) δ 3.75 (s, 8H), 4.68 (s, 2H), 7.02 (s, 1H), 7.49 (m, 1H), 7.92 (dt, 1H), 8.31 (d, 1H), 8.71 (d, 1H) ppm.

### 6-(chloromethyl)-2-pyridin-2-yl-pyrimidin-4-ol

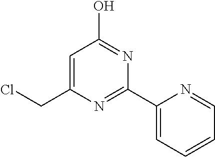

Sodium ethoxide (3.6 mmol, 245 mg) and methyl 4-chloroacetoacetate (3.3 mmol, 498 mg) were added to a solution of 2-pyridylamidine (3 mmol, 364 mg) in ethanol (10 ml) and the reaction mixture was heated to reflux. After three hours the reaction mixture was concentrated in vacuo and acidified with hydrochloric acid to yield the desired compound as a pale beige solid, (445 mg).

LCMS Spectrum: MH+ 222.48, Retention time 0.76, Method: Monitor Base

NMR Spectrum: ¹H NMR (300.132 MHz, DMSO) δ 4.36 (d, 2H), 6.32 (s, 1H), 7.65 (ddd, 1H), 8.04 (td, 1H), 8.28 (d, 1H), 8.74 (d, 1H), 11.17-12.28 (m, 1H) ppm.

The compounds in table 3 were prepared in an analogous manner to 4-morpholin-4-yl-6-(phenylsulfanylmethyl)-2-pyridin-2-yl-pyrimidine (example 26) by reacting the appropriate starting material with 4-(chloromethyl)-6-morpholin-4-yl-2-pyridin-2-yl-pyrimidine.

TABLE 3

| | | | | Retention |
|---|---|---|---|---|
| | | | LCMS | time |
| Example | Structure | NAME | MH+ | (min) |
| 27 | 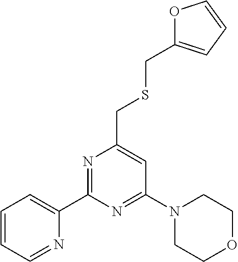 | 4-(2- furylmethylsulfanylmethyl)-6- morpholin-4-yl-2-pyridin-2-yl- pyrimidine | 369.74 | 1.07 |
| 28 | 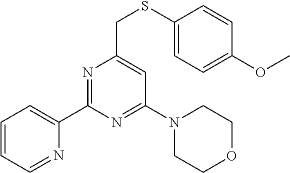 | 4-[(4- methoxyphenyl)sulfanylmethyl]- 6-morpholin-4-yl-2-pyridin- 2-yl-pyrimidine | 395.78 | 1.24 |
| 29 | 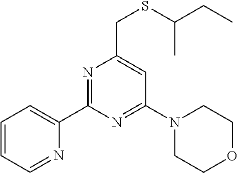 | 4-(butan-2-ylsulfanylmethyl)- 6-morpholin-4-yl-2-pyridin-2- yl-pyrimidine | 345.75 | 1.22 |
| 30 | 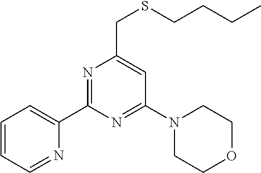 | 4-(butylsulfanylmethyl)-6- morpholin-4-yl-2-pyridin-2-yl- pyrimidine | 345.75 | 1.25 |
| 31 | 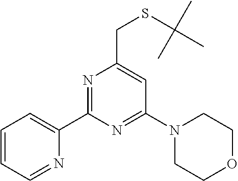 | 4-morpholin-4-yl-2-pyridin-2- yl-6-(tert- butylsulfanylmethyl)pyrimidine | 345.75 | 1.19 |
| 32 | 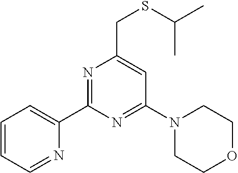 | 4-morpholin-4-yl-6-(propan-2- ylsulfanylmethyl)-2-pyridin-2- yl-pyrimidine | 331.72 | 1.09 |
| 33 | 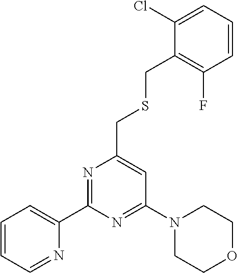 | 4-[(2-chloro-6-fluoro- phenyl)methylsulfanylmethyl]- 6-morpholin-4-yl-2-pyridin-2- yl-pyrimidine | 431.78 | 1.38 |
| 34 | 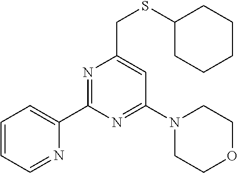 | 4-(cyclohexylsulfanylmethyl)- 6-morpholin-4-yl-2-pyridin-2- yl-pyrimidine | 371.82 | 1.4 |
| 35 | 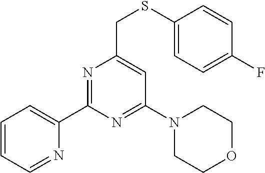 | 4-[(4- fluorophenyl)sulfanylmethyl]- 6-morpholin-4-yl-2-pyridin-2- yl-pyrimidine | 383.76 | 1.27 |
| 36 | 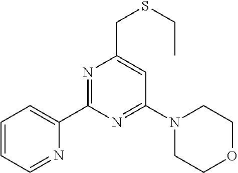 | 4-(ethylsulfanylmethyl)-6- morpholin-4-yl-2-pyridin-2-yl- pyrimidine | 317.69 | 0.96 |
| 37 | 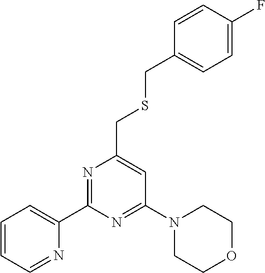 | 4-[(4- fluorophenyl)methylsulfanyl methyl]-6-morpholin-4-yl-2- pyridin-2-yl-pyrimidine | 397.8 | 1.27 |
| 38 | 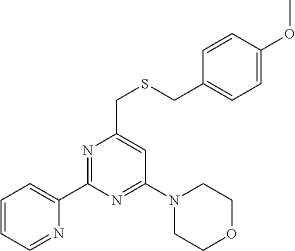 | 4-[(4- methoxyphenyl)methylsulfanyl methyl]-6-morpholin-4-yl-2- pyridin-2-yl-pyrimidine | 409.82 | 1.22 |
| 39 | 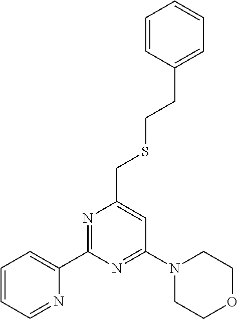 | 4-morpholin-4-yl-6- (phenethylsulfanylmethyl)-2- pyridin-2-yl-pyrimidine | 393.8 | 1.37 |
| 40 | 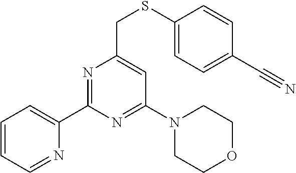 | 4-[(6-morpholin-4-yl-2- pyridin-2-yl-pyrimidin-4- yl)methylsulfanyl]benzonitrile | 390.79 | 1.28 |
| 41 | 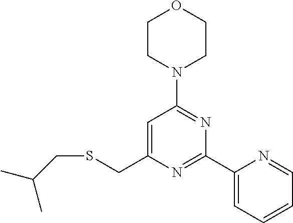 | 4-(2- methylpropylsulfanylmethyl)- 6-morpholin-4-yl-2-pyridin-2- yl-pyrimidine | 345.81 | 1.3 |
| 42 | 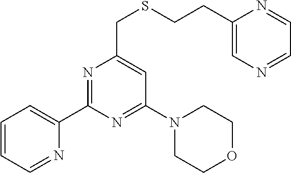 | 4-morpholin-4-yl-6-(2-pyrazin- 2-ylethylsulfanylmethyl)-2- pyridin-2-yl-pyrimidine | 395.58 | 2.38 |
| 43 | 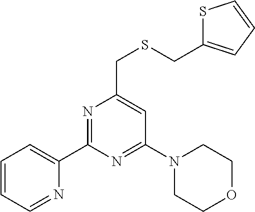 | 4-morpholin-4-yl-2-pyridin-2- yl-6-(thiophen-2- ylmethylsulfanylmethyl)pyrimidine | 385.54 | 3.09 |

### EXAMPLE 31

¹H NMR (400.132 MHz, DMSO) δ 1.32 (s, 9H), 3.65 (s, 8H), 3.72 (s, 2H), 6.83 (s, 1H), 7.41 (ddd, 1H), 7.85 (td, 1H), 8.23 (dt, 1H), 8.64 (ddd, 1H)

### EXAMPLE 39

¹H NMR (400.132 MHz, DMSO) δ 2.75-2.84 (m, 4H), 3.65 (s, 8H), 3.69 (s, 2H), 6.80 (s, 1H), 7.10-7.20 (m, 5H), 7.42 (ddd, 1H), 7.85 (td, 1H), 8.24 (dt, 1H), 8.65 (ddd, 1H)

### EXAMPLE 44

### 4-(benzenesulfonylmethyl)-6-morpholin-4-yl-2-pyridin-2-yl-pyrimidine

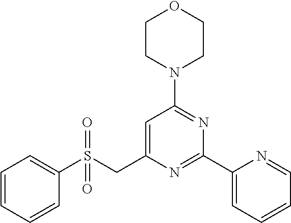

A solution of Oxone® (110 mg, 0.18 mmol) in water (2.5 ml) was added to a stirred solution of 4-morpholin-4-yl-6-(phenylsulfanylmethyl)-2-pyridin-2-yl-pyrimidine (example 26) (46.5 mg, 0.13 mmol) in ethanol (2.5 ml) at room temperature and stirring continued for 3 hours at room temperature. Water was then added (5 ml) and the organics extracted with DCM (3×10 ml). The combined organics were washed with brine, dried over magnesium sulfate, filtered and evaporated to dryness to afford crude product which was purified by basic preparative HPLC chromatography (gradient elution 25-45% MeCN in water) to yield the desired product obtained as an off white solid (28.4 mg, 55%).

LCMS Spectrum: MH+ 397.53 Retention time 1.70, Method: Monitor Base

NMR Spectrum: ¹H NMR (300.132 MHz, DMSO) δ 3.65-3.70 (m, 8H), 4.74 (s, 2H), 6.77 (s, 1H), 7.42-7.47 (m, 1H), 7.60-7.65 (m, 2H), 7.72-7.77 (m, 1H), 7.80-7.86 (m, 4H), 8.66 (ddd, 1H)

The compounds shown in table 4 were prepared in an analogous manner to 4-(benzenesulfonylmethyl)-6-morpholin-4-yl-2-pyridin-2-yl-pyrimidine (example 44) using the appropriate starting material from table 3. Where a starting material is not shown in table 3, it was prepared in an ananlogous manner to example 26 by replacing thiophenol with the appropriate reactant.

TABLE 4

| | | | | Retention |
|---|---|---|---|---|
| | | | LCMS | Time |
| Example | Structure | NAME | MH+ | (min) |
| 45 | 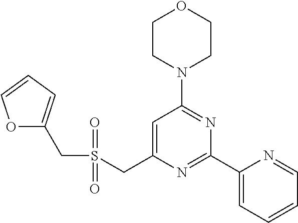 | 4-(2- furylmethylsulfonylmethyl)- 6-morpholin-4-yl-2-pyridin- 2-yl-pyrimidine | 401.6 | 3.27 |
| 46 | 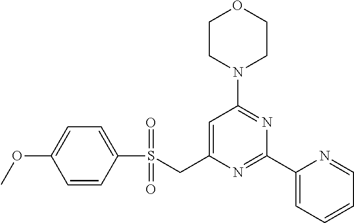 | 4-[(4- methoxyphenyl)sulfonylmethyl]- 6-morpholin-4-yl-2-pyridin-2- yl-pyrimidine | 427.6 | 3.41 |
| 47 | 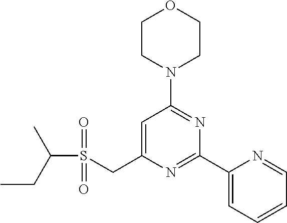 | 4-(butan-2-ylsulfonylmethyl)- 6-morpholin-4-yl-2-pyridin-2- yl-pyrimidine | 377.6 | 3.36 |
| 48 | 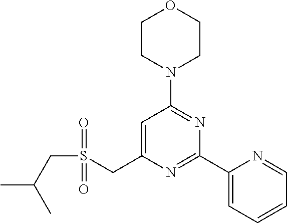 | 4-(2- methylpropylsulfonylmethyl)- 6-morpholin-4-yl-2-pyridin-2- yl-pyrimidine | 377.6 | 3.46 |
| 49 | 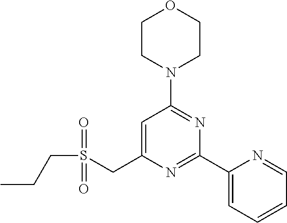 | 4-morpholin-4-yl-6- (propylsulfonylmethyl)-2- pyridin-2-yl-pyrimidine | 363.6 | 3.26 |
| 50 | 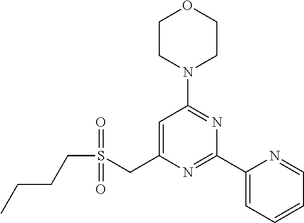 | 4-(butylsulfonylmethyl)-6- morpholin-4-yl-2-pyridin- 2-yl-pyrimidine | 377.6 | 3.48 |
| 51 | 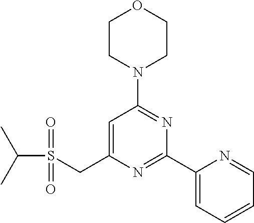 | 4-morpholin-4-yl-6-(propan- 2-ylsulfonylmethyl)-2- pyridin-2-yl-pyrimidine | 363.6 | 3.14 |
| 52 | 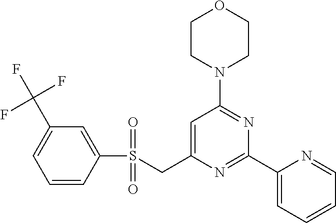 | 4-morpholin-4-yl-2-pyridin- 2-yl-6-[[3- (trifluoromethyl)phenyl] sulfonylmethyl]pyrimidine | 465.6 | 3.71 |
| 53 | 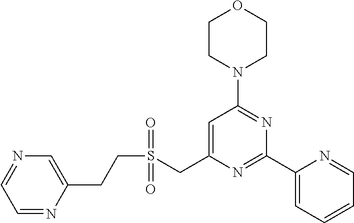 | 4-morpholin-4-yl-6- (2-pyrazin-2- ylethylsulfonylmethyl)- 2-pyridin-2-yl-pyrimidine | 427.6 | 3.09 |
| 54 | 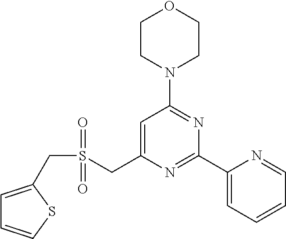 | 4-morpholin-4-yl-2- pyridin-2-yl-6-(thiophen-2- ylmethylsulfonylmethyl) pyrimidine | 417.6 | 3.48 |
| 55 | 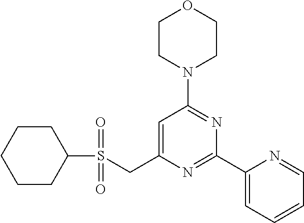 | 4- (cyclohexylsulfonylmethyl)- 6-morpholin-4-yl-2-pyridin- 2-yl-pyrimidine | 403.6 | 3.64 |
| 56 | 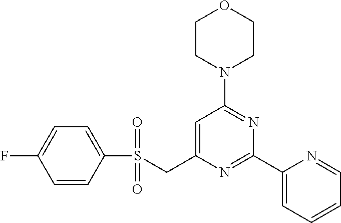 | 4-[(4- fluorophenyl)sulfonylmethyl]- 6-morpholin-4-yl-2-pyridin-2- yl-pyrimidine | 415.6 | 3.44 |
| 57 | 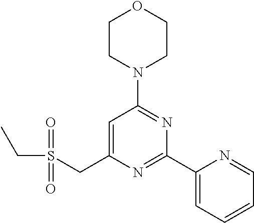 | 4-(ethylsulfonylmethyl)-6- morpholin-4-yl-2-pyridin- 2-yl-pyrimidine | 349.6 | 3.02 |
| 58 | 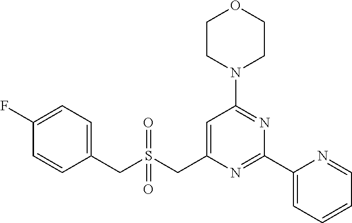 | 4-[(4-fluorophenyl) methylsulfonylmethyl]- 6-morpholin-4-yl-2- pyridin-2-yl-pyrimidine | 429.6 | 2 |
| 59 | 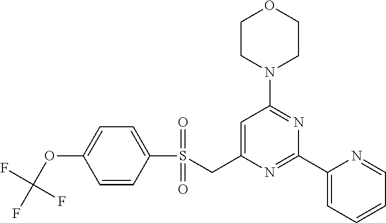 | 4-morpholin-4-yl-2-pyridin- 2-yl-6-[[4- (trifluoromethoxy)phenyl] sulfonylmethyl]pyrimidine | 481.6 | 3.82 |
| 60 | 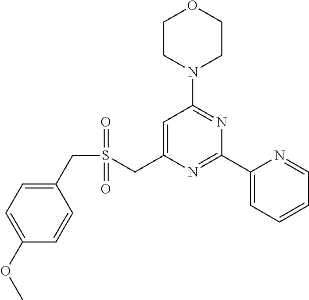 | 4-[(4-methoxyphenyl) methylsulfonylmethyl]- 6-morpholin-4-yl-2- pyridin-2-yl-pyrimidine | 441.6 | 3.58 |
| 61 | 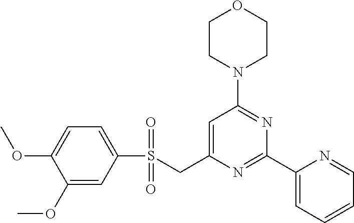 | 4-[(3,4-dimethoxyphenyl) sulfonylmethyl]-6- morpholin-4-yl-2-pyridin- 2-yl-pyrimidine | 457.6 | 1.64 |
| 62 | 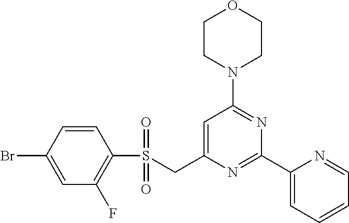 | 4-[(4-bromo-2-fluoro- phenyl)sulfonylmethyl]-6- morpholin-4-yl-2-pyridin- 2-yl-pyrimidine | 493.5/ 495.5 | 3.72 |
| 63 | 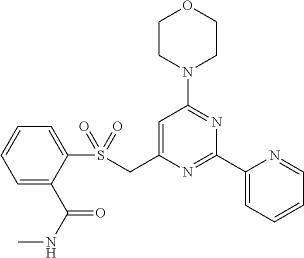 | N-methyl-2-[(6-morpholin-4- yl-2-pyridin-2-yl-pyrimidin-4- yl)methylsulfonyl]benzamide | 454.6 | 3.1 |
| 64 | 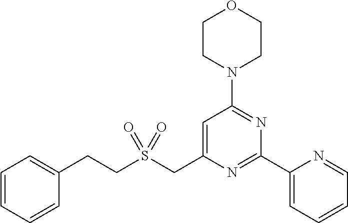 | 4-morpholin-4-yl-6- (phenethylsulfonylmethyl)- 2-pyridin-2-yl-pyrimidine | 425.60 | 3.8 |
| 65 | 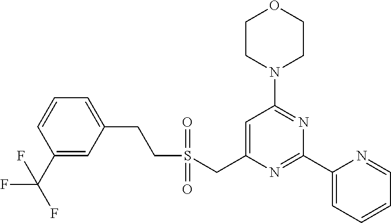 | 4-morpholin-4-yl-2-pyridin- 2-yl-6-[2-[3- (trifluoromethyl)phenyl] ethylsulfonylmethyl] pyrimidine | 394.54 | 4.0 |
| 66 | 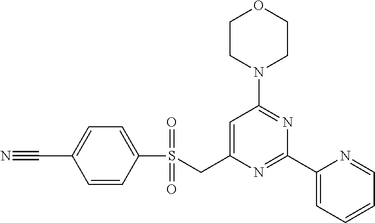 | 4-[(6-morpholin-4-yl-2- pyridin-2-yl-pyrimidin-4- yl)methylsulfonyl] benzonitrile | 422.54 | 3.34 |
| 67 | 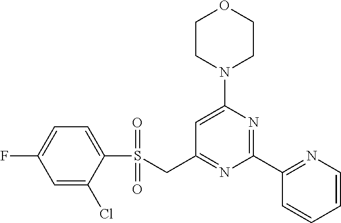 | 4-[(2-chloro-4-fluoro- phenyl)sulfonylmethyl]- 6-morpholin-4-yl-2- pyridin-2-yl-pyrimidine | 449.56 | 1.94 |

### EXAMPLE 45

¹H NMR (500.133 MHz, DMSO) δ 3.74 (s, 8H), 4.53 (s, 2H), 4.97 (s, 2H), 6.53 (d, 1H), 6.75 (d, 1H), 6.99 (s, 1H), 7.50 (m, 1H), 7.74 (s, 1H), 7.95 (td, 1H), 8.35 (d, 1H), 8.73 (d, 1H)

### EXAMPLE 46

¹H NMR (500.133 MHz, DMSO) δ 3.62 (m, 8H), 3.76 (s, 3H), 4.58 (s, 2H), 6.68 (s, 1H), 7.04 (d, 2H), 7.38 (m, 1H), 7.66 (d, 2H), 7.74 (t, 1H), 7.80 (d, 1H), 8.60 (d, 1H)

### EXAMPLE 47

¹H NMR (500.133 MHz, DMSO) δ 1.01 (t, 3H), 1.34 (d, 3H), 1.50 (m, 1H), 2.10 (m, 1H), 3.45 (m, 1H), 3.73 (s, 8H), 4.52 (s, 2H), 6.98 (s, 1H), 7.50 (m, 1H), 7.93 (td, 1H), 8.29 (d, 1H), 8.72 (d, 1H)

### EXAMPLE 48

¹H NMR (500.133 MHz, DMSO) δ 51.00 (d, 6H), 2.20-2.27 (m, 1H), 3.32 (d, 2H), 3.66 (s, 8H), 4.43 (s, 2H), 6.91 (s, 1H), 7.42 (ddd, 1H), 7.87 (td, 1H), 8.23 (dt, 1H), 8.64 (ddd, 1H)

### EXAMPLE 53

¹H NMR (500.133 MHz, DMSO) δ 3.39-3.42 (m, 2H), 3.74 (s, 8H), 3.90-3.93 (m, 2H), 4.64 (s, 2H), 7.02 (s, 1H), 7.49 (ddd, 1H), 7.90 (td, 1H), 8.53 (d, 1H), 8.59 (dd, 1H), 8.32 (dt, 1H), 8.66 (ddd, 1H), 8.76 (d, 1H)

### EXAMPLE 54

¹H NMR (500.133 MHz, DMSO) δ 3.67 (s, 8H), 4.41 (s, 2H), 5.05 (s, 2H), 6.91 (s, 1H), 7.03 (dd, 1H), 7.35 (d, 1H), 7.42-7.45 (m, 1H), 7.53 (dd, 1H), 7.89 (td, 1H), 8.29 (d, 1H), 8.66-8.67 (m, 1H)

### EXAMPLE 55

¹H NMR (500.133 MHz, DMSO) δ 1.08-1.40 (m, 6H), 1.78 (d, 2H), 2.16 (d, 2H), 3.42-3.47 (m, 1H), 3.66 (s, 8H), 4.41 (s, 2H), 6.90 (s, 1H), 7.43 (ddd, 1H), 7.88 (td, 1H), 8.23 (dt, 1H), 8.65 (ddd, 1H)

### EXAMPLE 57

¹H NMR (500.133 MHz, DMSO) δ 1.32 (t, 3H), 3.35 (q, 2H), 3.73 (s, 8H), 4.51 (s, 2H), 6.98 (s, 1H), 7.49 (ddd, 1H), 7.94 (td, 1H), 8.29 (d, 1H), 8.72 (d, 1H)

### EXAMPLE 68

### 4-[(3-methoxyphenoxy)methyl]-6-morpholin-4-yl-2-pyridin-2-yl-pyrimidine

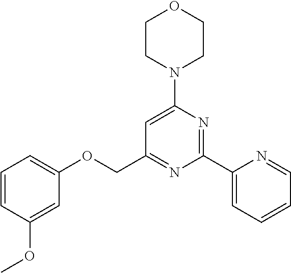

Sodium hydride (18 mg, 0.45 mmol) was added to a stirred solution of 3-methoxy phenol (56 mg, 0.45 mmol) in DMF (2 ml) at room temperature and stirring continued for 30 minutes. A solution of 4-(chloromethyl)-6-morpholin-4-yl-2-pyridin-2-yl-pyrimidine (87 mg, 0.30 mmol, from example 26) in DMF (1 ml) was then added quickly dropwise followed by a catalytic amount of sodium iodide. This reaction mixture was then stirred at room temperature for 5 minutes and then warmed to 70° C. for 1.5 hours. After being evaporated to dryness, the residue was partitioned between ethyl acetate and water and the combined organics dried over magnesium sulfate, filtered and evaporated under reduced pressure to afford the crude product, which was purified by basic preparative HPLC chromatography to obtain the desired product as a clear yellow gum (69 mg, 61. %).

LCMS Spectrum: MH+ 379.6 Retention time 2.20, Method: Monitor Base

NMR Spectrum: ¹H NMR (300.132 MHz, DMSO) δ 3.72 (s, 8H), 3.75 (s, 3H), 5.08 (s, 2H), 6.57 (m, 1H), 6.67 (m, 2H), 6.94 (s, 1H), 7.22 (t, 1H), 7.49 (ddd, 1H), 7.93 (td, 1H), 8.32 (d, 1H), 8.71 (d, 1H)

The compounds shown in table 5 were prepared in an analogous manner to 4-[(3-methoxyphenoxy)methyl]-6-morpholin-4-yl-2-pyridin-2-yl-pyrimidine (example 68) by to reacting the appropriate starting material with 4-(chloromethyl)-6-morpholin-4-yl-2-pyridin-2-yl-pyrimidine (from example 26).

TABLE 5

| | | | LCMS | Retention |
|---|---|---|---|---|
| Example | Structure | NAME | MH+ | time (min) |
| 69 | 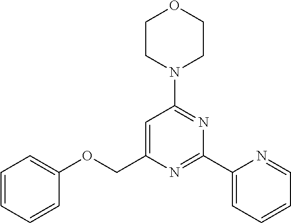 | 4-morpholin-4-yl-6- (phenoxymethyl)-2-pyridin- 2-yl-pyrimidine | 349.6 | 2.15 |
| 70 | 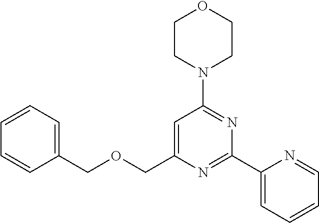 | 4-morpholin-4-yl-6- (phenylmethoxymethyl)-2- pyridin-2-yl-pyrimidine | 363.6 | 2.12 |
| 71 | 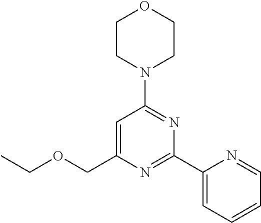 | 4-(ethoxymethyl)-6-morpholin- 4-yl-2-pyridin-2-yl-pyrimidine | 301.6 | 1.57 |
| 72 | 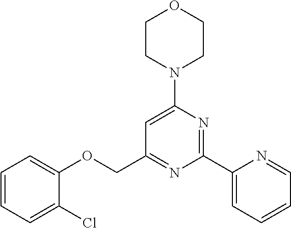 | 4-[(2-chlorophenoxy)methyl]- 6-morpholin-4-yl-2-pyridin-2- yl-pyrimidine | 383.6 | 2.38 |
| 73 | 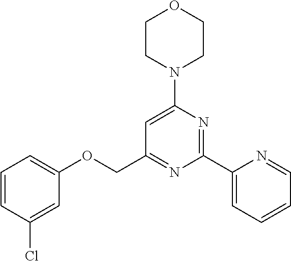 | 4-[(3-chlorophenoxy)methyl]- 6-morpholin-4-yl-2-pyridin-2- yl-pyrimidine | 383.6 | 2.40 |
| 74 | 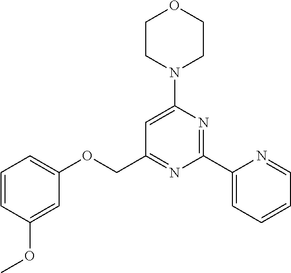 | 4-[(3-methoxyphenoxy)methyl]- 6-morpholin-4-yl-2-pyridin-2- yl-pyrimidine | 379.6 | 2.20 |
| 75 | 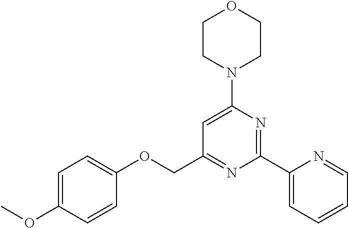 | 4-[(4-methoxyphenoxy)methyl]- 6-morpholin-4-yl-2-pyridin-2- yl-pyrimidine | 379.6 | 2.12 |
| 76 | 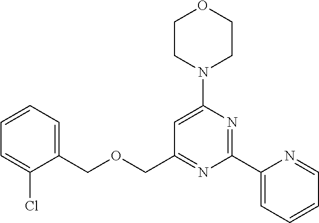 | 4-[(2- chlorophenyl)methoxymethyl]- 6-morpholin-4-yl-2-pyridin-2- yl-pyrimidine | 397.6 | 2.35 |
| 77 | 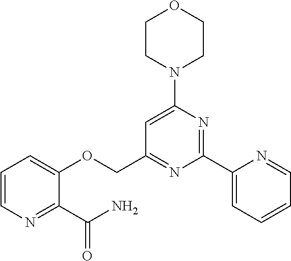 | 3-[(6-morpholin-4-yl-2-pyridin- 2-yl-pyrimidin-4- yl)methoxy]pyridine-2- carboxamide | 393.6 | 1.32 |
| 78 | 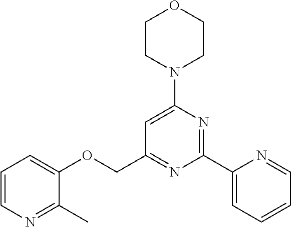 | 4-[(2-methylpyridin-3- yl)oxymethyl]-6-morpholin-4- yl-2-pyridin-2-yl-pyrimidine | 364.6 | 1.70 |
| 79 | 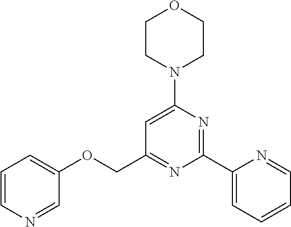 | 4-morpholin-4-yl-2-pyridin-2- yl-6-(pyridin-3- yloxymethyl)pyrimidine | 350.6 | 1.58 |

### EXAMPLE 69

¹H NMR (300.132 MHz, DMSO) δ 3.75 (s, 8H), 5.12 (s, 2H), 6.94 (s, 1H), 6.98 (t, 1H), 7.09 (d, 2H), 7.33 (t, 2H), 7.49 (m, 1H), 7.93 (dt, 1H), 8.32 (d, 1H), 8.71 (d, 1H),

### EXAMPLE 70

¹H NMR (300.132 MHz, DMSO) δ 3.75 (s, 8H), 4.56 (s, 2H), 4.69 (s, 2H), 6.84 (s, 1H), 7.29-7.50 (m, 6H), 7.91 (dt, 1H), 8.29 (d, 1H), 8.69 (d, 1H),

### EXAMPLE 71

¹H NMR (300.132 MHz, DMSO) δ 1.22 (t, 3H), 3.62 (q, 2H), 3.75 (s, 8H), 4.50 (s, 2H), 6.80 (s, 1H), 7.47 (m, 1H), 7.91 (dt, 1H), 8.29 (d, 1H), 8.69 (d, 1H),

### EXAMPLE 72

¹H NMR (300.132 MHz, DMSO) δ 3.76 (s, 8H), 5.23 (s, 2H), 6.97 (s, 1H), 7.01 (dt, 1H), 7.32 (m, 2H), 7.49 (m, 2H), 7.93 (dt, 1H), 8.32 (d, 1H), 8.71 (d, 1H),

### EXAMPLE 73

¹H NMR (300.132 MHz, DMSO) δ 53.77 (s, 8H), 5.16 (s, 2H), 6.97 (s, 1H), 7.07 (dt, 2H), 7.23 (t, 1H), 7.35 (t, 1H), 7.49 (m, 1H), 7.93 (dt, 1H), 8.32 (d, 1H), 8.71 (d, 1H),

### EXAMPLE 74

¹H NMR (300.132 MHz, DMSO) δ 3.72 (s, 8H), 3.75 (s, 3H), 5.08 (s, 2H), 6.57 (m, 1H), 6.67 (m, 2H), 6.94 (s, 1H), 7.22 (t, 1H), 7.49 (ddd, 1H), 7.93 (td, 1H), 8.32 (d, 1H), 8.71 (d, 1H)

### EXAMPLE 75

¹H NMR (300.132 MHz, DMSO) δ 3.72 (s, 11H), 5.04 (s, 2H), 6.96 (d, 5H), 7.49 (m, 1H), 7.93 (td, 1H), 8.32 (d, 1H), 8.71 (d, 1H)

### EXAMPLE 80

### N-benzyl-N-methyl-1-(6-morpholin-4-yl-2-pyridin-2-yl-pyrimidin-4-yl)methanamine

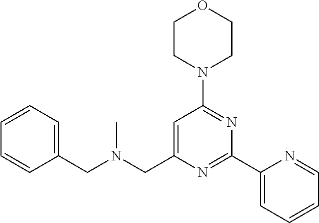

N-methyl benzylamine (25 mg, 0.2 mmol) and DIPEA (52 mg, 0.4 mmol) was added to 4-(chloromethyl)-6-morpholin-4-yl-2-pyridin-2-yl-pyrimidine (60 mg, 0.2 mmol, from example 26) in DMF (4 mL) and the reaction mixture was heated to 150° C. in the microwave for 20 minutes. After cooling, the product was purified directly by preparative HPLC (5-40% MeCN/H₂O) and evaporation afforded the desired compound as a gum, (25.3 mg).

LCMS Spectrum: MH+ 376.70, Retention time 2.14, Method: Monitor Base

NMR Spectrum: ¹H NMR (300.132 MHz, DMSO) δ 2.22 (s, 3H), 3.58 (s, 2H), 3.62 (s, 2H), 3.65-3.77 (m, 8H), 6.89 (s, 1H), 7.23-7.28 (m, 1H), 7.33 (d, 2H), 7.39 (t, 2H), 7.46 (dd, 1H), 7.91 (td, 1H), 8.30 (d, 1H), 8.70 (d, 1H)

### EXAMPLE 81

### N-[(6-morpholin-4-yl-2-pyridin-2-yl-pyrimidin-4-yl)methyl]propan-2-amine

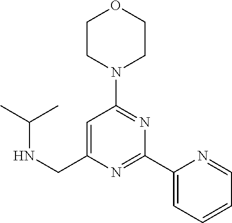

Isopropylamine (25 mg, 0.4 mmol) and DIPEA (52 mg, 0.4 mmol) were added to 4-(chloromethyl)-6-morpholin-4-yl-2-pyridin-2-yl-pyrimidine (60 mg, 0.2 mmol, from example 26) in DMF (4 mL) and the reaction mixture was heated to 150° C. in the microwave for 20 minutes. After cooling the product was purified directly by preparative HPLC (5-40% MeCN/H₂O) and evaporation afforded the desired compound as a gum, (32.6 mg).

LCMS Spectrum: MH+ 314.64, Retention time 1.71, Method: Monitor Base

NMR Spectrum: ¹H NMR (300.132 MHz, DMSO) δ 1.03 (s, 3H), 1.05 (s, 3H), 2.77 (septet, 1H), 3.31 (s, 2H), 3.71 (s, 8H), 6.88 (s, 1H), 7.46 (ddd, 1H), 7.90 (td, 1H), 8.31 (d, 1H), 8.69 (dd, 1H), 1×NH not observed.

The compounds shown in table 6 were prepared in an analogous manner to N-[(6-morpholin-4-yl-2-pyridin-2-yl-pyrimidin-4-yl)methyl]propan-2-amine (example 81) using 4-(chloromethyl)-6-morpholin-4-yl-2-pyridin-2-yl-pyrimidine (from example 26) and the appropriate amine

TABLE 6

| | | | LCMS | Retention |
|---|---|---|---|---|
| Example | Structure | NAME | MH+ | time (min) |
| 82 | 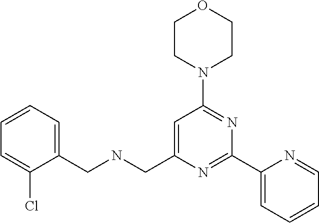 | 1-(2-chlorophenyl)-N-[(6- morpholin-4-yl-2-pyridin- 2-yl-pyrimidin-4- yl)methyl]methanamine | 396.62 | 2.03 |

### EXAMPLE 83

### 4-(benzenesulfonylmethyl)-5-fluoro-6-morpholin-4-yl-2-pyridin-2-yl-pyrimidine

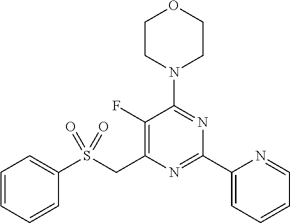

Benzene sulfinic acid sodium salt (32 mg, 0.19 mmol) was added to a stirring solution of 4-(chloromethyl)-5-fluoro-6-morpholin-4-yl-2-pyridin-2-yl-pyrimidine (50 mg, 0.16 mmol) in dry DMF. The mixture was heated to 80° C. for 1 hour and then concentrated. The residue was purified by flash chromatography—eluting with 0-10% MeOH/DCM to give 4-(benzenesulfonylmethyl)-5-fluoro-6-morpholin-4-yl-2-pyridin-2-yl-pyrimidine as a white solid (47.6 mg, 72%)

LCMS Spectrum: MH+ 415.41, Retention Time 1.44, Method: Monitor Acid

NMR Spectrum: ¹H NMR (300.132 MHz, DMSO) δ 3.72-3.84 (m, 8H), 4.88 (d, 2H), 7.48-7.54 (m, 1H), 7.63-7.73 (m, 2H), 7.77-7.93 (m, 5H), 8.72 (d, 1H)

### EXAMPLE 84

### 5-fluoro-4-(methylsulfonylmethyl)-6-morpholin-4-yl-2-pyridin-2-yl-pyrimidine

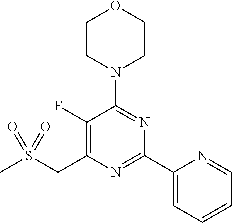

This compound was prepared using an analogous method to that used in example 83 for 4-(benzenesulfonylmethyl)-5-fluoro-6-morpholin-4-yl-2-pyridin-2-yl-pyrimidine using methanesulfinic acid sodium salt (20 mg, 0.19 mmol) to give 5-fluoro-4-(methylsulfonylmethyl)-6-morpholin-4-yl-2-pyridin-2-yl-pyrimidine as a white solid (20.5 mg, 36%).

LCMS Spectrum: MH+ 353.52, Retention Time 0.90, Method: Monitor Acid.

NMR Spectrum: ¹H NMR (300.132 MHz, DMSO) δ 3.25 (s, 3H), 3.72-3.79 (m, 4H), 3.81-3.87 (m, 4H), 4.68 (s, 2H), 7.47-7.53 (m, 1H), 7.90-7.98 (m, 1H), 8.27 (d, 1H), 8.71 (d, 1H)

The starting material. 4-(chloromethyl)-5-fluoro-6-morpholin-4-yl-2-pyridin-2-yl-pyrimidine was prepared as follows:

### 4-(chloromethyl)-5-fluoro-6-morpholin-4-yl-2-pyridin-2-yl-pyrimidine

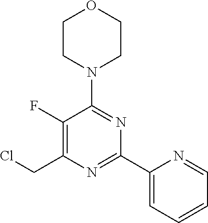

Selectfluor™ (1.35 g, 3.78 mmol) was added to a solution of 4-(chloromethyl)-6-morpholin-4-yl-2-pyridin-2-yl-pyrimidine (1 g, 3.44 mmol, from example 26) in methanol (25 ml) and was heated at 50° C. for 16 hours. Saturated sodium hydrogen carbonate (5 ml) was added to the reaction mixture and the methanol was removed in vacuo. Water (50 ml) was added to the aqueous residues and the resultant precipitate was filtered, washed with water and dried. This was purified by chromatography eluting with ethyl acetate to give 4-(chloromethyl)-5-fluoro-6-morpholin-4-yl-2-pyridin-2-yl-pyrimidine as a white solid (210 mg, 20%).

LCMS Spectrum: MH+ 309.35, Retention Time 1.34, Method: Monitor Acid.

NMR Spectrum: ¹H NMR (300.132 MHz, DMSO) δ 3.71-3.79 (m, 4H), 3.80-3.87 (m, 4H), 4.75 (d, 2H), 7.46-7.52 (m 1H), 7.89-7.97 (m, 1H), 8.27 (d, H), 8.71 (d, 1H)

### EXAMPLE 85

### 6-morpholin-4-yl-N-phenyl-2-pyridin-2-yl-pyrimidine-4-carboxamide

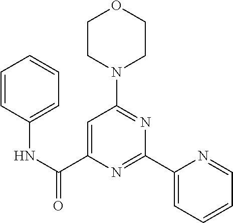

DIPEA (114 mg, 0.88 mmol), HATU (168 mg, 0.44 mmol) and aniline (41 mg, 0.44 mmol) were added to 6-morpholin-4-yl-2-pyridin-2-yl-pyrimidine-4-carboxylic acid (115 mg, 0.4 mmol) in THF (4 ml) and the reaction was stirred at room temperature for 2 hours, after which water was added. The resulting precipitate was collected by filtration and dried under vacuum to afford the title compound as a white solid, (87 mg).

LCMS Spectrum: MH+ 362.51, Retention time 2.39, Method: Monitor Base

NMR Spectrum: ¹H NMR (300.132 MHz, DMSO) δ 3.70-3.91 (m, 8H), 7.18 (t, 1H), 7.39-7.44 (m, 3H), 7.55 (ddd, 1H), 7.87 (d, 2H), 7.99 (td, 1H), 8.66 (d, 1H), 8.77 (d, 1H), 10.48 (s, 1H) ppm.

### EXAMPLE 86

### N,N-dimethyl-6-morpholin-4-yl-2-pyridin-2-yl-pyrimidine-4-carboxamide

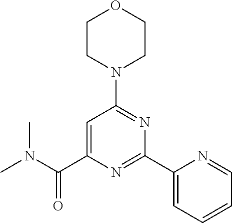

This compound was prepared in an analogous manner to that used in example 85 for 6-morpholin-4-yl-N-phenyl-2-pyridin-2-yl-pyrimidine-4-carboxamide using 6-morpholin-4-yl-2-pyridin-2-yl-pyrimidine-4-carboxylic acid.

LCMS Spectrum: MH+ 314.45, Retention time 1.26, Method: Monitor Base

NMR Spectrum: ¹H NMR (300.132 MHz, DMSO) δ 2.97 (s, 3H), 3.01 (s, 3H), 3.72 (s, 8H), 6.93 (s, 1H), 7.50 (ddd, 1H), 7.93 (td, 1H), 8.31 (d, 1H), 8.71 (d, 1H).

The starting material 6-morpholin-4-yl-2-pyridin-2-yl-pyrimidine-4-carboxylic acid was prepared as follows.

### 6-morpholin-4-yl-2-pyridin-2-yl-pyrimidine-4-carboxylic acid

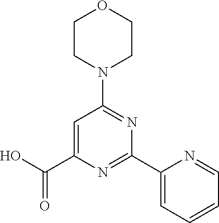

Methyl orotate (5 g, 29.41 mmol) was suspended in phosphorous oxychloride (50 ml) and the mixture was heated to reflux for 4 hours. After this time excess phosphorous oxychloride was removed under reduced pressure. The resulting dark residue was poured onto ice with vigorous stirring and the solution was left to stir until all the ice had melted. The crude product was then collected by filtration and the filtrate was extracted with ether (×2). The filtered product was added to the ether washings and dried over magnesium sulfate. The solution was then concentrated to give methyl 2,6-dichloropyrimidine-4-carboxylate (5.25 g, 25.37 mmol) as a yellow oil that solidified on standing. To this was added morpholine (2.005 g, 25.37 mmol) and THF (40 ml) and the mixture left for 2 hours at room temperature. The reaction was then evaporated to dryness to afford methyl 2-chloro-6-morpholin-4-yl-pyrimidine-4-carboxylate (5.41 g, 21 mmol)

LCMS Spectrum: MH+ 258.39, Retention time 1.56, Method: Monitor Base Methyl 2-chloro-6-morpholin-4-yl-pyrimidine-4-carboxylate (2.58 g, 10 mmol), 2-tributylstannyl pyridine (4.055 g, 11 mmol) and tetrakis(triphenylphosphine)palladium (0) (10 mol %, 1 mmol, 1.116 g) were suspended in THF (20 ml) and heated to 100° C. for 30 minutes in the microwave. To this mixture was added sodium hydroxide (20 ml) (4M in H₂O), and the reaction was stirred for 1 hour. The resulting precipitate was collected by filtration found to be the monosodium salt of 6-morpholin-4-yl-2-pyridin-2-yl-pyrimidine-4-carboxylic acid, (1.53 g).

LCMS Spectrum: (M+Na)+308.47, Retention Time 1.42, Method: Monitor Base

NMR Spectrum: ¹H NMR (300.132 MHz, D₂O) δ3.70-3.86 (m, 8H), 7.11 (s, 1H), 7.51 (ddd, 1H), 7.94 (td, 1H), 8.28 (d, 1H), 8.60 (d, 1H) ppm.

### EXAMPLE 87

### 5-[4-(Methylsulfonylmethyl)-6-morpholin-4-yl-pyrimidin-2-yl]-1,3-dihydroindol-2-one

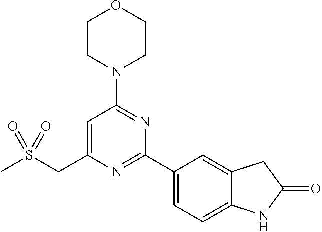

2-Chloro-4-(methylsulfonylmethyl)-6-morpholin-4-yl-pyrimidine (120 mg) was dissolved in a solvent mixture (18% DMF in 7:3:2 DME:Water:Ethanol) (7 mL). 5-(4,4,5,5-Tetramethyl-1,3,2-dioxaborolan-2-yl)-1,3-dihydroindol-2-one (303 mg), a 2M solution of sodium carbonate (2 mL) and dichlorobis(triphenylphosphine) palladium (40 mg) were then added to the solution and the mixture heated at 100° C. for 30 minutes in a microwave reactor. The reaction mixture was loaded onto a SCX-2 column (10 g), washed with methanol and removed with 7N ammonia in methanol. The material was concentrated in vacuo and purified by prep-HPLC (basic) to give the desired material as a white solid (18 mg).

Mass Spectrum; MH⁺ 389.

NMR Spectrum: ¹H NMR (DMSO-d₆) δ3.20 (3H, s), 3.57 (2H, s), 3.71-3.73 (8H, m), 4.48 (2H, s), 6.82 (1H, s), 6.91 (1H, d), 8.20 (1H, s), 8.23-8.25 (1H, m), 10.55 (1H, s)

The preparation of 5-(4,4,5,5-tetramethyl-1,3,2-dioxaborolan-2-yl)-1,3-dihydroindol-2-one is described below:

### 5-(4,4,5,5-Tetramethyl-1,3,2-dioxaborolan-2-yl)-1,3-dihydroindol-2-one

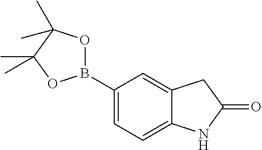

A mixture of 5-bromo-2,3-dihydroindol-2-one (500 mg), bis(pinacolato)diboron (899 mg) and potassium acetate (695 mg) in DMF (20 mL) was degassed for 5 minutes. 1,1′-Bis(diphenylphosphino)ferrocenedichloropalladium(II) dichloromethane adduct (78 mg) was added to the mixture and the reaction was heated to 80° C. and left to stir for 3 hours. The reaction mixture was filtered through Celite® and concentrated in vacuo. The residue was suspended in water (50 mL) and extracted with ethyl acetate (2×50 mL). The organics were dried (MgSO₄), filtered and concentrated in vacuo to give the desired material as a brown solid. (611 mg).

Mass Spectrum; M+H+MeCN⁺ 301.

NMR Spectrum: ¹H NMR (DMSO-d₅) δ 1.28 (12H, s), 3.47 (2H, s), 6.82-6.84 (1H, d), 7.51 (2H, m), 10.52 (1H, s)

The preparation of 2-chloro-4-(methylsulfonylmethyl)-6-morpholin-4-yl-pyrimidine is described below:

### 2-Chloro-4-(methylsulfonylmethyl)-6-morpholin-4-yl-pyrimidine

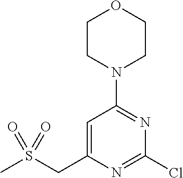

A suspension of 2,4-dichloro-6-(methylsulfonylmethyl)pyrimidine (10.56 g) in DCM (230 mL) was stirred magnetically and cooled to −5° C. Triethylamine (6.78 mL) was added followed by the dropwise addition of a solution of morpholine (3.85 mL) in DCM (30 mL) maintaining the reaction temperature below −5° C. The reaction was stirred at room temperature for 1 hour and then the organic mixture washed with water (300 mL). The organic phase was dried (MgSO₄), filtered and evaporated to a brown solid which was chromatographed on silica, eluting with 50% ethyl acetate in DCM, to give the desired material (6.81 g) as a white solid.

Mass Spectrum: MH+ 292.

NMR Spectrum: ¹H NMR (DMSO-d₆) δ3.12 (3H, s), 3.63 (4H, s), 3.68-3.70 (4H, m), 4.45 (2H, s), 6.96 (1H, s)

### 2,4-Dichloro-6-(methylsulfonylmethyl)pyrimidine

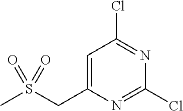

6-(Methylsulfonylmethyl)-1H-pyrimidine-2,4-dione (12.72 g) was suspended in phosphorus oxychloride (125 mL) and heated at reflux under nitrogen for 14 hours. The solution was cooled and concentrated in vacuo to. Iced water (250 mL) was slowly added to the residue and the product then extracted with DCM (3×200 mL). The organics were concentrated in vacuo to give the desired material as a brown solid (10.56 g).

Mass Spectrum: (M−H)⁻ 239.

NMR Spectrum: ¹HNMR (DMSO-d₆) δ3.14 (3H, s), 4.79 (2H, s), 7.88 (1H, s)

### 6-(Methylsulfonylmethyl)-1H-pyrimidine-2,4-dione

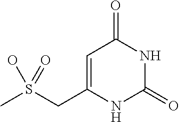

6-(Chloromethyl)uracil (10.00 g) was dissolved in DMF (300 mL) and methanesulfinic acid sodium salt (7.64 g) added. The reaction was heated at 125° C. for 1 hour. The reaction was allowed to cool, filtered and the filtrate concentrated in vacuo to give the desired material as a yellow solid (12.72 g).

NMR Spectrum: ¹H NMR (DMSO-d₆) δ 3.10 (3H, s), 4.27 (2H, s), 5.63 (1H, s), 10.94 (1H, s), 11.16 (1H, s).

### EXAMPLE 88

### Methyl 2-amino-5-[4-(methylsulfonylmethyl)-6-morpholin-4-yl-pyrimidin-2-yl]benzoate

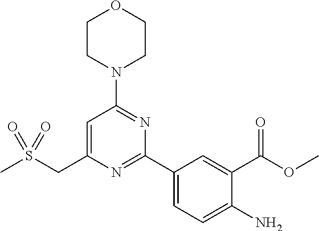

A mixture of methyl-2-amino-5-bromobenzoate (250 mg), potassium acetate (320 mg) and bis(pinacolato)diboron (332 mg) in 1,4-dioxane (10 mL) was degassed for 5 minutes. 1,1′-Bis(diphenylphosphino)ferrocenedichloropalladium(II) dichloromethane adduct (54 mg) was added and the reaction was heated to 80° C. for 2.5 hours. 2-Chloro-4-(methylsulfonylmethyl)-6-morpholin-4-yl-pyrimidine (381 mg), ethanol (0.75 mL), a 2M solution of sodium carbonate (2.7 mL) and additional 1,1′-bis(diphenylphosphino)ferrocenedichloropalladium(II) dichloromethane adduct (54 mg) were added and the heating was continued for a further 3.5 hours. The cooled reaction mixture was loaded on a SCX-2 (10 g), removed with 7N ammonia in methanol and the solution concentrated in vacuo. The residue was chromatographed on silica, eluting with 50% ethyl acetate in DCM, to give the desired material as a yellow solid (82 mg).

Mass Spectrum; MH+ 407

NMR Spectrum: ¹H NMR (DMSO-d₆) δ 3.22 (3H, s), 3.69 (4H, s), 3.73 (4H, s), 3.84 (3H, s), 4.49 (2H, s), 6.77 (1H, s), 6.87 (1H, d), 7.05 (2H, s), 8.24 (1H, d), 8.79 (1H, s)

### EXAMPLE 89

### [2-Methoxy-5-[4-(methylsulfonylmethyl)-6-morpholin-4-yl-pyrimidin-2-yl]phenyl]methanol

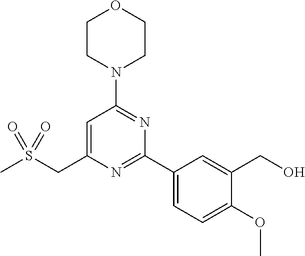

A mixture of 5-bromo-2-methoxybenzylalcohol (250 mg), potassium acetate (339 mg) and bis(pinacolato)diboron (352 mg) in 1,4-dioxane (10 mL) was degassed for 5 minutes. 1,1′-Bis(diphenylphosphino)ferrocenedichloropalladium(II) dichloromethane adduct (57 mg) was added and the reaction was heated to 80° C. for 3 hours. 2-Chloro-4-(methylsulfonylmethyl)-6-morpholin-4-yl-pyrimidine (337 mg), ethanol (0.75 mL), a 2M solution of sodium carbonate (2.7 mL) and additional 1,1′-bis(diphenylphosphino)ferrocenedichloropalladium(II) dichloromethane adduct (57 mg) were added and the heating was continued for a further 66 hours. The reaction mixture was cooled and concentrated in vacuo. The residue was partitioned between ethyl acetate (50 mL) and water (50 mL) and filtered. The organic phase was dried (MgSO₄), concentrated in vacuo and chromatographed on silica, eluting with 5% methanol in DCM. The chromatography was repeated and the residue triturated with diethyl ether to give the desired compound as a white solid (158 mg).

Mass Spectrum; MH+ 394

NMR Spectrum: ¹H NMR (DMSO-d₆) δ3.23 (3H, s), 3.73-3.74 (8H, m), 3.84 (3H, d), 4.51 (2H, s), 4.54 (2H, d), 5.08 (1H, t), 6.83 (1H, s), 7.00-7.06 (1H, m), 8.23-8.26 (1H, m), 8.41 (1H, d)

### EXAMPLE 90

### 2-Methyl-5-[4-(methylsulfonylmethyl)-6-morpholin-4-yl-pyrimidin-2-yl]-1H-benzoimidazole

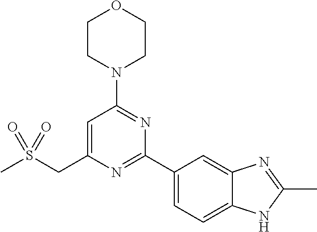

A mixture of 5-bromo-2-methyl-1H-benzoimidazole (250 mg), potassium acetate (349 mg) and bis(pinacolato)diboron (362 mg) in 1,4-dioxane (10 mL) was degassed for 5 minutes. 1,1′-Bis(diphenylphosphino)ferrocenedichloropalladium(10 dichloromethane adduct (59 mg) was added and the reaction was heated to 80° C. for 18 hours. 2-Chloro-4-(methylsulfonylmethyl)-6-morpholin-4-yl-pyrimidine (346 mg), ethanol (0.75 mL), a 2M solution of sodium carbonate (2.7 mL) and additional 1,1′-bis(diphenylphosphino)ferrocenedichloropalladium(II) dichloromethane adduct (59 mg) were added and the heating was continued for a further 3 hours. The cooled reaction mixture was concentrated in vacuo, dissolved in methanol and loaded onto a SCX-2 column (10 g). The column was washed with methanol and the compound removed with 7N ammonia in methanol. The solution was concentrated in vacuo and the residue chromatographed by prep-HPLC (basic) to give the desired compound as a grey solid (5 mg).

Mass Spectrum; MH+ 388.

The preparation of 5-bromo-2-methyl-1H-benzoimidazole is described below:

### 5-Bromo-2-methyl-1H-benzoimidazole

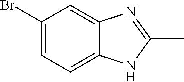

4-Bromobenzene-1,2-diamine (1 g) was dissolved in phosphorus oxychloride (10 mL). Acetic acid (0.297 mL) was added to the mixture at room temperature. The reaction was then heated to 95° C. for 2 hours. The reaction was allowed to cool and the excess phosphorus oxychloride was removed in vacuo. The reaction was quenched with water and evaporated to dryness. The residue was dissolved in methanol and loaded onto a SCX-2 column (20 g) and the compound removed with 7N ammonia in methanol. The solution was concentrated in vacuo and chromatographed on silica, eluting with 5% methanol in DCM, to give the desired material (731 mg) as a white solid.

Mass Spectrum: MH+ 213

NMR Spectrum: ¹H NMR (DMSO-d₆) δ2.62 (3H, s), 7.31-7.34 (1H, m), 7.39 (1H, d), 7.67 (1H, s)

### EXAMPLE 91

### 5-[4-(Methylsulfonylmethyl)-6-morpholin-4-yl-pyrimidin-2-yl]-1,3-dihydrobenzoimidazol-2-one

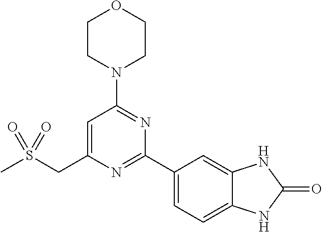

A mixture of 5-bromo-1,3-dihydrobenzoimidazol-2-one (250 mg), potassium acetate (346 mg) and bis(pinacolato)diboron (358 mg) in 1,4-dioxane (10 mL) was degassed for 5 minutes. 1,1′-Bis(diphenylphosphino)ferrocenedichloropalladium(II) dichloromethane adduct (58 mg) was added and the reaction was heated to 80° C. for 3 hours. 2-Chloro-4-(methylsulfonylmethyl)-6-morpholin-4-yl-pyrimidine (343 mg), ethanol (0.75 mL), a 2M solution of sodium carbonate (2.7 mL) and additional 1,1′-bis(diphenylphosphino)ferrocenedichloropalladium(II) dichloromethane adduct (58 mg) were added and the heating was continued for a further 18 hours. The cooled reaction mixture was concentrated in vacuo, dissolved in methanol and loaded onto a SCX-2 column (10 g). The column was washed with methanol and the compound removed with 7N ammonia in methanol. The solution was concentrated in vacuo and the residue chromatographed by prep-HPLC (basic) to give the desired compound as a white solid (26 mg).

Mass Spectrum; MH+ 390

NMR Spectrum: ¹HNMR (DMSO-d₆) δ3.21 (3H, s), 3.72 (8H, t), 4.50 (2H, s), 6.83 (1H, s), 7.01 (1H, d), 7.93 (1H, d), 8.04-8.07 (1H, m), 10.68 (1H, s), 10.81 (1H, s)

The preparation of 5-bromo-1,3-dihydrobenzoimidazol-2-one is described below:

### 5-Bromo-1,3-dihydrobenzoimidazol-2-one

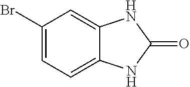

4-Bromobenzene-1,2-diamine (1 g) was dissolved in DCM (15 mL) and triethylamine (1.50 mL). Phosgene solution (5.3 mL) was added slowly to the solution at 0° C. The reaction was allowed to warm to room temperature and allowed to stir at room temperature for 2 hours. The reaction was quenched with water (2 mL) then evaporated to dryness. The residue was chromatographed on silica, eluting with 5% methanol in DCM to give the desired material (657 mg) as a white solid.

Mass Spectrum: MH+ 213

NMR Spectrum: ¹H NMR (DMSO-d₆) δ6.88 (1H, d), 7.06-7.10 (2H, m), 10.74 (2H, s)

### EXAMPLE 92

### [5-[4-(Methylsulfonylmethyl)-6-morpholin-4-yl-pyrimidin-2-yl]-1H-indazol-3-yl]methanol

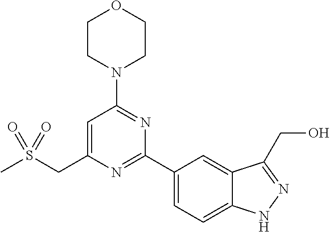

A mixture of (5-bromo-1H-indazol-3-yl)methanol (90 mg), potassium acetate (117 mg) and bis(pinacolato)diboron (121 mg) in 1,4-dioxane (5 mL) was degassed for 5 minutes. 1,1′-bis(diphenylphosphino)ferrocenedichloropalladium(II) dichloromethane adduct (20 mg) was added and the reaction was heated to 80° C. for 2.5 hours. 2-Chloro-4-(methylsulfonylmethyl)-6-morpholin-4-yl-pyrimidine (116 mg), ethanol (0.4 mL), a 2M solution of sodium carbonate (1.3 mL) and additional 1,1′-bis(diphenylphosphino)ferrocenedichloropalladium(II) dichloromethane adduct (20 mg) were added and the heating was continued for a further 3 hours. The cooled reaction mixture was concentrated in vacuo, dissolved in methanol and loaded onto a SCX-2 column (20 g). The column was washed with methanol and the compound removed with 7N ammonia in methanol. The solution was concentrated in vacuo and the residue chromatographed on silica, eluting with 0-5% methanol in DCM, to give the desired material (37 mg) as a white solid.

Mass Spectrum; MH+ 404

NMR Spectrum: ¹H NMR (DMSO-d₆) δ3.24 (3H, s), 3.76 (8H, s), 4.51-4.54 (2H, m), 4.84 (2H, d), 5.29 (1H, t), 6.87 (1H, s), 7.50-7.59 (1H, m), 8.39-8.42 (1H, m), 8.88 (1H, s), 12.93 (1H, s)

The preparation of (5-bromo-1H-indazol-3-yl)methanol is described below:

### (5-Bromo-1H-indazol-3-yl)methanol

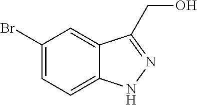

To a stirred solution of 5-bromo-1H-indazole-3-carbaldehyde (500 mg) in methanol (10 mL) and water (1 mL) at 0° C. was added sodium borohydride (337 mg) portion wise. The reaction was allowed to warm to room temperature and left to stir for 1 hour. The reaction was quenched with water and loaded onto a SCX-2 (10 g) column. The column was washed with methanol and product removed with 7N ammonia in methanol. The solution was concentrated in vacuo and the residue chromatographed on silica, eluting with 0-5% methanol in DCM, to give the desired material (90 mg) as a white solid.

Mass Spectrum: (M−H)⁻ 224

NMR Spectrum: ¹H NMR (DMSO-d₆) δ4.78 (2H, d), 5.26 (1H, t), 7.43-7.46 (1H, m), 7.47-7.50 (1H, m), 8.07 (1H, d), 12.97 (1H, s)

### EXAMPLE 93

### 6-[4-(Methylsulfonylmethyl)-6-morpholin-4-yl-pyrimidin-2-yl]chroman-4-ol

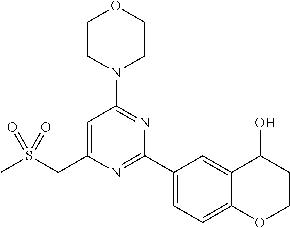

A mixture of 6-Bromochroman-4-ol (250 mg), potassium acetate (321 mg) and bis(pinacolato)diboron (333 mg) in 1,4-dioxane (10 mL) was degassed for 5 minutes. 1,1′-Bis(diphenylphosphino)ferrocenedichloropalladium(II) dichloromethane adduct (54 mg) was added and the reaction was heated to 80° C. for 2.5 hours. 2-Chloro-4-(methylsulfonylmethyl)-6-morpholin-4-yl-pyrimidine (319 mg), ethanol (0.75 mL), a 2M solution of sodium carbonate (2.7 mL) and additional 1,1′-bis(diphenylphosphino)ferrocenedichloropalladium(II) dichloromethane adduct (54 mg) were added and the heating was continued for a further 3 hours. The cooled reaction mixture was concentrated in vacuo, dissolved in methanol and loaded onto a SCX-2 column (20 g). The column was washed with methanol and the compound removed with 7N ammonia in methanol. The solution was concentrated in vacuo and the residue chromatographed on silica, eluting with 5% methanol in DCM, to give the desired material (113 mg) as a white solid.

Mass Spectrum; MH+ 406

NMR Spectrum: ¹H NMR (DMSO-d₆) δ1.90-1.94 (1H, m), 2.03-2.05 (1H, m), 3.21 (3H, s), 3.68-3.74 (8H, d), 4.25 (2H, d), 4.50 (2H, s), 4.70 (1H, q), 5.46 (1H, d), 6.83 (1H, d), 6.86 (1H, s), 8.14-8.16 (1H, m), 8.34 (1H, d)

### EXAMPLE 94

### 1-Acetyl-5-[4-(methylsulfonylmethyl)-6-morpholin-4-yl-pyrimidin-2-yl]-2H-indol-3-one

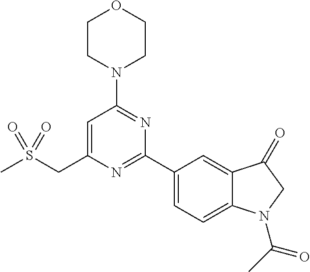

A mixture of 1-acetyl-5-bromo-1H-indol-3-ol (250 mg), potassium acetate (290 mg) and bis(pinacolato)diboron (300 mg) in 1,4-dioxane (10 mL) was degassed for 5 minutes. 1,1′-Bis(diphenylphosphino)ferrocenedichloropalladium(II) dichloromethane adduct (49 mg) was added and the reaction was heated to 80° C. for 3 hours. 2-Chloro-4-(methylsulfonylmethyl)-6-morpholin-4-yl-pyrimidine (288 mg), ethanol (0.75 mL), a 2M solution of sodium carbonate (2.7 mL) and additional 1,1′-bis(diphenylphosphino)ferrocenedichloropalladium(II) dichloromethane adduct (54 mg) were added and the heating was continued for a further 2.5 hours. The cooled reaction mixture was concentrated in vacuo and the residue chromatographed on silica, eluting with 5% methanol in DCM, to give the desired material (87 mg) as a white solid.

Mass Spectrum; MH+ 431

NMR Spectrum: ¹H NMR (DMSO-d₆) δ2.30 (3H, s), 3.21 (3H, s), 3.75 (8H, s), 4.54 (2H, s), 4.66 (2H, s), 6.92 (1H, s), 8.58-8.58 (2H, m), 8.71-8.74 (1H, m)

### EXAMPLE 95

### 1-Methyl-4-[4-(methylsulfonylmethyl)-6-morpholin-4-yl-pyrimidin-2-yl]piperazin-2-one

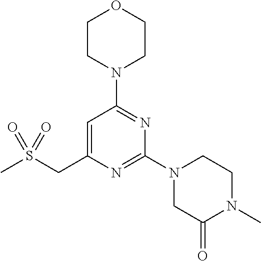

A mixture of 2-chloro-4-(methylsulfonylmethyl)-6-morpholin-4-yl-pyrimidine (200 mg), 1-methylpiperazin-2-one (157 mg) and sodium carbonate (146 mg) in DMA (4 mL) was heated in a microwave reactor at 160° C. for 10 minutes. The reaction mixture was loaded onto a SCX-2 column and product removed with 7N ammonia in methanol. The solution was evaporated to dryness and chromatographed on silica, eluting with 0-2.5% methanol in DCM, to give the desired material (179 mg) as a white solid.

Mass Spectrum; MH+ 370

NMR Spectrum: ¹H NMR (DMSO-d₆) δ2.89 (3H, s), 3.13 (3H, s), 3.38 (2H, t), 3.55-3.56 (4H, m), 3.67-3.68 (4H, m), 3.93 (2H, t), 4.19 (2H, s), 4.28 (2H, s), 6.28 (1H, s)

The following compound was prepared in an analogous fashion from 2-chloro-4-(methylsulfonylmethyl)-6-morpholin-4-yl-pyrimidine and the appropriate piperazine-2-one.

| | | | LCMS | |
|---|---|---|---|---|
| Example | Structure | NAME | MH+ | Notes |
| 96 | 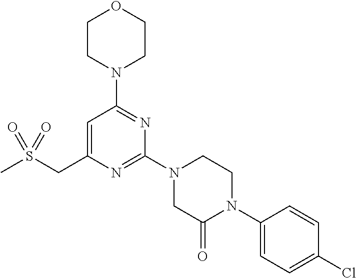 | 1-(4-Chlorophenyl)-4-[4- (methylsulfonylmethyl)-6- morpholin-4-yl-pyrimidin- 2-yl]piperazin-2-one | 466 | White solid (181 mg) |

### EXAMPLE 96

NMR Spectrum: ¹H NMR (DMSO-d₆) δ3.15 (3H, s), 3.59 (4H, d), 3.68-3.69 (4H, m), 3.79-3.81 (2H, d), 4.04-4.07 (2H, m), 4.30 (2H, s), 4.40 (2H, s), 6.31 (1H, s), 7.41-7.46 (2H, m), 7.47-7.49 (2H, m).

### EXAMPLE 97

### 2-[3-(4,4-Dimethyl-5H-1,3-oxazol-2-yl)-4-methoxy-phenyl]-4-(methylsulfonylmethyl)-6-morpholin-4-yl-pyrimidine

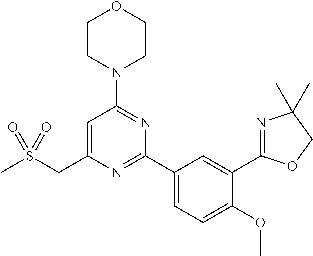

A mixture of 2-(5-bromo-2-methoxyphenyl)-4,4-dimethyl-4,5-dihydro-1,3-oxazole (250 mg), potassium acetate (259 mg) and bis(pinacolato)diboron (269 mg) in 1,4 dioxane (10 mL) was degassed for 5 minutes then 1,1′-bis(diphenylphosphino)ferrocenedichloropalladium(II) dichloromethane adduct added (44 mg). The reaction was heated to 80° C. for 2.5 hours. 2-Chloro-4-(methylsulfonylmethyl)-6-morpholin-4-yl-pyrimidine (257 mg), ethanol (0.75 mL), 2M sodium carbonate solution (2.7 mL) and 1,1′-bis(diphenylphosphino)ferrocenedichloropalladium(II) dichloromethane adduct (44 mg) were added and the heating was continued for 3 hours. The reaction mixture was concentrated in vacuo then dissolved in methanol. The solution was passed through a SCX-2 column, the column washed with methanol then the desired material eluted with 7N ammonia in methanol. The fractions were concentrated in vacuo then chromatographed on silica, eluting with 5% methanol in DCM, to give the desired compound (43 mg) as a white solid.

Mass Spectrum; MH+ 461

NMR Spectrum: ¹H NMR (DMSO-d₆) δ1.35 (6H, s), 3.23 (3H, s), 3.45 (2H, d), 3.74 (8H, d), 3.98 (3H, s), 4.53 (2H, s), 5.06 (1H, t), 6.87 (1H, s), 7.26 (1H, d), 8.15 (1H, s), 8.42-8.45 (1H, m), 8.85 (1H, d)

### EXAMPLE 98

### N-(1H-Benzoimidazol-5-yl)-2,6-dimorpholin-4-yl-pyrimidine-4-carboxamide

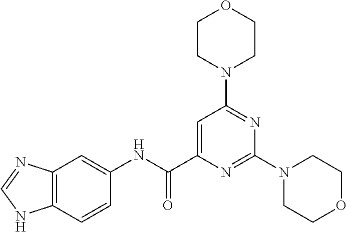

A mixture of 2,6-dimorpholin-4-ylpyrimidine-4-carboxylic acid (45 mg, 0.15 mmol), HATU (65 mg, 0.17 mmol) and 1H-benzoimidazol-5-amine (23 mg, 0.17 mmol) in DMF (1 mL) and triethylamine (0.054 mL, 0.31 mmol) was stirred at room temperature overnight. Water (4 mL) was added and the mixture extracted with ethyl acetate (3×4 mL). The combined organics were dried (MgSO₄) and concentrated in vacuo. The residue was chromatographed on silica, eluting with 10-45% ethyl acetate in isohexane, to give the desired material as a pale yellow solid (43.6 mg).

LCMS Spectrum: MH+ 410, Retention Time 2.05, Method: Monitor Acid

NMR Spectrum: ¹H NMR (399.9 MHz, CDCl₃) δ3.75 (m, 12H), 3.85-3.86 (m, 4H), 5.90 (s, 1H), 6.93 (m, 1H), 6.96 (m, 1H), 7.32 (m, 1H), 7.34 (s, 1H)

The following compounds were made in an analogous fashion from the commercially available 2,6-dimorpholin-4-ylpyrimidine-4-carboxylic acid and the appropriate amine.

| | | | | Retention |
|---|---|---|---|---|
| | | | LCMS | time |
| Example | Structure | NAME | MH+ | (min) |
| 99 | 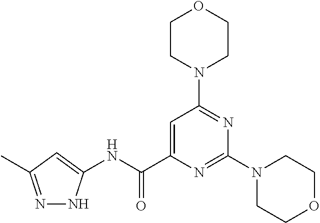 | N-(5-methyl-2H-pyrazol-3-yl)-2,6- dimorpholin-4-yl-pyrimidine-4- carboxamide | 373 | 1.60 |
| 100 | 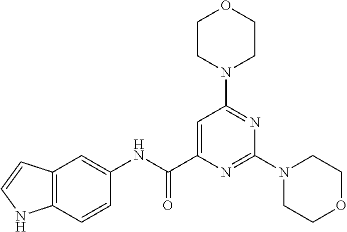 | N-(1H-indol-5-yl)-2,6-dimorpholin- 4-yl-pyrimidine-4-carboxamide | 409 | 2.06 |
| 101 | 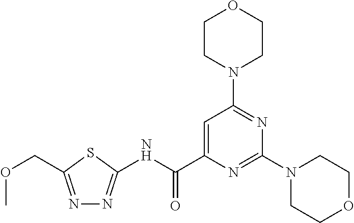 | N-[5-(methoxymethyl)-1,3,4- thiadiazol-2-yl]-2,6-dimorpholin- 4-yl-pyrimidine-4-carboxamide | 422 | 1.97 |

### EXAMPLE 99

¹H NMR (399.9 MHz, CDCl₃) δ2.34 (s, 3H), 3.67 (m, 4H), 3.75-3.80 (m, 12H), 6.58 (s, 1H), 6.82 (s, 1H), 9.99 (s, 1H)

### EXAMPLE 100

¹H NMR (399.9 MHz, CDCl₃) δ 3.67 (m, 4H), 3.80 (m, 12H), 6.57 (m, 1H), 6.9 (s, 1H), 7.22 (m, 1H), 7.39 (d, 1H), 7.45 (m, 1H), 8.09 (d, 1H), 8.15 (s, 1H), 9.78 (s, 1H)

### EXAMPLE 101

¹H NMR (399.9 MHz, DMSO-d₆) δ 3.48 (s, 3H), 3.69 (m, 4H), 3.78 (m, 12H), 4.81 (s, 2H), 6.30 (s, 1H), 10.92 (s, 1H)

### EXAMPLE 102

### 5-[4-(Methylsulfonylmethyl)-6-morpholin-4-yl-pyrimidin-2-yl]-1H-indazole

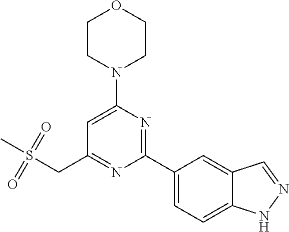

1-(4-Methylphenyl)sulfonyl-5-[4-(methylsulfonylmethyl)-6-morpholin-4-yl-pyrimidin-2-yl]indazole (95 mg, 0.18 mmol) and 1.0 M tetrabutylammonium fluoride solution in tetrahydrofuran (1.0 mL, 1.0 mmol) and tetrahydrofuran (5 mL) were heated together at 50° C. for 2 hours. The solvent was evaporated and the residue partitioned between water and dichloromethane. The organic solution was further washed with water, dried over magnesium sulfate, filtered and concentrated and the residue purified using reverse phase preparative HPLC (basic conditions) to afford the title compound, 36 mg.

LCMS Spectrum: MH+ 374, Retention Time 1.28, Method: Monitor Acid

NMR Spectrum: ¹H NMR (300.132 MHz, DMSO) δ 3.17 (3H, s), 3.68 (8H, s), 4.45 (2H, s), 6.79 (1H, s), 7.53 (1H, d), 8.14 (1H, s), 8.32 (1H, dd), 8.73 (1H, s), 13.12 (1H, s).

The starting material 1-(4-methylphenyl)sulfonyl-5-[4-(methylsulfonylmethyl)-6-morpholin-4-yl-pyrimidin-2-yl]indazole was prepared as follows:

### 1-(4-Methylphenyl)sulfonyl-5-[4-(methylsulfonylmethyl)-6-morpholin-4-yl-pyrimidin-2-yl]indazole

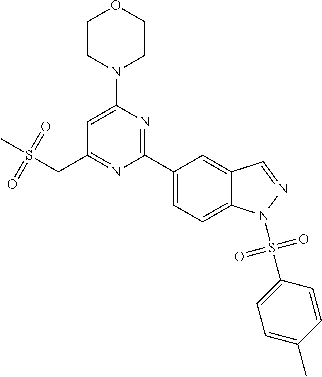

1-(4-Methylphenyl)sulfonyl-5-(4,4,5,5-tetramethyl-1,3,2-dioxaborolan-2-yl)indazole (209 mg, 0.53 mmol), 2-chloro-4-(methylsulfonylmethyl)-6-morpholin-4-yl-pyrimidine (44 mg, 0.15 mmol), 2M aqueous sodium carbonate solution (1 mL), dichlorobis(triphenylphosphine) Palladium (II) (15 mg) and 18% dimethyl formamide in 7:3:2 dimethoxyethane:water:ethanol (3.5 mL) were heated in a microwave reactor at 100° C. for 10 minutes. The reaction mixture was partitioned between dichloromethane and water. The organic solution was dried over magnesium sulfate, filtered and concentrated. The residue was purified by chromatography on silica eluting with ethyl acetate to yield the desired compound as a brown solid, (112 mg).

LCMS Spectrum: MH+ 528, Retention Time 2.55, Method: Monitor Acid

NMR Spectrum: ¹H NMR (500.133 MHz, DMSO) δ 2.31 (3H, s), 3.20 (3H, s), 3.27 (4H, s), 3.30 (4H, s), 4.52 (2H, s), 6.91 (1H, s), 7.39 (2H, d), 7.82 (2H, d), 8.21 (1H, d), 8.63 (1H, d), 8.64 (1H, s), 8.79 (1H, s)

### 1-(4-Methylphenyl)sulfonyl-5-(4,4,5,5-tetramethyl-1,3,2-dioxaborolan-2-yl)indazole

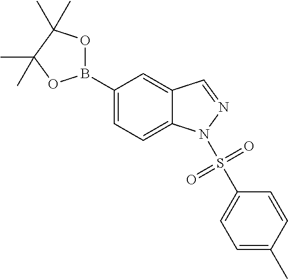

5-Bromo-1-(4-methylphenyl)sulfonyl-indazole (3.0 g, 8.54 mmol), potassium acetate (2.52 g, 25.62 mmol), Bis(pinacolato)diboron (3.04 g, 11.96 mmol) and 1,1′-Bis(diphenylphosphino)ferrocenedichloropalladium (II) (375 mg, 0.51 mmol) in 1,4 dioxane (45 mL) was stirred at 80° C. under an inert atmosphere for 48 hours. The solvent was removed by evaporation and the residue taken up in methanol and filtered. The filtrate was concentrated to yield the desired compound as a brown solid, (4.1 g)

LCMS Spectrum: MH+ 399, Retention Time 3.27, Method: Monitor Acid

### 5-Bromo-1-(4-methylphenyl)sulfonyl-indazole

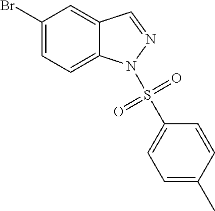

A solution of 5-bromo-1H-indazole (3.8 g, 19.29 mmol, CAS number 53857-57-1) in dimethyl formamide (25 mL) was added to a mixture of 60% sodium hydride in oil (771 mg, 19.29 mmol) in dimethylformamide (25 mL) at 0° C. under an inert atmosphere and stirred for 30 minutes. Tosyl chloride (5.15 g, 27.0 mmol) was added and stirred at room temperature for 18 hours. Reaction mixture was poured into ice/water with vigorous stirring and the product extracted into ethyl acetate. The organic solution was washed with brine, dried over magnesium sulfate, filtered and evaporated. The residue was dissolved in dichloromethane and filtered through a silica pad. The filtrate was concentrated and the residue triturated with diethyl ether and the solid collected by filtration to yield the desired compound, (6.37 g).

LCMS Spectrum: MH+ 353, Retention Time 2.92, Method: Monitor Acid

NMR Spectrum; ¹H NMR (300.132 MHz, DMSO) δ 2.34 (s, 3H), 7.40 (d, 2H), 7.76-7.85 (m, 3H), 8.05-8.14 (m, 2H), 8.50 (s, 1H)

### EXAMPLE 103

### 3-methyl-5-[4-(methylsulfonylmethyl)-6-morpholin-4-yl-pyrimidin-2-yl]-1H-indazole

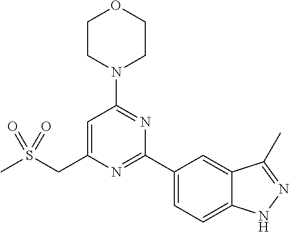

Tetrabutylammonium fluoride (1M solution in THF, 2 mL) was added to a solution of 3-methyl-1-(4-methylphenyl)sulfonyl-5-[4-(methylsulfonylmethyl)-6-morpholin-4-yl-pyrimidin-2-yl]indazole (AZ12581939) (26 mg, 0.05 mmol) in THF (2 mL). Warmed to 50° C. for 3 hours, poured into water and extracted well with DCM. The organic phase was washed with water (3×) and dried over MgSO₄, filtered and evaporated under reduced pressure. Purification on silica (Gradient elution 50% ethyl acetate/50% iso-hexane to 100% ethyl acetate) gave the title compound as a light brown solid (10.4 mg, 54%).

LCMS Spectrum: MH+ 388.56 Retention Time 2.46, Method: Monitor Early Acid

NMR Spectrum: ¹H NMR (300.132 MHz, DMSO) δ 2.55 (3H, s), 3.24 (3H, s), 3.75 (8H, s), 4.53 (2H, s), 6.86 (1H, s), 7.52 (1H, d), 8.38 (1H, dd), 8.69 (1H, s). 12.80 (1H, s)

The starting material, 3-methyl-1-(4-methylphenyl)sulfonyl-5-[4-(methylsulfonylmethyl)-6-morpholin-4-yl-pyrimidin-2-yl]indazole was prepared as follows

### 3-Methyl-1-(4-methylphenyl)sulfonyl-5-[4-(methylsulfonylmethyl)-6-morpholin-4-yl-pyrimidin-2-yl]indazole

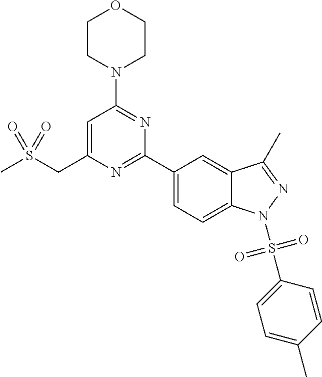

2-Chloro-4-(methylsulfonylmethyl)-6-morpholin-4-yl-pyrimidine (146 mg, 0.50 mmol), 3-s methyl-1-(4-methylphenyl)sulfonyl-5-(4,4,5,5-tetramethyl-1,3,2-dioxaborolan-2-yl)indazole (413 mg, 1 mmol), 2M sodium carbonate solution (2 mL) and Dichlorobis(triphenylphosphine)Pd(11) (40 mg) in a solution of 18% DMF in DME/H2O/EtOH (7:3:2) (7 mL) were irradiated in a microwave tube for 10 minutes at 100° C. The reaction was then evaporated to dryness under reduced pressure and the residue partitioned between DCM and water. The aqueous phase was extracted twice with DCM and the combined organics washed with water, saturated NaHCO₃ solution and brine. The solution was then dried over MgSO₄, filtered and evaporated under reduced pressure. The residue was then purified on a SCX2 column, eluting with Methanol followed by 4% NH4OH in methanol to elute the title compound which was finally obtained (after evaporation) as an off white solid (26 mg, 9%).

LCMS Spectrum: MH+ 542.59 Retention Time 2.18, Method: Monitor Mid Acid

NMR Spectrum: ¹H NMR (300.132 MHz, DMSO) δ 2.33 (3H, s), 2:56 (3H, s), 3.21 (3H, s), 3.75 (8H, s), 4.54 (2H, s), 6.92 (1H, s), 7.39 (2H, d), 7.81 (2H, d), 8.18 (1H, d), 8.63-8.66 (2H, m)

### 3-Methyl-1-(4-methylphenyl)sulfonyl-5-(4,4,5,5-tetramethyl-1,3,2-dioxaborolan-2-yl)indazole

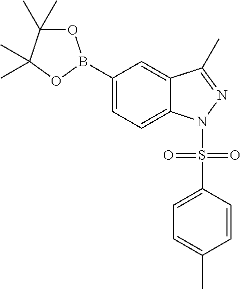

Anhydrous 1,4 dioxane (20 mL) was added to 5-bromo-3-methyl-1-(4-methylphenyl)sulfonyl-indazole (876.6 mg, 2.4 mmol), Bis(pinacolto)diboran (701 mg, 2.76 mmol), Dppf (40 mg, 0.072 mmol), PdCl2(dppf) (58.8 mg, 0.072 mmol) and Potassium acetate (707 mg, 7.2 mmol). The mixture was degassed 3 times before allowing to heat to reflux under nitrogen for 2 hrs. The reaction was then cooled and evaporated to dryness under reduced pressure. The residue was partitioned between ethyl acetate and water. The organic phase was washed with water (2×), then 1M HCl (2×) and finally brine. The solution was then dried over MgSO₄, filtered and evaporated to dryness to give a brown solid (1.07 g). This was then applied to a silica column (20 g). Gradient elution 90% iso-hexane/10% ethyl acetate->50% iso-hexane/50% ethyl acetate gave the title compound as an off white solid (0.94 g, 95%).

LCMS Spectrum: MH+ 413.57 Retention Time 3.22, Method: Monitor Acid

NMR Spectrum: ¹H NMR (300.132 MHz, DMSO) δ 1.32 (12H, s), 2.32 (3H, s), 7.37 (2H, d), 7.76 (2H, d), 7.91 (1H, d), 8.09-8.12 (2H, m) (1× CH3 obscured by DMSO peak).

### 5-Bromo-3-methyl-1-(4-methylphenyl)sulfonyl-indazole

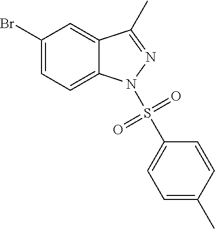

Sodium Hydride (60% dispersion in oil, 440 mg, 11 mmol) in anhydrous DMF (25 mL) under Nitrogen was cooled to 0° C. (ice/water bath). 5-bromo-3-methyl-1H-indazole (2.115 g, 10 mmol, prepared according to WO 2003/051366 Example 102C) was added dropwise as a solution in DMF (10 mL). After 30 minutes, tosyl chloride (2.67 g, 14 mmol) was added in one portion. Reaction mixture was allowed to warm to room temp and then stirred overnight. Reaction was quenched with ice/water. Extracted with ethyl acetate (3×). Washed with water and brine. Dried over MgSO₄, filtered and evaporated under reduced pressure to give a cream solid. Triturated with a small volume of ether (removes colour and minor impurities). Dried under vacuum to give the title compound as a white solid (2.9 g, 79%).

LCMS Spectrum: MH+ 365.35/367.38 Retention Time 2.82, Method: Monitor Acid

NMR Spectrum: ¹H NMR (300.132 MHz, DMSO) δ 2.33 (3H, s), 2.47 (3H, s), 7.38 (2H, d), 7.76-7.80 (3H, m), 8.03 (1H, d), 8.10 (1H, d)

### EXAMPLE 104

### 5-[2-(Methylsulfonylmethyl)-6-morpholin-4-yl-pyrimidin-4-yl]-1H-indole

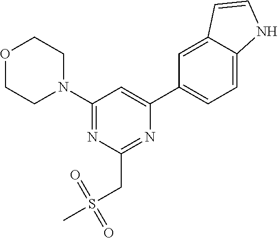

5-[6-Chloro-2-(methylsulfonylmethyl)pyrimidin-4-yl]-1H-indole (110 mg, 0.34 mmol) and morpholine (3 mL) were heated in a microwave reactor at 120° C. for 10 minutes. The reaction solution was purified using reverse phase preparative HPLC (basic conditions) to afford the title compound, (35 mg).

LCMS Spectrum: MH+ 373, Retention Time 1.38, Method: Monitor Acid

NMR Spectrum: ¹H NMR (300.132 MHz, DMSO) δ 3.17 (3H, s), 3.73 (8H, s), 4.52 (2H, s), 6.54 (1H, dd), 7.28 (1H, s), 7.41 (1H, m), 7.48 (1H, d), 7.95 (1H, dd), 8.44 (1H, s), 11.27 (1H, s)

The starting material 5-[6-chloro-2-(methylsulfonylmethyl)pyrimidin-4-yl]-1H-indole was prepared as follows:

### 5-[6-Chloro-2-(methylsulfonylmethyl)pyrimidin-4-yl]-1H-indole

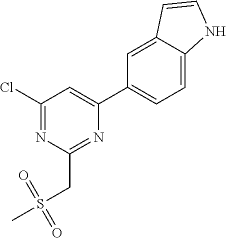

4,6-Dichloro-2-(methylsulfonylmethyl)pyrimidine (82 mg, 0.34 mmol), Indole-5-boronic acid (55 mg, 0.34 mmol), 2M aqueous sodium carbonate solution (1 mL), dichlorobis(triphenylphosphine) Palladium (II) (15 mg) and 18% DMF in 7:3:2 dimethoxyethane:water:ethanol (3.5 mL) were heated in a microwave reactor at 100° C. for 10 minutes. The reaction mixture was partitioned between ethyl acetate and water. The organic solution was dried over magnesium sulfate, filtered and concentrated in vacuo to yield the desired compound as a pale green gum, (149 mg).

LCMS Spectrum: MH+ 322, Retention Time 2.08, Method: Monitor Acid

### 4,6-Dichloro-2-(methylsulfonylmethyl)pyrimidine

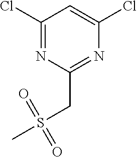

2-(Methylsulfonylmethyl)pyrimidine-4,6-diol (2.1 g, 5.0 mmol) and phosphorous oxychloride (20 mL) were heated at reflux for 4 hours. The resultant solution was concentrated in vacuo and azeotroped with toluene. The residue was partitioned between dichloromethane and ice cold water. The organic solution was dried by filtering through a PTFE frit then concentrated in vacuo. The residue was purified by flash chromatography on silica gel, eluting with hexane:ethyl acetate to yield the desired product as a white solid, 87 mg

LCMS Spectrum: MH+ 241, Retention Time 1.75, Method: Monitor Early

NMR Spectrum: ¹H NMR (300.132 MHz, CDCl₃) δ3.19 (3H, s), 4.56 (2H, s), 7.43 (1H, s)

### 2-(Methylsulfonylmethyl)pyrimidine-4,6-diol

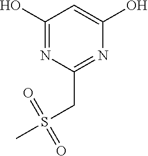

2-Methylsulfonylethanimidamide (172 mg, 1.00 mmol), potassium carbonate (143 mg, 1.05 mmol) and diethyl malonate (1 mL) were stirred and heated at 150° C. for two hours. The reaction mixture was cooled and diluted with diethyl ether and the solid collected by filtration and dried to yield the desired product as a white solid, (294 mg).

LCMS Spectrum: MH+ 205, Retention Time 0.43, Method: Monitor early

### 2-Methylsulfonylethanimidamide

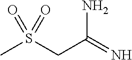

2-Methanesulfonylacetonitrile (11.9 g, 100.0 mmol) was stirred in ethanol and the mixture cooled on ice. Hydrogen chloride gas was bubbled through the mixture and the solid gradually dissolved. After saturating the solvent with hydrogen chloride the solution was stirred at room temperature overnight. The mixture was diluted with ether and the white precipitate collected by filtration and dried. The solid imidoylether was stirred in ethanol (200 mL) and 7M ammonia in methanol (13 mL, 0.1 mmol) was added and the mixture stirred at room temperature for 48 hours. The mixture was concentrated to half volume and the solid collected by filtration and dried to yield the desired product as a white solid, (15.35 g).

NMR Spectrum: ¹H NMR (300.132 MHz, D₂O) δ3.30 (3H, s), 4.69 (2H, s)

### EXAMPLE 105

### 5-[4-(Methylsulfonylmethyl)-6-morpholin-4-yl-pyrimidin-2-yl]-1H-benzoimidazole

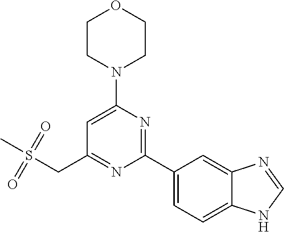

Trimethyl-[2-[[5-[4-(methylsulfonylmethyl)-6-morpholin-4-yl-pyrimidin-2-yl]benzoimidazol-1-yl]methoxy]ethyl]silane (53 mg, 0.11 mmol) and 2M aqueous hydrochloric acid (3 mL) in ethanol, were heated in a microwave reactor at 100° C. for 10 minutes. The reaction was then evaporated to a white solid which was purified by reverse phase preparative HPLC (basic conditions) to yield the title compound as white solid (17 mg).

LCMS Spectrum: MH+ 347, Retention Time 0.91, Method: Monitor Acid

NMR Spectrum: ¹H NMR (500.133 MHz, DMSO) δ 3.23 (3H, s), 3.73 (8H, s), 4.51 (2H, s), 6.85 (1H, s), 7.64 (1H, d), 8.26 (1H, d), 8.30 (1H, s), 8.59 (1H, s), 12.60 (1H, s)

The starting material trimethyl-[2-[[5-[4-(methylsulfonylmethyl)-6-morpholin-4-yl-pyrimidin-2-yl]benzoimidazol-1-yl]methoxy]ethyl]silane was prepared as follows:

### Trimethyl-[2-[[5-[4-(methylsulfonylmethyl)-6-morpholin-4-yl-pyrimidin-2-yl]benzoimidazol-1-yl]methoxy]ethyl]silane

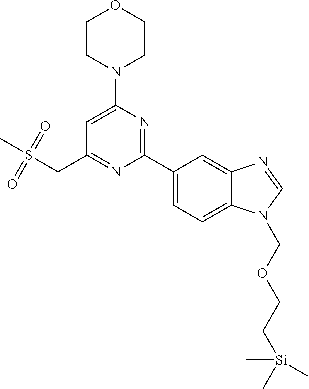

Trimethyl-[2-[[5-(4,4,5,5-tetramethyl-1,3,2-dioxaborolan-2-yl)benzoimidazol-1-yl]methoxy]ethyl]silane (57 mg, 0.15 mmol), 2-chloro-4-(methylsulfonylmethyl)-6-morpholin-4-yl-pyrimidine (44 mg, 0.15 mmol), 2M aqueous sodium carbonate solution (1 mL), dichlorobis(triphenylphosphine) Palladium (II) (15 mg) and 18% dimethyl formamide in 7:3:2 dimethoxyethane:water:ethanol (3.5 mL) were heated in a microwave reactor at 160° C. for 3.5 minutes. The reaction mixture was partitioned between dichloromethane and water. The organic solution was dried over magnesium sulfate, filtered and concentrated. The residue was purified by chromatography on silica eluting with ethyl acetate to yield the desired compound as a brown solid, (54 mg).

LCMS Spectrum: MH+ 504, Retention Time 2.10, Method: Monitor Acid

Trim ethyl-[2-[[5-(4,4,5,5-tetramethyl-1,3,2-dioxaborolan-2-yl)benzoimidazol-1-yl]methoxy]ethyl]silane

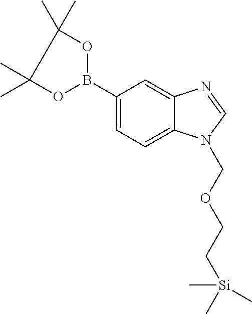

2-[(5-Bromobenzoimidazol-1-yl)methoxy]ethyl-trimethyl-silane (1.42 g, 4.33 mmol) potassium acetate (849 mg, 8.66 mmol), Bis(pinacolato)diboron (1.32 g, 5.20 mmol) and 1,1′-Bis(diphenylphosphino)ferrocenedichloropalladium (II) (71 mg, 0.09 mmol) in 1,4 dioxane (25 mL) was stirred at reflux under an inert atmosphere for 24 hours. The reaction mixture was concentrated and the residue taken up in ethyl acetate and filtered. The filtrate was washed with brine, dried over magnesium sulfate, filtered and evaporated. The residue was chromatographed on silica eluting with ethyl acetate to yield the desired compound as a pale green solid, (1.45 g).

LCMS Spectrum: MH+ 375, Retention Time 2.76, Method: Monitor Acid

### 2-[(5-Bromobenzoimidazol-1-yl)methoxy]ethyl-trimethyl-silane

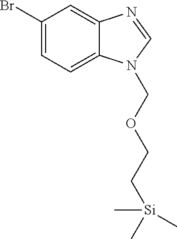

A solution of 5-bromo-benzimidazole (2.96 g, 15 mmol, CAS number 4887-88-1) in dimethyl formamide (15 mL) was added dropwise to a suspension of 60% sodium hydride in oil (660 mg, 16.5 mmol) in dimethyl formamide (20 mL) under an inert atmosphere and stirred for 30 minutes. The reaction mixture was cooled to 0° C. and a solution of 2-(trimethylsilyl)ethoxymethyl chloride (2.74 g, 16.5 mmol) in dimethyl formamide (15 mL) was added dropwise and the mixture stirred at room temperature for 18 hours. The reaction mixture was poured into ice water with stirring and the product extracted into ethyl acetate.

The organic solution was dried over magnesium sulfate, filtered and concentrated. The residue was chromatographed on silica eluting with 70% ethyl acetate in hexane. Product fractions concentrated to a pale yellow oil which was a mixture of tautomers of the desired compound, (2.83 g).

LCMS Spectrum: MH+ 329, Retention Time 2.79, Method: Monitor Acid

### EXAMPLE 106

### 4-[4-(methylsulfonylmethyl)-6-morpholin-4-yl-pyrimidin-2-yl]-1H-indole

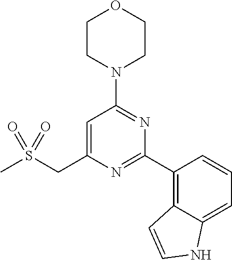

4-[6-[(Methylsulfonyl)methyl]-2-(methylthio)pyrimidin-4-yl]morpholine (151 mg, 0.5 mmol), indole-4-boronic acid (141 mg, 1.1 mmol), copper(I) thiophene-2-carboxylate (248 mg, 1.3 mmol), palladium tetrakis triphenylphosphine (47 mg, 0.04 mmol), zinc acetate (175 mg, 1.1 mmol) and 1,4-dioxane added (5 mL) were added to a microwave vessel. The system was degassed with nitrogen, sealed and heated in a microwave reactor at 130° C. for 45 minutes. The reaction was poured into water and extracted with ethyl acetate, washed with water, brine and dried over magnesium sulfate. The product was further purified using reverse phase preparative HPLC to afford the title compound, (43 mg).

LCMS Spectrum: MH+ 373, Retention Time 2.60, Method: Monitor Acid

NMR Spectrum: ¹H NMR (300.132 MHz, DMSO) δ 3.20 (d, 3H), 3.75 (s, 8H), 4.56 (s, 2H), 6.87 (s, 1H), 7.19 (t, 1H), 7.38 (d, 1H), 7.44 (t, 2H), 7.54 (d, 1H), 8.07 (dd, 1H), 11.36 (s, 1H)?

3-[4-(Methylsulfonylmethyl)-6-morpholin-4-yl-pyrimidin-2-yl]-5,7-diazabicyclo[4.3.0]nona-1,3,5,8-tetraene shown below was prepared in an analogous manner using 5,7-diazabicyclo[4.3.0]nona-1,3,5,8-tetraen-3-ylboronic acid and 4-[6-[(methylsulfonyl)methyl]-2-(methylthio)pyrimidin-4-yl]morpholine

| | | | | Retention | |
|---|---|---|---|---|---|
| | | | LCMS | Time | |
| Ex. | Structure | NAME | MH+ | (min) | Notes |
| 107 | 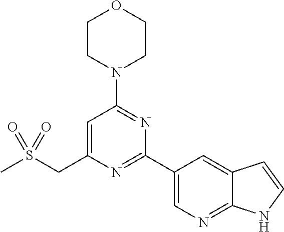 | 3-[4-(Methylsulfonylmethyl)- 6-morpholin-4-yl-pyrimidin- 2-yl]-5,7- diazabicyclo[4.3.0]nona- 1,3,5,8-tetraene | 374.4 | 1.64 | Zinc acetate was not added to this reaction |

### EXAMPLE 108

### 4-[4-(Methylsulfonylmethyl)-6-morpholin-4-yl-pyrimidin-2-yl]aniline

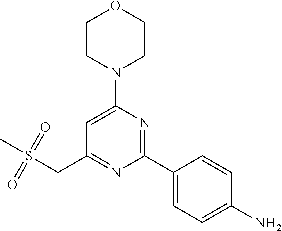

2-Methylsulfanyl-4-(methylsulfonylmethyl)-6-morpholin-4-yl-pyrimidine (1.00 g, 3.3 mmol), 4-aminophenylboronic acid (904 mg, 6.60 mmol), Copper(I) thiophene-2-carboxylate (1.64 g, 8.58 mmol), Pd(PPh₃)₄ (153 mg, 0.04 equiv., 0.13 mmol) were added to a microwave vessel and 1,4-Dioxane (20 mL) added. The system was degassed with N₂, sealed and heated in a microwave reactor at 130° C. for 1 hour. Upon cooling the reaction was poured into water and the resulting precipitate was collected by filtration and dried under vacuum to afford the title compound as an off-white solid. (988 mg)

LCMS Spectrum: MH+ 349.41, Retention Time 1.43, Method: Monitor Acid

NMR Spectrum: ¹H NMR (300.132 MHz, DMSO) δ 63.20 (3H, s), 3.61-3.83 (8H, m), 4.43 (2H, s), 5.57 (1H, s), 6.60 (2H, d), 6.70 (1H, s), 8.04 (2H, d)

### EXAMPLE 109

### 2-(1H-Indol-5-yl)-6-morpholin-4-yl-pyrimidine-4-carboxylic acid

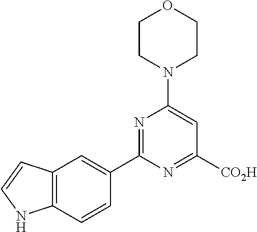

Methyl 2-chloro-6-morpholin-4-yl-pyrimidine-4-carboxylate (10.0 g, 38.91 mmol CAS number 107973-01-3), 1H-indol-5-ylboronic acid (9.7 g, 60.31 mmol), dichlorobis(triphenylphosphine)palladium (II) (2.1 g, 2.92 mmol) and sodium carbonate (2M in water, 100 mL) in 18% DMF in dimethoxyethane:water:ethanol (7:3:2) (320 mL) were heated in a microwave in 8 batches at 120° C. for 30 minutes. The combined batches were evaporated, taken to pH=2 with 2N HCl, stirred for 30 minutes and a solid was filtered off. This was dried overnight at 40° C. to give the title compound, (17 g).

LCMS Spectrum: MH+ 325, Retention Time 1.23, Method: Monitor Acid

NMR spectrum ¹H NMR (300.132 MHz, DMSO) δ3.70-3.83 (8H, m), 6.56-6.57 (1H, m), 7.18 (1H, s), 7.39-7.40 (1H, m), 7.45 (1H, d), 8.22-8.25 (1H, m), 8.70 (1H, s), 11.24 (1H, s).

### EXAMPLE 110

### [2-(1H-Indol-5-yl)-6-morpholin-4-yl-pyrimidin-4-yl]methanol

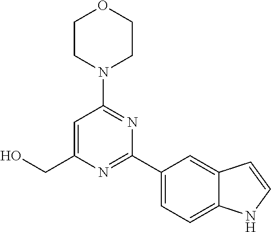

2-(1H-Indol-5-yl)-6-morpholin-4-yl-pyrimidine-4-carboxylic acid (14.0 g, 38.89 mmol) in THF (600 mL) was treated with lithium aluminium hydride (1.0M in tetrahydrofuran) (117 mL, 116.67 mmol) at 0° C. and stirred. After 5 h the mixture was treated with water (4.43 mL), then 15% NaOH (4.43 mL), then water (13.30 mL) and the mixture diluted with ethyl acetate (200 mL) and stirred for 35 minutes. The organics were evaporated and the residue was purified by SCX chromatography to give crude product. The foam was purified by MPLC [35-90% ethyl acetate:iso-hexane] to give the title compound, (5.58 g).

LCMS Spectrum: MH+ 310, Retention Time 1.03, Method: Monitor Acid

NMR spectrum ¹H NMR (300.132 MHz, DMSO) δ 3.73-3.82 (8H, m), 4.54 (2H, d), 5.44 (1H, t), 6.57-6.61 (1H, m), 6.76 (1H, s), 7.41-7.44 (1H, m), 7.47 (1H, d), 8.20-8.24 (1H, m), 8.66 (1H, s), 11.24 (1H, s).

### EXAMPLE 111

### 5-[4-Morpholin-4-yl-6-(morpholin-4-ylmethyl)pyrimidin-2-yl]-1H-indole

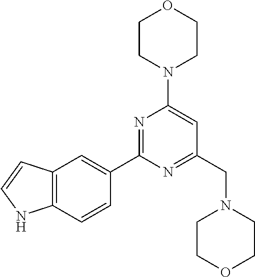

[2-(1H-Indol-5-yl)-6-morpholin-4-yl-pyrimidin-4-yl]methanol (100 mg, 0.32 mmol, from example 110) was suspended in dichloromethane (2 mL) and treated with methane sulfonylchloride (0.038 mL, 0.48 mmol) and triethylamine (0.068 mL, 0.48 mmol). The mixture was stirred overnight and then treated with morpholine (1 mL) and again stirred overnight. The solution was evaporated and purified by preparative HPLC [5-95% MeCN:water] to give the title compound, (10 mg).

LCMS Spectrum: MH+ 379, Retention Time 1.03, Method: Monitor Acid

NMR spectrum ¹H NMR (300.132 MHz, DMSO) δ 3.32-3.41 (4H, m), 3.69-3.79 (8H, m), 3.86-3.94 (4H, m), 4.35 (2H, s), 6.52-6.57 (1H, m), 6.78 (1H, d), 7.38-7.42 (1H, m), 7.46 (1H, d), 8.20 (1H, d), 8.68 (1H, s).

### EXAMPLE 112

### N-[[2-(1H-indol-5-yl)-6-morpholin-4-yl-pyrimidin-4-yl]methyl]-1-(4-methoxyphenyl)methanamine

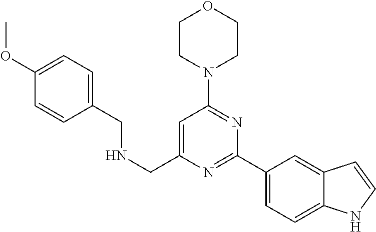

To a solution of 5-[4-(methylsulfonyloxymethyl)-6-morpholin-4-yl-pyrimidin-2-yl]-1H-indole in DCM (4 mL, assumed to contain 50 mg of material) was added a solution of 4-methoxybenzylamine (28 mg) and DIPEA (0.040 mL) in DCM (2 mL) added to it. The reaction stirred at room temperature overnight then NMP (1 mL) added and the DCM removed in vacuo. DIPEA (0.030 mL) and a couple of crystals of potassium iodide were added and the mixture heated in a microwave reactor at 100° C. for 10 minutes. The mixture was evaporated, loaded onto an SCX-2 column, the column washed with methanol and then the product eluted with 7N ammonia in methanol. The fractions were concentrated in vacuo and the residue purified by prep-HPLC (acid) to give the desired compound as a solid (20 mg).

LCMS Spectrum: MH+ 430, Retention Time 1.40, Method: Monitor Acid

NMR spectrum ¹H NMR (400.132 MHz, DMSO) δ 3.74 (8H, s), 3.79 (3H, s), 4.15 (2H, s), 4.27 (2H, s), 6.55 (1H, d), 6.75 (1H, s), 7.03 (2H, d), 7.41 (1H, d), 7.45 (1H, s), 7.47 (1H, s), 7.49 (2H, d), 8.27 (1H, dd), 8.74 (1H, d), 9.32 (1H, s)

The following compound was prepared in an analogous fashion using the appropriate amine

| | | | | Retention | |
|---|---|---|---|---|---|
| | | | LCMS | Time | |
| Ex. | Structure | NAME | MH+ | (min) | Notes |
| 113 | 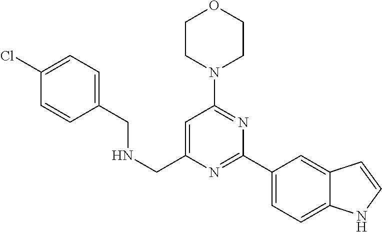 | 1-(4-Chlorophenyl)-N- [[2-(1H-indo1-5-yl)-6- morpholin-4-yl- pyrimidin-4- yl]methyl]methanamine | 434 | 1.50 | Additional heating at reflux for 4 hours following the overnight stirring |

### EXAMPLE 113

NMR spectrum ¹H NMR (400.132 MHz, DMSO) δ 3.74 (8H, s), 4.19 (2H, s), 4.34 (2H, s), 6.55 (1H, d), 6.75 (1H, s), 7.41 (1H, t), 7.46 (1H, d), 7.55 (2H, d), 7.59 (2H, d), 8.27 (1H, d), 8.74 (1H, dd), 9.45 (1H, bs)

The preparation of 5-[4-(methylsulfonyloxymethyl)-6-morpholin-4-yl-pyrimidin-2-yl]-1H-indole is described below.

### 5-[4-(Methylsulfonyloxymethyl)-6-morpholin-4-yl-pyrimidin-2-yl]-1H-indole

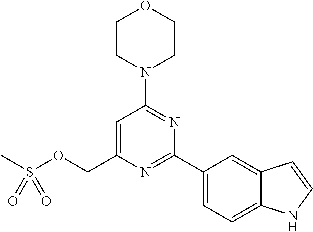

[2-(1H-Indol-5-yl)-6-morpholin-4-yl-pyrimidin-4-yl]methanol (200 mg) and triethylamine (0.135 mL) in DCM (5 mL) was stirred at room temperature and methanesulfonyl chloride (0.075 mL) added dropwise. The reaction was stirred for 1 hour, extra DCM (5 mL) and water (5 mL) added. The organic phase was separated, extra DCM (5 mL) added then the organics washed with brine (5 mL), dried (Na₂SO₄) and filtered. The reaction was assumed to have been quantitative and the mixture was diluted to 20 mL total volume with additional DCM (assumed to contain a total of 250 mg of material). This material was used without further purification or characterisation.

### EXAMPLE 114

### 5-[4-[(2-Methylpyridin-3-yl)oxymethyl]-6-morpholin-4-yl-pyrimidin-2-yl]-1H-indole

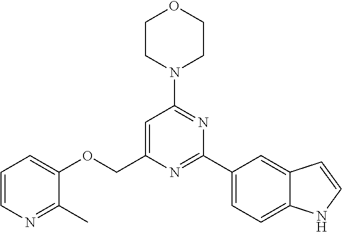

To a stirred solution of [2-(1H-Indol-5-yl)-6-morpholin-4-yl-pyrimidin-4-yl]methanol (from example 110, 40.6 mg, 0.13 mmol) and triethylamine (0.027 mL, 0.195 mmol) in DCM (5 mL) at room temperature was added methanesulfonyl chloride (0.015 mL, 0.195 mmol) dropwise. The reaction was then stirred for 1 hour, and then diluted with DCM (5 mL), washed with water (5 mL), brine (5 mL), dried (Na₂SO₄) and filtered, and evaporated to give the crude mesylate. A solution of 3-hydroxy-2-methylpyridine (22 mg, 0.19 mmol) in DMF (2 mL) was added to sodium hydride (8 mg of 60% dispersion in oil, 0.19 mmol) stirred in DMF (1 mL) at room temperature. After stirring for 5 minutes, the mesylate (50 mg, 0.13 mmol) was added in DCM (4 mL), then stirring was continued at room temperature overnight. Solvent was removed in vacuo, then water was added (10 mL), and the aqueous extracted into ethyl acetate (2×20 mL 1×10 mL) and DCM (10 mL). The combined organic extract was washed with water (5 mL) and brine (5 mL), dried (MgSO₄) and evaporated to give a gummy solid. The crude material was purified on a 10 g Isolute silica gel column, eluted with 2% methanol/DCM to give a white solid (22 mg).

LCMS Spectrum: MH+ 402, Retention Time 1.01, Method: Monitor Acid

NMR spectrum ¹H NMR (400.132 MHz, DMSO) δ 3.28 or 3.31 (3H, s), 3.73 (8H, s), 5.15 (2H, s), 6.55 (1H, d), 6.75 (1H, s), 7.19-7.22 (1H, m), 7.38 (1H, t), 7.44 (1H, d), 7.45 (1H, d), 8.05 (1H, d), 8.18 (1H, d), 8.63 (1H, d), 11.22 (1H, s)

### EXAMPLE 115

### 5-[4-(Methoxymethyl)-6-morpholin-4-yl-pyrimidin-2-yl]-1H-indole

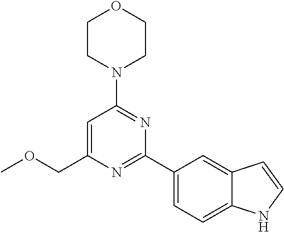

To a stirred solution of [2-(1H-indol-5-yl)-6-morpholin-4-yl-pyrimidin-4-yl]methanol (from example 110, 47 mg, 0.15 mmol) and triethylamine (0.031 mL, 0.225 mmol) in DCM (5 mL) at room temperature was added methanesulfonyl chloride (0.017 mL, 0.225 mmol) dropwise. The reaction was then stirred for 1 hour, and then diluted with DCM (5 mL), washed with water (5 mL), brine (5 mL), dried (Na₂SO₄) and filtered, and evaporated to give the crude mesylate. This was then dissolved in MeCN (1 mL) and added to a to solution of sodium methoxide (26 mg, 0.46 mmol) in methanol (3 mL) at room temperature and stirred for 30 hrs. Solvent was removed in vacuo and the crude material was purified on a silica gel column, eluted with 25% ethyl acetate in DCM to give the title compound as a solid (27 mg).

LCMS Spectrum: MH+ 325, Retention Time 2.01, Method: Monitor Base

NMR spectrum ¹H NMR (300.13 MHz, DMSO-d₆) δ3.43 (3H, s), 3.72 (8H, s), 4.42 (2H, s), 6.54 (1H, s), 6.62 (1H, s), 7.37 (1H, t), 7.42 (1H, d), 8.14-8.18 (1H, m), 8.60 (1H, s), 11.20 (1H, s)

### EXAMPLE 116

### 5-[4-(2-Furylmethylsulfonylmethyl)-6-morpholin-4-yl-pyrimidin-2-yl]-1H-indole

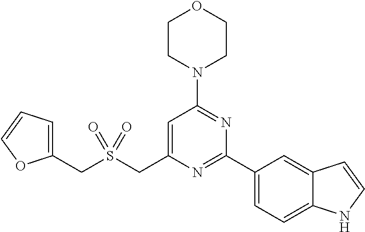

To a stirred solution of 5-[4-(2-furylmethylsulfanylmethyl)-6-morpholin-4-yl-pyrimidin-2-yl]-1H-indole (47 mg,) in dioxane/methanol (3 mL/0.5 mL) at room temperature was added 3-chloroperbenzoic acid (43 mg, 0.17 mmol), followed immediately by 1N sodium hydroxide solution (0.180 mL, 0.17 mmol). After 2 hrs 40 minutes, further 3-chloroperbenzoic acid (17 mg, 0.07 mmol) was added, washed in with a little methanol (<0.2 mL) followed immediately by 1M sodium hydroxide solution (0.070 mL, 0.07 mmol). The reaction was stirred for a further 40 minutes, then loaded onto an SCX-3 column (pre-treated with 30 mL methanol). The column was washed through with methanol (30 mL), then product was eluted with 10% 7N ammonia in methanol/methanol (60 mL). Evaporation gave a brown gum that was purified by prep HPLC to give the product as a colourless solid (15 mg, 55%).

LCMS Spectrum: MH+ 439, Retention Time 2.28, Method: Monitor Acid

The following compounds were prepared in an analogous fashion from the appropriate sulfides.

| | | | | Retention | |
|---|---|---|---|---|---|
| | | | LCMS | Time | |
| Ex | Structure | NAME | MH+ | (min) | Notes |
| 117 | 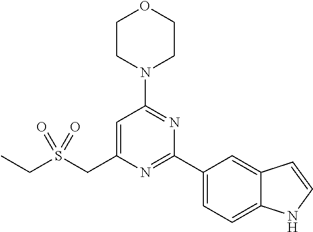 | 5-[4- (Ethylsulfonylmethyl)-6- morpholin-4-yl-pyrimidin- 2-yl]-1H-indole | (Basic) 387 | 1.99 | |
| 118 | 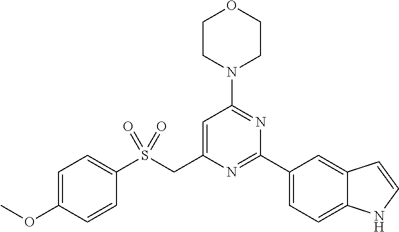 | 5-[4-[(4-Methoxyphenyl) sulfonylmethyl]-6- morpholin-4-yl-pyrimidin- 2-yl]-1H-indole | 465 | 1.67 | |
| 119 | 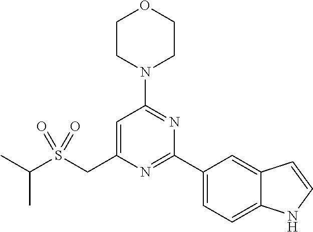 | 5-[4-Morpholin-4-yl-6- (propan-2-ylsulfonylmethyl) pyrimidin-2-yl]-1H-indole | (Basic) 401 | 2.16 | |
| 120 | 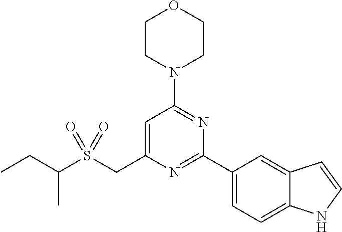 | 5-[4-(Butan-2- ylsulfonylmethyl)-6- morpholin-4-yl-pyrimidin- 2-yl]-1H-indole | (Basic) 415 | 2.35 | |
| 121 | 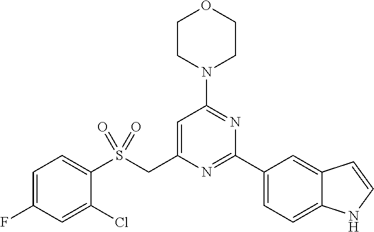 | 5-[4-[(2-Chloro-4-fluoro- phenyl)sulfonylmethyl]-6- morpholin-4-yl-pyrimidin- 2-yl]-1H-indole | (Basic) 487 | 2.55 | |

The starting material 5-[4-(2-furylmethylsulfanylmethyl)-6-morpholin-4-yl-pyrimidin-2-yl]-1H-indole was prepared as follows

### 5-[4-(2-Furylmethylsulfanylmethyl)-6-morpholin-4-yl-pyrimidin-2-yl]-1H-indole

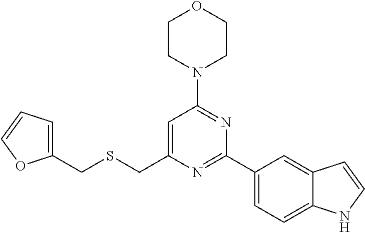

Sodium ethoxide (18 mg, 0.26 mmol) was added to a stirred solution of furfuryl mercaptan (30 mg, 0.26 mmol) in acetonitrile (4 mL) at room temperature in an MPS tube under to nitrogen. After 70 minutes stirring, a solution of 5-[4-(methylsulfonyloxymethyl)-6-morpholin-4-yl-pyrimidin-2-yl]-1H-indole (from example 112, 60 mg, 0.15 mmol) in acetonitrile (1 mL) was added and the reaction was then stirred at RT for 65 hrs. The reaction mixture was then loaded onto an SCX-3 column (pre-treated with 25 mL methanol). The column was washed with methanol (25 mL) to elute non-basic material, before eluting with 10% 7N ammonia in methanol/methanol (60 mL). Evaporation gave the sulfide as a gum (47 mg).

LCMS Spectrum: MH+ 407, Retention Time 2.60, Method: Monitor Base

The following sulfides were prepared in an analogous fashion from 5-[4-(methylsulfonyloxymethyl)-6-morpholin-4-yl-pyrimidin-2-yl]-1H-indole (from example 112) and the appropriate thiol.

| | | | Retention | |
|---|---|---|---|---|
| | | LCMS | Time | |
| Structure | NAME | MH+ | (min) | Notes |
| 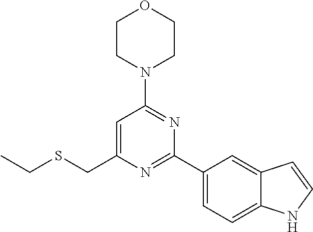 | 5-[4-(Ethylsulfanylmethyl)- 6-morpholin-4-yl-pyrimidin- 2-yl]-1H-indole | (Basic) 407 | 2.60 | |
| 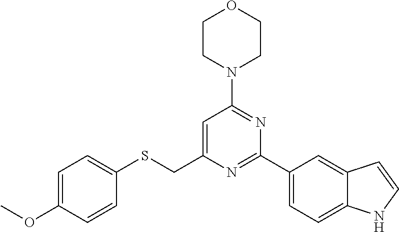 | 5-[4-[(4-Methoxyphenyl) sulfanylmethyl]-6- morpholin-4-yl-pyrimidin- 2-yl]-1H-indole | (Basic) 433 | 2.66 | Used 0.15 mmol thiol and 0.13 mesylate |
| 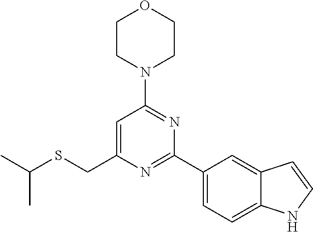 | 5-[4-Morpholin-4-yl-6- (propan-2- ylsulfanylmethyl)pyrimidin- 2-yl]-1H-indole | (Basic) 369 | 2.62 | |
| 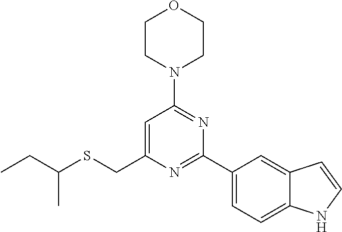 | 5-[4-(Butan-2- ylsulfanylmethyl)-6- morpholin-4-yl-pyrimidin- 2-yl]-1H-indole | (Basic) 383 | 2.77 | |
| 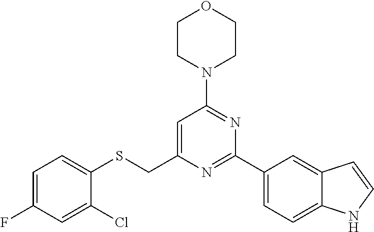 | 5-[4-[(2-Chloro-4-fluoro- phenyl)sulfanylmethyl]-6- morpholin-4-yl-pyrimidin- 2-yl]-1H-indole | (Basic) 455 | 2.89 | |

### EXAMPLE 122

### 2-[[2-(1H-Indol-5-yl)-6-morpholin-4-yl-pyrimidin-4-yl]methylsulfonyl]-N,N-dimethyl-acetamide

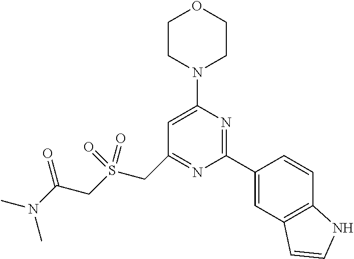

The [2-(1H-indol-5-yl)-6-morpholin-4-yl-pyrimidin-4-yl]methylsulfanylmethanimidamide 2,2,2-trifluoroacetic acid salt (0.080 g, 0.11 mmol) in DMF (2 mL) was added to a solution of 2-bromo-N,N-dimethyl-acetamide (0.11 mmol) in DMF (1 mL). This solution was treated with sodium hydroxide (35 mg, 0.87 mmol) in water (1 mL) and shaken for 1 hour. The solvent was evaporated. The residue was dissolved in ethyl acetate/water/brine (4 mL:2 mL:1 mL) with sonication and stirring. The organics were separated off and aqueous layer given another ethyl acetate extraction (2 mL). The combined organics were evaporated and purified by preparative. HPLC to give the sulfide which was dissolved in dioxane:water (3 mL:0.5 mL) and treated with 3-chloroperbenzoic acid (0.056 g, 0.13 mmol) and immediately sodium permanganate (0.027 g, 0.17 mmol). The mixture was stirred at room temperature for about 1 hour. The mixture was purified by SCX chromatography to give the title compound, (9 mg).

LCMS Spectrum: MH+ 444, Retention Time 1.27, Method: Monitor Acid

The following compounds were prepared in an analogous fashion from [2-(1H-indol-5-yl)-6-morpholin-4-yl-pyrimidin-4-yl]methylsulfanylmethanimidamide 2,2,2-trifluoroacetic acid salt and the appropriate alkyl halide.

| | | | | Retention |
|---|---|---|---|---|
| | | | LCMS | Time |
| Ex. | Structure | NAME | MH+ | (min) |
| 123 | 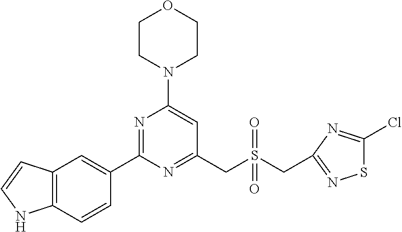 | 5-[4-[(5-Chloro-1,2,4-thiadiazol- 3-yl)methylsulfonylmethyl]-6- morpholin-4-yl-pyrimidin-2-yl]- 1H-indole | 492 | 1.5 |
| 124 | 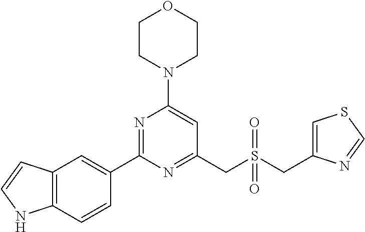 | 5-[4-Morpholin-4-yl-6-(1,3-thiazol-4- ylmethylsulfonylmethyl)pyrimidin- 2-yl]-1H-indole | 456 | 1.34 |
| 125 | 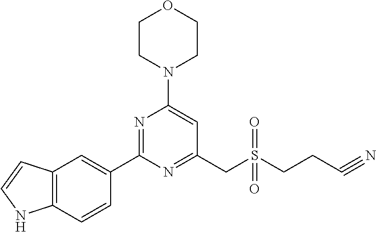 | 3-[[2-(1H-Indol-5-yl)-6- morpholin-4-yl-pyrimidin-4- yl]methylsulfonyl]propanenitrile | 412 | 0.99 |
| 126 | 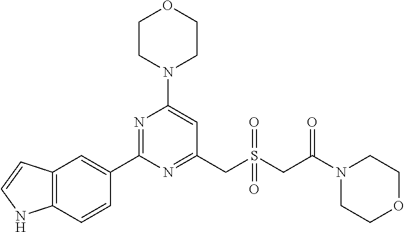 | 2-[[2-(1H-Indol-5-yl)-6- morpholin-4-yl-pyrimidin-4- yl]methylsulfonyl]-1-morpholin- 4-yl-ethanone | 486 | 1.34 |
| 127 | 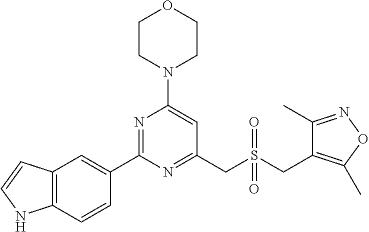 | 5-[4-[(3,5-dimethyl-1,2-oxazol- 4-yl)methylsulfonylmethyl]-6- morpholin-4-yl-pyrimidin-2-yl]- 1H-indole | 468 | 1.73 |
| 128 | 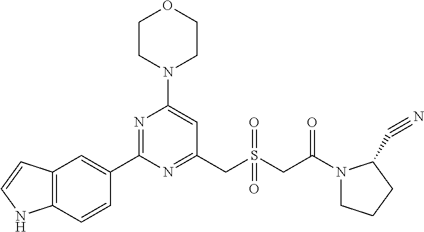 | (2S)-1-[2-[[2-(1H-Indol-5-yl)- 6-morpholin-4-yl-pyrimidin-4- yl]methylsulfonyl]acetyl]pyrrolidine- 2-carbonitrile | 495 | 1.51 |
| 129 | 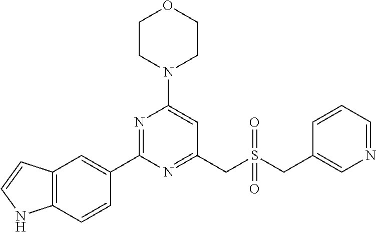 | 5-[4-Morpholin-4-yl-6-(pyridin-3- ylmethylsulfonylmethyl)pyrimidin- 2-yl]-1H-indole | 450 | 1.32 |
| 130 | 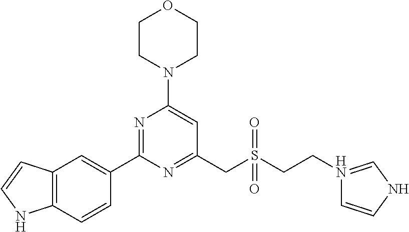 | 5-[4-(2-imidazol-1- ylethylsulfonylmethyl)-6- morpholin-4-yl-pyrimidin-2-yl]- 1H-indole | 453 | 0.93 |
| 131 | 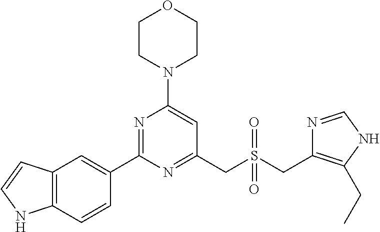 | 5-[4-[(5-Ethyl-1H-imidazol-4- yl)methylsulfonylmethyl]-6- morpholin-4-yl-pyrimidin-2-yl]- 1H-indole | 467 | 1.04 |
| 132 | 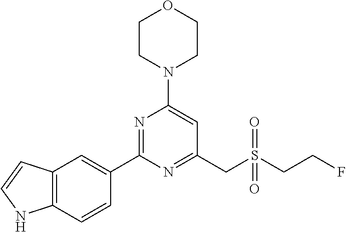 | 5-[4-(2- Fluoroethylsulfonylmethyl)-6- morpholin-4-yl-pyrimidin-2-yl]- 1H-indole | 403 (M − H)⁻ | 0.83 |
| 133 | 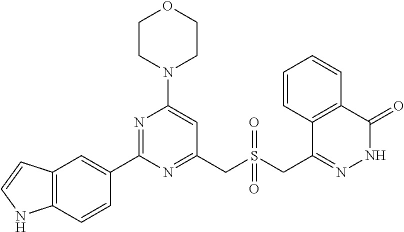 | 4-[[2-(1H-Indol-5-yl)-6- morpholin-4-yl-pyrimidin-4- yl]methylsulfonylmethyl]- 2H-phthalazin-1-one | 517 | 1.52 |
| 134 | 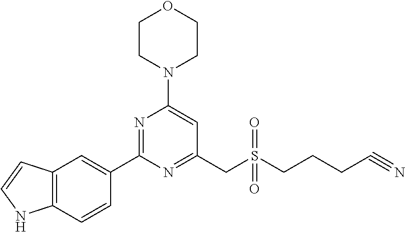 | 4-[[2-(1H-Indol-5-yl)-6- morpholin-4-yl-pyrimidin-4- yl]methylsulfonyl]butanenitrile | 426 | 1.48 |
| 135 | 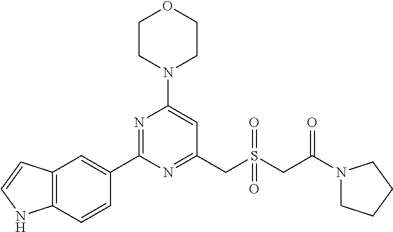 | 2-[[2-(1H-Indol-5-yl)-6- morpholin-4-yl-pyrimidin-4- yl]methylsulfonyl]-1- pyrrolidin-1-yl-ethanone | 470 | 1.39 |
| 136 | 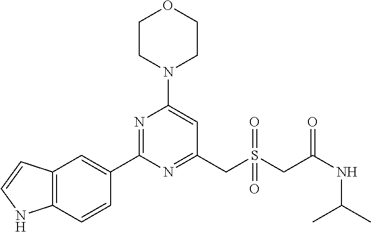 | 2-[[2-(1H-Indol-5-yl)-6- morpholin-4-yl-pyrimidin-4- yl]methylsulfonyl]-N- propan-2-yl-acetamide | 458 | 1.44 |
| 137 | 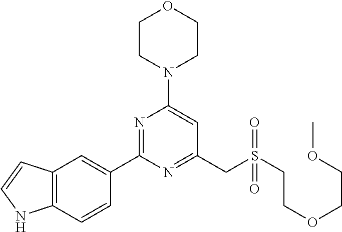 | 5-[4-[2-(2- methoxyethoxy)ethylsulfonylmethyl]- 6-morpholin-4-yl-pyrimidin-2- yl]-1H-indole | 461 | 1.31 |
| 138 | 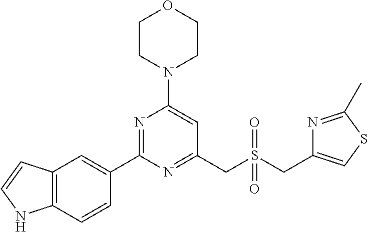 | 5-[4-[(2-methyl-1,3-thiazol-4- yl)methylsulfonylmethyl]-6- morpholin-4-yl-pyrimidin-2- yl]-1H-indole | 470 | 1.43 |
| 139 | 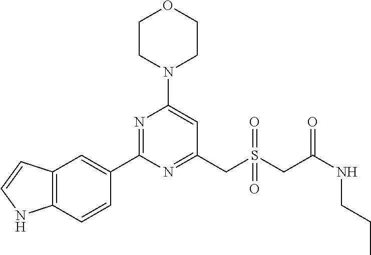 | 2-[[2-(1H-Indol-5-yl)-6- morpholin-4-yl-pyrimidin-4- yl]methylsulfonyl]-N- propyl-acetamide | 458 | 1.45 |
| 140 | 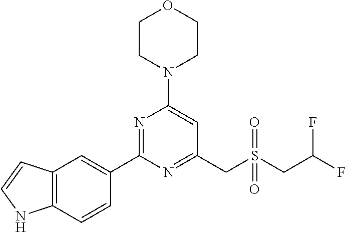 | 5-[4-(2,2- Difluoroethylsulfonylmethyl)- 6-morpholin-4-yl-pyrimidin-2- yl]-1H-indole | 423 | 1.47 |
| 141 | 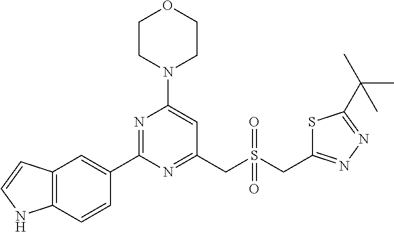 | 5-[4-Morpholin-4-yl-6-[(5- tert-butyl-1,3,4-thiadiazol-2- yl)methylsulfonylmethyl]pyrimidin- 2-yl]-1H-indole | 513 | 1.92 |
| 142 | 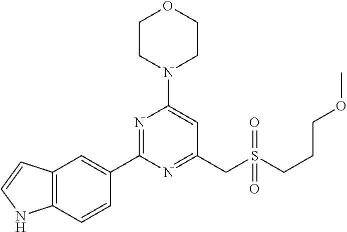 | 5-[4-(3- Methoxypropylsulfonylmethyl)- 6-morpholin-4-yl-pyrimidin-2- yl]-1H-indole | 431 | 1.47 |
| 143 | 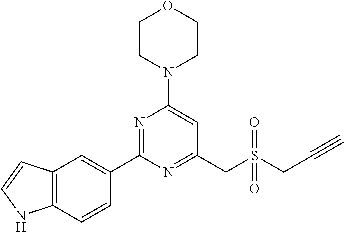 | 5-[4-Morpholin-4-yl-6-(prop-2- ynylsulfonylmethyl)pyrimidin- 2-yl]-1H-indole | 397 | 1.45 |
| 144 | 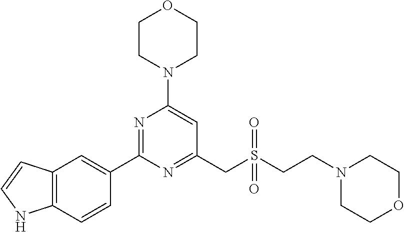 | 5-[4-morpholin-4-yl-6-(2- morpholin-4- ylethylsulfonylmethyl)pyrimidin- 2-yl]-1H-indole | 472 | 1.01 |
| 145 | 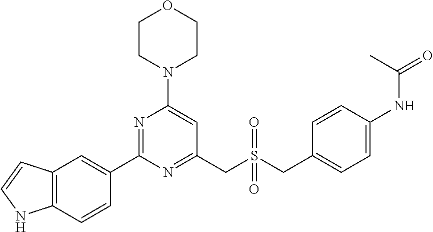 | N-[4-[[2-(1H-Indol-5-yl)-6- morpholin-4-yl-pyrimidin-4- yl]methylsulfonylmethyl] phenyl]acetamide | 506 | 1.47 |
| 146 | 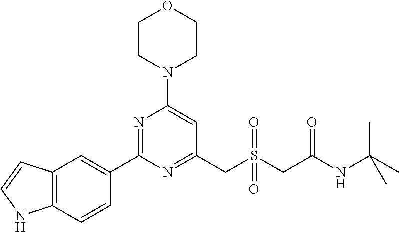 | 2-[[2-(1H-Indol-5-yl)-6- morpholin-4-yl-pyrimidin- 4-yl]methylsulfonyl]-N-tert- butyl-acetamide | 472 | 1.62 |
| 147 | 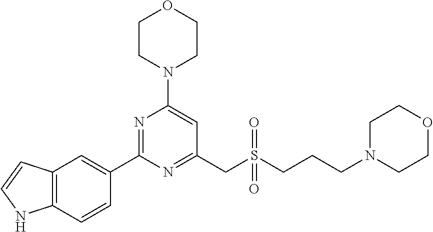 | 5-[4-Morpholin-4-yl-6-(3- morpholin-4- ylpropylsulfonylmethyl)pyrimidin- 2-yl]-1H-indole | 486 | 0.91 |
| 148 | 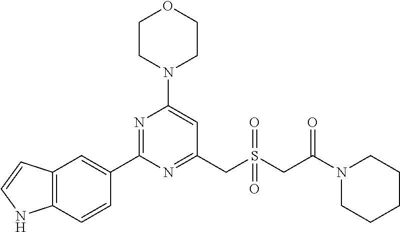 | 2-[[2-(1H-Indol-5-yl)-6- morpholin-4-yl-pyrimidin- 4-yl]methylsulfonyl]-1- (1-piperidyl)ethanone | 484 | 1.57 |
| 149 | 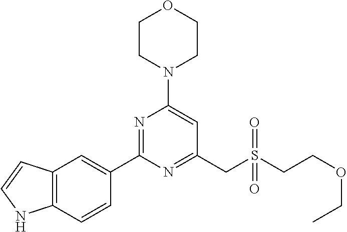 | 5-[4-(2- Ethoxyethylsulfonylmethyl)- 6-morpholin-4-yl-pyrimidin- 2-yl]-1H-indole | 431 | 1.48 |
| 150 | 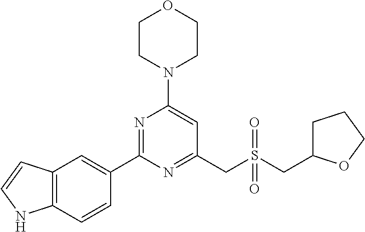 | 5-[4-morpholin-4-yl-6-(oxolan- 2-ylmethylsulfonylmethyl) pyrimidin-2-yl]-1H-indole | 443 | 1.44 |
| 151 | 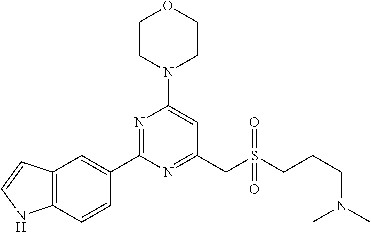 | 3-[[2-(1H-Indol-5-yl)-6- morpholin-4-yl-pyrimidin- 4-yl]methylsulfonyl]-N,N- dimethyl-propan-1-amine | 444 | 0.87 |
| 152 | 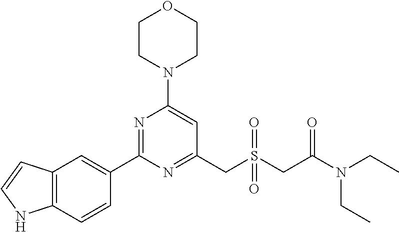 | N,N-Diethyl-2-[[2-(1H-indol- 5-yl)-6-morpholin-4-yl-pyrimidin- 4-yl]methylsulfonyl]acetamide | 472 | 1.50 |
| 153 | 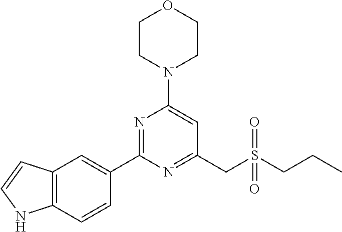 | 5-[4-Morpholin-4-yl-6- (propylsulfonylmethyl)pyrimidin- 2-yl]-1H-indole | 401 | 1.55 |
| 154 | 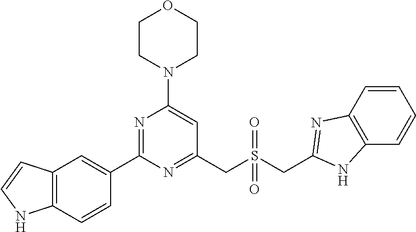 | 2-[[2-(1H-indol-5-yl)-6- morpholin-4-yl-pyrimidin- 4-yl]methylsulfonylmethyl]- 1H-benzoimidazole | 489 | 1.43 |
| 155 | 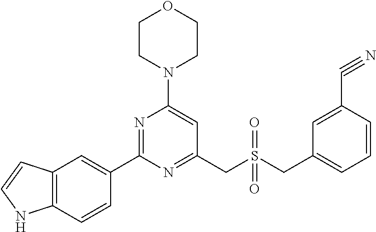 | 3-[[2-(1H-Indol-5-yl)-6- morpholin-4-yl-pyrimidin-4- yl]methylsulfonylmethyl]benzonitrile | 474 | 1.93 |
| 156 | 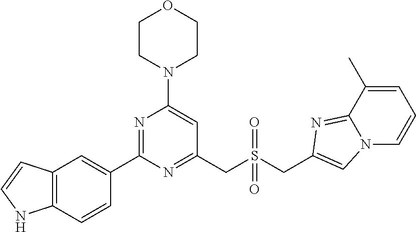 | 8-[[2-(1H-Indol-5-yl)-6- morpholin-4-yl-pyrimidin-4- yl]methylsulfonylmethyl]-5-methyl- 1,7-diazabicyclo[4.3.0]nona- 2,4,6,8-tetraene | 503 | 1.22 |
| 157 | 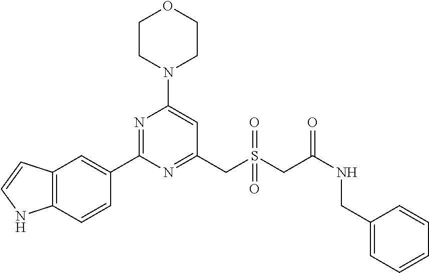 | N-Benzyl-2-[[2-(1H-indol-5- yl)-6-morpholin-4-yl-pyrimidin-4- yl]methylsulfonyl]acetamide | 506 | 1.75 |
| 158 | 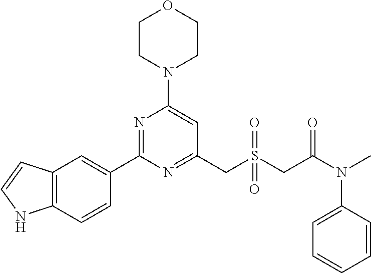 | 2-[[2-(1H-Indol-5-yl)-6- morpholin-4-yl-pyrimidin-4- yl]methylsulfonyl]-N-methyl- N-phenyl-acetamide | 506 | 1.76 |
| 159 | 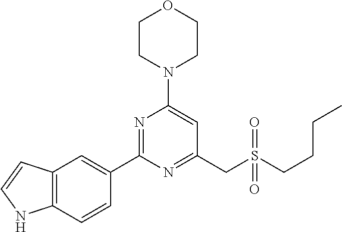 | 5-[4-(Butylsulfonylmethyl)- 6-morpholin-4-yl-pyrimidin- 2-yl]-1H-indole | 415 | 1.80 |
| 160 | 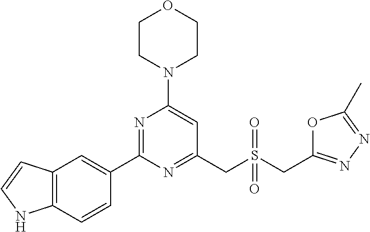 | 5-[4-[(5-Methyl-1,3,4-oxadiazol- 2-yl)methylsulfonylmethyl]-6- morpholin-4-yl-pyrimidin-2- yl]-1H-indole | 455 | 1.60 |
| 161 | 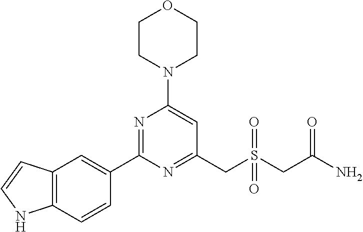 | 2-[[2-(1H-Indol-5-yl)-6- morpholin-4-yl-pyrimidin-4- yl]methylsulfonyl]acetamide | 416 | 1.22 |
| 162 | 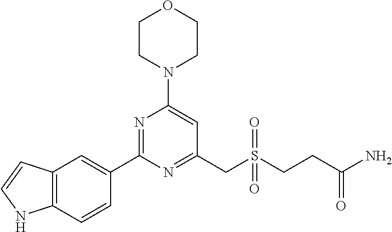 | 3-[[2-(1H-Indol-5-yl)-6- morpholin-4-yl-pyrimidin-4- yl]methylsulfonyl]propanamide | 430 | 1.21 |
| 163 | 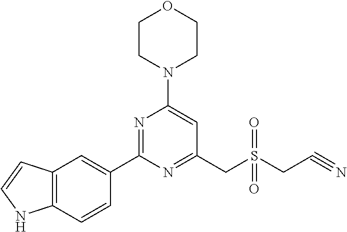 | 2-[[2-(1H-Indol-5-yl)-6- morpholin-4-yl-pyrimidin-4- yl]methylsulfonyl]acetonitrile | 396 (M − H)⁻ | 1.90 |
| 164 | 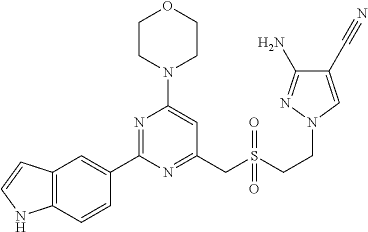 | 5-Amino-1-[2-[[2-(1H-Indol- 5-yl)-6-morpholin-4-yl- pyrimidin-4- yl]methylsulfonyl]ethyl] pyrazole-4-carbonitrile | 493 | 1.65 |
| 165 | 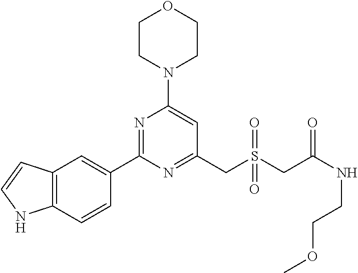 | 2-[[2-(1H-Indol-5-yl)-6- morpholin-4-yl-pyrimidin- 4-yl]methylsulfonyl]-N-(2- methoxyethyl)acetamide | 474 | 1.38 |
| 166 | 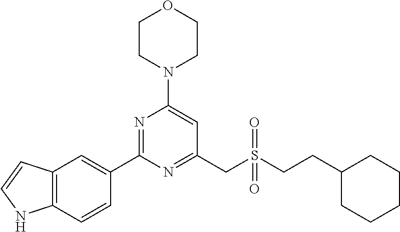 | 5-[4-(2- cyclohexylethylsulfonylmethyl)- 6-morpholin-4-yl-pyrimidin-2- yl]-1H-indole | 469 | 2.45 |
| 167 | 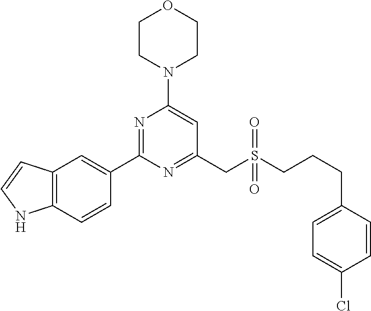 | 5-[4-[3-(4- Chlorophenyl)propylsulfonyl- methyl]-6-morpholin-4-yl- pyrimidin-2-yl]-1H-indole | 512 | 2.40 |
| 168 | 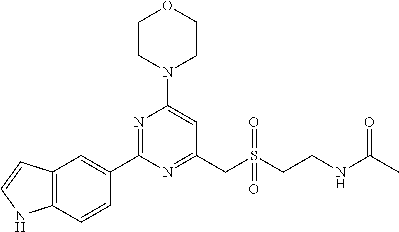 | N-[2-[[2-(1H-Indol-5-yl)-6- morpholin-4-yl-pyrimidin-4- yl]methylsulfonyl]ethyl]acetamide | 444 | 1.26 |
| 169 | 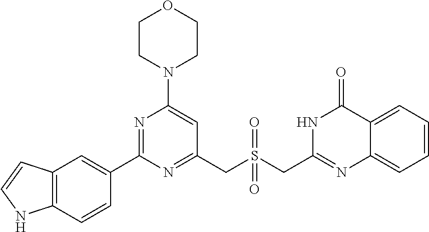 | 2-[[2-(1H-Indol-5-yl)-6- morpholin-4-yl-pyrimidin- 4-yl]methylsulfonylmethyl]- 3H-quinazolin-4-one | 517 | 1.68 |
| 170 | 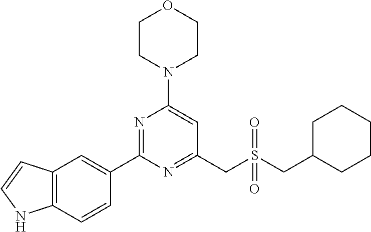 | 5-[4- (Cyclohexylmethylsulfonylmethyl)- 6-morpholin-4-yl-pyrimidin-2-yl]- 1H-indole | 455 | 2.19 |
| 171 | 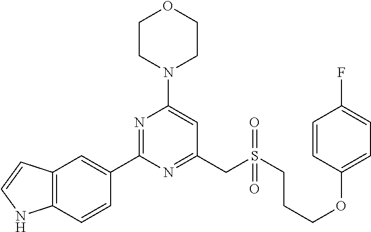 | 5-[4-[3-(4-Fluorophenoxy) propylsulfonylmethyl]- 6-morpholin-4-yl- pyrimidin-2-yl]-1H-indole | 511 | 2.26 |
| 172 | 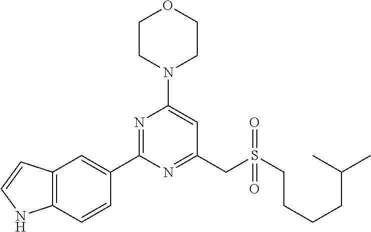 | 5-[4-(5- Methylhexylsulfonylmethyl)- 6-morpholin-4-yl-pyrimidin- 2-yl]-1H-indole | 457 | 2.41 |

The starting material [2-(1H-indol-5-yl)-6-morpholin-4-yl-pyrimidin-4-yl]methylsulfanylmethanimidamide 2,2,2-trifluoroacetic acid salt was prepared as follows:

### [2-(1H-indol-5-yl)-6-morpholin-4-yl-pyrimidin-4-yl]methylsulfanylmethanimidamide 2,2,2-trifluoroacetic acid salt

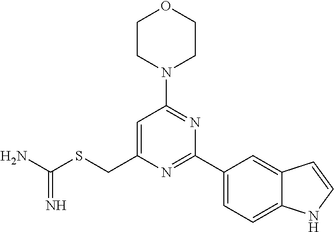

[2-(1H-Indol-5-yl)-6-morpholin-4-yl-pyrimidin-4-yl]methanol (from example 110, 3.27 g, 10.55 mmol) was suspended in DCM and treated with methane sulfonylchloride (1.23 mL, 15.82 mmol) and triethylamine (2.21 mL, 15.82 mmol). After 15 minutes the suspension was evaporated to crude material and redissolved in ethanol (25 mL). Thiourea (0.882 g, 11.60 mmol) was added and the reaction heated at 70° C. for 30 minutes. The majority of the ethanol was removed by distillation. The residue was triturated with ether and the solvent discarded. This trituration was repeated twice more to give the crude product as a solid. This was purified by preparative HPLC to give the desired compound, (1.16 g).

LCMS Spectrum: MH+ 369, Retention Time 1.14, Method: Monitor Acid

NMR spectrum ¹H NMR (DMSO-d₆) δ3.68-3.80 (8H, m), 4.42 (2H, s), 6.56 (1H, s), 6.80 (1H, s), 7.40-7.44 (1H, m), 7.46 (1H, d), 8.03-8.08 (1H, m), 8.52 (1H, s), 9.33 (1H, s), 9.84 (1H, s), 11.29 (1H, s).

### EXAMPLE 173

### 4-Morpholin-4-yl-2-pyridin-2-yl-6-(tert-butylsulfonylmethyl)pyrimidine

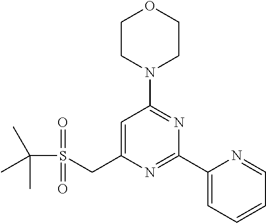

Prepared in an analogous fashion to Example 44, 4-(benzenesulfonylmethyl)-6-morpholin-4-yl-2-pyridin-2-yl-pyrimidine, from the appropriate sulfide.

LCMS Spectrum: MH+ 377.6 Retention Time 3.16, Method: Monitor Base

NMR spectrum: ¹H NMR (500.133 MHz, DMSO) δ 1.40 (9H, s), 3.73 (8H, s), 4.51 (2H, s), 6.95 (1H, s), 7.48-7.51 (1H, m), 7.94 (1H, dt), 8.31 (1H, d), 8.71-8.73 (1H, m)

The starting sulfides were prepared in an analogous fashion to Example 26, 4-morpholin-4-yl-6-(phenylsulfanylmethyl)-2-pyridin-2-yl-pyrimidine, by reacting the appropriate thiol with 4-(chloromethyl)-6-morpholin-4-yl-2-pyridin-2-yl-pyrimidine (from example 26).

| | | | Retention | |
|---|---|---|---|---|
| | | LCMS | Time | |
| Structure | NAME | MH+ | (min) | NMR |
| 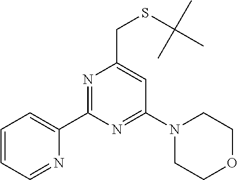 | 4-Morpholin-4-yl-2- pyridin-2-yl-6-(tert- butylsulfanylmethyl) pyrimidine | 346 | 1.19 | ¹H NMR (400.133 MHz, DMSO) δ 1.28 (9H, s), 3.65 (8H, s), 3.75 (2H, s), 6.83 (1H, s), 7.48-7.51 (1H, m), 7.86 (1H, dt), 8.23 (1H, d), 8.64-8.67 (1H, m) |

### EXAMPLE 174

### 2-Methyl-5-[4-(methylsulfonylmethyl)-6-morpholin-4-yl-pyrimidin-2-yl]-1H-indole

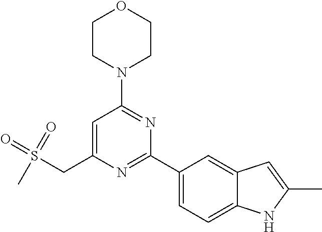

2-Chloro-4-(methylsulfonylmethyl)-6-morpholin-4-yl-primidine (292 mg, 1 mmol), 2-methyl-1(4-methylphenyl)sulfonyl-5-(4,4,5,5,-tetramethyl-1,3,2-dioxaborolan-2-yl)indole (617 mg, 1.5 mmol), 2M aqueous sodium carbonate solution (1 mL), dichloro-bis-(triphenylphosphine) palladium(II) (20 mg) and 18% DMF in 7:3:2 DME:Water:Ethanol (3.5 mL) were placed in a microwave tube and heated to 125° C. for 30 minutes. The solvent was then evaporated and the residue partitioned between water and DCM. The layers were then separated and the aqueous phase extracted with DCM. The combined organic extracts were dried (MgSO₄) and evaporated to afford an oil. This was dissolved in methanol/water mixture and treated with sodium hydroxide solution (2M, 6 mL) for four hours. The reaction was neutralised with hydrochloric acid (2M) and evaporated. The crude solid was purified by prep HPLC to afford the title compound as a white solid (30 mg).

LCMS Spectrum: MH+ 387.60, Retention Time 1.97, Method: Monitor Base

NMR Spectrum: ¹H NMR (300.132 MHz, CDCl₃) δ3.09 (3H, s), 3.48 (3H, s), 3.68-3.91 (8H, m), 4.27 (2H, s), 6.42 (1H, s), 6.52 (1H, s), 8.21 (1H, d), 8.29 (1H, dd), 8.40 (1H, d).

The starting material 2-methyl-1-(4-methylphenyl)sulfonyl-5-(4,4,5,5-tetra methyl-1,3,2-dioxaborolan-2-yl)indole was prepared as follows.

### 2-Methyl-1-(4-methylphenyl)sulfonyl-5-(4,4,5,5-tetramethyl-1,3,2-dioxaborolan-2-yl)indole

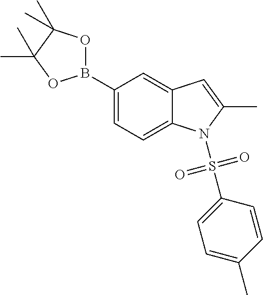

5-Bromo-2-methyl-1-(4-methylphenyl)sulfonyl-indole (1.095 g, 3 mmol), bis(pinacolato)diboron (915 mg, 3.6 mmol), palladium dichloride di(dppf) dichloromethane complex (25 mg, 0.03 mmol) and potassium acetate (588 mg, 6 mmol) were suspended in dioxane (20 mL) and heated to 80° C. for 10 hours. The reaction mixture was then applied to a column of silica gel, and purified by flash chromatography (0-10% EtOAc/iHexane) to afford the title compound as a waxy solid (951 mg).

NMR Spectrum: ¹H NMR (300.132 MHz, CDCl₃) δ1.28 (12H, s), 2.26 (3H, s), 2.52 (3H, s), 6.26 (1H, s), 7.11 (2H, d), 7.57 (2H, d), 7.62 (1H, dd), 7.81 (1H, s), 8.07 (1H, d)

### 5-Bromo-2-methyl-1-(4-methylphenyl)sulfonyl-indole

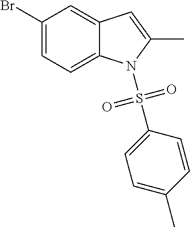

2-Methyl-5-bromoindole (5 g, 23.8 mmol) was dissolved in DMF (50 mL) and sodium hydride (1.05 g, 26.18 mmol) was then added portion wise to the solution. After 30 minutes, toluenesulfonyl chloride (5 g, 26.18 mmol) was added and the reaction was allowed to stir at room temperature for 6 hours. The reaction was then poured into water and extracted into ethyl acetate. The combined organic extracts were dried over MgSO₄ and evaporated to afford a solid. This was purified by flash chromatography (0-5% Ethylacetate/isohexane) to afford the title compound as a light brown solid (5.23 g).

LCMS: M+H⁺ 364.27, Retention Time 3.29, Method: Monitor Base

NMR Spectrum: ¹H NMR (300.132 MHz, DMSO-d₆) δ2.32 (3H, s), 2.59 (3H, s), 6.55 (1H, s), 7.37 (2H, d), 7.41 (1H, dd), 7.69 (1H, d), 7.74 (2H, d), 7.97 (1H, d)

### EXAMPLE 175

### 4-[(5-Methyl-2H-pyrazol-3-yl)oxymethyl]-6-morpholin-4-yl-2-pyridin-2-yl-pyrimidine

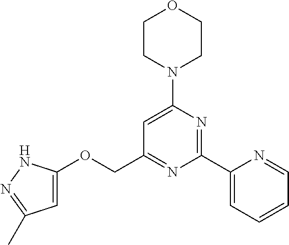

Prepared in an analogous fashion to Example 68, 4-[(3-methoxyphenoxy)methyl]-6-morpholin-4-yl-2-pyridin-2-yl-pyrimidine, from 4-(chloromethyl)-6-morpholin-4-yl-2-pyridin-2-yl-pyrimidine (from example 26) and the appropriate starting material.

LCMS Spectrum: MH+ 353.6 Retention Time 1.59, Method: Monitor Base

NMR Spectrum: ¹H NMR (300.132 MHz, DMSO-d₆) δ2.16 (s, 3H), 3.70 (s, 8H), 5.09 (s, 2H), 5.56 (s, 1H), 6.82 (s, 1H), 7.48 (m, 1H), 7.92 (td, 1H), 8.31 (d, 1H), 8.70 (d, 1H), 11.57 (s, 1H)

### EXAMPLE 176

### 2-(3-Furyl)-4-(methylsulfonylmethyl)-6-morpholin-4-yl-pyrimidine

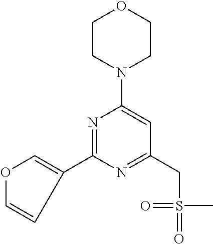

Prepared in an analogous fashion to Example 1,4-(methylsulfonylmethyl)-6-morpholin-4-yl-2-thiophen-3-yl-pyrimidine, from 2-methylsulfanyl-4-(methylsulfonylmethyl)-6-morpholin-4-yl-pyrimidine and the appropriate boronic acid.

LCMS Spectrum: MH+ 324.5, Retention Time 1.63, Method: Monitor Base

### EXAMPLE 177

### 4-(Methylsulfonylmethyl)-6-morpholin-4-yl-2-naphthalen-1-yl-pyrimidine

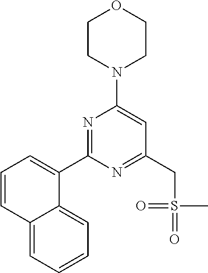

Prepared in an analogous fashion to Example 1, 4-(methylsulfonylmethyl)-6-morpholin-4-yl-2-thiophen-3-yl-pyrimidine, from 2-methylsulfanyl-4-(methylsulfonylmethyl)-6-morpholin-4-yl-pyrimidine and the appropriate boronic acid.

LCMS Spectrum: MH+ 384.6, Retention Time 2.16, Method: Monitor Base

1. A compound of formula (I) 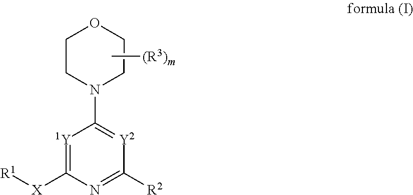
or a salt, ester or prodrug thereof; wherein
m is 0, 1, 2, 3 or 4;
X is a linker group selected from —CR⁴═CR⁵—, —CR⁴═CR⁵CR⁶R⁷—, —CR⁶R⁷CR⁵═CR⁴—, —C≡C—, —C≡CCR⁶R⁷—, —CR⁶R⁷C≡C—, —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)CR⁶R⁷—, —NR⁴C(O)NR⁵CR⁶R⁷—, —NR⁴S(O)₂CR⁶R⁷—, —S(O)₂NR⁴CR⁶R⁷—, —C(O)NR⁴—, —NR⁴C(O)—, —NR⁴C(O)NR⁵—, —S(O)₂NR⁴— and —NR⁴S(O)₂—;
¹Y and Y² are independently N or CR⁸ provided that one of ¹Y and Y² is N and the other is CR⁸;
R¹ is a group selected from C₁₋₆alkyl, C₂₋₆alkenyl, C₂₋₆alkynyl, carbocyclyl, carbocyclylC₁₋₆alkyl, heterocyclyl and heterocyclylC₁₋₆alkyl, which group is optionally substituted by one or more substituent group selected from halo, cyano, nitro, R⁹, —OR⁹, —SR⁹, —SOR⁹, —SO₂R⁹, —COR⁹, —CO₂R⁹, —CONR⁹R¹⁰, —NR⁹R¹⁰, —NR⁹COR¹⁰, —NR⁹CO₂R¹⁰, —NR⁹CONR¹⁰R¹⁵, —NR⁹COCONR¹⁰R¹⁵ and —NR⁹SO₂R¹⁰;
R² is a group selected from C₁₋₆alkyl, carbocyclyl and heterocyclyl which group is optionally substituted by one or more substituent group independently selected from halo, cyano, nitro,
—R¹¹, —OR¹¹, —SR¹¹, —SOR¹¹, —SO₂R¹¹, —COR¹¹, —CO₂R¹¹, —CONR¹¹R¹², —NR¹¹R¹², —NR¹¹COR¹², and —NR¹¹COCONR¹²R¹⁶;
each R³, when present, is independently selected from halo, cyano, nitro, —R¹³, —OR¹³, —SR¹³, —SOR¹³, —SO₂R¹³, —COR¹³, —CO₂R¹³, —CONR¹³R¹⁴, —NR¹³R¹⁴, —NR¹³COR¹⁴; —NR¹³CO₂R¹⁴ and —NR¹³SO₂R¹⁴;
R⁴ and R⁵ are independently hydrogen or C₁₋₆alkyl;
or R¹ and R⁴ together with the atom or atoms to which they are attached form a 5- to 10-membered carbocyclic or heterocyclic ring wherein 1, 2 or 3 ring carbon atoms is optionally replaced with N, O or S and which ring is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R⁶ and R⁷ are independently selected from hydrogen, halo, cyano, nitro and C₁₋₆alkyl;
R⁸ is selected from hydrogen, halo, cyano and C₁₋₆alkyl;
R⁹ and R¹⁰ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹¹ and R¹² are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
R¹³, R¹⁴, R¹⁵ and R¹⁶ are independently hydrogen or a group selected from C₁₋₆alkyl, carbocyclyl, carbocyclylC₁₋₆alkyl, heterocyclyl and heterocyclylC₁₋₆alkyl which group is optionally substituted by one or more substituent groups selected from halo, cyano, nitro, hydroxy, C₁₋₆alkyl, C₁₋₆alkoxy, haloC₁₋₆alkyl, haloC₁₋₆alkoxy, hydroxyC₁₋₆alkyl, hydroxyC₁₋₆alkoxy, C₁₋₆alkoxyC₁₋₆alkyl, C₁₋₆alkoxyC₁₋₆alkoxy, amino, C₁₋₆alkylamino, bis(C₁₋₆alkyl)amino, aminoC₁₋₆alkyl, (C₁₋₆alkyl)aminoC₁₋₆alkyl, bis(C₁₋₆alkyl)aminoC₁₋₆alkyl, cyanoC₁₋₆alkyl, C₁₋₆alkylsulfonyl, C₁₋₆alkylsulfonylamino, C₁₋₆alkylsulfonyl(C₁₋₆alkyl)amino, sulfamoyl, C₁₋₆alkylsulfamoyl, bis(C₁₋₆alkyl)sulfamoyl, C₁₋₆alkanoylamino, C₁₋₆alkanoyl(C₁₋₆alkyl)amino, carbamoyl, C₁₋₆alkylcarbamoyl and bis(C₁₋₆alkyl)carbamoyl;
provided that when X is —C(O)NH—, R¹ is not the group 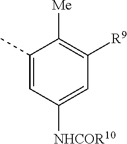
for use as a medicament in the treatment of proliferative disease.

2. A compound of formula (I) according to claim 1 wherein X is a linker group selected from —NR⁴CR⁶R⁷—, —OCR⁶R⁷—, —SCR⁶R⁷—, —S(O)CR⁶R⁷—, —S(O)₂CR⁶R⁷—, —C(O)NR⁴CR⁶R⁷—, —NR⁴C(O)NR⁵CR⁶R⁷—, —S(O)₂NR⁴CR⁶R⁷—, —NR⁴C(O)—, —C(O)NR⁴—, —S(O)₂NR⁴— and —NR⁴S(O)₂— for use as a medicament in the treatment of proliferative disease.

3. A compound of formula (I) according to claim 1 wherein X is a linker group selected from —SCR⁶R⁷—, —S(O)CR⁶R⁷— and —S(O)₂CR⁶R⁷— for use as a medicament in the treatment of proliferative disease.

4. A compound of formula (I) according to any one of claims 1 to 3 wherein R⁴ is hydrogen or methyl for use as a medicament in the treatment of proliferative disease.

5. A compound of formula (I) according to any one of claims 1 to 4 wherein R⁵ is hydrogen or methyl for use as a medicament in the treatment of proliferative disease.

6. A compound of formula (I) according to any one of claims 1 to 5 wherein R⁶ is hydrogen or methyl for use as a medicament in the treatment of proliferative disease.

7. A compound of formula (I) according to any one of claims 1 to 6 wherein R⁷ is hydrogen or methyl for use as a medicament in the treatment of proliferative disease.

8. A compound of formula (I) according to any one of claims 1 to 7 wherein R¹ is a group selected from C₁₋₄alkyl, C₃₋₆cycloalkyl, aryl, C₃₋₆cycloalkylC₁₋₄alkyl, arylC₁₋₄alkyl, cycloheteroalkyl, heteroaryl, cycloheteroalkylC₁₋₄alkyl, heteroarylC₁₋₄alkyl, which group is optionally substituted by one or more substituent group selected from halo, cyano, nitro, R⁹, —OR⁹, —COR⁹, —CONR⁹R¹⁰, —NR⁹R¹⁰ and —NR⁹COR¹⁰ for use as a medicament in the treatment of proliferative disease.

9. A compound of formula (I) according to claim 8 wherein R¹ is a group selected from methyl, ethyl, n-propyl, isopropyl, n-butyl, sec-butyl, isobutyl, tert-butyl, cyclohexyl, —CH₂CN, —CH₂C(O)NH₂, —CH₂CH₂NC(O)CH₃, phenyl, 4-fluorophenyl, 2-chlorophenyl, 3-chlorophenyl, 2-chloro-6-fluorophenyl, 3-chloro-4-fluorophenyl, 4-bromo-2-fluorophenyl, 4-trifluoromtheylphenyl, 4-trifluoromethoxyphenyl, 4-cycanophenyl, 3-methoxyphenyl, 4-methoxyphenyl, 3,4-dimethoxyphenyl, 4-(N-methylaminocarbonyl)phenyl, benzyl, 4-fluorobezyl, 2-chlorobenzyl, 2-chloro-6-fluorobenzyl, 4-methoxybenzyl, phenethyl, 3-trifluorophenethyl, furan-2-ylmethyl, thien-2-ylmethyl, 2-pyrazin-2-ylethyl, pyidin-3-yl, 2-methylpyridin-3-yl and 2-aminocarbonylpyridin-3-yl for use as a medicament in the treatment of proliferative disease.

10. A compound of formula (I) according to any one of claims 1 to 9 wherein R² is selected from aryl and heteroaryl which group is optionally substituted by one or more substituent group independently selected from halo, cyano, nitro, —R¹¹, —OR¹¹, —COR¹¹, —CONR¹¹R¹², —NR¹¹R¹² and —NR¹¹COR¹² for use as a medicament in the treatment of proliferative disease.

11. A compound of formula (I) according claim 10 wherein R² is selected from phenyl, naphthyl, pyrrolyl, imidazolyl, pyrazolyl, furanyl, thienyl, pyridinyl, pyrimidinyl, pyridazinyl, azaindolyl, indolyl, quinolinyl, benzimidazolyl, benzofuranyl, dibenzofuranyl, benzothienyl which group is optionally substituted by one or more substituent group independently selected from halo, cyano, nitro, —R¹¹, —OR¹¹, —COR¹¹, —CONR¹¹R¹², —NR¹¹R¹² and —NR¹¹COR¹² for use as a medicament in the treatment of proliferative disease.

12. A compound of formula (I) according claim 11 wherein R² is
3-(hydroxymethyl)phenyl, 4-(hydroxymethyl)phenyl, 4-(cyanomethyl)phenyl, 3,4-dimethoxyphenyl, 3-fluoro-4-methoxyphenyl, 4-phenoxyphenyl, 3-pyrrolidin-lylphenyl, 3-(aminocarbonyl)phenyl, 4-(dimethylaminocarbonyl)phenyl, furan-3-yl, thien-3-yl, 5-(hydroxymethyl)thien-2-yl, pyridin-2-yl, pyridin-4-yl, 2-methoxypyridin-5-yl, 2-methoxypyrimidin-5-yl, 2-methoxynaphth-6-yl, 5,7-diazabicyclo[4.3.0]nona-2,4,8,10-tetraenyl, azaindolyl, indol-5-yl, 1-methylindol-5-yl, quinolin-6-yl, benzimidazolyl, benzofuran-2-yl, dibenzofuran-1-yl and benzothien-3-yl for use as a medicament in the treatment of proliferative disease.

13. A compound of formula (I) according claim 12 wherein R² is azaindolyl, indol-5-yl, benzimidazolyl, 3-hydroxyphenyl, 4-hydroxyphenyl, 3-hydroxymethylphenyl or 4-hydroxymethylphenyl for use as a medicament in the treatment of proliferative disease.

14. A compound of formula (I) according to any one of claims 1 to 13 wherein ¹Y is CR⁸ and Y² is N for use as a medicament in the treatment of proliferative disease.

15. A compound of formula (I) according to claim 14 wherein ¹Y is CH or CF and Y² is N for use as a medicament in the treatment of proliferative disease.

16. A compound of formula (I) according to claim 15 wherein ¹Y is CH and Y² is N for use as a medicament in the treatment of proliferative disease.

17. A compound of formula (I) according to any one of claims 1 to 16 wherein m is 0 so that R³ is absent for use as a medicament in the treatment of proliferative disease.

18. The use of a compound of formula (I) or a pharmaceutically acceptable salt thereof as defined in any one of claims 1 to 17 in the manufacture of a medicament for use in the treatment of proliferative disease.

19. The use of a compound of formula (I), or a pharmaceutically acceptable salt thereof, as defined in any one of claims 1 to 17 for the production of an anti-proliferative effect in a warm-blooded animal such as man.

20. The use of a compound of formula (I), or a pharmaceutically acceptable salt thereof, as defined in any one of claims 1 to 17 in the manufacture of a medicament for use in the production of an anti-proliferative effect in a warm-blooded animal such as man.

21. A method for producing an anti-proliferative effect in a warm-blooded animal, such as man, in need of such treatment which comprises administering to said animal an effective amount of a compound of formula (I), or a pharmaceutically acceptable salt thereof, as defined in any one of claims 1 to 17.

22. A method for treating cancer, inflammatory diseases, obstructive airways diseases, immune diseases or cardiovascular diseases in a warm blooded animal such as man that is in need of such treatment which comprises administering an effective amount of a compound of formula (I), or a pharmaceutically acceptable salt thereof, as defined in any one of claims 1 to 17.

23. A compound of formula (I) as defined in any one of claims 1 to 17 provided that the compound of formula (I) is not:
4-{6-[(methylthio)methyl]-2-methylpyrimidin-4-yl}morpholine;
4-(6-{[(4-chlorophenyl)thio]methyl}-2-methylpyrimidin-4-yl)morpholine;
4-(6-{[(4-chlorophenyl)thio]methyl}-2-methylpyrimidin-4-yl)-2,6-dimethylmorpholine;
4-{6-[(phenylsulfinyl)methyl]-2-methylpyrimidin-4-yl}morpholine;
4-(6-{[(4-chlorophenyl)sulfinyl]methyl}-2-methylpyrimidin-4-yl)morpholine;
4-{6-[(phenylsulfonyl)methyl]-2-methylpyrimidin-4-yl}morpholine;
4-(6-{[(4-chlorophenyl)sulfonyl]methyl}-2-methylpyrimidin-4-yl)morpholine;
4-{6-[(methylthio)methyl]-2-phenylpyrimidin-4-yl}morpholine;
4-{6-[(phenylthio)methyl]-2-phenylpyrimidin-4-yl}morpholine;
4-(6-{[(4-chlorophenyl)thio]methyl}-2-phenylpyrimidin-4-yl)morpholine;
4-(6-{[(4-chlorobenzyl)thio]methyl}-2-phenylpyrimidin-4-yl)morpholine;
4-(6-{[(4-chlorobenzyl)thio]methyl}-2-phenylpyrimidin-4-yl)-2,6-dimethylmorpholine;
4-{6-[(methylsulfinyl)methyl]-2-phenylpyrimidin-4-yl}morpholine;
4-{6-[(phenylsulfinyl)methyl]-2-phenylpyrimidin-4-yl}morpholine;
4-(6-{[(4-chlorophenyl)sulfinyl]methyl}-2-phenylpyrimidin-4-yl)morpholine;
4-{6-[(methylsulfonyl)methyl]-2-phenylpyrimidin-4-yl}morpholine;
4-{6-[(phenylsulfonyl)methyl]-2-phenylpyrimidin-4-yl}morpholine;
4-{6-[(methylthio)methyl]-2-pyridin-2-ylpyrimidin-4-yl}morpholine;
4-{6-[(phenylthio)methyl]-2-pyridin-4-ylpyrimidin-4-yl}morpholine;
4-{6-[(4-chlorophenyl)thio]methyl}-2-pyridin-2-ylpyrimidin-4-yl)morpholine;
4-{6-[(methylsulfonyl)methyl]-2-pyridin-3-ylpyrimidin-4-yl}morpholine;
4-{6-[(methylsulfonyl)methyl]-2-pyridin-4-ylpyrimidin-4-yl}morpholine;
4-{6-[(phenylsulfonyl)methyl]-2-pyridin-2-ylpyrimidin-4-yl}morpholine;
4-{6-[(phenylsulfonyl)methyl]-2-pyridin-3-ylpyrimidin-4-yl}morpholine;
4-{6-[(phenylsulfonyl)methyl]-2-pyridin-4-ylpyrimidin-4-yl}morpholine;
4-{6-[(methoxy)methyl]-2-methylpyrimidin-4-yl}morpholine;
4-{6-[(methoxy)methyl]-2-phenylpyrimidin-4-yl}morpholine;
4-{6-[(methoxy)methyl]-2-phenylpyrimidin-4-yl}-2,6-dimethylmorpholine;
4-{6-[(phenoxy)methyl]-2-(6-methylpyrid-2-yl)pyrimidin-4-yl}-2,6-dimethylmorpholine;
N-[5-[[3-(1-cyano-1-methylethyl)benzoyl]amino]-2-methylphenyl]-2,6-di-4-morpholinyl-4-pyrimidinecarboxamide;
N-[5-[[3-(1-cyano-1-methylethyl)benzoyl]amino]-2-methylphenyl]-6-(4-morpholinyl)-2-(trifluoromethyl)-4-pyrimidinecarboxamide;
N-[4-fluoro-3-[(pyrazinyloxy)methyl]phenyl]-2,6-di-4-morpholinyl-4-pyrimidinecarboxamide;
4-[2-methyl-6-[(1E)-2-[3-(trifluoromethyl)phenyl]ethenyl]-4-pyrimidinyl]-morpholine;
4-[6-methyl-2-[(1E)-2-[3-(trifluoromethyl)phenyl]ethenyl]-4-pyrimidinyl]-morpholine;
3,4,5-trimethoxy-N-[4-methyl-6-(4-morpholinyl)-2-pyrimidinyl]-benzamide;
N-(2,3-dimethyl-1H-indol-5-yl)-2,6-di-4-morpholinyl-4-pyrimidinecarboxamide;
N-(2,3-dimethyl-1H-indol-5-yl)-4,6-di-4-morpholinyl-2-pyridinecarboxamide;
N-(3,4-dimethylphenyl)-2,6-di-4-morpholinyl-4-pyrimidinecarboxamide;
N-[3-(aminocarbonyl)phenyl]-2,6-di-4-morpholinyl-4-pyrimidinecarboxamide;
N-(4,6-di-4-morpholinyl-2-pyridinyl)-N′-(3-methylphenyl)-urea;
N-(2,3-dimethyl-1H-indol-5-yl)-4,6-di-4-morpholinyl-2-pyridinecarboxamide;
4,6-di-4-morpholinyl-N-(1,2,3-trimethyl-1H-indol-5-yl)-2-pyridinecarboxamide;
N-(2,3-dimethyl-1H-indol-5-yl)-2-[(2R,6S)-2,6-dimethyl-4-morpholinyl]-6-(4-morpholinyl)-4-pyrimidinecarboxamide;
2,6-di-4-morpholinyl-N-(1,2,3-trimethyl-1H-indol-5-yl)-4-pyrimidinecarboxamide;
N-[3-(dimethylamino)phenyl]-2,6-di-4-morpholinyl-4-pyrimidinecarboxamide;
N-[3,4,5-trimethoxyphenyl]-2,6-di-4-morpholinyl-4-pyrimidinecarboxamide;
2,6-di-4-morpholinyl-N-(6,7,8,9-tetrahydro-5H-benzocyclohepten-6-yl)-4-pyrimidinecarboxamide; and
4-[2-methyl-6-[2-(5-nitro-2-furyl)vinyl]-4-pyrimidinyl]-morpholine.

24. A pharmaceutical composition comprising a compound of formula (I) as defined in claim 23, or a pharmaceutically acceptable salt thereof, in association with a pharmaceutically acceptable diluent or carrier.

25. A compound of formula (I) as defined in claim 23, or a pharmaceutically acceptable salt thereof, for use as a medicament.

26. A process for preparing a compound of formula (I) as defined in claim 1, wherein X is —S(O)₂CR⁶R⁷—, by reacting a compound of formula (I), wherein X is —SCR⁶R⁷—, with an oxidising agent (for example by using Oxone® at room temperature in a mixed solvent system of water and ethanol).

27. A process for preparing a compound of formula (I) as defined in claim 1, wherein X is —X¹CR⁶R⁷— and X¹ is —NR⁴—, —O—, —S—, —S(O)—, or —S(O)₂—, 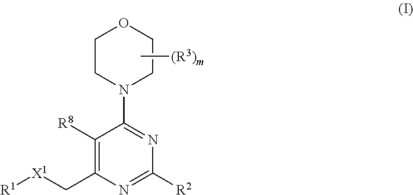
comprising reaction a compound of formula (II), wherein L¹ is a leaving group (such as halo (for example chloro), tosyl, mesyl etc.,) 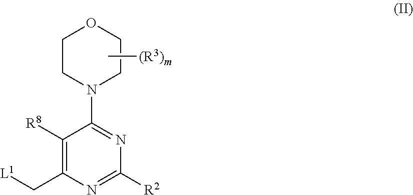
with a compound of formula (III)
R¹—X¹H (III)
(optionally in the presence of a suitable base such as triethylamine and a solvent such as tetrahydrofuran or N,N-dimethylformamide).

28. A process for preparing compound of formula (I) as defined in claim 1, wherein X is —S(O)₂CR⁶R⁷—, comprising reacting a compound of formula (IX) 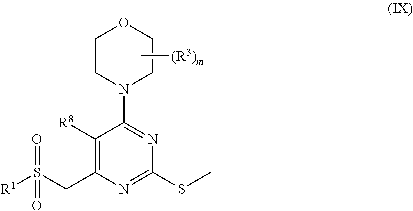
with a suitable organo-metallic reagent (such as the activated ester of boronic acid R²B(OR)₃ wherein R is C₁₋₄alkyl such as methyl), in the presence of a suitable metal catalyst (such as palladium or copper).

29. A process for preparing a compound of formula (I) as defined in claim 1, wherein X is —C(O)NR⁴CR⁶R⁷—, —NR⁴C(O)NR⁵CR⁶R⁷— or —S(O)₂NR⁴CR⁶R⁷— comprising reacting a compound of formula (I) wherein X is —NH₂CR⁶R⁷— 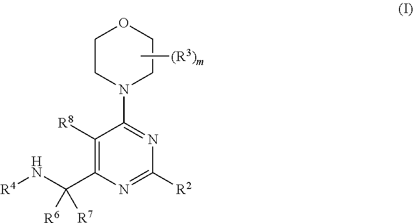
with a compound of formula (XVI) selected from 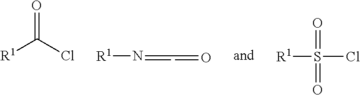
optionally in the presence of a suitable base (such as triethylamine).

30. A process for preparing a compound of formula (I) as defined in claim 1, wherein X is —C(O)NR⁴—, —NR⁴C(O)NR⁵— or —S(O)₂NR⁴—, comprising reacting a compound of formula (XV) 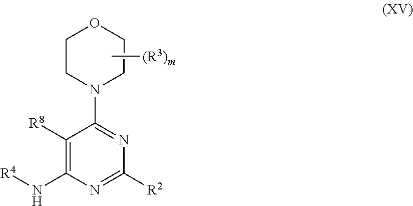
with a compound of formula (XVI) selected from 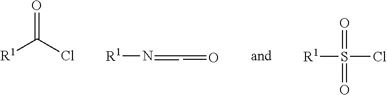
in the presence of a suitable base (such as triethylamine).

31. A process for preparing a compound of formula (I) as defined in claim 1, comprising reacting a compound of formula (XXIII) 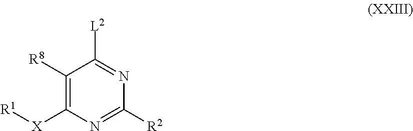
with a compound of formula (V)

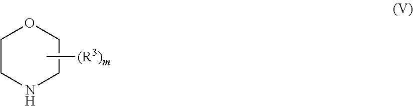

32. A process for preparing a compound of formula (I) as defined in claim 1, wherein X is —NR⁴C(O)— comprising reacting a compound of formula (XVII) 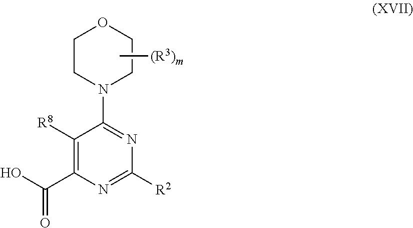
with an amine R⁴NH₂ and a suitable activating reagent such as O-(7-azabenzotriazol-1-yl)-N,N,N′,N′-tetramethyluronium hexafluorophosphate using a base such as diisopropylethyl amine and a solvent such as tetrahydrofuran.

33. A process for preparing a compound of formula (I) as defined in claim 1, wherein X is —S(O)₂CR⁶R⁷—, 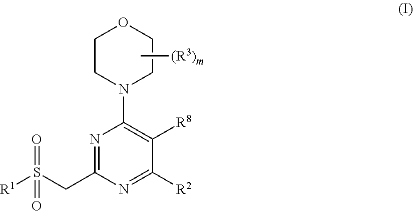
comprising reacting a compound of formula (I), wherein X is —SCR⁶R⁷— 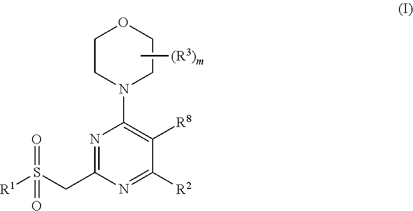
with an oxidising agent (for example by using Oxone® at room temperature in a mixed solvent system of water and ethanol).

34. A process for preparing a compound of formula (I) as defined in claim 1, wherein X is —X¹CR⁶R⁷ and X¹ is —NR⁴—, —O—, —S—, —S(O)—, comprising reacting a compound of formula (XXVIII) 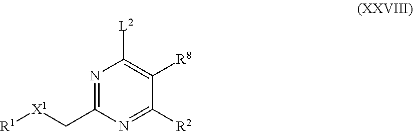
with a compound of formula (V)

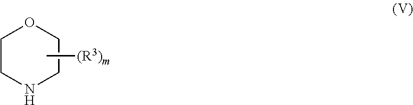

35. A process for preparing a compound of formula (I) as defined in claim 1, wherein X is —C(O)NR⁴CR⁶R⁷—, —NR⁴C(O)NR⁵CR⁶R⁷— or —S(O)₂NR⁴CR⁶R⁷— comprising reacting a compound of formula (I) wherein X is —NH₂CR⁶R⁷— 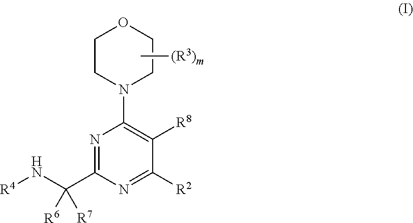
with a compound of formula (XVI) selected from 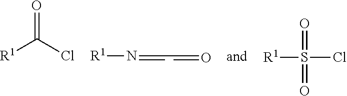
in the presence of a suitable base such as triethylamine.

36. A process for preparing a compound of formula (I) as defined in claim 1, wherein X is —C(O)NR⁴—, —NR⁴C(O)NR⁵— or —S(O)₂NR⁴— comprising reacting a compound of formula (XXXII) 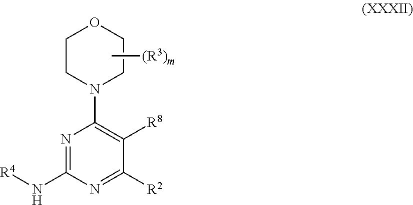
with a compound of formula (XVI) selected from

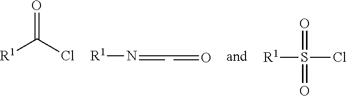

37. A process for preparing a compound of formula (I) as defined in claim 1, wherein X is —X¹CR⁶R⁷— and X¹ is —NR⁴—, —O—, —S—, —S(O)—, or —S(O)₂— comprising reaction a compound of formula (XXXVII), wherein L¹ is a leaving group (such as halo (for example chloro), tosyl, mesyl etc.,) 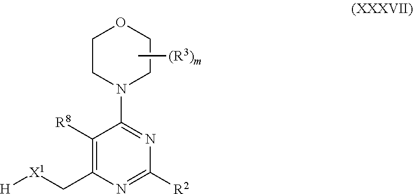
with a compound of formula (XXXVIII)
R¹-L¹ (XXXVIII)
in the presence of a suitable base (such as triethylamine or sodium hydride and a solvent such as tetrahydrofuran or N,N-dimethylformamide).

38. A process for preparing a compound of formula (I) as defined in claim 1, wherein X is —X¹CR⁶R⁷— and X¹ is —S— comprising reaction a compound of formula (XXXIX), 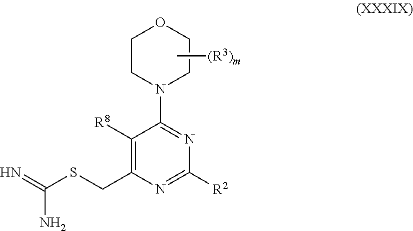
with a compound of formula (XXXVIII)
R¹-L¹ (XXVIII)
in the presence of a suitable base (such as sodium hydroxide) and a solvent (such as N,N-dimethylformamide).

39. A process for preparing a compound of formula (I) as defined in claim 1, wherein X is —X¹CR⁶R⁷— and X¹ is —NR⁴—, —O—, —S—, —S(O)—, or —S(O)₂— comprising reacting a compound of formula (XXXX), 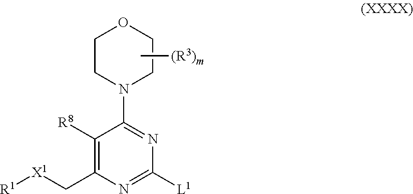
with a suitable organo-metallic reagent (such as a the activated ester of boronic acid to R²B(OR)₃ wherein R is C₁₋₄alkyl such as methyl), in the presence of a suitable metal catalyst (such as palladium or copper) using a solvent (such as 1,4-dioxane).

40. A process for preparing a compound of formula (I) as defined in claim 1, wherein X is —NR⁴C(O)—, —NR⁴C(O)CR⁶R⁷—, —NR⁴S(O)₂—, or —NR⁴S(O)₂CR⁶R⁷—, comprising reacting a compound of formula (XXXXVIII), 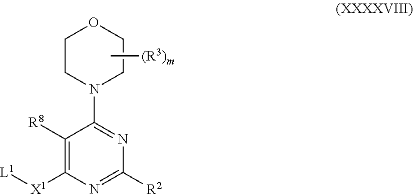
wherein X¹ is —C(O)—, —C(O)CR⁶R⁷—, —S(O)₂—, or —S(O)₂CR⁶R⁷— and L¹ is a suitable leaving group (such as chloro or an activated ester),
with an amine of formula (XXXXIX), 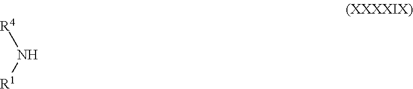
in the presence of a suitable base (such as triethylamine).

41. A process for preparing a compound of formula (I) as defined in claim 1, wherein X is —NR⁴CHR₆— comprising reacting a compound of formula (XXXXX) 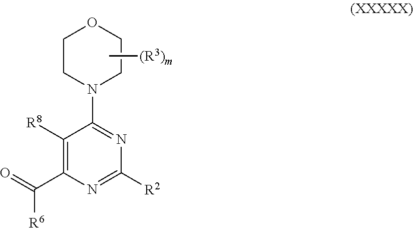
with an amine of formula (XXXXIX) 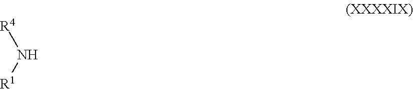
in the presence of a suitable reducing agent (such as NaCNBH₃).
